(19) United States
(12) Patent Application Publication
(10) Pub. No.: US 2026/0207628 A1
(43) Pub. Date: Jul. 23, 2026

(54) 25-HYDROXY-CHOLEST-5-EN-3-SULFATE CHOLINE, FORMULATIONS THEREOF AND METHODS FOR PREPARING, AND USES OF SAME

(71) Applicant: Durect Corporation, Cupertino, CA (US)

(72) Inventors: Andrew R. Miksztal, Palo Alto, CA (US); Huey-Ching Su, San Jose, CA (US); Wendy Chao, San Jose, CA (US); Travis Lee Houston, Lafayette, IN (US); Prasad V. Challapalli, Bear, DE (US); Asma Ibrahim Patel, Leicester (GB); Nayan Solanki, Vancouver, WA (US)

(21) Appl. No.: 19/142,082

(22) PCT Filed: Dec. 22, 2023
(86) PCT No.: PCT/US2023/085845

§ 371 Date: Jun. 20, 2025

Related U.S. Application Data
(60) Provisional application No. 63/435,151, filed on Dec. 23, 2022.

(51) Int. Cl. A61K 31/575; A61K 9/20

(52) U.S. Cl. CPC A61K 31/575; A61K 9/2009; A61K 9/2013; A61K 9/2018; A61K 9/2027; A61K 9/2054

(57) ABSTRACT

25HC3S choline and crystalline 25HC3S choline are described herein. Pharmaceutical formulations of 25HC3S choline such as with crystalline 25HC3S choline and methods of treating or preventing disease with same such as nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, alcoholic fatty liver disease (AFLD), alcoholic steatohepatitis (ASH), leptin resistance, leptin deficiency, a diabetes condition, an autoimmune condition, an inflammatory condition, a neurological condition, Epstein Barr virus-related growth, and conditions related to fat accumulation and inflammation are further disclosed herein. Methods for preparing 25HC3S, including crystalline 25HC3S choline, are also provided.

### CROSS REFERENCE TO RELATED APPLICATION

The present application claims priority under 35 U.S.C. § 119(e) of U.S. Provisional Application No. 63/435,151, filed Dec. 23, 2022, the disclosure of which is expressly incorporated by reference herein in its entirety.

### INTRODUCTION

It has been shown previously that cholesterol metabolite 5-cholesten-3β-25-diol-3-sulfate (“25HC3S”) decreases lipid biosynthesis and increases cholesterol secretion and degradation, and may be useful for the treatment and prevention of one or more of nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, alcoholic fatty liver disease (AFLD), alcoholic steatohepatitis (ASH), leptin resistance, leptin deficiency, a diabetes condition, an autoimmune condition, an inflammatory condition, a neurological condition, Epstein Barr virus-related growth, and conditions related to fat accumulation and inflammation.

Cholesterol is used by the body for the manufacture and repair of cell membranes, and the synthesis of steroid hormones and vitamin D, and is transformed to bile acids in the liver. There are both exogenous and endogenous sources of cholesterol. The average American consumes about 450 mg of cholesterol each day and produces an additional 500 mg to 1,000 mg in the liver and other tissues. Another source is the 500 mg to 1,000 mg of biliary cholesterol that is secreted into the intestine daily, and about 50 percent is reabsorbed (enterohepatic circulation).

High serum lipid levels (hypercholesterolemia and hypertriglyceridemia) are associated with the accumulation of cholesterol in arterial walls, and can result in NAFLD and atherosclerosis. The plaques that characterize atherosclerosis inhibit blood flow and promote clot formation, and can ultimately cause death or severe disability via heart attacks and/or stroke. A number of therapeutic agents for the treatment of hyperlipidemia have been developed and are widely prescribed by physicians. Unfortunately, only about 35% of patients are responsive to the currently available therapies.

Non-alcoholic fatty liver disease (NAFLD) is the most common liver disease in the United States. This condition is associated with obesity, type-II adult-onset diabetes, sedentary lifestyle, and diets high in fat. The earlier stage of NAFLD, fatty liver, is potentially reversible when proper treatment steps are taken. However, left unchecked, it can progress to inflammation of liver cells (non-alcoholic steatohepatitis, or NASH) which is much more difficult to treat. Without treatment, NASH can result in irreversible scarring of liver tissue (steatonecrosis), with the potential to cause cirrhosis, liver failure, and liver cancer.

Certain pharmaceutically acceptable salts, such as a sodium salt, of 25HC3S have been disclosed (e.g., U.S. Pat. No. 10,144,759 and Ogawa et al., Steroids, 74, 81-87 (2009)). Different salts of 25HC3S may bring different practical benefits and disadvantages, for instance in relation to their amenability to processing into desired pharmaceutical formulations, their clinical efficacy in addressing particular pathological indications, and the like. WO 2022/272103, which is incorporated herein by reference, discloses 25HC3S choline and crystalline 25HC3S choline. Herein, the inventors disclose 25HC3S choline formulations, amorphous 25HC3S choline, spray-dried-dispersions of 25HC3S choline, micronized 25HC3S choline, and methods of treatment.

### SUMMARY

In some aspects of the present disclosure, pharmaceutical compositions comprising 25HC3S choline are provided.

In additional aspects of the disclosure, tablets comprising 25HC3S are provided.

In some aspects of the disclosure, amorphous 25HC3S choline is provided.

In other aspects of the disclosure, spray-dried 25HC3S choline is provided.

In other aspects of the disclosure, micronized 25HC3S choline is provided.

In additional aspects of the disclosure, a mesophase of 25HC3S choline is provided.

In further aspects of the disclosure, methods of treating or preventing one or more of nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, alcoholic fatty liver disease (AFLD), alcoholic steatohepatitis (ASH), leptin resistance, leptin deficiency, a diabetes condition, an autoimmune condition, an inflammatory condition, a neurological condition, Epstein Barr virus-related growth, and conditions related to fat accumulation and inflammation, comprising administering to a patient in need thereof an effective amount of a pharmaceutical composition comprising 25HC3S choline, including one or more of crystalline, amorphous, mesophase or spray-dried 25HC3S choline, are provided.

In still further aspects of the disclosure, processes of preparing a pharmaceutical composition comprising 25HC3S choline, including one or more of crystalline 25HC3S choline or amorphous 25HC3S choline, mesophase or spray-dried 25HC3S choline, and optionally one or more pharmaceutically acceptable excipients, are provided.

### BRIEF DESCRIPTION OF THE FIGURES

FIG. 1 is an x-ray powder diffraction (XRPD) diffractogram of crystalline 25HC3S choline.

FIG. 2 is a peak-picked XRPD diffractogram of crystalline 25HC3S choline.

FIG. 3 is an XRPD diffractogram overlay of crystalline 25HC3S choline before and after a dynamic vapor sorption (DVS) experiment.

FIG. 4 is indexing results for crystalline 25HC3S choline.

FIG. 5 is a differential scanning calorimetry (DSC) thermogram and a thermogravimetric analysis (TGA) thermogram for crystalline 25HC3S choline.

FIG. 6 is a ¹H-NMR spectrum of 25HC3S choline in solution.

FIG. 7 is a DVS isotherm of crystalline 25HC3S choline.

FIG. 8 is a DVS isotherm of crystalline 25HC3S sodium.

FIG. 9 is a drug release profile of a spray-dried dispersion of 25HC3S choline in a 12.5 mg tablet.

FIG. 10 is a drug release profile of a micronized crystalline 25HC3S choline in a 12.5 mg tablet.

FIG. 11 is a drug release profile of a micronized crystalline 25HC3S choline in a 12.5 mg tablet.

FIG. 12A is set of DSC thermograms of copovidone showing 1—reversing heat flow; 2—non-reversing heat flow; and 3—total heat flow (all of 1, 2, 3 are normalized).

FIG. 12 B is set of DSC thermograms of HPMC E5 showing 1—reversing heat flow; 2—non-reversing heat flow; and 3—total heat flow (all of 1, 2, 3 are normalized).

FIG. 12C is a set of DSC thermograms of 25HC3S choline showing 1—reversing heat flow; 2—non-reversing heat flow; and 3—total heat flow (all of 1, 2, 3 are normalized).

FIG. 13A is an x-ray powder diffraction pattern of spray-dried 25HC3S choline.

FIG. 13B is an x-ray powder diffraction pattern of a spray-dried composition of 25HC3S choline and copovidone.

FIG. 13C is an x-ray powder diffraction pattern of a spray-dried composition of 25HC3S choline and HPMC E5.

FIG. 14 is a set of polarized light micrographs of spray-dried dispersions of neat 25HC3S choline and spray-dried dispersions of 25HC3S choline with copovidone and HPMC E5 as indicated.

FIG. 15 is a drug release profile of neat 25HC3S choline compared with solid dispersions.

FIG. 16A is an x-ray powder diffraction overlay of a spray-dried dispersion of 25HC3S choline and copovidone at 2° C.-8° C. at T=0; T=14 days and T=28 days.

FIG. 16B is an x-ray powder diffraction overlay of a spray-dried dispersion of 25HC3S choline and copovidone at 25° C./60% RH at T=0; T=14 days and T=28 days.

FIG. 16C is an x-ray powder diffraction overlay of a spray-dried dispersion of 25HC3S choline and copovidone at 40° C./75% RH at T=0; T=14 days and T=28 days.

FIG. 17A is an x-ray powder diffraction overlay of a spray-dried dispersion of 25HC3S choline and HPMC at 2° C.-8° C. at 0, 14, and 28 days.

FIG. 17B is an x-ray powder diffraction overlay of a spray-dried dispersion of 25HC3S choline and HPMC at 25° C./60% RH at 0, 14, and 28 days.

FIG. 17C is an x-ray powder diffraction overlay of a spray-dried dispersion of 25HC3S choline and HPMC at 40° C./75% RH at 0, 14, and 28 days.

FIG. 18 is a DSC thermogram of a solid dispersion of 25HC3S choline and copovidone at 1—initial; 2—14 days at 25° C./60% RH; 3—14 days at 2-8° C.; 4—14 days at 40° C./75% RH.

FIG. 19 is a DSC thermogram of a solid dispersion of 25HC3S choline and HPMC E5 at 1—initial; 2—14 days at 25° C./60% RH; 3—14 days at 2-8° C.; 4—14 days at 40° C./75% RH.

FIG. 20 is a Dynamic Vapor Sorption Curve for a solid-dispersion of 25HC3S choline and copovidone.

FIG. 21 is a Dynamic Vapor Sorption Curve for a solid-dispersion of 25HC3S choline and HPMC E5.

FIG. 22 is a drug release profile at time 0 and 4 weeks for solid dispersions of 25HC3S choline with either copovidone or HPMC E5. Drug release was conducted in 0-30 minutes in 450 mL FaSSGF and 30-90 minutes FaSSGF+double strength FaSSIF in a total of 900 mL of media.

FIG. 23 is a drug release profile of tablets containing a solid dispersion of copovidone and 25HC3S choline. Drug release was conducted in 0-30 minutes of FaSSGF and 90 minutes in FaSSGF+double strength FaSSIF.

FIG. 24 is a drug release profile of two 25HC3S choline spray dried dispersion tablets in the presence of: (1) 10% NaCl/20% crospovidone; and (2) 5% SLS/20% crospovidone and 25HC3S choline. Drug release was conducted in 0-30 minutes of FaSSGF and 90 minutes in FaSSGF+double strength FaSSIF.

FIG. 25 is a drug release profile of spray-dried dispersion tablets in biorelevant and QC media. Drug release for biorelevant media was conducted in 0-30 minutes FaSSGF and 90 minutes in FaSSGF+double strength FaSSIF. Drug release for QC media was conducted in 0.5% SLS in 0.1 N HCl.

FIG. 26 is a process flow diagram for making spray-dried dispersions of 25HC3S choline and a polymer.

FIG. 27 is a process flow diagram for making spray-dried dispersion tablets of 25HC3S choline.

FIG. 28 is a drug release profile of micronized 25HC3S choline tablets. Drug release was conducted in 0.5% SDS in 900 mL 0.1 N HCl.

FIG. 29 is a drug release profile of micronized 25HC3S choline tablets. Drug release was conducted in 0-30 minutes in 450 mL FaSSGF and 30-90 minutes in FaSSGF+double strength FaSSIF in 900 mL media.

FIG. 30 is a drug release profile of micronized 25HC3S choline tablets. Drug release was conducted in 0.5% SDS in 900 mL 0.1 N HCl.

FIG. 31 is a drug release profile of micronized 25HC3S choline tablets. Drug release was conducted in 0-30 minutes in 450 mL FaSSGF and 30-90 minutes in FaSSGF+double strength FaSSIF in total 900 mL media.

FIG. 32 is a drug release profile of micronized 25HC3S choline tablets. Drug release was conducted in 0-30 minutes 450 mL FaSSGF and 30-90 minutes in FaSSGF+double strength FaSSIF in total 900 mL media.

FIG. 33 is a drug release profile of micronized 25HC3S choline tablets in QC media (0.5% SLS in 0.1 N HCl).

FIG. 34 is a drug release profile of micronized 25HC3S choline tablets in 0.5% or 1.0% SLS in 900 mL of 0.1 N HCl.

FIG. 35 is a drug release profile of micronized 25HC3S choline tablets in 0.5% or 2.0% SLS in 900 mL of 0.1 N HCl.

FIG. 36 is a process flow diagram for making a blend for 25HC3S choline tablets.

FIG. 37 is a process flow diagram for making a blend for 25HC3S choline tablets.

FIG. 38 is an x-ray powder diffraction pattern of 25HC3S choline mesophase 1—formed from EtOH evaporation, driest sample, limited lamellae in upper portions of vial gel in base of vial; 2—formed upon isolation of water activity sample; 3—formed from slurrying in water.

### DETAILED DESCRIPTION

### Crystalline 25-hydroxy-3β-cholesten-5-en-3-sulfate (25HC3S) choline

As described herein, the compound 25-hydroxy-3β-cholesten-5-en-3-sulfate (25HC3S) refers to [(3S,10R,13R,17R)-17-[(1R)-5-hydroxy-1,5-dimethyl-hexyl]-10,13-dimethyl-2,3,4,7,8,9,11,12,14,15,16,17-dodecahydro-1H-cyclopenta[a]phenanthren-3-yl]sulfate, the compound of Formula 1:

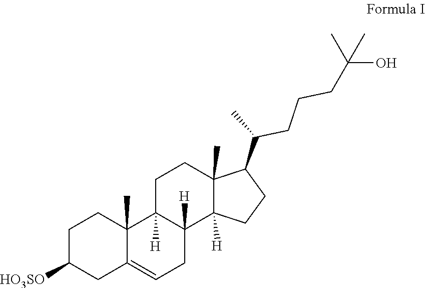

The term “25HC3S choline” means the choline salt of 25HC3S. The term “crystalline 25HC3S choline” means the crystalline choline salt of 25HC3S, i.e., the choline salt of 25HC3S in crystalline form. Choline is a quaternary ammonium compound. It is often available as choline hydroxide. 25HC3S choline has the following structure:

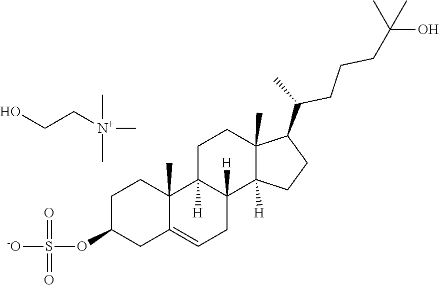

Choline is an essential nutrient that is naturally present in some foods and available as a dietary supplement. Choline is a source of methyl groups needed for many steps in metabolism. The body needs choline to synthesize phosphatidylcholine and sphingomyelin, two phospholipids associated with cell membranes, and to produce the neurotransmitter acetylcholine. Choline deficiency is associated with undesirable clinical indications, including the manifestation of the conditions described herein.

Crystalline 25HC3S choline is readily analyzed or characterized by x-ray powder diffraction. An x-ray powder diffraction pattern is an x-y graph with °2θ (diffraction angle) on the x-axis and intensity on the y-axis. The x-axis can also be in the form of d-spacings which is related to the diffraction angle via the Bragg's law whereby 2d sin θ=nλ where d is the d-spacing and λ is the wavelength of the incident x-ray wave. The pattern contains peaks which may be used to characterize crystalline 25HC3S choline. Unless otherwise specified, peaks are referred to by their position on the x-axis and not their y-axis intensity. It can also occur that due to sample orientation, a peak that is present in one sample on one instrument may not be present in another sample taken on a different instrument due to the orientation of the sample with respect to the instrument.

The data from x-ray powder diffraction may be used in multiple ways to characterize crystalline forms. For example, the entire x-ray powder diffraction pattern output from a diffractometer may be used to characterize crystalline 25HC3S choline. A smaller subset of such data, however, may also be, and typically is, suitable for characterizing crystalline 25HC3S choline. For example, a collection of one or more peaks from such a pattern may be used to characterize crystalline 25HC3S choline. In the present application, all reported peak values are in °2θ with Cu-Kα radiation, e.g., as set forth in Example 24 and Example 25. Indeed, often even a single x-ray powder diffraction peak may be used to characterize such a crystalline form. When crystalline 25HC3S choline herein is characterized by “one or more peaks” of an x-ray powder diffraction pattern and such peaks are listed, what is generally meant is that any combination of the peaks listed may be used to characterize crystalline 25HC3S choline. Further, the fact that other peaks are present in the x-ray powder diffraction pattern, generally does not negate or otherwise limit that characterization.

In addition to the variability in peak intensity, there may also be variability in the position of peaks on the x-axis. This variability can, however, typically be accounted for when reporting the positions of peaks for purposes of characterization. Such variability in the position of peaks along the x-axis may derive from several sources (e.g., sample preparation, orientation and size, particle size, moisture content, solvent content, instrument and experimental parameters, data analysis software). For example, samples of the same crystalline material prepared under different conditions may yield slightly different diffractograms, and different x-ray instruments may operate using different parameters, and these may lead to slightly different diffraction patterns from the same crystalline solid.

Due to such sources of variability, it is common to recite x-ray diffraction peaks using the word “about” prior to the peak value in °2θ. For purposes of data reported herein, that value is generally ±0.2°2θ. This generally means that on a well-maintained instrument one would expect the variability in peak measurement to be ±0.2°2θ on the same instrument. Unless specified otherwise, x-ray powder diffraction peaks cited herein are generally reported with this variability of ±0.2°2θ and are generally intended to be reported with such a variability whenever disclosed herein whether the word “about” is present or not, however, variability may, in some instances, be higher depending on instrumentation conditions. Furthermore, in additional embodiments of the present disclosure, the variability in a quoted peak value or grouping of quoted peak values in °2θ is ±0.1°2θ, or even ±0.05°2θ, rather than ±0.2°2θ.

The x-ray powder diffraction data from crystalline 25HC3S choline may be used to index the corresponding unit cell. “Indexing,” as used herein, generally refers to the process of determining the size and shape of the crystallographic unit cell given the peak positions in a diffraction pattern. The term gets its name from the assignment of Miller index labels to individual peaks. For example, if all of the peaks in a pattern are indexed by a single unit cell, this can be strong evidence that the sample contains a single crystalline phase. Given the indexing solution, the unit cell volume may be calculated directly and can be useful to determine their solvation states. Indexing may also be a description of a crystalline form and provides a concise summary of all available peak positions for that phase at a particular thermodynamic state point.

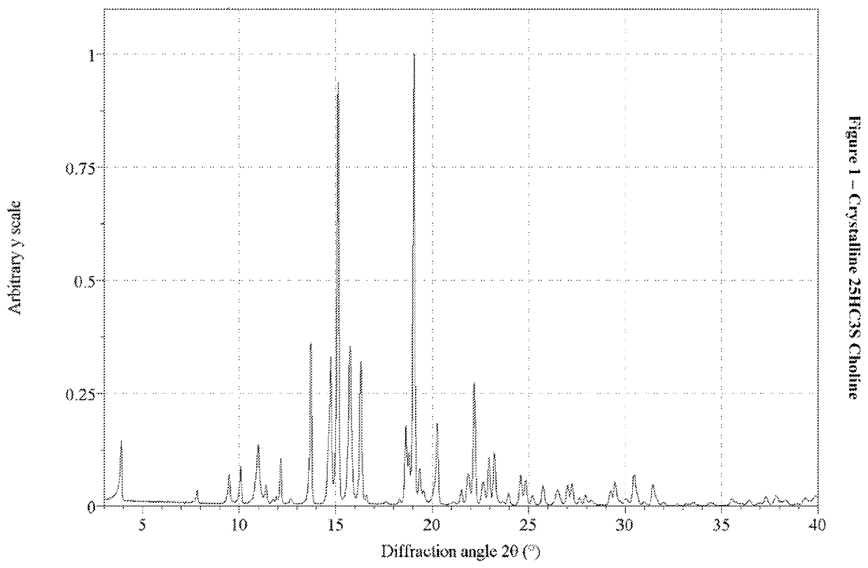

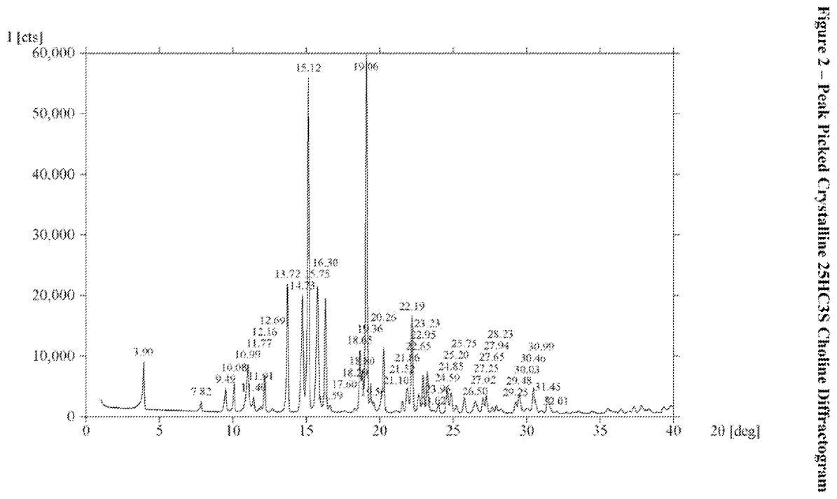

25HC3S choline, including crystalline 25HC3S choline, may be prepared as set forth in Example 28. An x-ray powder diffraction pattern of crystalline 25HC3S choline can be found in FIG. 1 and a peak-picked version in FIG. 2. Table 1 shows picked peaks from FIG. 2.

TABLE 1
Peaks of Crystalline 25HC3S Choline of FIG. 2

| | | Intensity |
|---|---|---|
| °2θ | d space (Å) | (%) |
| 3.90 ± 0.20 | 22.638 + 1.160 | 15 |
| 7.82 ± 0.20 | 11.296 ± 0.288 | 4 |
| 9.49 ± 0.20 | 9.312 ± 0.196 | 8 |
| 10.08 ± 0.20 | 8.768 ± 0.174 | 10 |
| 10.99 ± 0.20 | 8.044 ± 0.146 | 15 |
| 11.40 ± 0.20 | 7.756 ± 0.136 | 5 |
| 11.77 ± 0.20 | 7.513 ± 0.127 | 2 |
| 11.91 ± 0.20 | 7.425 ± 0.124 | 3 |
| 12.16 ± 0.20 | 7.273 ± 0.119 | 11 |
| 12.69 ± 0.20 | 6.970 ± 0.109 | 2 |
| 13.72 ± 0.20 | 6.449 ± 0.094 | 37 |
| 14.73 ± 0.20 | 6.009 ± 0.081 | 33 |
| 15.12 ± 0.20 | 5.855 ± 0.077 | 93 |
| 15.75 ± 0.20 | 5.622 ± 0.071 | 36 |
| 16.30 ± 0.20 | 5.434 ± 0.066 | 33 |
| 16.59 ± 0.20 | 5.339 ± 0.064 | 3 |
| 17.60 ± 0.20 | 5.035 ± 0.057 | 2 |
| 18.29 ± 0.20 | 4.847 ± 0.053 | 2 |
| 18.65 ± 0.20 | 4.754 ± 0.051 | 19 |
| 18.80 ± 0.20 | 4.716 ± 0.050 | 13 |
| 19.06 ± 0.20 | 4.653 ± 0.048 | 100 |
| 19.36 ± 0.20 | 4.581 ± 0.047 | 9 |
| 19.56 ± 0.20 | 4.535 ± 0.046 | 4 |
| 20.26 ± 0.20 | 4.380 ± 0.043 | 19 |
| 21.10 ± 0.20 | 4.207 ± 0.039 | 2 |
| 21.52 ± 0.20 | 4.126 ± 0.038 | 4 |
| 21.86 ± 0.20 | 4.063 ± 0.037 | 8 |
| 22.19 ± 0.20 | 4.003 ± 0.036 | 28 |
| 22.65 ± 0.20 | 3.923 ± 0.034 | 6 |
| 22.95 ± 0.20 | 3.872 ± 0.033 | 12 |
| 23.23 ± 0.20 | 3.826 ± 0.032 | 13 |
| 23.62 ± 0.20 | 3.764 ± 0.031 | 2 |
| 23.96 ± 0.20 | 3.711 ± 0.031 | 4 |
| 24.59 ± 0.20 | 3.617 ± 0.029 | 8 |
| 24.85 ± 0.20 | 3.580 ± 0.028 | 6 |
| 25.20 ± 0.20 | 3.531 ± 0.028 | 3 |
| 25.75 ± 0.20 | 3.457 ± 0.026 | 5 |
| 26.50 ± 0.20 | 3.361 ± 0.025 | 4 |
| 27.02 ± 0.20 | 3.297 ± 0.024 | 5 |
| 27.25 ± 0.20 | 3.270 ± 0.024 | 6 |
| 27.65 ± 0.20 | 3.224 ± 0.023 | 3 |
| 27.94 ± 0.20 | 3.191 ± 0.022 | 3 |
| 28.23 ± 0.20 | 3.159 ± 0.022 | 2 |
| 29.25 ± 0.20 | 3.051 ± 0.020 | 4 |
| 29.48 ± 0.20 | 3.028 ± 0.020 | 6 |
| 30.03 ± 0.20 | 2.973 ± 0.019 | 2 |
| 30.46 ± 0.20 | 2.932 ± 0.019 | 8 |
| 30.99 ± 0.20 | 2.883 ± 0.018 | 2 |
| 31.45 ± 0.20 | 2.842 ± 0.018 | 6 |
| 32.01 ± 0.20 | 2.794 ± 0.017 | 2 |

Crystalline 25HC3S choline may be characterized by various analytical techniques, including by x-ray powder diffraction. The x-ray powder diffraction pattern of crystalline 25HC3S choline or portions thereof, may be used to identify crystalline 25HC3S choline. Crystalline 25HC3S choline contains various x-ray powder diffraction peaks which alone or together may help identify the presence of crystalline 25HC3S choline.

In some cases, crystalline 25HC3S choline may be characterized by an x-ray powder diffraction pattern having a peak at about 3.9°2θ. In these and other cases, crystalline 25HC3S choline may be further characterized by one or more peaks at about 7.8°2θ, about 9.5°2θ, about 10.1°2θ, about 11.0°2θ, about 12.2°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having a peak at about 7.8°2θ. In these and other cases, crystalline 25HC3S choline may be further characterized by one or more peaks at about 3.9°2θ, about 9.5°2θ, about 10.1°2θ, about 11.0°2θ, about 12.2°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having a peak at about 9.5°2θ. In these and other cases, crystalline 25HC3S choline may be further characterized by one or more peaks at about 3.9°2θ, about 7.8°2θ, about 10.1°2θ, about 11.0°2θ, about 12.2°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having a peak at about 10.1°2θ. In these and other cases, crystalline 25HC3S choline may be further characterized by one or more peaks at about 3.9°2θ, about 7.8°2θ, about 9.5°2θ, about 11.0°2θ, about 12.2°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having a peak at about 11.0°2θ. In these and other cases, crystalline 25HC3S choline may be further characterized by an x-ray powder diffraction pattern having one or more peaks at about 3.9°2θ, about 7.8°2θ, about 9.5°2θ, about 10.1°2θ, about 12.2°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having a peak at about 12.2°2θ. In these and other cases, crystalline 25HC3S choline may be further characterized by an x-ray powder diffraction pattern having one or more peaks at about 3.9°2θ, about 7.8°2θ, about 9.5°2θ, about 10.1°2θ, about 11.0°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having a peak at about 13.7°2θ. In these and other cases, crystalline 25HC3S choline may be further characterized by an x-ray powder diffraction pattern having one or more peaks at about 3.9°2θ, about 7.8°2θ, about 9.5°2θ, about 10.1°2θ, about 11.0°2θ, about 12.2°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having a peak at about 14.7°2θ. In these and other cases, crystalline 25HC3S choline may be further characterized by an x-ray powder diffraction pattern having one or more peaks at about 3.9°2θ, about 7.8°2θ, about 9.5°2θ, about 10.1°2θ, about 11.0°2θ, about 12.2°2θ, about 13.7°2θ, about 15.1°2θ, about 15.8°2θ, about 16.3°2θ, and about 19.1°2θ.

In some cases, crystalline 25HC3S choline may be characterized by an x-ray powder diffraction pattern having a peak at about 15.1°2θ. In these and other cases, crystalline 25HC3S choline may be further characterized by an x-ray powder diffraction pattern having one or more peaks at about 3.9°2θ, about 7.8°2θ, about 9.5°2θ, about 10.1°2θ, about 11.0°2θ, about 12.2°2θ, about 13.7°2θ, about 14.7°2θ, about 15.8°2θ, about 16.3°2θ, and about 19.1°2θ.

In some cases, crystalline 25HC3S choline may be characterized by an x-ray powder diffraction pattern having a peak at about 15.8°2θ. In these and other cases, crystalline 25HC3S choline may be further characterized by an x-ray powder diffraction pattern having one or more peaks at about 3.9°2θ, about 7.8°2θ, about 9.5°2θ, about 10.1°2θ, about 11.0°2θ, about 12.2°2θ, about 13.7°2θ, about 14.7°2θ, about 15.1°2θ, about 16.3°2θ, and about 19.1°2θ.

In some cases, crystalline 25HC3S choline may be characterized by an x-ray powder diffraction pattern having a peak at about 16.3°2θ. In these and other cases, crystalline 25HC3S choline may be further characterized by an x-ray powder diffraction pattern having one or more peaks at about 3.9°2θ, about 7.8°2θ, about 9.5°2θ, about 10.1°2θ, about 11.0°2θ, about 12.2°2θ, about 13.7°2θ, about 14.7°2θ, about 15.1°2θ, about 15.8°2θ, and about 19.1°2θ.

In some cases, crystalline 25HC3S choline may be characterized by an x-ray powder diffraction pattern having a peak at about 19.1°2θ. In these and other cases, crystalline 25HC3S choline may be further characterized by an x-ray powder diffraction pattern having one or more peaks at about 3.9°2θ, about 7.8°2θ, about 9.5°2θ, about 10.1°2θ, about 11.0°2θ, about 12.2°2θ, about 13.7°2θ, about 14.7°2θ, about 15.1°2θ, about 15.8°2θ, and about 16.3°2θ.

In some cases, crystalline 25HC3S choline may be characterized by an x-ray powder diffraction pattern having peaks at about 3.9°2θ and about 7.8°2θ. In these and other cases, crystalline 25HC3S choline may be further characterized by an x-ray powder diffraction pattern having one or more peaks at about 9.5°2θ, about 10.1°2θ, about 11.0°2θ, about 12.2°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having peaks at about 3.9°2θ, about 7.8°2θ, and about 9.5°2θ. In these and other cases, crystalline 25HC3S choline may be further characterized by an x-ray powder diffraction pattern having one or more peaks at about 10.1°2θ, about 11.0°2θ, about 12.2°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having peaks at about 3.9°2θ, about 7.8°2θ, about 9.5°2θ, and about 10.1°2θ. In these and other cases, crystalline 25HC3S choline may be further characterized by an x-ray powder diffraction pattern having one or more peaks at about 11.0°2θ, about 12.2°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having peaks at about 3.9°2θ, about 7.8°2θ, about 9.5°2θ, about 10.1°2θ, and about 11.0°2θ. In these and other cases, crystalline 25HC3S choline may be further characterized by an x-ray powder diffraction pattern having one or more peaks at about 12.2°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having peaks at about 3.9°2θ, about 7.8°2θ, about 9.5°2θ, about 10.1°2θ, about 11.0°2θ, and about 12.2°2θ. In these and other cases, crystalline 25HC3S choline may be further characterized by an x-ray powder diffraction pattern having one or more peaks at about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having peaks at about 3.9°2θ, about 7.8°2θ, about 9.5°2θ, about 10.1°2θ, about 11.0°2θ, about 12.2°2θ, and about 13.7°2θ. In these and other cases, crystalline 25HC3S choline may be further characterized by an x-ray powder diffraction pattern having one or more peaks at about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having peaks at about 3.9°2θ, about 7.8°2θ, about 9.5°2θ, about 10.1°2θ, about 11.0°2θ, about 12.2°2θ, about 13.7°2θ, and about 14.7°2θ. In these and other cases, crystalline 25HC3S choline may be further characterized by an x-ray powder diffraction pattern having one or more peaks at about 15.1°2θ, about 15.8°2θ, about 16.3°2θ, and about 19.1°2θ.

In some cases, crystalline 25HC3S choline may be characterized by an x-ray powder diffraction pattern having peaks at about 3.9°2θ, about 7.8°2θ, about 9.5°2θ, about 10.1°2θ, about 11.0°2θ, about 12.2°2θ, about 13.7°2θ, about 14.7°2θ, and about 15.1°2θ. In these and other cases, crystalline 25HC3S choline may be further characterized by an x-ray powder diffraction pattern having one or more peaks at about 15.8°2θ, about 16.3°2θ, and about 19.1°2θ.

In some cases, crystalline 25HC3S choline may be characterized by an x-ray powder diffraction pattern having peaks at about 3.9°2θ, about 7.8°2θ, about 9.5°2θ, about 10.1°2θ, about 11.0°2θ, about 12.2°2θ, about 13.7°2θ, about 14.7°2θ, about 15.1°2θ, and about 15.8°2θ. In these and other cases, crystalline 25HC3S choline may be further characterized by an x-ray powder diffraction pattern having one or more peaks at about 16.3°2θ and about 19.1°2θ.

In some cases, crystalline 25HC3S choline may be characterized by an x-ray powder diffraction pattern having peaks at about 3.9°2θ, about 7.8°2θ, about 9.5°2θ, about 10.1°2θ, about 11.0°2θ, about 12.2°2θ, about 13.7°2θ, about 14.7°2θ, about 15.1°2θ, about 15.8°2θ, and about 16.3°2θ. In these and other cases, crystalline 25HC3S choline may be further characterized by an x-ray powder diffraction pattern having a peak at about 19.1°2θ.

In some cases, crystalline 25HC3S choline may be characterized by an x-ray powder diffraction pattern having peaks at about 3.9°2θ, about 7.8°2θ, about 9.5°2θ, about 10.1°2θ, about 11.0°2θ, about 12.2°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having one or more peaks at about 7.8°2θ, about 9.5°2θ, about 10.1°2θ, about 11.0°2θ, about 12.2°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having one or more peaks at about 9.5°2θ, about 10.1°2θ, about 11.0°2θ, about 12.2°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having one or more peaks at about 10.1°2θ, about 11.0°2θ, about 12.2°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having one or more peaks at about 11.0°2θ, about 12.2°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having one or more peaks at about 12.2°2θ, about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having one or more peaks at about 13.7°2θ, about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having one or more peaks at about 14.7°2θ, about 15.1°2θ, about 15.8°2θ, about 16.3°2θ, and about 19.1°2θ.

In some cases, crystalline 25HC3S choline may be characterized by an x-ray powder diffraction pattern having one or more peaks at about 15.1°2θ, about 15.8°2θ, about 16.3°2θ, and about 19.1°2θ.

In some cases, crystalline 25HC3S choline may be characterized by an x-ray powder diffraction pattern having one or more peaks at about 15.8°2θ, about 16.3°2θ, and about 19.1°2θ.

In some cases, crystalline 25HC3S choline may be characterized by an x-ray powder diffraction pattern having one or more peaks at 16.3° 20 and about 19.1°2θ.

In some cases, crystalline 25HC3S choline may be characterized by an x-ray powder diffraction pattern having a peak at about 19.1°2θ.

In some cases, crystalline 25HC3S choline may be characterized by an x-ray powder diffraction pattern having substantially the same pattern as that found in FIG. 1.

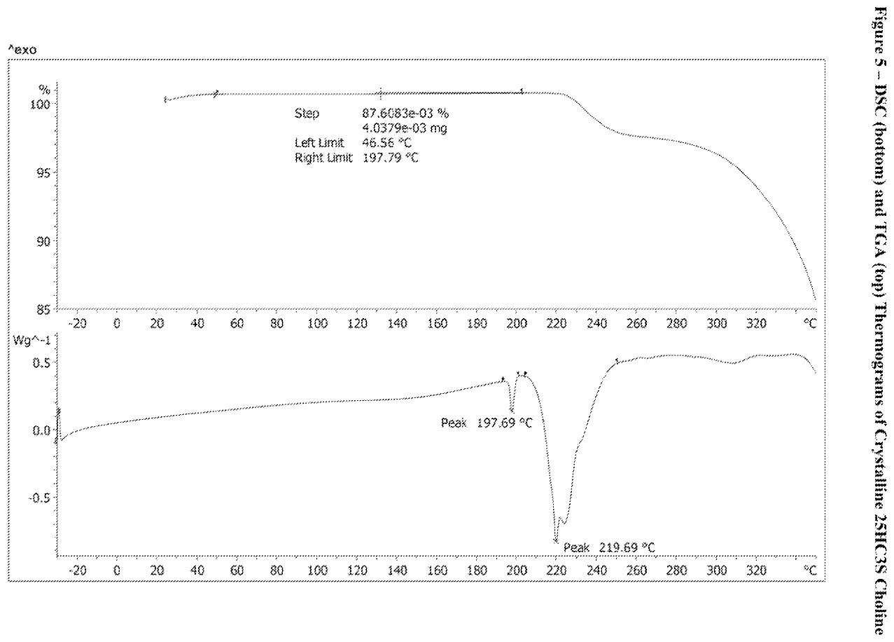

A DSC thermogram of crystalline 25HC3S choline indicated endothermic peaks at about 198° C. and about 220° C. The TGA thermogram of FIG. 5 indicates that there is negligible weight loss up to 198° C. Exemplary means for measuring any characterizing DSC endothermic peaks, and any characterizing DSC thermograms, are set out in Examples 21 and 23. In the context of DSC measurements, there is also variability and the term “about” means ±1° C., and such variability is to be understood whether a DSC measurement is prefaced by “about” or not unless specified otherwise.

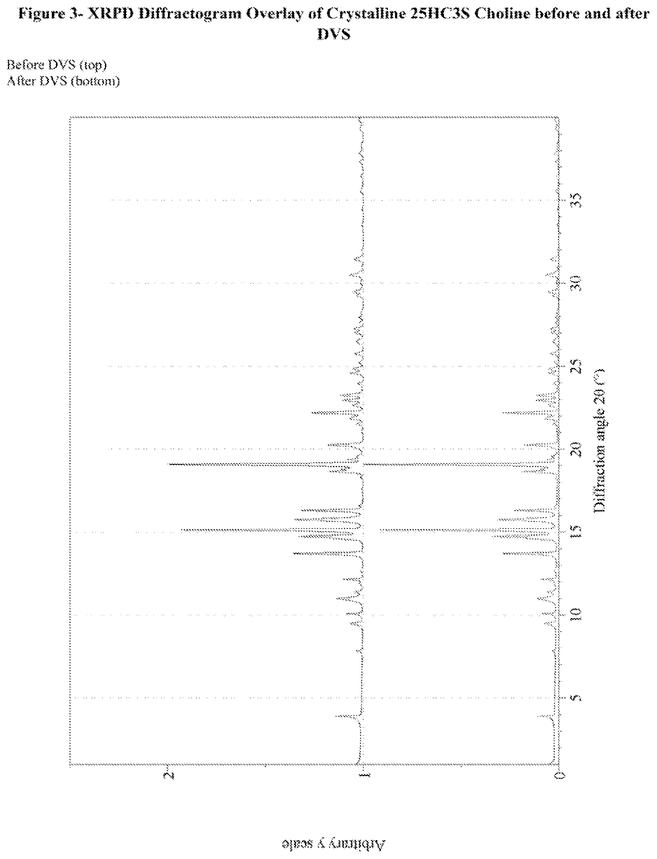

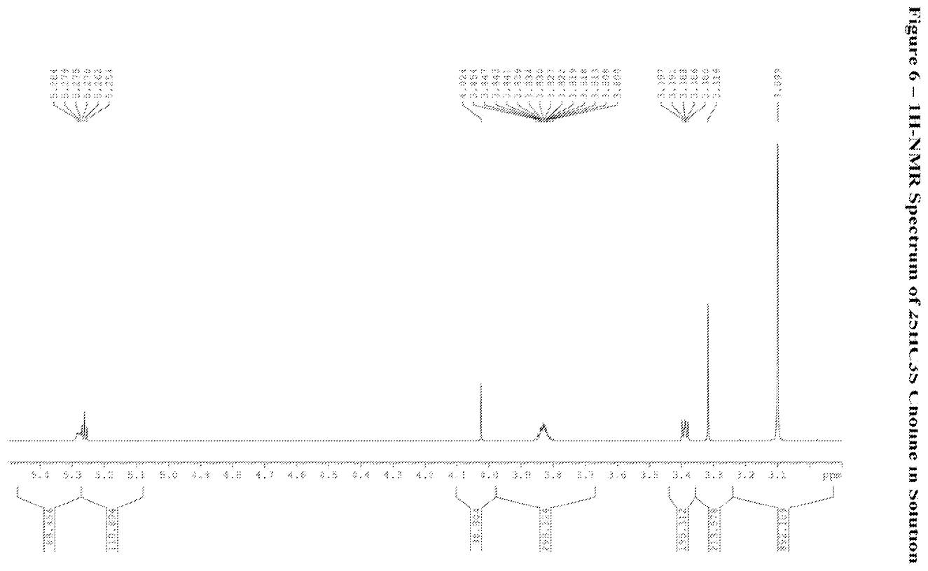

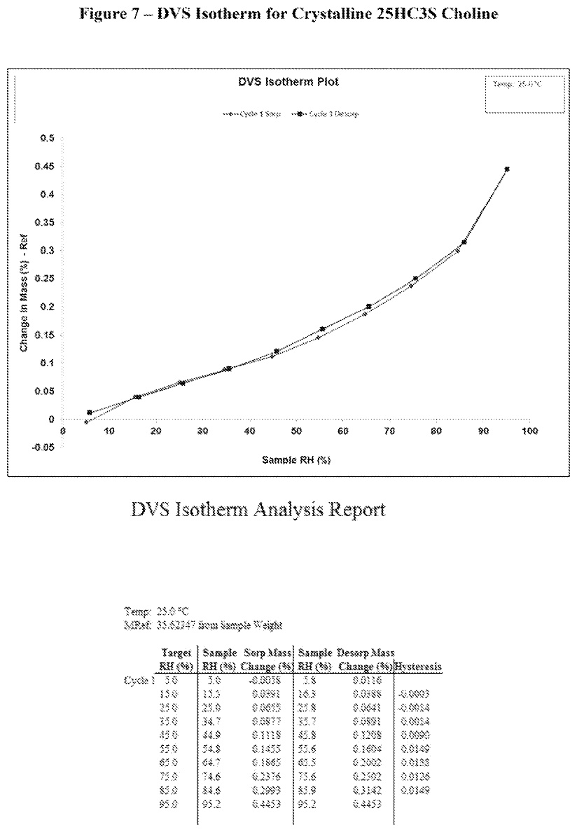

Without being bound by theory, it is believed that crystalline 25HC3S choline is an anhydrate, meaning that there is no water of crystallization in the unit cell. This does not preclude the possibility of other water being present in a solid comprising crystalline 25HC3S choline. In addition, crystalline 25HC3S choline is not appreciably hygroscopic up to about 95% relative humidity, increasing in weight by only about 0.5% up to this relative humidity as evidenced by a dynamic vapor sorption experiment according to Example 26 and whose results are shown in FIG. 7. Further, the x-ray powder diffraction pattern of crystalline 25HC3S choline does not appreciably change after DVS as shown in FIG. 3. Only a 0.5% weight gain was observed on going from 5% to 95% relative humidity and a 0.5% weight loss was observed on the return to 5% relative humidity indicating no hysteresis. Such low hygroscopicity indicates good stability under such stresses, which, as further discussed elsewhere herein, may make it suitably stable for pharmaceutical processing. Indeed, the present disclosure thus further includes stable crystalline 25HC3S choline. Such stability includes, for example, sufficiently stable crystalline 25HC3S choline to be formulated for patient delivery. The ¹H-NMR spectrum is, other than a peak at 5.3 ppm, consistent with structure as seen in FIG. 6.

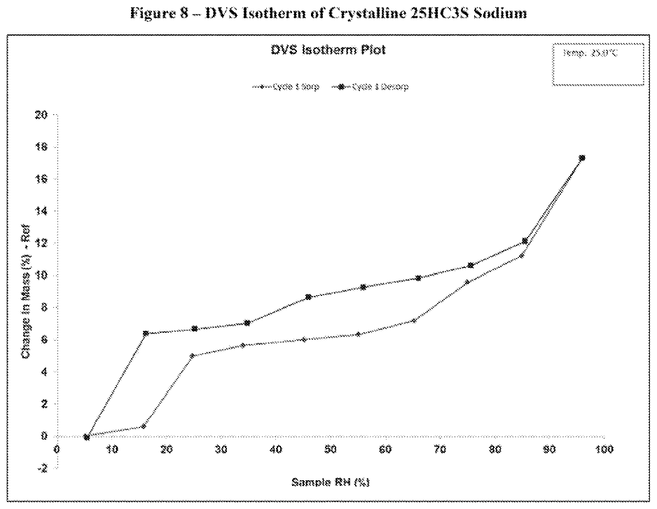

The choline salt has the additional advantage over the sodium salt and some other salts of 25HC3S in that the choline counterion has additional beneficial properties. For example, choline is an essential nutrient and lack of choline has been indicated as a cause of fat and cholesterol build up in the liver. Further, 25HC3S choline forms crystals of better quality and diffraction than those of the prior art. Lastly, crystalline 25HC3S choline is less hygroscopic, and thus more physically stable than, for example, crystalline 25HC3S sodium. Crystalline 25HC3S sodium stabilizes as a hydrate when exposed to humid conditions. In particular, monohydrates, dihydrates, and variable hydrates of crystalline 25HC3S sodium have been prepared. Form I, a hydrate, has been found to be hygroscopic and may form a liquid crystal at high water activities (e.g., above 0.73). Another hydrate, Form II, is stable at relative humidities between about 21% and about 30%. FIG. 8 shows a DVS isotherm of a manufactured batch of crystalline 25HC3S sodium containing both Form I and Form II and shows significant water uptake until about 95% relative humidity. By comparison, under conditions going up to about 95% relative humidity, only about 0.5% water by weight is absorbed indicating crystalline 25HC3S choline is stable as an anhydrate.

Substantially pure crystalline 25HC3S choline is further disclosed. “Substantially pure,” as described herein, generally refers to a form herein that is present without any appreciable amounts, other than potentially trace levels of other forms of 25HC3S choline. Examples of trace levels include not more than about 10%, 5%, 2%, 1.5%, 1%, 0.5%, 0.25%, 0.1%, or less in total relative to the total amount (based on weight) of 25HC3S choline present.

Processes of preparing 25HC3S choline are further described herein. In some cases, one may first prepare a sodium salt of 25HC3S. Examples of such preparation are set forth herein. The sodium salt of 25HC3S, which may be crystalline, may be converted into, for example, a triethylammonium salt as described in Example 27. The triethylammonium salt may then be used to create 25HC3S choline as set forth in Example 28.

The preparation of the triethylammonium salt of 25HC3S may be accomplished, for example, by passing a mixture of triethylammonium chloride and triethylamine through a column and treating with a solvent such as an alcohol until neutral pH. Separately, crystalline 25HC3S sodium may be dissolved in a solvent such as an alcohol. The solution may then be passed through the same column previously exposed to triethylamine and combined with the triethylammonium solution. Isolating resulting solids such as under vacuum or by drying may then provide crystalline 25HC3S triethylammonium salt which may be homogenized, for example, with a mortar and pestle. A suitable alcohol for this process includes methanol.

25HC3S choline, including crystalline 25HC3S choline, may be prepared by starting with 25HC3S sodium, converting to a second salt of 25HC3S such as the triethylammonium salt, and then converting that second salt of 25HC3S to 25HC3S choline, including crystalline 25HC3S choline. The preparation of crystalline 25HC3S choline may be accomplished by preparing a suspension of a triethylammonium salt of 25HC3S in a suitable solvent such as acetonitrile and treating with a choline source such as aqueous choline hydroxide to form 25HC3S choline including crystalline 25HC3S choline. The 25HC3S choline may be purified such as by rinsing with a suitable solvent. Additional processing such as drying under vacuum or otherwise may also be performed. The disclosure further includes crystalline 25HC3S choline made by the processes described herein.

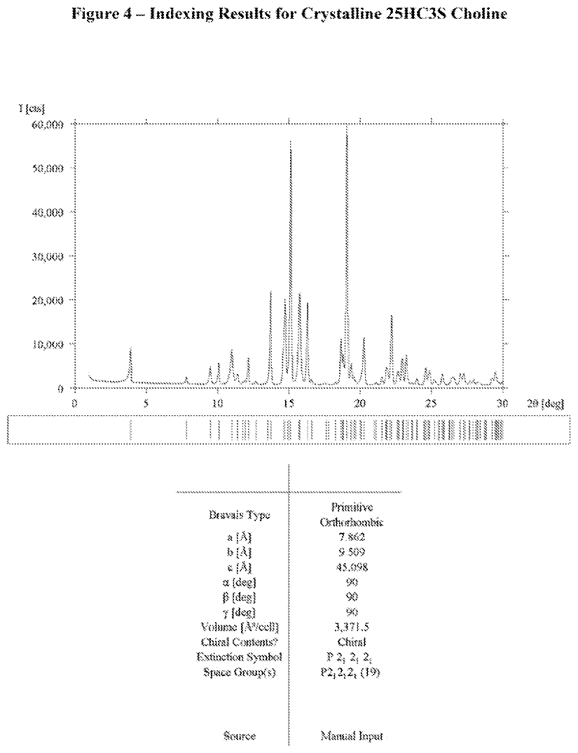

The x-ray powder diffraction pattern of crystalline 25HC3S choline was successfully indexed, indicating the pattern represents a single crystalline phase with the results set forth in FIG. 4. The indexing result reveals crystalline 25HC3S choline to have an orthorhombic cell with a cell volume of 3371.5 Å³, consistent with an anhydrous form. Several cell parameters are set forth in Table 2 below.

TABLE 2
Indexing Summary for Crystalline 25HC3S Choline

| | Primitive |
|---|---|
| Bravais Type | Orthorhombic |
| a[Å] | 7.862 |
| b[Å] | 9.509 |
| c[Å] | 45.098 |
| α | 90 |
| β | 90 |
| γ | 90 |
| Volume [Å³/cell] | 3,371.5 |
| Chiral Contents? | Chiral |
| Extinction Symbol | P 2₁ 2₁ 2₁ |
| Space Group(s) | P2₁2₁2₁ (19) |
| Source | Manual Input |

As discussed elsewhere herein, and demonstrated in the Examples, the 25HC3S choline of the disclosure has surprisingly low hygroscopicity, including in comparison to other salt forms of 25HC3S. The 25HC3S choline can therefore advantageously manufactured and may be utilized in the preparation of pharmaceutical formulations, and particularly in the preparation of dosage forms for oral administration (e.g., solid dosage forms, such as tablets, capsules (each of which includes immediate release, sustained release or timed release formulations), pills, powders, or granules.

Still further, the 25HC3S choline of the disclosure also advantageously provides supplementary choline to patients suffering from the conditions targeted by the 25HC3S. As discussed elsewhere herein, choline deficiency can contribute to these conditions and it may be advantageous to provide choline alongside the 25HC3S in the course of therapy. Hence, the 25HC3S choline surprisingly and beneficially combines advantageous salt form properties, contributing for instance to the preparation of oral dosage forms particularly well suited for treating certain conditions, along with intrinsic ability beneficially to provide choline supplementation in course of conducting methods of treatment using the said oral dosage forms. As discussed elsewhere herein, and demonstrated in the Examples, 25HC3S choline formulations of the present disclosure provide surprisingly increased exposure relative to the salt.

The present disclosure further includes methods and uses for treating and/or preventing diseases (e.g., in humans) such as one or more of nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, alcoholic fatty liver disease (AFLD), alcoholic steatohepatitis (ASH), leptin resistance, leptin deficiency, a diabetes condition, an autoimmune condition, an inflammatory condition, a neurological condition, Epstein Barr virus-related growth, and conditions related to fat accumulation and inflammation with effective amounts 25HC3S choline, including crystalline 25HC3S choline and/or pharmaceutical compositions comprising crystalline 25HC3S choline, amorphous choline, spray-dried choline, or micronized choline of the present disclosure.

The present disclosure further includes pharmaceutical compositions comprising 25HC3S choline and one or more excipients. The 25HC3S may be crystalline 25HC3S choline or amorphous 25HC3S choline. In many embodiments the 25HC3S choline is spray dried. In many embodiments, the 25HC3 choline may be micronized, such as by milling.

In many embodiments of the disclosure, the pharmaceutical composition comprises 25HC3S choline, such as one or more of crystalline, a mesophase, amorphous, spray dried, and micronized, and one or more fillers.

The amount of 25HC3S choline in the pharmaceutical composition expressed in weight percent typically ranges from about 1 wt % to about 90 wt %, about 5 wt % to about 40 wt %, and about 10 wt % to about 20 wt %.

As used herein, the term “filler” refers to a component that is incorporated into a composition (e.g., a tablet or capsule dosage form) to increase volume or weight any pharmaceutically acceptable filler that may be used in the practice of the present disclosure. Fillers are preferably pharmaceutically acceptable, e.g., acceptable for oral administration.

Examples of pharmaceutically acceptable fillers include, but are not limited to, talc, calcium carbonate (e.g., granules or powder), calcium phosphate (e.g., dibasic calcium phosphate (such as calcium phosphate, dibasic, anhydrous and calcium phosphate, dibasic, dihydrate), and tribasic calcium phosphate), calcium sulfate (e.g., granules or powder), calcium lactate, magnesium carbonate, magnesium oxide, sodium chloride, simethicone, a polysaccharide, pullulan, cellulose (e.g., microcrystalline cellulose (such as Avicel PH101 or Celphere CP-305), silicified microcrystalline cellulose, and powdered cellulose), cellaburate, ethylcellulose, cellulose acetate, copolymer of ethyl acrylate and methyl methacrylate, copolymer of methacrylic acid and methyl methacrylate, amino methacrylate copolymer, dextrate, dextrin, kaolin, mannitol, silicic acid, sorbitol, starch (e.g., Starch 1500, corn starch, pregelatinized starch, pregelatinized modified starch, pea starch, hydroxypropyl pea starch, potato starch, hydroxypropyl potato starch, tapioca starch, wheat starch, hydrogenated starch hydrolysate, modified starch, sterilizable maize starch, pregelatinized starch), alpha-lactalbumin, sugars or sugar alcohols (e.g., mannitol, isomalt, sorbitol, dextrose, polydextrose, xylitol, sucrose, lactose (such as lactose monohydrate, anhydrous lactose, and spray-dried lactose), compressible sugar, confectioner's sugar, and sugar spheres, corn syrup, corn syrup solids, glucose, fructose, galactose, trehalose, maltose (such as isomalt), maltodextrin, raffinose, mannitol, maltitol, melezitose, stachyose, lactitol, sorbitol, erythritol, palatinit, xylitol, myo-inositol), ammonium alginate, calcium silicate, fumaric acid, glyceryl palmitostearate, triglyceride, polymethacrylate, sodium alginate, medium-chain triglyceride, sulfobutylether R-cyclodextrin, and tragacanth and mixtures thereof. In certain embodiments, fillers include microcrystalline cellulose and lactose monohydrate, and mixtures thereof.

In many embodiments the one or more fillers comprises a polysaccharide, cellulose, microcrystalline cellulose, or a sugar, and mixtures thereof. In many embodiments, the sugar is chosen from lactose or lactose monohydrate.

The amount of filler in the pharmaceutical composition expressed in weight percent typically ranges from about 5 wt % to about 95 wt %, including about 30 wt % to about 60 wt % and about 40 wt % to about 80 wt %.

In some embodiments, pharmaceutical compositions of the disclosure comprise one or more fillers wherein the one or more fillers comprises microcrystalline cellulose ranging from about 5 wt % to about 95 wt %, including about 20 wt % to about 60 wt %.

In some embodiments, pharmaceutical compositions of the disclosure comprise one or more fillers wherein the one or more fillers comprise lactose monohydrate ranging from about 5 wt % to about 95 wt %, and including about 5 wt % to about 40 wt %.

In many embodiments of the disclosure, the pharmaceutical composition comprises 25HC3S choline, such as one or more of crystalline, amorphous, a mesophase, spray dried, and micronized 25HC3S choline, and one or more surfactants. In many embodiments, the pharmaceutical compositions of the disclosure comprise both fillers and surfactants. For example, the pharmaceutical compositions of the disclosure may comprise both at least one filler and at least one surfactant.

As used herein, the term “surfactant” refers to a compound having polar and non-polar regions. Surfactants may aggregate in solution to form micelles into which 25HC3S choline may partition and be solubilized. Surfactants are preferably pharmaceutically acceptable, such as, for example, acceptable for oral administration. The surfactants may be ionic or non-ionic.

Examples of surfactants include, but are not limited to, poloxamers or pluronics (e.g., poloxamer 188, poloxamer 335 and poloxamer 407), polyoxylglyceride, polyethylene glycols, polyethylene glycol monostearate, polysorbate (e.g., polysorbate 80, polysorbate 60, polysorbate 40, polysorbate 20), docusate sodium, benzalkonium chloride, sodium lauryl sulfate, sodium dodecyl sulfate, glyceryl monooleate, polyethoxylated and hydrogenated castor oil, wax, emulsifying wax, phospholipid, benzethonium chloride, cetrimide, cetylpyridinium chloride, xanthan gum, lauric acid, myristyl alcohol, butylparaben, ethylparaben, methylparaben, propylparaben, sorbic acid, polyoxyethylene alkyl ether, polyoxyethylene castor oil, polyoxyethylene sorbitan fatty acid ester, polyoxyethylene stearate, sorbitan ester, triethyl citrate, vitamin E polyethylene glycol succinate, and glycine.

Surfactants are often found in oral formulations. Examples of surfactants further include behenoyl polyoxylglyceride, caprylocaproyl polyoxylglyceride, cetylpyridinium chloride, lauroyl polyoxylglyceride, linoleoyl polyoxylglyceride, octoxynol 9, oleoyl polyoxylglyceride, polyoxyl 15 hydroxystearate, nonoxynol 9, pullulan, polyoxyl lauryl ether, polyoxyl stearyl ether, polyoxyl 10 oleyl ether, polyoxyl 20 cetostearyl. Examples include ethers, polyoxyl 20 cetyl ether, polyoxyethylene hydrogenated castor oil, sorbitan monolaurate, sorbitan monooleate, sorbitan monopalmitate, sorbitan monostearate, stearoyl polyoxylglyceride, sorbitan sesquioleate, sorbitan trioleate, tyloxapol, polyoxyl stearate (e.g., polyoxyl 40 stearate and polyoxyl 50 stearate), polyoxyethylene polyoxypropylene glycol, and sucrose fatty acid esters. A skilled artisan would understand that the one or more surfactants includes mixtures of the surfactants disclosed herein.

Preferred examples include sodium lauryl sulfate, polysorbate 80, polysorbate 60, polyoxyethylene hydrogenated castor oil, polyoxyethylene polyoxypropylene glycol, and sucrose fatty acid ester (sodium lauryl sulfate, polysorbate 80, polysorbate 60, polyoxyethylene; or two or more surfactants selected from hydrogenated castor oil, polyoxyethylene polyoxypropylene glycol, and sucrose fatty acid ester may be used in combination). In particular embodiments, surfactants are chosen from sodium lauryl sulfate and polysorbate 80. In other embodiments, surfactants include sugar fatty acid esters (wherein, for example, two or more surfactants selected from sodium lauryl sulfate, polysorbate 80, and sucrose fatty acid ester may be used in combination) and mixtures thereof. In many embodiments, surfactants include sodium lauryl sulfate.

The amount of surfactant in the pharmaceutical composition expressed in weight percent typically ranges from about 0.5 wt % to about 25 wt %, such as about 1% to about 20 wt %, about 2 wt % to about 10 wt %, about 3 wt % to about 9 wt %, about 4 wt % to about 8 wt %, and about 5 wt % to about 7 wt %.

In some embodiments, the one or more surfactants comprise sodium lauryl sulfate present in an amount ranging from about 0.5 wt % to about 25 wt %, such as about 1% to about 20 wt %, about 2 wt % to about 10 wt %, about 3 wt % to about 9 wt %, about 4 wt % to about 8 wt %, and about 5 wt % to about 7 wt %.

In many embodiments of the disclosure, the pharmaceutical composition comprises 25HC3S choline, such as one or more of crystalline, amorphous, a mesophase, spray dried, and micronized 25HC3S choline, and one or more disintegrants. In some embodiments, the pharmaceutical compositions comprise two or more disintegrants. In many embodiments, the pharmaceutical compositions of the disclosure comprise two or more of fillers, surfactants, and disintegrants. For example, the pharmaceutical compositions of the disclosure may comprise two or more of at least one filler, at least one surfactant, and at least one disintegrant.

As used herein, the term “disintegrant” refers to a component that assists breakup or disintegration into small units/fragments of a composition such as a tablet which may facilitate faster dissolution. For example, when disintegrants come in contact with water or stomach or intestinal fluid, they typically absorb liquid and start to swell, dissolve, or form gels. When a pharmaceutical composition is a tablet, this may cause the tablet structure to rupture and disintegrate, making increased surfaces for improved dissolution of the active ingredient, such as 25HC3S choline. Disintegrants are preferably pharmaceutically acceptable, e.g., acceptable for oral administration.

Examples of disintegrants include cross-linked starch, sodium starch glycolate, cross-linked cellulose, cross-linked carboxymethylcellulose, the sodium salt of cross-linked carboxymethylcellulose, polyvinylpyrrolidone, cross-linked polyvinylpyrrolidone, crospovidone. chitosan hydrochloride, corn starch, docusate sodium, magnesium aluminum silicate, starch, modified starch, pregelatinized starch, poloxamer, croscarmellose sodium, pregelatinized modified starch, hydroxypropyl starch, cellulose, methylcellulose, sodium carboxymethyl cellulose, powdered cellulose, low-substituted hydroxypropyl cellulose calcium carboxymethyl cellulose, substituted hydroxypropyl cellulose, microcrystalline cellulose, calcium cellulose glycolate, carmellosum calcium, alginates (e.g., sodium alginate, calcium alginate, and calcium sodium alginate), alginic acid, silicon dioxide, colloidal silicon dioxide, glycine, guar gum, polacrilin potassium, and sodium bicarbonate. A skilled artisan would understand that the one or more disintegrants includes mixtures of the disintegrants disclosed herein.

In some embodiments, the pharmaceutical compositions of the disclosure comprise 25HC3S choline and two or more disintegrants comprising croscarmellose sodium, meglumine, and cross-linked polyvinylpyrrolidone.

In some embodiments, pharmaceutical compositions of the disclosure comprise 25HC3S choline and three or more disintegrants.

The amount of disintegrant in the pharmaceutical composition expressed in weight percent typically ranges from about 0.5 wt % to about 25 wt % including from about 0.5 wt % to about 15 wt %. In some cases, the amount of disintegrant in the pharmaceutical composition expressed in weight percent ranges from about 10 wt % to about 30 wt % including from about 15 wt % to about 25 wt %.

In some embodiments, the one or more disintegrants comprise a water insoluble cross-linked polyvinylpyrrolidone. In some embodiments, the one or more disintegrants comprise a water soluble cross-linked polyvinylpyrrolidone. In certain instances, the cross-linked polyvinylpyrrolidone is crospovidone.

In some embodiments, the one or more disintegrants comprise crospovidone in an amount ranging from about 0.5 wt % to about 25 wt %, such as about 1% to about 20 wt %, about 2 wt % to about 10 wt %, about 3 wt % to about 9 wt %, about 4 wt % to about 8 wt %, and about 5 wt % to about 7 wt %. In some cases, the amount of crospovidone in the pharmaceutical composition expressed in weight percent ranges from about 10 wt % to about 30 wt % including from about 15 wt % to about 25 wt %.

The disintegrants of the pharmaceutical composition of the disclosure may be used to achieve a release profile wherein at least 20%, at least 30%, or at least 50% or more of the 25HC3S choline in a pharmaceutical compositions, such as a tablet, is released within 45 minutes of (1) administration to a human; or (2) being placed in a USP Apparatus 2 having 900 mL of dissolution medium that is 2.0% sodium dodecyl sulfate in 0.1 N HCl with a paddle speed of 75 rpm and a vessel temperature of 37° C.; or (3) being placed in a USP Apparatus 2 with 0-30 minutes in 450 mL FaSSGF and 30-45 minutes FaSSGF+double strength FaSSIF in a total of 900 mL of media (i.e., 450 mL of double strength FaSSIF is added to the initial 450 mL of FaSSGF) with pH adjusted to 6.5; with a paddle speed of 75 rpm and a vessel temperature of 37° C.

In many embodiments of the disclosure, the pharmaceutical composition comprises 25HC3S choline, such as one or more of crystalline, amorphous, a mesophase, spray dried, and micronized 25HC3S choline, and one or more glidants. In many embodiments, the pharmaceutical compositions of the disclosure comprise two or more of fillers, surfactants, disintegrants, and glidants. For example, the pharmaceutical compositions of the disclosure may comprise two or more of at least one filler, at least one surfactant, at least one disintegrant, and at least one glidant.

As used herein, the term “glidant” refers to a component used to promote flow properties, such as for use with tablet granulation or with powdered materials by decreasing interparticle friction and cohesion. Glidants are typically added in the dry state during the lubrication step before compression. In addition to promoting powder flow, glidants may reduce the caking or clumping that can occur when powders are stored in bulk. Glidants may also reduce the incidence of bridging during the emptying of powder hoppers and powder processing. Glidants are preferably pharmaceutically acceptable, e.g., acceptable for oral administration.

Examples of glidants include silicon dioxide, colloidal silicon dioxide, hydrophobic colloidal silica, colloidal anhydrous silica, magnesium trisilicate, tribasic calcium phosphate, calcium silicate, magnesium silicate, colloidal silicon dioxide, magnesium oxide, cellulose, sodium stearate, cellulose, powdered cellulose, starch, and talc. A skilled artisan would understand that the one or more glidants includes mixtures of the glidants disclosed herein.

In many embodiments, glidants are selected from silicon dioxide and/or colloidal silicon dioxide.

The amount of glidant in the pharmaceutical composition expressed in weight percent typically ranges from about 0.2 wt % to about 10 wt %, such as about 0.5 wt % to about 9 wt %, about 1 wt % to about 8 wt %, about 2 wt % to about 7 wt %, and about 3 wt % to about 6 wt %. In some cases, the amount of glidant in the pharmaceutical composition expressed in weight percent ranges from about 0.2 wt % to about 5 wt %, such as about 0.5 wt % to about 4 wt %, and about 1 wt % to about 3 wt %.

In some embodiments, the one or more glidants comprise silicon dioxide in an amount ranging from about 0.2 wt % to about 10 wt %, such as about 0.5 wt % to about 9 wt %, about 1 wt % to about 8 wt %, about 2 wt % to about 7 wt %, and about 3 wt % to about 6 wt % wt %. In some cases, the amount of silicon dioxide in the pharmaceutical composition expressed in weight percent ranges from about 0.2 wt % to about 5 wt %, such as about 0.5 wt % to about 4 wt %, and about 1 wt % to about 3 wt %.

In many embodiments of the disclosure, the pharmaceutical composition comprises 25HC3S choline, such as one or more of crystalline, amorphous, a mesophase, spray dried, and micronized 25HC3S choline, and one or more lubricants. In many embodiments, the pharmaceutical compositions of the disclosure comprise two or more of fillers, surfactants, disintegrants, glidants, and lubricants. For example, the pharmaceutical compositions of the disclosure may comprise two or more of at least one filler, at least one surfactant, at least one disintegrant, at least one glidant, and at least one lubricant.

As used herein, the term “lubricant” refers to a component used to reduce frictional forces. For instance, lubricants may reduce frictional forces between particle-particle as well as particles and metal-contact surfaces such as, for example, with manufacturing equipment such as tablet punches and dies used in the manufacture of solid dosage forms. Lubricants may prevent adhesion of tablet material to the surface of dies and punches, reduce interparticle friction, facilitate the ejection of tablets from die cavities, and may improve the rate of flow of the tablet granulation. Before compaction, liquid lubricants may be absorbed into the tablet granule matrix. Lubricants are preferably pharmaceutically acceptable, e.g., acceptable for oral administration.

Examples of lubricants include magnesium stearate, aluminum stearate, calcium stearate, zinc stearate, sodium stearate, stearic acid, magnesium silicate, polyethylene glycol, glyceryl behenate, mineral oil, light mineral oil, sodium stearyl fumarate, talc, hydrogenated vegetable oil, sodium lauryl sulfate, magnesium lauryl sulfate, behenoyl polyoxylglyceride, glyceryl dibehenate, lauric acid, glyceryl monostearate, glyceryl tristearate, myristic acid, palmitic acid, poloxamer, polysorbate 20, polyoxyl 10 oleyl ether, polyoxyl 15 hydroxystearate, polysorbate 40, polyoxyl 20 cetostearyl ether, polyoxyl 40 stearate, polysorbate 60, polysorbate 80, potassium benzoate, sodium benzoate, sorbitan monolaurate, sorbitan monooleate, sorbitan monopalmitate, sorbitan monostearate, sorbitan sesquioleate, sorbitan trioleate, glyceryl palmitostearate, hydrogenated castor oil, triglyceride, medium-chain triglyceride, sodium chloride, and polyethylene glycol 3350. A skilled artisan would understand that the one or more lubricants includes mixtures of the lubricants disclosed herein.

The amount of lubricant in the pharmaceutical composition expressed in weight percent typically ranges from about 0.1 wt % to about 5 wt %, including about 0.5 wt % to about 2 wt %.

In some embodiments, the one or more lubricants comprise magnesium stearate, e.g., in an amount ranging from about 0.1 wt % to about 5 wt %, including about 0.5 wt % to about 2 wt %.

In many embodiments of the disclosure, the pharmaceutical composition comprises 25HC3S choline, such as one or more of crystalline, amorphous, a mesophase, spray dried, and micronized 25HC3S choline, and one or more binders. In many embodiments, the pharmaceutical compositions of the disclosure comprise two or more of fillers, surfactants, disintegrants, glidants, lubricants, and binders. For example, the pharmaceutical compositions of the disclosure may comprise two or more of at least one filler, at least one surfactant, at least one disintegrant, at least one glidant, at least one lubricant, and at least one binder.

As used herein, the term “binder” refers to a component used to facilitate the agglomeration of powder into granules. For instance, binders may be incorporated into formulations to facilitate the agglomeration of powder into granules during mixing with a granulating fluid such as water, hydroalcoholic mixtures, or other solvents. Binders may be added either dry or in liquid form during wet granulation to form granules or to promote cohesive compacts for directly compressed tablets. Binders may be used to impart cohesive qualities to the powdered material. Binders may impart cohesiveness to tablet formulations to ensure the tablet remains intact after compression, as well as improving the free-flowing qualities of granules of desired hardness and size. Binders are preferably pharmaceutically acceptable, e.g., acceptable for oral administration.

Examples of binders include polyvinylpyrrolidone, copovidone, carbomer, corn starch, pregelatinized starch, carboxymethylcellulose sodium, hydroxypropyl methylcellulose, polyethylene glycol, hydroxyethyl cellulose, hydroxypropyl cellulose, hydroxyethylmethyl cellulose, calcium carboxymethylcellulose, calcium cellulose glycolate, guar galactomannan, ethylcellulose, chitosan, chitosan hydrochloride, dextrin, low-substituted hydroxypropyl cellulose, hydroxypropyl starch, ceratonia, inulin, magnesium aluminum silicate, maltodextrin, methylcellulose, dextrate, polyethylene oxide, povidone, sodium alginate, starch, glucose, sucrose, compressible sugar, zein, gelatin, polymethacrylate, sorbitol, liquid glucose, acacia, agar, alginic acid, calcium carbonate, calcium lactate, carrageenan, cellulose acetate phthalate, cottonseed oil, dextrose, glyceryl behenate, guar gum, hydrogenated vegetable oil, hypromellose, lactose, maltose, microcrystalline cellulose, pectin, poloxamer, polycarbophil, polydextrose, stearic acid, sunflower oil, tricaprylin, and vitamin E polyethylene glycol succinate. A skilled artisan would understand that the one or more binders includes mixtures of the binders disclosed herein.

The amount of binder in the pharmaceutical composition expressed in weight percent typically ranges from about 0.2 wt % to about 40 wt %, such as about 1 wt % to about 35 wt %, about 2 wt % to about 30 wt %, and about 5 wt % to about 25 wt %.

In many embodiments of the disclosure, the pharmaceutical composition comprises 25HC3S choline, such as one or more of crystalline, amorphous, a mesophase, spray dried, and micronized 25HC3S choline, and one or more plasticizers. In many embodiments, the pharmaceutical compositions of the disclosure comprise two or more of fillers, surfactants, disintegrants, glidants, lubricants, binders, and plasticizers. For example, the pharmaceutical compositions of the disclosure may comprise two or more of at least one filler, at least one surfactant, at least one disintegrant, at least one glidant, at least one lubricant, at least one binder, and at least one plasticizer.

Examples of plasticizers include acetyltributyl citrate, acetyltriethyl citrate, benzyl benzoate, cellulose acetate phthalate, chlorbutanol, dextrin, dibutyl phthalate, dibutyl sebacate, diethyl phthalate, dimethyl phthalate, glycerin, glycerin monostearate, hypromellose phthalate, mannitol, mineral oil, lanolin alcohol, palmitic acid, petrolatum, polyethylene glycol, polymethacrylate, polyvinyl acetate phthalate, propylene glycol, pyrrolidone, sorbitol, stearic acid, triacetin, tributyl citrate, triethanolamine, and triethyl citrate. A skilled artisan would understand that the one or more plasticizers includes mixtures of the plasticizers disclosed herein.

In many embodiments of the disclosure, the pharmaceutical composition comprises 25HC3S choline, such as one or more of crystalline, amorphous, a mesophase, spray dried, and micronized 25HC3S choline, and one or more antioxidants. In many embodiments, the pharmaceutical compositions of the disclosure comprise two or more of fillers, surfactants, disintegrants, glidants, lubricants, binders, plasticizers, and antioxidants. For example, the pharmaceutical compositions of the disclosure may comprise two or more of at least one filler, at least one surfactant, at least one disintegrant, at least one glidant, at least one lubricant, at least one binder, at least one plasticizer, and at least one antioxidant.

As used herein, the term “antioxidant” refers to a component used to reduce the oxidation of active substances and/or excipients in a pharmaceutical composition. A skilled artisan would understand that an antioxidant may reduce the oxidation of active substances and/or excipients.

Examples of antioxidants include alpha tocopherol, ascorbic acid, ascorbyl palmitate, butylated hydroxytoluene (BHT), butylated hydroxy anisole (BHA), citric acid monohydrate, citric acid, cysteine, erythorbic acid, fumaric acid, guaiac resin, malic acid, methionine, monothioglycerol, phosphoric acid, potassium, metabisulfite, propionic acid, propyl gallate, sesamol, sodium ascorbate, sodium bisulfite, sodium formaldehyde sulfoxylate, sodium metabisulfite, sodium sulfite, sodium thiosulfate, tartaric acid, tertiary butyl hydroquinone, thiol derivatives, thymol, and tocopherol. A skilled artisan would understand that the one or more antioxidants includes mixtures of the antioxidants disclosed herein.

In many embodiments of the disclosure, the pharmaceutical composition comprises 25HC3S choline, such as one or more of crystalline, amorphous, a mesophase, spray dried, and micronized 25HC3S choline, and one or more pH adjusting agents. In many embodiments, the pharmaceutical compositions of the disclosure comprise two or more of fillers, surfactants, disintegrants, glidants, lubricants, binders, plasticizers, antioxidants, and pH adjusting agents. For example, the pharmaceutical compositions of the disclosure may comprise two or more of at least one filler, at least one surfactant, at least one disintegrant, at least one glidant, at least one lubricant, at least one binder, at least one plasticizer, at least one antioxidant, and at least one pH adjusting agent.

As used herein the term “pH adjusting agent” refers to a component which may be used to adjust the pH of a pharmaceutical composition to assist in achieving a desired pH.

Examples of pH adjusting agents include acidifying agents and alkalizing agents. Examples of acidifying agents include acetic acid (glacial), citric acid (monohydrate), hydrochloric acid, acetic acid, fumaric acid, lactic acid, nitric acid, propionic acid, tartaric acid, sodium phosphate monobasic, and phosphoric acid. Examples of alkalizing agents include monoethanolamine, meglumine, sodium borate, sodium phosphate dibasic, ammonium carbonate, potassium hydroxide, sodium carbonate, trolamine, diethanolamine, sodium bicarbonate, and sodium hydroxide. A skilled artisan would understand that the one or more pH adjusting agents includes mixtures of the pH adjusting agents disclosed herein.

The amount of pH adjusting agent in the pharmaceutical composition expressed in weight percent typically ranges from about 0.1 wt % to about 50 wt %, such as about 1 wt % to about 40 wt %, about 2 wt % to about 30 wt %, and about 5 wt % to about 25 wt %.

The pharmaceutical compositions of the present disclosure may be in the form of orally administered solid dosage forms. Such solid dosage forms may be in the form of tablets, capsules, treches, lozenges, or pellets. In many solid oral dosage forms, the pharmaceutical composition comprising the 25HC3S choline and the one or more excipients contains a coating.

As used herein, the term “coating” refers to a covering. A coating may comprise a substance used to coat tablets or particles. For example, a tablet coating may be a covering over a tablet, used to mask the taste, make it easier to swallow, or protect the 25HC3S choline inside. A tablet coating may be applied to make the tablet smoother and easier to swallow. A tablet coating may color and protect the tablet, and mask a bad taste. A coating may protect a tablet ingredient from deterioration by moisture in the air. A coating formulation may comprise a polymer, colorant, plasticizer, and solvent (or vehicle). Coatings may comprise film formers, which may be enteric or non-enteric, solvents, plasticizers, colorants, and opaquant-extenders. Coatings are preferably pharmaceutically acceptable, e.g., acceptable for oral administration.

Examples of materials used in coatings include carnauba wax, cellulose acetate, cellulose acetate phthalate, ceresin, cetyl alcohol, chitosan, ethylcellulose, fructose, gelatin, glycerin, glyceryl behenate, glyceryl palmitostearate, hydroxyethyl cellulose, hydroxyethylmethyl cellulose, hydroxypropyl cellulose, hypromellose, hypromellose phthalate, isomalt, latex particles, glucose, liquid glucose, macrogol 400, maltitol, maltodextrin, methylcellulose, microcrystalline wax, paraffin, poloxamer, polydextrose, polyethylene glycol, polyethylene oxide, poly-DL-(lactic acid), polyvinyl acetate phthalate, polyvinyl alcohol, povidone, shellac, shellac with stearic acid, surface color agents, titanium oxide, tributyl citrate, triethyl citrate, vanillin, wax, white wax, xylitol, yellow wax, zein, polysorbate, acetyltriethyl citrate, carboxymethylcellulose calcium, carboxymethylcellulose sodium, and sucrose. A skilled artisan would understand that the coating materials include mixtures of the coating materials disclosed herein.

The amount of coating materials in the pharmaceutical composition expressed in weight percent typically ranges from about 0.1 wt % to about 10 wt %, such as about 0.5 wt % to about 9 wt %, about 1 wt % to about 8 wt %, about 2 wt % to about 7 wt %, and about 3 wt % to about 6 wt %.

In many embodiments of the disclosure, the pharmaceutical composition comprises 25HC3S choline, such as one or more of crystalline, amorphous, a mesophase, spray dried, and micronized 25HC3S choline, and one or more colorants. Examples of colorants include dyes, lakes, inorganic pigments, and natural colorants. Additional examples include titanium dioxide, iron oxide, riboflavin, caramel, ferric oxide, aluminum oxide, FD & C Red #40/Allura Red AC, amaranth, FD & C Blue #1/Brilliant Blue FCF, canthaxanthin, carmine, carmoisine (azorubine), curcumin (tumeric), FD & C Red #3/erythrosine, Fast Green FCF, Green S (Lissamine Green), D & C Red #30/helendon pink, FD & C Blue #2/indigo carmine, iron oxide black, iron oxide red, D & C Red #7/Lithol Rubin BK, Patent Blue V, D & C Red #28/Phloxine B, iron oxide yellow, D & C Red #27/Phloxine O, Ponceau 4R (Cochineal Red A), Quinoline Yellow WS, D & C Yellow #10, riboflavin (lactoflavin), FD & C Yellow #5/tartrazine, and FD & C Yellow #6/Sunset Yellow FCF. A skilled artisan would understand that the one or more colorants includes mixtures of the colorants disclosed herein.

The amount of colorant in the pharmaceutical composition expressed in weight percent typically ranges from about 0.01 wt % to about 5 wt %, such as about 0.1 wt % to 4 wt % and about 0.2 wt % to 3 wt %.

In many embodiments of the disclosure, the pharmaceutical composition comprises 25HC3S choline, such as one or more of crystalline, amorphous, a mesophase, spray dried, and micronized 25HC3S choline, and one or more flavorants. Examples of flavorants include vanillin, peppermint flavor powder, berry flavor powder, strawberry flavor powder, orange flavor powder, lemon flavor powder, orange essence, ethyl maltol, eucalyptus oil, isobutyl alcohol, sodium succinate, adipic acid, almond oil, anethole, benzaldehyde, denatonium benzoate, ethyl acetate, ethyl vanillin, ethylcellulose, fructose, fumaric acid, L-glutamic acid hydrochloride, lactitol, leucine, malic acid, maltol, menthol, racementhol, methionine, methyl salicylate, monosodium glutamate, peppermint oil, liquid strawberry flavor, peppermint spirit, racemethionine, rose oil, rose water, sodium acetate, sodium lactate, tartaric acid, thymol, inulin, isomalt, and neohesperidin dihydrochalcone. A skilled artisan would understand that the one or more flavorants includes mixtures of the flavorants disclosed herein.

The amount of flavorant in the pharmaceutical composition expressed in weight percent typically ranges from about 0.1 wt % to about 10 wt %, such as about 0.5 wt % to about 9 wt %, about 1 wt % to about 8 wt %, about 2 wt % to about 7 wt %, and about 3 wt % to about 6 wt %.

In many embodiments of the disclosure, the pharmaceutical composition comprises 25HC3S choline, such as one or more of crystalline, amorphous, a mesophase, spray dried, and micronized 25HC3S choline, and one or more sweetener. Examples of sweeteners include sucralose, saccharin, saccharin calcium, saccharin sodium, neotame, sucrose, acesulfame potassium, aspartame, aspartame acesulfame, corn syrup, corn syrup solids, dextrate, dextrose, erythritol, fructose, galactose, glucose, glycerin, inulin, invert sugar, isomalt, lactitol, maltitol, maltose, mannitol, sorbitol, hydrogenated starch hydrolysate, compressible sugar, confectioner's sugar, tagatose, trehalose, and xylitol. A skilled artisan would understand that the one or more sweeteners includes mixtures of the sweeteners disclosed herein.

The amount of sweetener in the pharmaceutical composition expressed in weight percent typically ranges from about 0.1 wt % to about 20 wt %, such as about 0.5 wt % to about 15 wt %, about 1 wt % to about 10 wt %, about 2 wt % to about 9 wt %, and about 3 wt % to about 8 wt %.

In many embodiments, the 25HC3S choline of the disclosure is crystalline. For example, the 25HC3S choline may be micronized. In many embodiments, the 25HC3S choline is amorphous. In many embodiments, the 25HC3S is spray dried. Amorphous 25HC3S choline and spray-dried 25HC3S choline may be formulated to make pharmaceutical compositions of the disclosure. Amorphous 25HC3S choline may be made by spray drying. It is also within the scope of the disclosure to make spray-dried dispersions of 25HC3S choline comprising one or more plasticizers. The disclosure further includes pharmaceutical compositions comprising micronized 25HC3S choline. The spray-dried 25HC3S choline dispersions may have a particle size distribution ranging from about 0.1 microns to about 100 microns, such as about 0.2 microns to about 50 microns, and about 1 micron to about 10 microns at D10, as measured by laser diffraction. The spray-dried 25HC3S choline dispersions may have a particle size distribution ranging from about 0.1 microns to about 100 microns, such as about 0.2 microns to about 50 microns, and about 1 micron to about 10 microns at D50, as measured by laser diffraction. The spray-dried 25HC3S choline dispersions may have a particle size distribution ranging from about 0.1 microns to about 100 microns, such as about 0.2 microns to about 50 microns, and about 1 micron to about 10 microns at D90, as measured by laser diffraction.

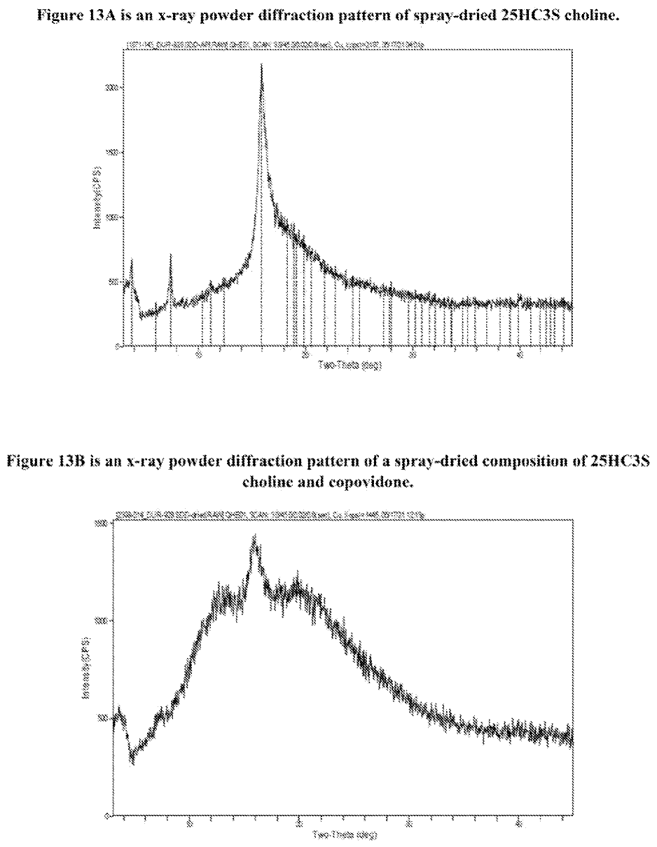

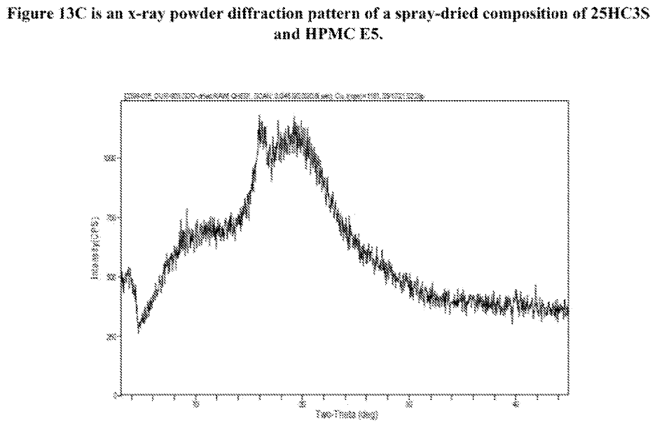

Amorphous 25HC3S choline may be characterized by an x-ray powder diffraction having a pattern substantially the same as that as FIG. 13A. The amorphous 25HC3S choline of FIG. 13A was prepared by spray drying. In some embodiments, amorphous 25HC3S may be obtained by spray drying a solution of 25HC3S in a suitable solvent system. In some embodiment, the suitable solvent system comprises an alcohol, such as methanol, and water.

In many embodiments, the pharmaceutical compositions comprise spray-dried dispersions of 25HC3S choline. Dispersions are usually multiple-component compositions comprising an active pharmaceutical ingredient and one or more plasticizers. In many embodiments, the disclosure provides for pharmaceutical compositions comprising spray-dried dispersions of 25H3CS choline and one or more plasticizers wherein the one or more plasticizers are polymers. Polymers include water-soluble or partially water-soluble polymers and pH-dependent enteric polymers. Water-soluble or partially water-soluble polymers include but are not limited to, cellulose derivatives (e.g., hydroxypropylmethylcellulose (HPMC), hydroxypropylcellulose (HPC)) or ethylcellulose; polyvinylpyrrolidones (PVP); polyethylene glycols (PEG), polyvinyl alcohols (PVA), acrylates, such as polymethacrylate (e.g., Eudragit® E); cyclodextrins (e.g., cyclodextin) and copolymers and derivatives thereof, including for example PVP-VA (polyvinylpyrollidone-vinyl acetate). Examples of pH-dependent enteric polymers include, but are not limited to, cellulose derivatives (e.g., cellulose acetate phthalate (CAP)), hydroxypropyl methyl cellulose phthalates (HPMCP), hydroxypropyl methyl cellulose acetate succinate (HPMCAS), carboxymethylcellulose (CMC) or a salt thereof (e.g., a sodium salt such as (CMC-Na)); cellulose acetate trimellitate (CAT), hydroxypropylcellulose acetate phthalate (HPCAP), hydroxypropylmethyl-cellulose acetate phthalate (HPMCAP), and methylcellulose acetate phthalate (MCAP), or polymethacrylates (e.g., Eudragit® S). Exemplary polymers include copovidone and HPMC. Copovidone is also knows as Poly(1-vinylpyrrolidone-co-vinyl acetate) with CAS #25086-89-9. In many embodiments of the disclosure, the polymer is a copovidone with a ratio of 1-vinyl-2-pyrrolidone to vinyl acetate in a weight ratio of 6:4. In these and other embodiments, the HPMC is another polymer wherein the methoxyl content ranges from about 28 wt % to about 30 wt % and the hydroxypropoxyl content ranges from about 7 wt % to about 12%.

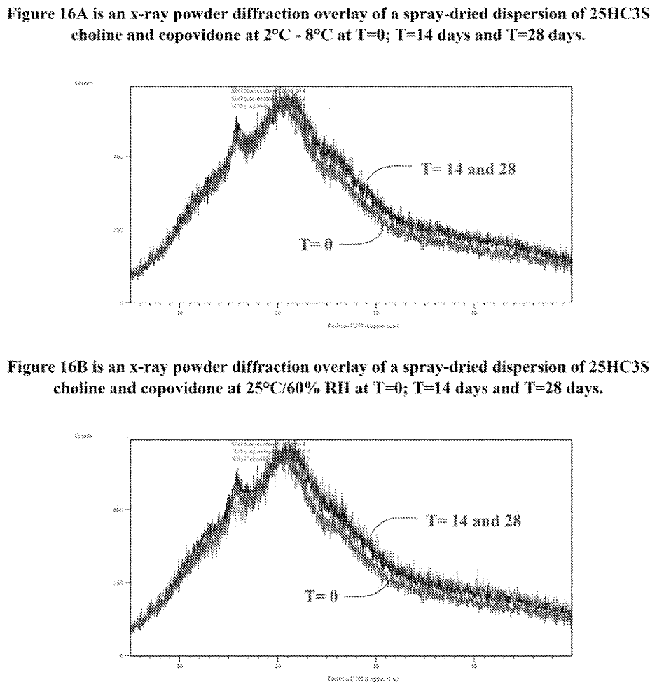

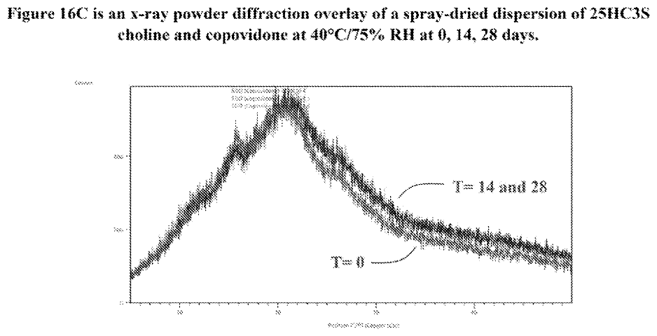

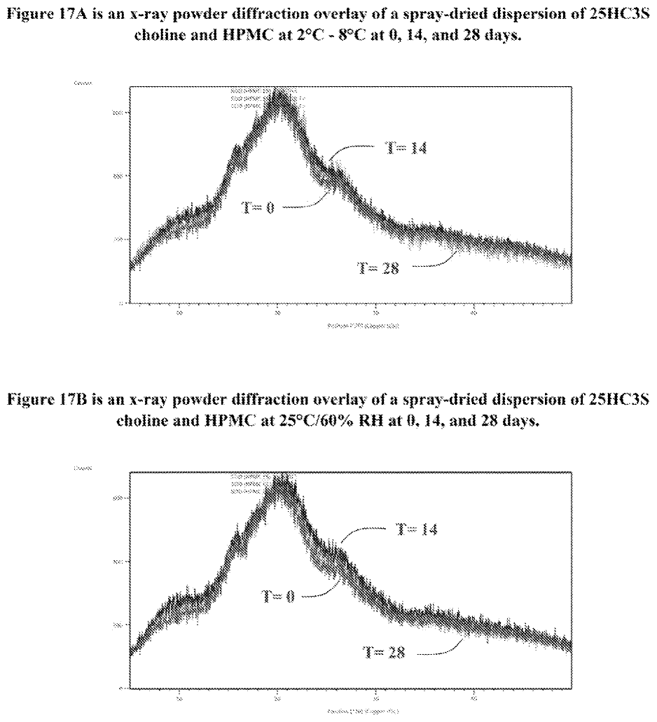

Spray-dried dispersions may be characterized by x-ray powder diffraction. FIG. 13B is an x-ray powder diffraction pattern of a spray-dried composition of 25HC3S choline and copovidone, and FIG. 13C is an x-ray powder diffraction pattern of a spray-dried composition of 25HC3S choline and HPMC E5. Combinations of 25HC3S and one or more polymers may be spray-dried to create spray-dried dispersions comprising 25HC3S choline and one or more polymers. Such dispersions have also been shown to be stable. For example, FIG. 16A is an x-ray powder diffraction corresponding to a spray-dried dispersion of copovidone and 25HC3S choline indicating the amorphous nature of the dispersion.

In FIG. 16A the storage conditions were between 2° C. and 8° C., and the measurements were made initially (T=0) and at days 14 and 28 (T=14; T=28). As can be seen from FIG. 16A, at all three time points, the solid dispersion is stable. The same can be seen from FIG. 16B (25° C./60% Relative Humidity (RH)) and FIG. 16C (40° C./75% RH). In these and other embodiments, the mass ratio of 1-vinyl-2-pyrrolidone and vinyl acetate in copovidone is 6:4.

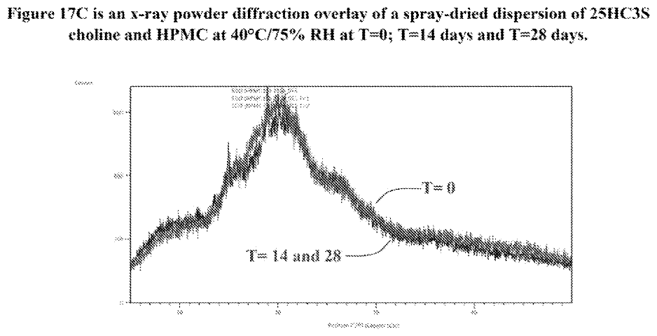

In other embodiments, spray-dried dispersions of 25HC3S choline and hydroxypropyl methylcellulose (also called Hypromellose or HPMC). In some embodiments, the HPMC used is HPMC E5, where E5 is a grade of HPMC. X-ray powder diffraction patterns of 25HC3S choline and HPMC E5 under various conditions are set forth in FIGS. 17A, 17B, and 17C, and are also shown to be stable. In these and other embodiments, the HPMC has a methoxyl content ranging from about 28 wt % to about 30 wt % and a hydroxypropyl content ranging from about 7 wt % to about 12 wt %. Also provided herein are solid-dispersions comprising 25HC3S choline, copovidone, and HPMC.

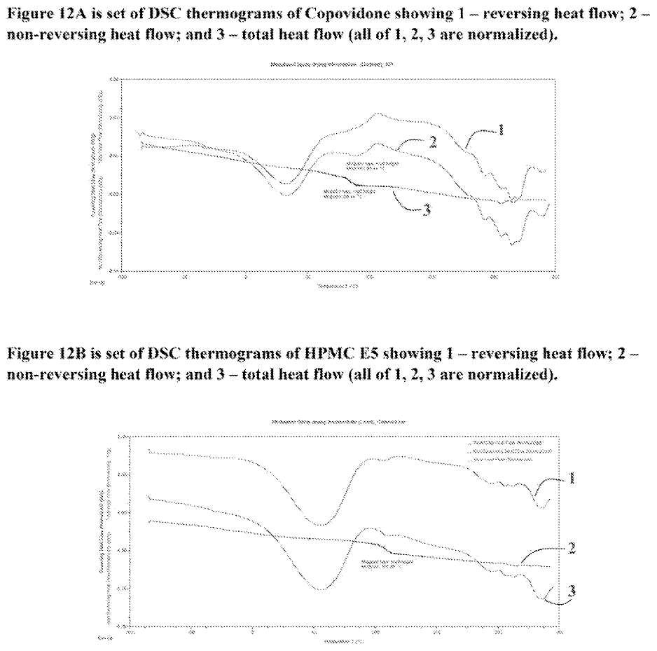

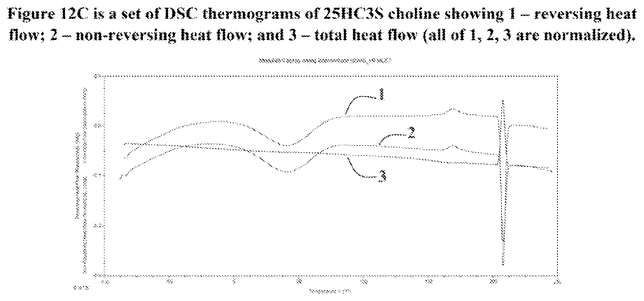

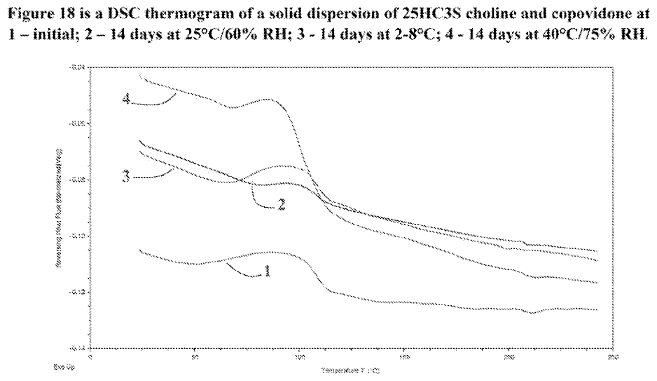

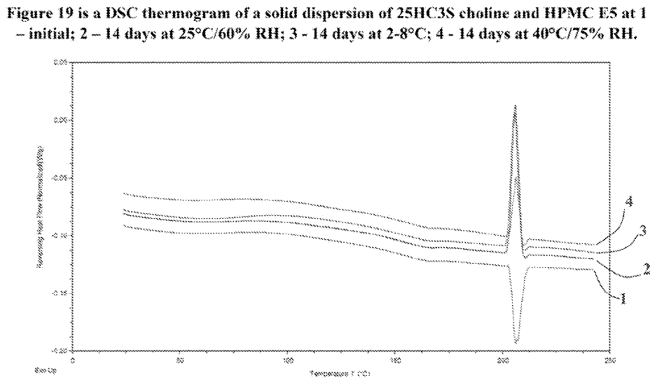

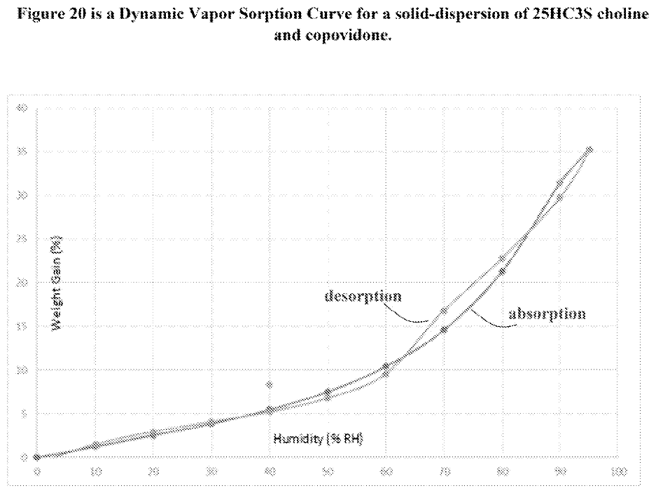

Differential scanning calorimetry may be used to analyze spray-dried dispersions. FIGS. 12A, 12B, and 12C are DSC thermograms of copovidone, HPMC E5, each with choline and neat 25HC3S choline respectively. No melting endotherm was observed in any thermogram. DSC thermograms of spray-dried dispersions can be found in FIG. 18 (25HC3S choline and copovidone) and FIG. 19 (25HC3S choline and HPMC E5) under stability (1—initial; 2—25° C./60% RH; 3—2-8° C.; 4-40° C./75% RH).

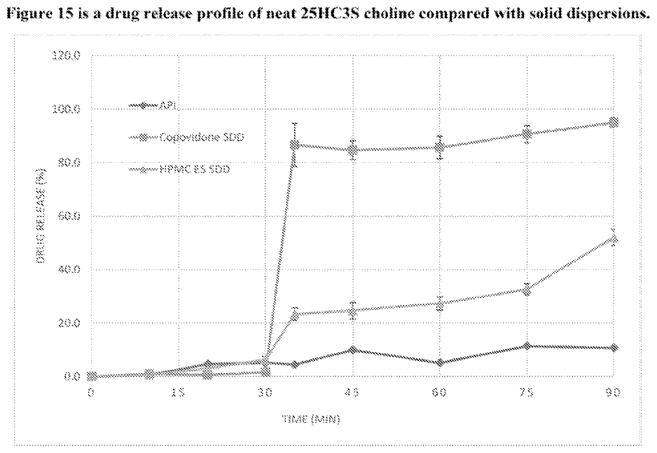

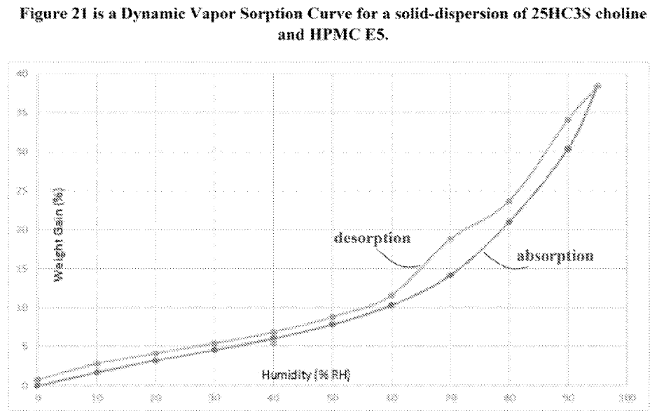

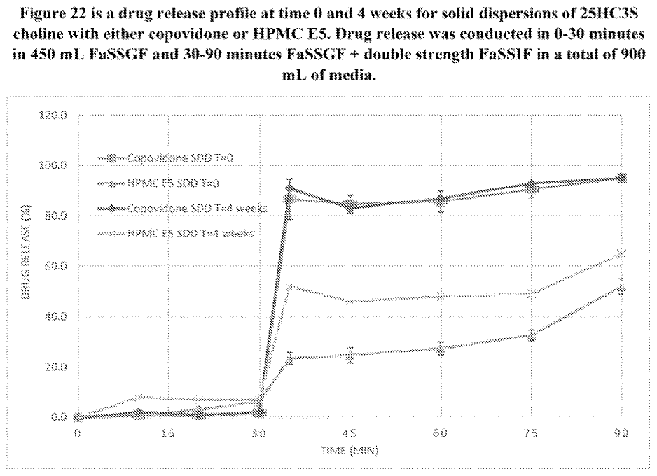

Dynamic Vapor Sorption curves in FIG. 20 (copovidone and 25HC3S choline spray-dried dispersion and FIG. 21 (HPMC E5 and 25HC3S choline spray-dried dispersion). Example 32 discusses spray-dried dispersions 25HC3S choline and HPMC E5. FIG. 15 provides drug release profiles comparing spray-dried dispersions to neat 25HC3S choline showing a substantial increase in drug release using spray-dried dispersions. FIG. 22 shows drug release at an initial time point and then four weeks later. Drug release at four weeks was found to be the same or higher for both spray-dried dispersions which is indicative of a sustained dissolution advantage with stability.

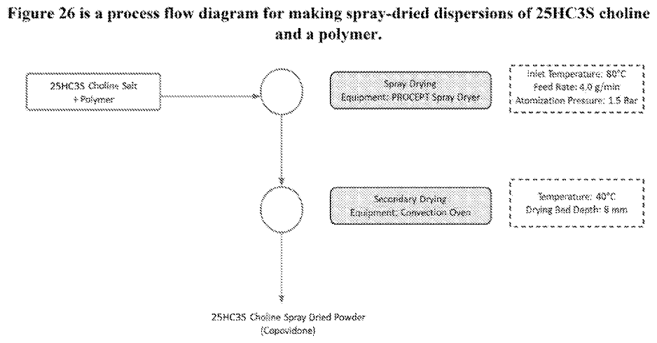

Spray-dried dispersions of 25HC3S choline may be manufactured in accordance with Example 32 and as diagramed in FIG. 26. Pharmaceutical compositions, such as tablets, may also be made from spray-dried dispersions. Examples of such tablets may be found in Examples 34, 35, and 36.

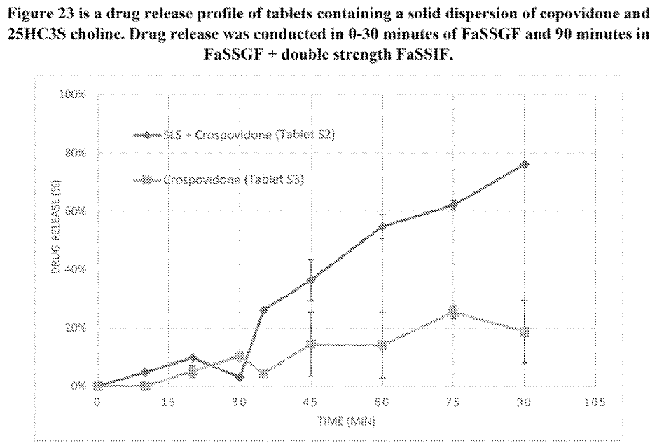

In Example 34, drug release profiles of tablets so manufactured are set forth in FIG. 23. Tablet S2 contained sodium lauryl sulfate as an intragranular excipient whereas Tablet S3 did not. Tablet S2 had a greater drug release than Tablet S3.

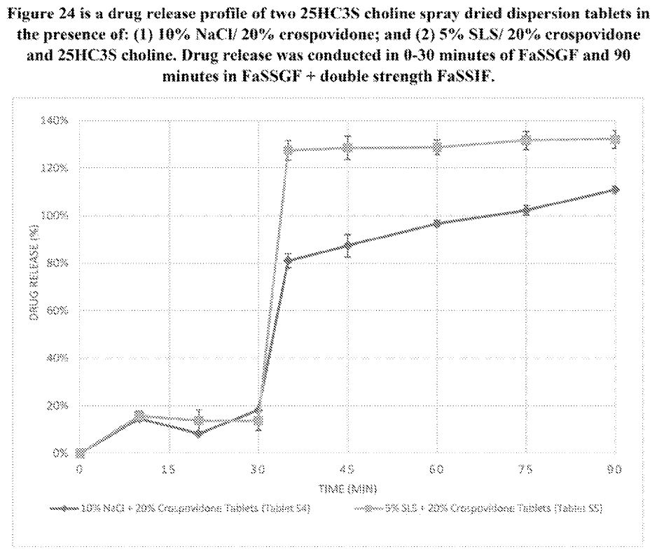

In Example 35, Tablet S1 contained no sodium lauryl sulfate as an intragranular excipient and 10 wt % sodium chloride whereas Tablet S2 contained 5.0 wt % sodium lauryl sulfate and no sodium chloride. As FIG. 24 shows, Tablet S2 had an overall faster release profile.

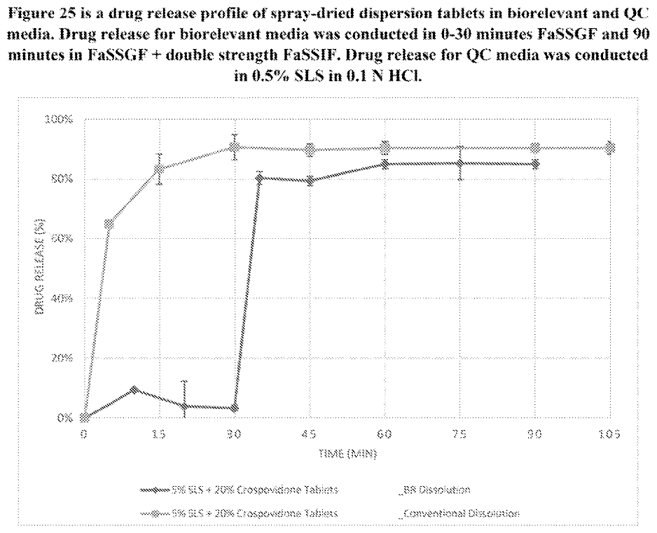

In Example 36, a batch of tablets was made having the ingredients set forth in Table D6. The batch contained 5.0 wt % sodium lauryl sulfate whose release profile can be found in FIG. 25 in two different release media—“Biorelevant” media (bottom) and a QC media (top). Both curves show substantial release.

Biorelevant media as used herein is a combination of 2 different media used at different times. The first is in FaSSGF which stands for Fasted-state simulated gastric fluid and is used for 30 minutes of drug release. Afterwards, drug release is measured with addition of double strength FaSSIF which is Fasted-state simulated intestinal fluid. A skilled artisan would understand that the double strength FaSSIF is added at 30 minutes.

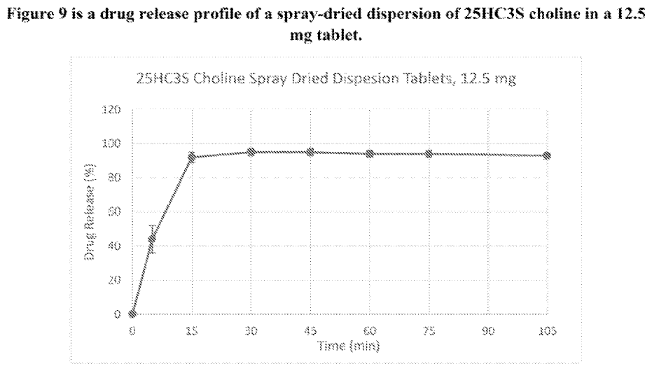

In additional embodiments of the disclosure, tablets made with the composition set forth in Table TO below were measured for drug release using parameters set forth in Table TOA below with the results set forth in FIG. 9.

TABLE T0

| | | |
|---|---|---|
| Description | (% w/w) | mg/tablet |

Intra-Granular

| | | |
|---|---|---|
| 25HC3S Choline Spray Dried Intermediate¹ | 22.22 | 50.00² |
| Microcrystalline Cellulose (Avicel PH-101) | 37.32 | 83.97³ |
| Lactose Monohydrate (Fast Flow 316) | 12.46 | 28.04 |
| Sodium Lauryl Sulphate | 5.00 | 11.25 |
| Crospovidone | 5.00 | 11.25 |
| Colloidal Silicon Dioxide | 1.00 | 2.25 |
| Magnesium Stearate | 0.50 | 1.13 |

Extra-Granular

| | | |
|---|---|---|
| Crospovidone | 15.00 | 33.75 |
| Colloidal Silicon Dioxide | 1.00 | 2.25 |
| Magnesium Stearate | 0.50 | 1.13 |
| Total | 100 | 225.00 |

¹25HC3S Choline Spray Dried Dispersion contains 25HC3S Choline:Copovidone (25.0:75.0 w/w).
²25HC3S Choline Spray Dried Dispersion is adjusted for molecular weight of API and Assay value of SDD.
³Avicel PH-101 is adjusted based on use-as-value to maintain the percent composition to 100%.

TABLE T0A

| Description | Parameters |
|---|---|
| Apparatus: | USP Apparatus II (Paddles) |
| Temperature: | 37.0° C. ± 0.5° C. |
| Medium: | 2.0% Sodium Lauryl Sulfate in |
| | 0.1 molar Hydrochloric Acid |
| Medium Volume: | 900 mL |
| Rotation Speed | 75 rpm (250 rpm from 75 minutes to |
| | 105 minutes) |
| Sampling | 5, 15, 30, 45, 60, 75, and 105 minutes |
| Timepoints: | |
| Sampling Volume: | 5 mL |

In further embodiments of the disclosure the pharmaceutical compositions comprising spray-dried dispersions of 25HC3S choline and one or more plasticizers may be configured, such as by choice of plasticizer and amount used, to release 80% or more of 25HC3S choline within 5 minutes after (1) administration to a human; or (2) being placed in a USP Apparatus 2 having 900 mL of dissolution medium that is 2.0% sodium dodecyl sulfate in 0.1 N HCl with a paddle speed of 75 rpm and a vessel temperature of 37° C.; or (3) being placed in a USP Apparatus 2 having 450 mL FaSSGF with a paddle speed of 75 rpm and a vessel temperature of 37° C., or up to 40% within the first 5 minutes of (1) administration to a human; (2) or being placed in a USP Apparatus 2 having 900 mL of dissolution medium that is 2.0% sodium dodecyl sulfate in 0.1 N HCl with a paddle speed of 75 rpm and a vessel temperature of 37° C.; or (3) being placed in a USP Apparatus 2 having 450 mL FaSSGF with a paddle speed of 75 rpm and a vessel temperature of 37° C.

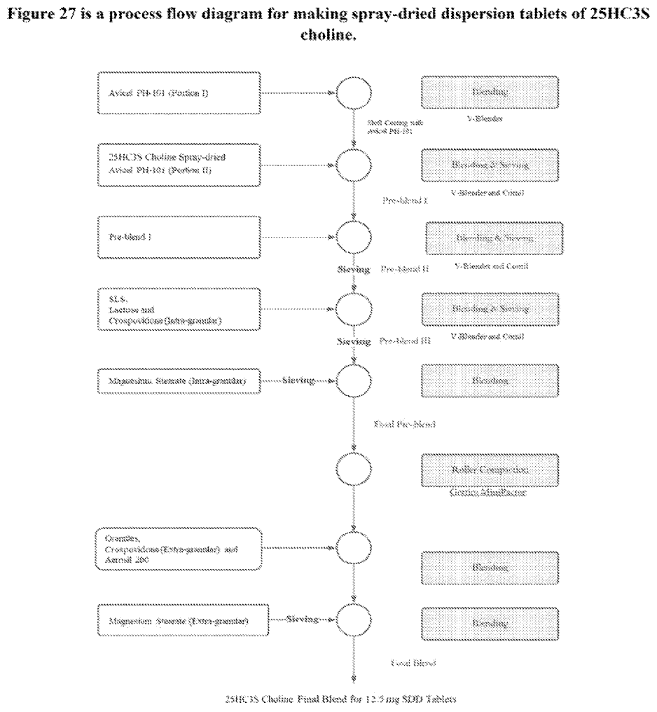

When configured as tablets, in many embodiments, the tablets contain one or more pharmaceutically acceptable excipients. In some embodiments, the excipients comprise one or more of microcrystalline cellulose, lactose monohydrate, crospovidone, sodium lauryl sulfate, colloidal silicon dioxide, and magnesium stearate. The tablets may be prepared by forming granules with a spray-dried dispersion of 25HC3S choline and one or more polymers and one or more pharmaceutically acceptable excipients. Such excipients when used to make granules may be referred to as intragranular excipients. FIG. 27 is a process flow diagram showing the manufacture of a “Final pre-blend” which uses the intragranular excipients prior to making the “Final Blend” for tablets with extra-granular excipients. Accordingly, tablets containing spray-dried dispersions of 25HC3S choline may contain both intragranular and extragranular excipients. In many embodiments, extragranular excipients comprise one or more of colloidal silicon dioxide, crospovidone, and magnesium stearate.

In many embodiments of the disclosure, micronized 25HC3S choline is provided.

Micronized 25HC3S choline may be prepared, for example, in accordance with Example 39. The particle size for such micronized 25HC3S materials may have a distribution of 2 to 4 microns at D50. The particle size for micronized 25HC3S choline may have a distribution ranging from about 0.1 microns to about 50 microns, such as about 0.2 microns to about 25 microns, and about 1 micron to about 10 microns at D10, as measured by laser diffraction. The particle size for micronized 25HC3S choline may have a distribution ranging from about 0.1 microns to about 50 microns, such as about 0.2 microns to about 25 microns, and about 1 micron to about 10 microns at D50, as measured by laser diffraction. The particle size for micronized 25HC3S choline may have a distribution ranging from about 0.1 microns to about 50 microns, such as about 0.2 microns to about 25 microns, and about 1 micron to about 10 microns at D90, as measured by laser diffraction. The disclosure further provides for pharmaceutical compositions comprising micronized 25HC3 choline and one or more pharmaceutically acceptable excipients. In many embodiments, the pharmaceutical compositions are solid oral dosage forms. Examples of such solid oral dosage forms include capsules and tablets.

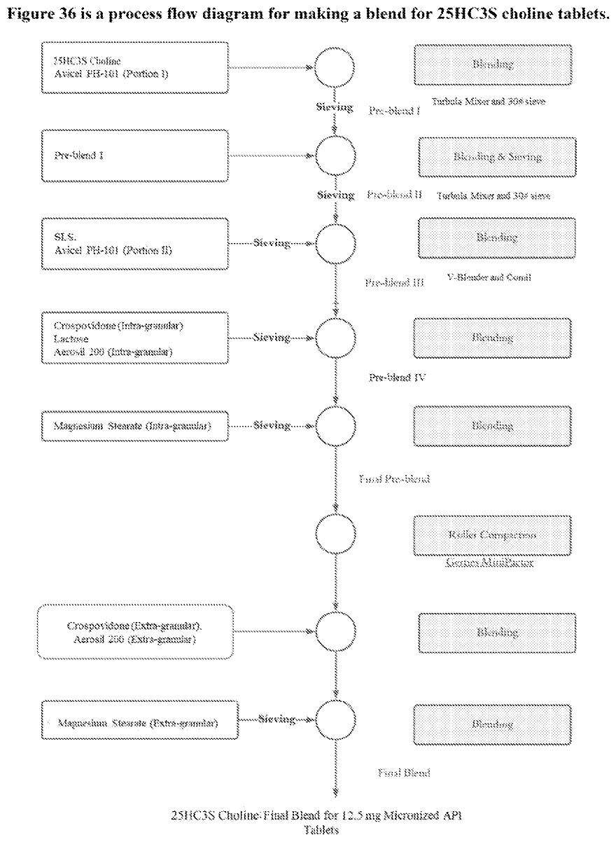

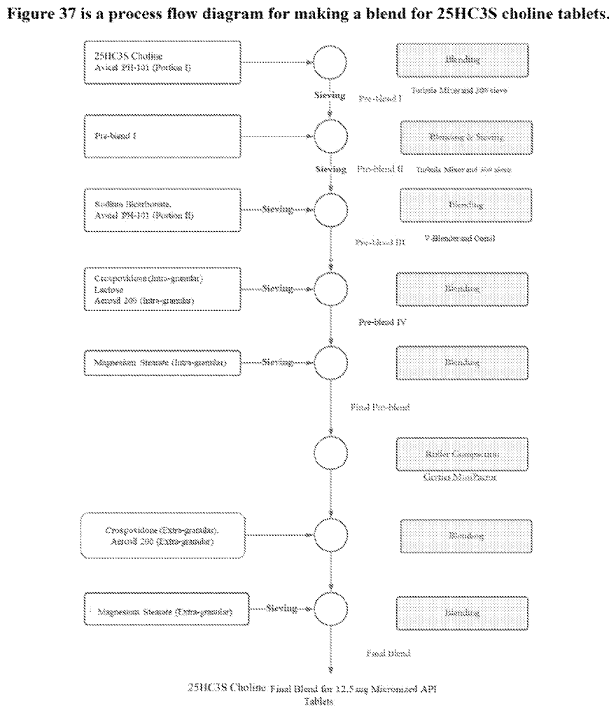

When in tablet form, the tablets may be manufactured in accordance with FIG. 36 and FIG. 37, for example. In each of FIGS. 36 and 37, the manufacture comprises making one or more pre-blends with intragranular excipients followed by combining with extragranular excipients to form granules to make tablets. Examples of intragranular excipients include meglumine, microcrystalline cellulose, lactose monohydrate, sodium lauryl sulfate, polysorbate 80, polyethylene glycol, crospovidone, copovidone, colloidal silicon dioxide, magnesium stearate, and sodium bicarbonate. A skilled artisan would understand that the intragranular excipients include mixtures of the intragranular excipients disclosed herein. Examples of extragranular excipients include croscarmellose sodium, colloidal silicon dioxide, crospovidone, and magnesium stearate. A skilled artisan would understand that the extragranular excipients include mixtures of the extragranular excipients disclosed herein.

In some embodiments, the tablets have a hardness ranging from 3 KP to 9 KP, such as from 4 KP to 8 KP, or 5 KP to 7 KP.

Examples 37, 38, and 39 show the preparation of various tablets of the disclosure using micronized 25HC3S choline.

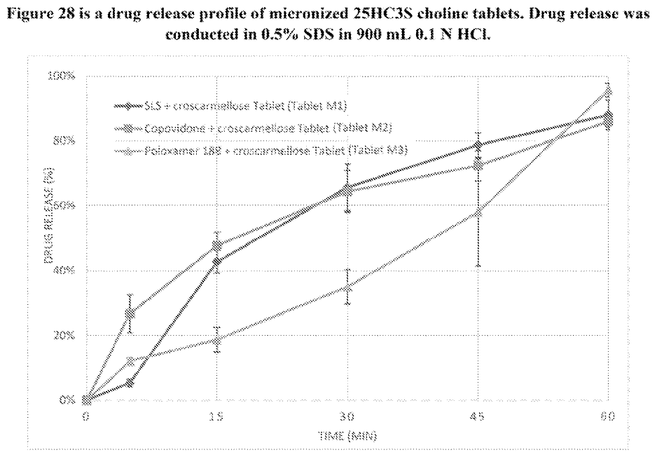

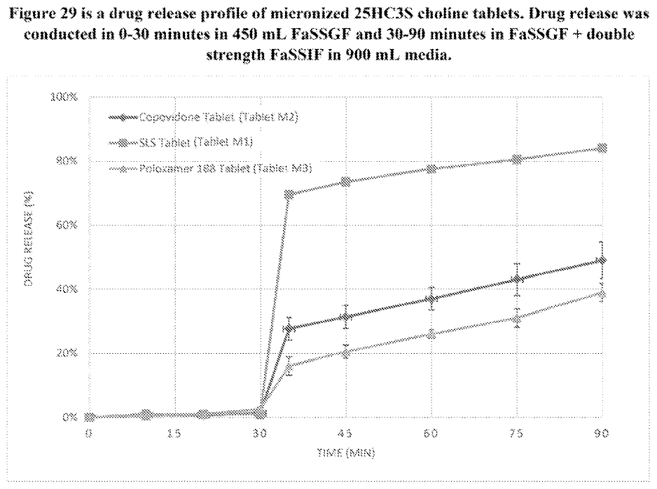

In Example 37, drug release profiles of tablets so manufactured are set forth in FIG. 28 and FIG. 29. Tablet M1 contained sodium lauryl sulfate as an intragranular excipient whereas Tablet M2 and Tablet M3 did not. Tablet M1 had a greater drug release in FIG. 29 in biorelevant media than either of Tablets M2 or M3.

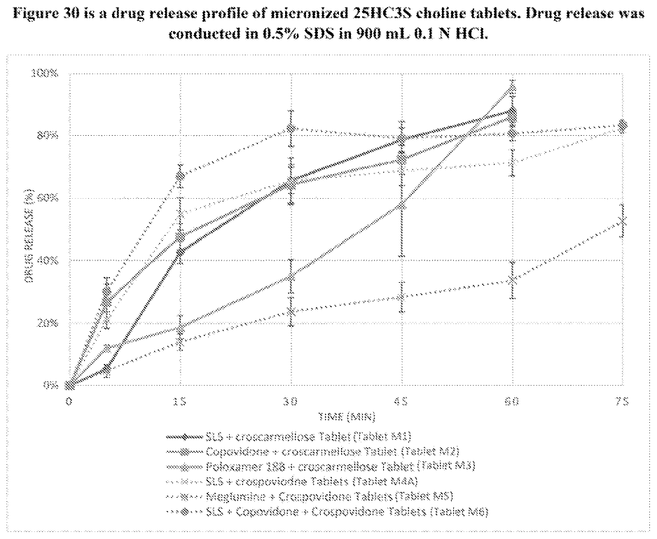

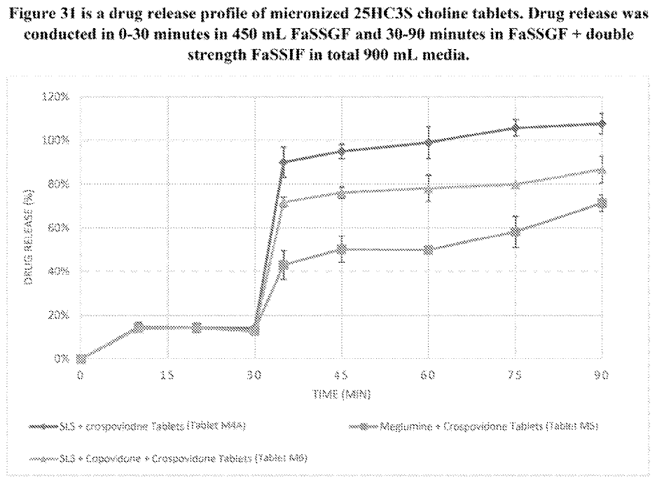

In Example 38, Tablet M4A, with crospovidone and sodium lauryl sulfate, had the greatest drug release as set forth in FIG. 31. FIG. 30 is release in 0.5% SDS and 0.1N HCl which does not have Tablet M4A data.

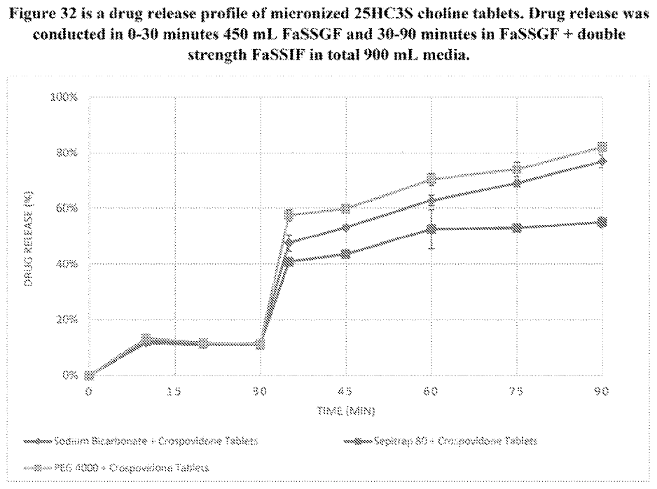

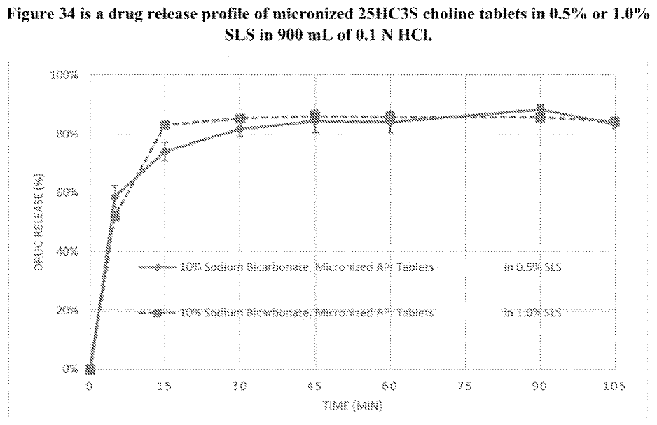

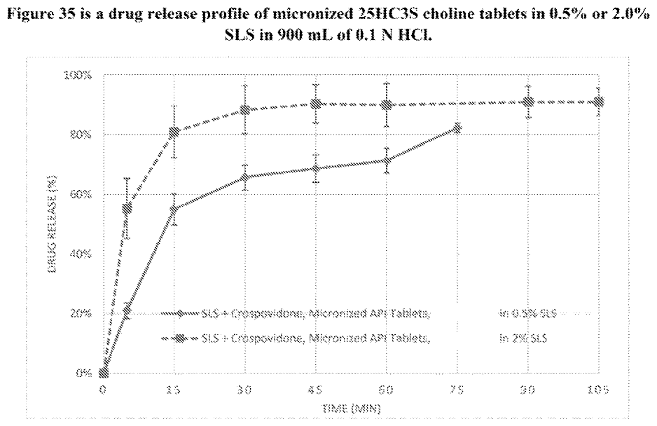

In Example 39, a batch of tablets was made having the ingredients set forth in Table D9. Various release profiles are set forth in FIGS. 32, 33, 34, and 35.

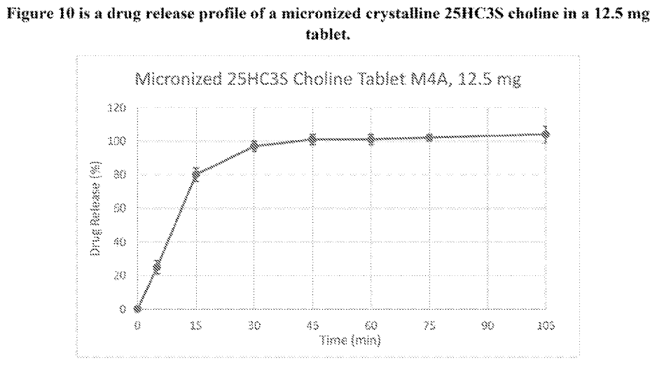

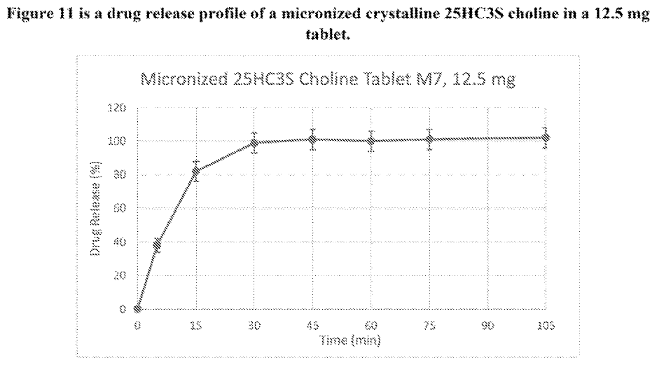

In additional embodiments of the disclosure, tablets made with the composition set forth in Table T1 below were measured for drug release using the parameters in Table T2 below with the results set forth in FIG. 10. Likewise, tablets made with the composition set forth in Table T3 below were measured for drug release using the parameters in Table T4 below with the results set forth in FIG. 11.

TABLE T1

| | | |
|---|---|---|
| Description | (% w/w) | mg/tablet |

Intra-Granular

| | | |
|---|---|---|
| 25HC3S Choline drug substance ¹ | 12.50 | 12.50 |
| Microcrystalline Cellulose | 52.10 | 52.10 |
| Lactose Monohydrate | 17.40 | 17.40 |
| Sodium Lauryl Sulfate | 5.00 | 5.00 |
| Crospovidone | 7.50 | 7.50 |
| Colloidal Silicon Dioxide | 1.00 | 1.00 |
| Magnesium Stearate | 0.50 | 0.50 |

Extra-Granular

| | | |
|---|---|---|
| Crospovidone | 2.50 | 2.50 |
| Colloidal Silicon Dioxide | 1.00 | 1.00 |
| Magnesium Stearate | 0.50 | 0.50 |
| Total | 100.00 | 100.00 |

¹ The target weight shown is the free acid form, this is adjusted for the choline salt form and purity of the lot of drug substance, a corresponding adjustment is made to the microcrystalline cellulose

TABLE T2

| Description | Parameters |
|---|---|
| Apparatus: | USP Apparatus II (Paddles) |
| Temperature: | 37.0° C. ± 0.5° C. |
| Medium: | 2.0% Sodium Lauryl Sulfate in 0.1 molar |
| | Hydrochloric Acid |
| Medium Volume: | 900 mL |
| Rotation Speed | 75 rpm (250 rpm from 75 minutes to |
| | 105 minutes) |
| Sampling | 5, 15, 30, 45, 60, 75, and 105 minutes |
| Timepoints: | |
| Sampling Volume: | 5 mL |

TABLE T3

| | | |
|---|---|---|
| Description | (% w/w) | mg/tablet |

Intra-Granular

| | | |
|---|---|---|
| 25HC3S Choline drug substance ¹ | 12.50 | 12.50 |
| Microcrystalline Cellulose | 48.40 | 48.40 |
| Lactose Monohydrate | 16.10 | 16.10 |
| Sodium Bicarbonate | 10.00 | 10.00 |
| Crospovidone | 7.50 | 7.50 |
| Colloidal Silicon Dioxide | 1.00 | 1.00 |
| Magnesium Stearate | 0.50 | 0.50 |

Extra-Granular

| | | |
|---|---|---|
| Crospovidone | 2.50 | 2.50 |
| Colloidal Silicon Dioxide | 1.00 | 1.00 |
| Magnesium Stearate | 0.50 | 0.50 |
| Total | 100.00 | 100.00 |

¹ The target weight shown is the free acid form, this is adjusted for the choline salt form and purity of the lot of drug substance, a corresponding adjustment is made to the microcrystalline cellulose

TABLE T4

| Description | Parameters |
|---|---|
| Apparatus: | USP Apparatus II (Paddles) |
| Temperature: | 37.0° C. ± 0.5° C. |
| Medium: | 2.0% Sodium Lauryl Sulfate in 0.1 |
| | molar Hydrochloric Acid |
| Medium Volume: | 900 mL |
| Rotation Speed | 75 rpm (250 rpm from 75 minutes to |
| | 105 minutes) |
| Sampling | 5, 15, 30, 45, 60, 75, and 105 minutes |
| Timepoints: | |
| Sampling Volume: | 5 mL |

Also included within the disclosure are mesophases of 25HC3S choline. As used herein, a mesophase is a liquid crystalline phase of a material.

The compositions comprising micronized 25HC3S choline and one or more pharmaceutically excipients may be configured such that 70% or more of 25HC3S choline is released within 15 minutes of (1) administration to a human; or (2) being placed in a USP Apparatus 2 having 900 mL of dissolution medium that is 2.0% sodium dodecyl sulfate in 0.1 N HCl with a paddle speed of 75 rpm and a vessel temperature of 37° C.; or (3) being placed in a USP Apparatus 2 having 450 mL FaSSGF with a paddle speed of 75 rpm and a vessel temperature of 37° C.; and/or 90% or more of release occurs within 30 minutes of (1) administration to a human; or (2) being placed in a USP Apparatus 2 having 900 mL of dissolution medium that is 2.0% sodium dodecyl sulfate in 0.1 N HCl with a paddle speed of 75 rpm and a vessel temperature of 37° C.; or (3) being placed in a USP Apparatus 2 having 450 mL FaSSGF with a paddle speed of 75 rpm and a vessel temperature of 37° C.; and/or not more than 40% is released within the first 10 minutes of (1) administration to a human; or (2) being placed in a USP Apparatus 2 having 900 mL of dissolution medium that is 2.0% sodium dodecyl sulfate in 0.1 N HCl with a paddle speed of 75 rpm and a vessel temperature of 37° C.; or (3) being placed in a USP Apparatus 2 having 450 mL FaSSGF with a paddle speed of 75 rpm and a vessel temperature of 37° C.

Also included within the scope of the disclosure are methods of treating disease with 25HC3S choline of the disclosure including pharmaceutical compositions of the disclosure comprising 25HC3S choline and one or more pharmaceutically acceptable excipients. Such diseases include of nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, alcoholic fatty liver disease (AFLD), alcoholic steatohepatitis (ASH), leptin resistance, leptin deficiency, a diabetes condition, an autoimmune condition, an inflammatory condition, a neurological condition, Epstein Barr virus-related growth, and conditions related to fat accumulation and inflammation. Examples of diabetes conditions that may be treated include one or more of insulin resistance, insulin insufficiency, diabetes, and prediabetes. Examples of autoimmune conditions that may be treated include one or more of hepatitis, multiple sclerosis, systemic lupus erythematosus, and rheumatoid arthritis. Examples of inflammatory conditions that may be treated include one or more of dental pulp inflammation, periodontal disease, skin inflammation, psoriasis, ulcerative colitis, osteoarthritis, inflammatory bowel disease (IBD), Crohn's disease, irritable bowel syndrome (IBS), Alzheimer's disease, Parkinson's disease, pancreatitis (acute and/or chronic), hepatitis (viral and/or non-viral), atherosclerosis, myocarditis, idiopathic pulmonary disorder (IPD), chronic obstructive pulmonary disorder (COPD), pneumonia, chronic inflammatory lung disease, bronchitis, asthma, chronic kidney disease (CKD), nephritis, sepsis, ankylosing spondylitis, diverticulitis, and fibromyalgia. Examples of neurological conditions that may be treated include one or more of depression, neurodegenerative disease, multiple sclerosis, Parkinson's disease, spinocerebellar degeneration, Friedreich ataxia, ataxia-telangiectasia, progressive supranuclear palsy, Huntington's disease, striatonigral degeneration, olivopontocerebellar atrophy, Shy-Drager syndrome, schizophrenia, schizoaffective disorder, manic-depression (bipolar) disorder, disturbed or abnormal circadian entrainment, childhood Alice in Wonderland syndrome, childhood acute cerebellar ataxia, and Alzheimer's disease. A skilled artisan would understand that the methods of treating diseases include treating one or more of the diseases disclosed herein. The 25HC3S choline of the disclosure may be amorphous, spray-dried, in a mesophase, or micronized and dosed compositions may be solid oral dosage forms such as tablets containing one or more pharmaceutically acceptable excipients.

### Methods for Preparing 25-hydroxy-cholesten-5-en-3-sulfate (25HC3S)

Various methods for preparing 25-hydroxy-cholesten-5-en-3-sulfate, such as 25-hydroxy-3β-cholesten-5-en-3-sulfate (25HC3S), are described herein. There are also other methods of making 25HC3S not described herein. Although many of the teachings herein involve a sulfate in the 3β position, the teachings of the present disclosure are also generally applicable to a sulfate in the 3a position. The components used in each step of the subject methods for preparing 25-hydroxy-3β-cholesten-5-en-3-sulfate described herein may be a purified composition or a crude composition as desired. The term “purified” is used in its conventional sense to refer to a composition where at least some isolation or purification process has been conducted, such as for example, filtration or aqueous workup of a reaction mixture. In certain instances, purification includes at least one of liquid chromatography, recrystallization, distillation (e.g., azeotropic distillation) and other type of compound purification. For example, compounds as described herein may be purified by chromatographic means, such as high performance liquid chromatography (HPLC), supercritical fluid chromatography (SFC), thin layer chromatography, flash column chromatography and ion exchange chromatography. Any suitable stationary phase can be used, including normal and reversed phases as well as ionic resins. Mobile phases may be chosen from polar solvents and non-polar solvents. In some cases, the mobile phase includes a polar solvent. In some cases, the polar solvent is chosen from chloroform, dichloromethane, tetrahydrofuran, dichloroethane, acetone, dioxane, ethyl acetate, dimethylsulfoxide, aniline, diethylamine, nitromethane, acetonitrile, pyridine, isopropanol, ethanol, methanol, ethylene glycol, acetic acid and water. In some cases, the mobile phase includes a non-polar solvent. In some cases, the non-polar solvent is chosen from diethyl ether, toluene, benzene, pentane, hexanes, cyclohexane, petroleum ether and carbon tetrachloride. See, e.g., Introduction to Modern Liquid Chromatography, 2nd Edition, ed. L. R. Snyder and J. J. Kirkland, John Wiley and Sons, 1979; and Thin Layer Chromatography, ed. E. Stahl, Springer-Verlag, New York, 1969.

In some cases, a reaction mixture is used in a subsequent step in the methods described herein as a crude mixture where no purification or other workup of the reaction mixture has been conducted. In certain instances, the crude mixture includes the compound of interest in sufficient purity such as where the reaction mixture includes the compound of interest in a purity of 70% or greater, such as 75% or greater, such as 80% or greater, such as 85% or greater, such as 90% or greater, such as 95% or greater, such as 97% or greater, such as 99% or greater, such as 99.5% or greater, such as 99.9% or greater, such as 99.99% or greater and including 99.999% or greater, relative to the crude reaction mixture (apart from solvent when present), as determined by chromatography (e.g., HPLC or SFC), nuclear magnetic resonance spectroscopy (e.g., ¹H NMR or ¹³C NMR) or a combination thereof. In some cases, the compound of interest is present in the reaction mixture in an amount that is 30 wt % or greater relative to the crude reaction mixture (apart from solvent when present), such as 40 wt % or greater, such as 50 wt % or greater, such as 60 wt % or greater, such as 70 wt % or greater, such as 75 wt % or greater, such as by 80 wt % or greater, such as 85 wt % or greater, such as 90 wt % or greater, such as 95 wt % or greater, such as 97 wt % or greater, such as 99 wt % or greater, such as 99.5 wt % or greater, such as 99.9 wt % or greater, such as 99.99 wt % or greater and including 99.999 wt % or greater relative to the crude reaction mixture, and may range from 5 wt % to 99.999 wt %, such as 30 wt % to 99.99 wt %, 40 wt % to 99.9 wt %, 50 wt % to 99 wt %, 70 wt % to 95 wt %, 75 wt % to 90 wt %, 80 wt % to 99 wt %, or 80 wt % to 95 wt %. In some cases, the compound of interest is present at 30 mol % or greater in the crude reaction mixture (apart from solvent when present), such as 40 mol % or greater, such as 50 mol % or greater, such as 60 mol % or greater, such as 70 mol % or greater, such as 75 mol % or greater, such as by 80 mol % or greater, such as 85 mol % or greater, such as 90 mol % or greater, such as 95 mol % or greater, such as 97 mol % or greater, such as 99 mol % or greater, such as 99.5 mol % or greater, such as 99.9 mol % or greater, such as 99.99 mol % or greater and including 99.999 mol % or greater relative to the crude reaction mixture, and may range from 30 mol % to 99.999 mol %, such as 50 mol % to 99 mol %, 70 mol % to 95 mol %, 75 mol % to 90 mol %, 80 mol % to 99 mol %, or 80 mol % to 95 mol %.

Methods for preparing a metal salt of 25-hydroxy-3β-cholesten-5-en-3-sulfate ([(3S,10R,13R,17R)-17-[(1R)-5-hydroxy-1,5-dimethyl-hexyl]-10,13-dimethyl-2,3,4,7,8,9,11,12,14,15,16,17-dodecahydro-1H-cyclopenta[a]phenanthren-3-yl]sulfate metal salt) according to the present disclosure include contacting 25-hydroxy-(3β)-cholest-5-en-3-ol with a sulfating agent to produce a 25-hydroxy-(3β)-cholest-5-en-3-sulfate organic cationic salt; and contacting the 25-hydroxy-(3β)-cholest-5-en-3-sulfate organic cationic salt with at least one metal salt to produce the 5-cholesten-3β,25-diol 3-sulfate metal salt can be achieved such as in (Scheme Ia).

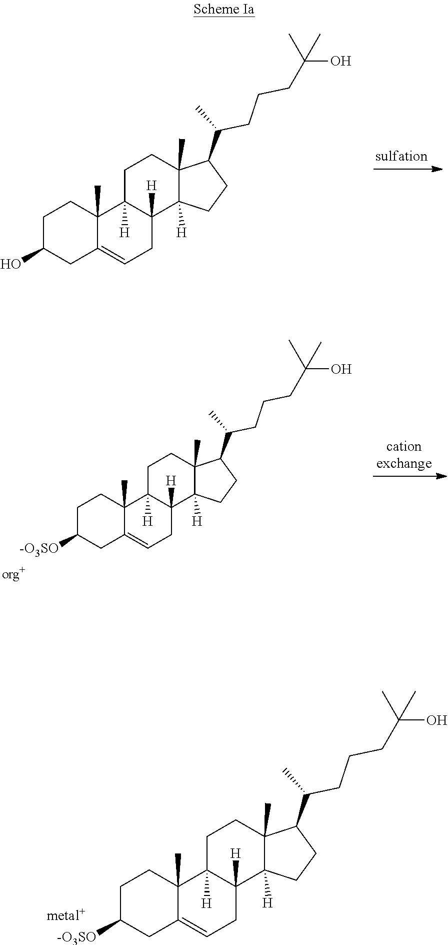

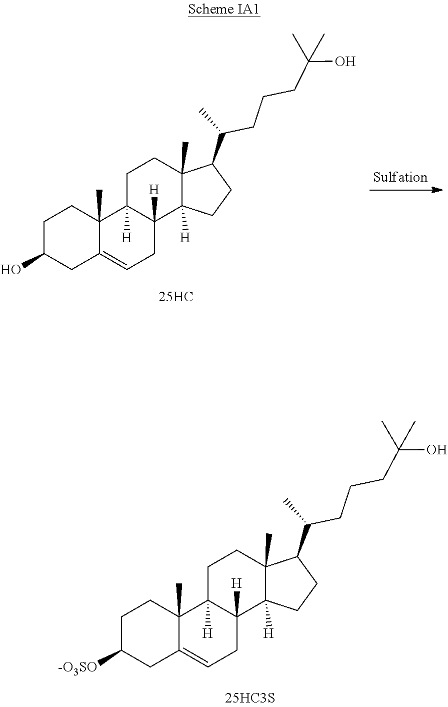

The 25-hydroxy-(3β)-cholest-5-en-3-ol may be sulfated by contacting with a sulfating agent (Scheme IA1). In some cases, the sulfating reagent is chosen from sulfur trioxide complexes, sulfuric acid compounds, sulfonic acid compounds, and sulfonate compounds. In some cases, the sulfating reagent is chosen from sulfur trioxide dimethyl formamide, sulfur trioxide triethylamine, and sulfur trioxide trimethylamine. In some cases, the sulfating reagent includes from sulfuric acid and acetic anhydride and pyridine. In some cases, the sulfating reagent includes sulfur trioxide triethylamine and pyridine. In some cases, the sulfating reagent is chosen from 1) chlorosulfonic acid and pyridine and 2) chlorosulfonic acid and 2,6-lutidine. In some cases, the sulfating reagent is ethyl chlorosulfonate.

The 25-hydroxy-(3β)-cholest-5-en-3-ol may be sulfated at a temperature that ranges from −10° C. to 50° C., such as from −5° C. to 45° C., such as from −4° C. to 40° C., such as from −3° C. to 35° C., such as from −2° C. to 30° C., such as from −1° C. to 25° C., and including from 0° C. to 20° C. The reaction may be carried out for a duration that ranges from 0.1 hours to 72 hours, such as from 0.2 hours to 48 hours, such as from 0.3 hours to 24 hours, such as from 0.4 hours to 21 hours, such as from 0.5 hours to 20 hours, such as from 0.6 hours to 19 hours, such as from 0.7 hours to 18 hours, such as from 0.8 hours to 17 hours, such as from 0.9 hours to 16 hours and including from 1 hour to 15 hours. The amount of sulfating agent used relative to the 25-hydroxy-(3β)-cholest-5-en-3-ol may vary and may be 0.001 equivalents or more, such as 0.01 equivalents or more, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more, and including 10 equivalents or more, and may range from 0.001 equivalents to 10 equivalents, such as 0.1 equivalents to 10 equivalents, 0.1 equivalents to 8 equivalents, 0.1 equivalents to 5 equivalents, 0.5 equivalents to 10 equivalents, 0.5 equivalents to 8 equivalents, 0.5 equivalents to 5 equivalents, 0.9 equivalents to 10 equivalents, 0.9 equivalents to 8 equivalents, 0.9 equivalents to 5 equivalents, 1.3 equivalents to 10 equivalents, 1.3 equivalents to 8 equivalents, 1.3 equivalents to 5 equivalents, 1.5 equivalents to 10 equivalents, 1.5 equivalents to 8 equivalents, 1.5 equivalents to 5 equivalents, 2 equivalents to 10 equivalents, 2 equivalents to 8 equivalents, 2 equivalents to 5 equivalents, or 1 equivalent to 2 equivalents, 1 equivalents to 1.5 equivalents, or 1.1 to 1.2 equivalents, relative to the 25-hydroxy-(3β)-cholest-5-en-3-ol.

In some cases, methods include sulfating the 25-hydroxy-(3β)-cholest-5-en-3-ol in at least one solvent where the 25-hydroxy-(3β)-cholest-5-en-3-sulfate product exhibits low solubility. In some cases, the 25-hydroxy-(3β)-cholest-5-en-3-ol is sulfated in at least one solvent where the 25-hydroxy-(3β)-cholest-5-en-3-sulfate product exhibits a solubility of 100 mmol/L or less, such as 90 mmol/L or less, such as 80 mmol/L or less, such as 70 mmol/L or less, such as 60 mmol/L or less, such as 50 mmol/L or less, such as 40 mmol/L or less, such as 30 mmol/L or less, such as 20 mmol/L or less, such as 10 mmol/L or less, and including sulfating the 25-hydroxy-(3β)-cholest-5-en-3-ol in at least one solvent where the 25-hydroxy-(3β)-cholest-5-en-3-sulfate product exhibits a solubility of 5 mmol/L or less. In some cases, the 25-hydroxy-(3β)-cholest-5-en-3-ol is sulfated in at least one solvent where 25-hydroxy-(3β)-cholest-5-en-3-sulfate product precipitates after formation. In some cases, the at least one solvent is chosen from chloroform, methylene chloride, acetone, acetonitrile, toluene, tetrahydrofuran, and methyltetrahydrofuran.

In some cases, methods include sulfating the 25-hydroxy-(3β)-cholest-5-en-3-ol in a manner sufficient to reduce or eliminate bis-sulfation of the 25-hydroxy-(3β)-cholest-5-en-3-ol. In some instances, the 25-hydroxy-(3β)-cholest-5-en-3-ol is sulfated and a bis-sulfate product (i.e., 5-cholesten-3β-25-diol-disulfate, Structure IA) is formed in an amount that is 10 wt % or less of the reaction product formed by contacting the 25-hydroxy-(3β)-cholest-5-en-3-ol with the sulfating agent, such as 9 wt % or less, such as 8 wt % or less, such as 7 wt % or less, such as 6 wt % or less, such as 5 wt % or less, such as 4 wt % or less, such as 3 wt % or less, such as 2 wt % or less, such as 1 wt % or less, such as 0.5 wt % or less, such as 0.1 wt % or less, such as 0.01 wt % or less, such as 0.001 wt % or less, and including where the 25-hydroxy-(3β)-cholest-5-en-3-ol is sulfated and the bis-sulfate product is formed in an amount that is 0.0001 wt % or less, and may range from 10 wt % to 0.001 wt %, such as 10 wt % to 0.1 wt %, 10 wt % to 1 wt %, 10 wt % to 2 wt %, 8 wt % to 0.001 wt %, 8 wt % to 0.1 wt %, 8 wt % to 1 wt %, 8 wt % to 2 wt %, 6 wt % to 0.001 wt %, 6 wt % to 0.1 wt %, 6 wt % to 1 wt %, 6 wt % to 2 wt %, 4 wt % to 0.001 wt %, 4 wt % to 0.1 wt %, 4 wt % to 1 wt %, 4 wt % to 2 wt %, 3 wt % to 0.001 wt %, 3 wt % to 0.1 wt %, 3 wt % to 1 wt %, 2 wt % to 0.001 wt %, 2 wt % to 0.1 wt %, or 2 wt % to 1 wt %.

In some cases, the ratio by weight of the 25-hydroxy-(3β)-cholest-5-en-3-sulfate to the 5-cholesten-3β-25-diol-disulfate formed is 10:1 or more, such as 25:1 or more, such as 50:1 or more, such as 100:1 or more, such as such as 250:1 or more, such as 500:1 or more, such as 1000:1 or more, such as 2500:1 or more, such as 5000:1 or more, such as 10,000:1 or more, such as 25,000:1 or more, such as 50,000:1 or more, such as 100,000:1 or more, such as 10⁶:1 or more, such as 10⁷:1 or more, such as 10⁸:1 or more, and including where the ratio by weight of the 25-hydroxy-(3β)-cholest-5-en-3-sulfate to the 5-cholesten-3β-25-diol-disulfate formed is 10⁹:1 or more, and may range from a ratio by weight of 10:1 to a ratio by weight of 10⁹:1, such as a ratio of weight of 10:1 to a ratio of weight of 10⁶:1, a ratio of weight of 10:1 to a ratio of weight of 10³:1, a ratio of weight of 10:1 to a ratio of weight of 100:1, a ratio of weight of 100:1 to a ratio of weight of 10⁹:1, a ratio of weight of 100:1 to a ratio of weight of 10⁶:1, a ratio of weight of 100:1 to a ratio of weight of 103:1, a ratio of weight of 250:1 to a ratio of weight of 10⁹:1, a ratio of weight of 250:1 to a ratio of weight of 10⁶:1, a ratio of weight of 250:1 to a ratio of weight of 10³:1, a ratio of weight of 500:1 to a ratio of weight of 10⁹:1, a ratio of weight of 500:1 to a ratio of weight of 10⁶:1, a ratio of weight of 500:1 to a ratio of weight of 10³:1, a ratio of weight of 10³:1 to a ratio of weight of 10⁹:1, a ratio of weight of 10³:1 to a ratio of weight of 10⁶:1, or a ratio of weight of 250:1 to a ratio of weight of 10³:1.

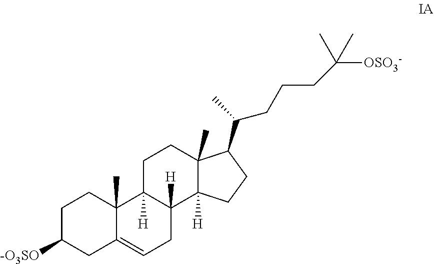

In some cases, the 5-cholesten-3β-25-diol-disulfate formed when sulfating 25-hydroxy-(3β)-cholest-5-en-3-ol remains solubilized in the at least one solvent. In some cases, the 5-cholesten-3β-25-diol-disulfate has high solubility in the at least one solvent. In some instances, the 5-cholesten-3β-25-diol-disulfate exhibits a solubility of 500 mmol/L or more in the at least one solvent, such as 600 mmol/L or more, such as 700 mmol/L or more, such as 800 mmol/L or more, such as 900 mmol/L, or more and including a solubility of 1 mol/L or more in the at least one solvent.

In certain cases, methods further include separating the 25-hydroxy-(3β)-cholest-5-en-3-sulfate product from the bis-sulfate product (i.e., 5-cholesten-3β-25-diol-disulfate). In some cases, the 25-hydroxy-(3β)-cholest-5-en-3-sulfate product is separated from the bis-sulfate product by vacuum filtration. In some cases, the 25-hydroxy-(3β)-cholest-5-en-3-sulfate product is separated from the bis-sulfate product by recrystallization of the 25-hydroxy-(3β)-cholest-5-en-3-sulfate product. In some cases, the 25-hydroxy-(3β)-cholest-5-en-3-sulfate product is separated from the bis-sulfate product by chromatography (e.g., silica column).

In some cases, the 25-hydroxy-(3β)-cholest-5-en-3-ol is sulfated in a reaction mixture having a pH that ranges from 5.0 to 8.0, such as a pH from 5.1 to 7.9, such as a pH from 5.2 to 7.8, such as a pH from 5.3 to 7.7, such as a pH from 5.4 to 7.6, such as a pH from 5.5 to 7.5, such as a pH from 5.6 to 7.4, such as a pH from 5.7 to 7.3, such as a pH from 5.8 to 7.2, such as a pH from 5.9 to 7.1, and including sulfating the 25-hydroxy-(3β)-cholest-5-en-3-ol in a reaction mixture having a pH of from 6.0 to 7.0.

In some cases, 25-hydroxy-(3β)-cholest-5-en-3-ol is sulfated in the presence of a 25-hydroxy-(3β)-cholest-5-en-3-sulfate organic cationic salt. In certain cases, the 25-hydroxy-(3β)-cholest-5-en-3-sulfate organic cationic salt is present as particles (e.g., seed crystals of 25-hydroxy-(3β)-cholest-5-en-3-sulfate organic cationic salt produced in a previous reaction or purified reaction batch). In some cases, sulfating 25-hydroxy-(3β)-cholest-5-en-3-ol in the presence of 25-hydroxy-(3β)-cholest-5-en-3-sulfate organic cationic salt (e.g., as particles) is sufficient to reduce the solubility of 25-hydroxy-(3β)-cholest-5-en-3-sulfate organic cationic salt produced by reaction of the sulfating agent with 25-hydroxy-(3β)-cholest-5-en-3-ol as compared to the solubility when the 25-hydroxy-(3β)-cholest-5-en-3-sulfate organic cationic salt is not present. In certain cases, the solubility of 25-hydroxy-(3β)-cholest-5-en-3-sulfate organic cationic salt produced in the reaction mixture is reduced as compared to the solubility when the added 25-hydroxy-(3β)-cholest-5-en-3-sulfate organic cationic salt is not present by 5% or more, such as by 10% or more, such as by 25% or more, such as by 50% or more, such as by 75% or more, such as by 90% or more and including by reducing the solubility of the produced 25-hydroxy-(3β)-cholest-5-en-3-sulfate organic cationic salt by 99% or more. The size of the particles of 25-hydroxy-(3β)-cholest-5-en-3-sulfate organic cationic salt added to the reaction mixture may vary and may have a dimension (e.g., length, width or diameter) of 0.01 mm or more, such as 0.025 mm or more, such as 0.05 mm or more, such as 0.075 mm or more, such as 0.1 mm or more, such as 0.25 mm or more, such as 0.5 mm or more, such as 0.75 mm or more, such as 1 mm or more, such as 2 mm or more, such as 3 mm or more, such as 4 mm or more and including 5 mm or more. In some cases, the particles of 25-hydroxy-(3β)-cholest-5-en-3-sulfate organic cationic salt are added to the reaction mixture immediately after contacting the sulfating agent with the 25-hydroxy-(3β)-cholest-5-en-3-ol. In some cases, the particles of 25-hydroxy-(3β)-cholest-5-en-3-sulfate organic cationic salt are added to the reaction mixture 1 minute or more after contacting the sulfating agent with the 25-hydroxy-(3β)-cholest-5-en-3-ol, such as 5 minutes or more, such as 10 minutes or more, such as 15 minutes or more, such as 20 minutes or more, such as 30 minutes or more, such as 40 minutes or more, such as 50 minutes or more and including adding the particles of 25-hydroxy-(3β)-cholest-5-en-3-sulfate organic cationic salt to the reaction mixture 60 minutes or more after contacting the sulfating agent with the 25-hydroxy-(3β)-cholest-5-en-3-ol.

In certain cases, the sulfating agent is characterized prior to contacting with the 25-hydroxy-(3β)-cholest-5-en-3-ol. In some cases, characterizing the sulfating agent includes determining the extent of degradation of the sulfating agent prior to contacting with the 25-hydroxy-(3β)-cholest-5-en-3-ol. In certain cases, determining the extent of degradation of the sulfating reagent includes determining the amount of impurity in the sulfating reagent prior to contacting with the 25-hydroxy-(3β)-cholest-5-en-3-ol.

In some instances, the degradation of the sulfating agent is determined by proton nuclear magnetic resonance spectroscopy (¹H-NMR). Proton NMR spectroscopy of the sulfating agent may be conducted in at least one deuterated solvent. In certain cases, the at least one deuterated solvent is deuterated acetone ((CD₃)₂CO). In certain cases, the at least one deuterated solvent is not deuterated benzene (C₆D₆). In certain cases, the at least one deuterated solvent is not deuterated acetonitrile (CD₃CN). In certain cases, the at least one deuterated solvent is not deuterated chloroform (CD₃Cl).

In some instances, methods for determining the extent of degradation include integrating one or more peaks in the ¹H-NMR spectrum at a chemical shift of from 9.2 ppm to 9.3 ppm and calculating the impurity level of the sulfating agent based on the integrated peaks. In certain instances, methods for determining the extent of degradation include integrating one or more peaks in the ¹H-NMR spectrum at a chemical shift of about 9.25 ppm and calculating the impurity level of the sulfating agent based on the integrated peaks. In some cases, the sulfating agent is contacted with the 25-hydroxy-(3β)-cholest-5-en-3-ol when the impurity level of the sulfating agent is below a predetermined threshold, such as where the impurity level is 25% or less as determined by integrating one or more peaks in the proton NMR spectrum at a chemical shift of from 9.2 ppm to 9.3 ppm, such as 24% or less, such as 23% or less, such as 22% or less, such as 21% or less, such as 20% or less, such as 19% or less, such as 18% or less, such as 17% or less, such as 16% or less, such as 15% or less, such as 14% or less such as 13% or less, such as 12% or less, such as 11% or less, such as 10% or less, such as 9% or less, such as 8% or less, such as 7% or less, such as 6% or less, such as 5% or less, such as 4% or less, such as 3% or less such as 2% or less and including where the impurity level is 1% or less as determined by integrating one or more peaks in the proton NMR spectrum at a chemical shift of from 9.2 ppm to 9.3 ppm. In some cases, the sulfating agent is not contacted with the 25-hydroxy-(3β)-cholest-5-en-3-ol when the impurity level is above a predetermined threshold, such as where the impurity level is 25% or more as determined by integrating one or more peaks in the proton NMR spectrum at a chemical shift of from 9.2 ppm to 9.3 ppm, such as 26% or more, such as 27% or more, such as 28% or more, such as 29% or more, such as 30% or more, such as 31% or more, such as 32% or more, such as 33% or more, such as 34% or more and including where the impurity level is 35% or more as determined by integrating one or more peaks in the proton NMR spectrum at a chemical shift of from 9.2 ppm to 9.3 ppm.

In certain cases, the generated 25-hydroxy-(3β)-cholest-5-en-3-sulfate product includes one or more byproducts. In some cases, the byproduct is 5-cholesten-3β-25-diol-disulfate. In some cases, 5-cholesten-3β-25-diol-disulfate byproduct is present in the composition produced by sulfation of 25-hydroxy-(3β)-cholest-5-en-3-ol in an amount relative to the 25-hydroxy-(3β)-cholest-5-en-3-sulfate of 10 wt % or less, such as 9 wt % or less, such as 8 wt % or less, such as 7 wt % or less, such as 6 wt % or less, such as 5 wt % or less, such as 4 wt % or less, such as 3 wt % or less, such as 2 wt % or less, such as 1 wt % or less, such as 0.5 wt % or less, such as 0.1 wt % or less, such as 0.01 wt % or less, such as 0.001 wt % or less, and including where 5-cholesten-3β-25-diol-disulfate byproduct is present in the composition produced by sulfation of 25-hydroxy-(3β)-cholest-5-en-3-ol in an amount of 0.001 wt % or less, and may range from 0.1 wt % to 50 wt %, such as 0.5 wt % to 20 wt % or 1 wt % to 12 wt %. In some cases, the ratio by weight of the 25-hydroxy-(3β)-cholest-5-en-3-sulfate to the 5-cholesten-30-25-diol-disulfate byproduct formed is 10:1 or more, such as 25:1 or more, such as 50:1 or more, such as 100:1 or more, such as such as 250:1 or more, such as 500:1 or more, such as 1000:1 or more, such as 2500:1 or more, such as 5000:1 or more, such as 10,000:1 or more, such as 25,000:1 or more, such as 50,000:1 or more, such as 100,000:1 or more, such as 10⁶:1 or more, such as 10⁷:1 or more, such as 10⁸:1 or more, and including where the ratio by weight of the 25-hydroxy-(3β)-cholest-5-en-3-sulfate to the 5-cholesten-3β-25-diol-disulfate formed is 10⁹:1 or more. In some cases, the ratio by weight of the 25-hydroxy-(3β)-cholest-5-en-3-sulfate and the 5-cholesten-3β-25-diol-disulfate formed ranges from 10:1 to 10⁹:1, such as from 100:1 to 10⁸:1, such as from 1000:1 to 10⁷:1, and including from 10000:1 to 10⁶:1.

Aspects of the present disclosure also include compositions having 25-hydroxy-(3β)-cholest-5-en-3-sulfate and 5-cholesten-3β-25-diol-disulfate that is present in the composition in an amount relative to the 25-hydroxy-(3β)-cholest-5-en-3-sulfate of 10 wt % or less, such as 9 wt % or less, such as 8 wt % or less, such as 7 wt % or less, such as 6 wt % or less, such as 5 wt % or less, such as 4 wt % or less, such as 3 wt % or less, such as 2 wt % or less, such as 1 wt % or less, such as 0.5 wt % or less, such as 0.1 wt % or less, such as 0.01 wt % or less, such as 0.001 wt % or less, and including 0.001 wt % or less, and may range from 10 wt % to 0.001 wt %, such as 10 wt % to 0.1 wt %, 10 wt % to 1 wt %, 10 wt % to 2 wt %, 8 wt % to 0.001 wt %, 8 wt % to 0.1 wt %, 8 wt % to 1 wt %, 8 wt % to 2 wt %, 6 wt % to 0.001 wt %, 6 wt % to 0.1 wt %, 6 wt % to 1 wt %, 6 wt % to 2 wt %, 4 wt % to 0.001 wt %, 4 wt % to 0.1 wt %, 4 wt % to 1 wt %, 4 wt % to 2 wt %, 3 wt % to 0.001 wt %, 3 wt % to 0.1 wt %, 3 wt % to 1 wt %, 2 wt % to 0.001 wt %, 2 wt % to 0.1 wt %, or 2 wt % to 1 wt %.

In some cases, compositions include a ratio by weight of the 25-hydroxy-(3β)-cholest-5-en-3-sulfate and the 5-cholesten-3β-25-diol-disulfate of 10:1 or more, such as 25:1 or more, such as 50:1 or more, such as 100:1 or more, such as such as 250:1 or more, such as 500:1 or more, such as 1000:1 or more, such as 2500:1 or more, such as 5000:1 or more, such as 10,000:1 or more, such as 25,000:1 or more, such as 50,000:1 or more, such as 100,000:1 or more, such as 10⁶:1 or more, such as 10⁷:1 or more, such as 10⁸:1 or more, and including where the ratio by weight of the 25-hydroxy-(3β)-cholest-5-en-3-sulfate to the 5-cholesten-3β-25-diol-disulfate in the composition is 10⁹:1 or more. In some cases, compositions include a ratio by weight of the 25-hydroxy-(3β)-cholest-5-en-3-sulfate and the 5-cholesten-3β-25-diol-disulfate that ranges from 10:1 to 10⁹:1, such as from 100:1 to 10⁸:1, such as from 1000:1 to 10⁷:1, and including from 10000:1 to 10⁶:1.

In some cases, the byproduct is sulfated desmosterol (Structure IB).

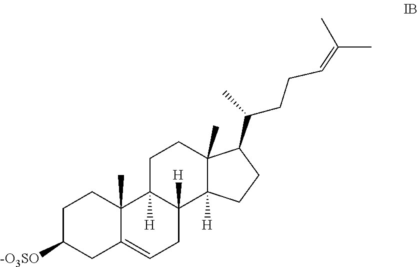

In some cases, sulfated desmosterol ([(3S,8S,9S,10R,13R,14S,17R)-17-[(1R)-1,5-dimethylhex-4-enyl]-10,13-dimethyl-2,3,4,7,8,9,11,12,14,15,16,17-dodecahydro-1H-cyclopenta[a]phenanthren-3-yl]sulfate) is present in the composition produced by sulfation of 25-hydroxy-(3β)-cholest-5-en-3-ol in an amount relative to the 25-hydroxy-(3β)-cholest-5-en-3-sulfate of 10 wt % or less, such as 9 wt % or less, such as 8 wt % or less, such as 7 wt % or less, such as 6 wt % or less, such as 5 wt % or less, such as 4 wt % or less, such as 3 wt % or less, such as 2 wt % or less, such as 1 wt % or less, such as 0.5 wt % or less, such as 0.1 wt % or less, such as 0.01 wt % or less, such as 0.001 wt % or less, and including where sulfated desmosterol is present in the composition produced by sulfation of 25-hydroxy-(3β)-cholest-5-en-3-ol in an amount relative to the 25-hydroxy-(3β)-cholest-5-en-3-sulfate of 0.001 wt % or less, and may range from 0.1 wt % to 10 wt %, such as 0.2 wt % to 5 wt % or 0.3 wt % to 3 wt %. In some cases, the ratio by weight of the 25-hydroxy-(3β)-cholest-5-en-3-sulfate to the sulfated desmosterol formed is 10:1 or more, such as 25:1 or more, such as 50:1 or more, such as 100:1 or more, such as such as 250:1 or more, such as 500:1 or more, such as 1000:1 or more, such as 2500:1 or more, such as 5000:1 or more, such as 10,000:1 or more, such as 25,000:1 or more, such as 50,000:1 or more, such as 100,000:1 or more, such as 10⁶:1 or more, such as 10⁷:1 or more, such as 10⁸:1 or more, and including where the ratio by weight of the 25-hydroxy-(3β)-cholest-5-en-3-sulfate to the sulfated desmosterol formed is 109.1 or more. In some cases, the ratio by weight of the 25-hydroxy-(3β)-cholest-5-en-3-sulfate and the sulfated desmosterol formed ranges from 10:1 to 10⁹:1, such as from 100:1 to 10⁸:1, such as from 1000:1 to 10⁷:1 and including from 10000:1 to 10⁶:1.

Aspects of the present disclosure also include compositions having 25-hydroxy-(3β)-cholest-5-en-3-sulfate and sulfated desmosterol that is present in the composition in an amount relative to the 25-hydroxy-(3β)-cholest-5-en-3-sulfate of 10 wt % or less, such as 9 wt % or less, such as 8 wt % or less, such as 7 wt % or less, such as 6 wt % or less, such as 5 wt % or less, such as 4 wt % or less, such as 3 wt % or less, such as 2 wt % or less, such as 1 wt % or less, such as 0.5 wt % or less, such as 0.1 wt % or less, such as 0.01 wt % or less, such as 0.001 wt % or less, and including 0.001% w/w or less relative to the 25-hydroxy-(3β)-cholest-5-en-3-sulfate, and may range from 10 wt % to 0.001 wt %, such as 10 wt % to 0.1 wt %, 10 wt % to 1 wt %, 10 wt % to 2 wt %, 8 wt % to 0.001 wt %, 8 wt % to 0.1 wt %, 8 wt % to 1 wt %, 8 wt % to 2 wt %, 6 wt % to 0.001 wt %, 6 wt % to 0.1 wt %, 6 wt % to 1 wt %, 6 wt % to 2 wt %, 4 wt % to 0.001 wt %, 4 wt % to 0.1 wt %, 4 wt % to 1 wt %, 4 wt % to 2 wt %, 3 wt % to 0.001 wt %, 3 wt % to 0.1 wt %, 3 wt % to 1 wt %, 2 wt % to 0.001 wt %, 2 wt % to 0.1 wt %, or 2 wt % to 1 wt %.

In some cases, compositions include a ratio by weight of the 25-hydroxy-(3β)-cholest-5-en-3-sulfate and the sulfated desmosterol of 10:1 or more, such as 25:1 or more, such as 50:1 or more, such as 100:1 or more, such as such as 250:1 or more, such as 500:1 or more, such as 1000:1 or more, such as 2500:1 or more, such as 5000:1 or more, such as 10,000:1 or more, such as 25,000:1 or more, such as 50,000:1 or more, such as 100,000:1 or more, such as 10⁶:1 or more, such as 10⁷:1 or more, such as 10⁸:1 or more, and including where the ratio by weight of the 25-hydroxy-(3β)-cholest-5-en-3-sulfate to the sulfated desmosterol in the composition is 10⁹:1 or more. In some cases, compositions include a ratio by weight of the 25-hydroxy-(3β)-cholest-5-en-3-sulfate and the sulfated desmosterol that ranges from 10:1 to 10⁹:1, such as from 100:1 to 10⁸:1, such as from 1000:1 to 10⁷:1 and including from 10000:1 to 10⁶:1.

In some cases, the byproduct of sulfating the 25-hydroxy-(3β)-cholest-5-en-3-ol that is present in the 25-hydroxy-(3β)-cholest-5-en-3-sulfate composition is a thermal degradation product. In some cases, the byproduct is identified by relative retention time when the components of the 25-hydroxy-(3β)-cholest-5-en-3-sulfate composition are separated by liquid chromatography (e.g., HPLC). In certain cases, the byproduct is sulfated desmosterol, a compound having a retention time of about 18.3 minutes when the components of the 25-hydroxy-(3β)-cholest-5-en-3-sulfate composition are separated by HPLC operating at about 45° C. with a C8 stationary phase and separates the components of the composition with a first mobile phase comprising a buffer (e.g., an aqueous buffer of sodium phosphate) and a second mobile phase comprising one or more organic solvents (see e.g., Tables 3 and 4 below). In some cases, the first mobile phase is an aqueous buffer. In certain cases, the first mobile phase includes sodium phosphate. In some cases, the second mobile phase is chosen from one or more of methoxypropyl acetate, acetonitrile and methanol. In some cases, the flow rate of the first mobile phase is about 1.0 mL/minute. In some cases, the flow rate of the second mobile phase is about 1.0 mL/minute or more. In some cases, 25-hydroxy-(3β)-cholest-5-en-3-sulfate has a retention time of about 7.7 minutes under the same HPLC conditions. In some cases, the byproduct is a compound having a retention time of about 37.7 minutes when the components of the 25-hydroxy-(3β)-cholest-5-en-3-sulfate composition are separated by HPLC operating at about 45° C. with a C8 stationary phase and separates the components of the composition with a first mobile phase comprising a buffer (e.g., an aqueous buffer of sodium phosphate) and a second mobile phase comprising one or more organic solvents (see e.g., Tables 3 and 4 below). While not wishing to be bound by theory, it is believed that the compound having a retention time of about 37.7 minutes is desmosterol. In some cases, the first mobile phase is an aqueous buffer. In certain cases, the first mobile phase includes sodium phosphate. In some cases, the second mobile phase is chosen from one or more of methoxypropyl acetate, acetonitrile and methanol. In some cases, the flow rate of the first mobile phase is about 1.0 mL/minute. In some cases, the flow rate of the second mobile phase is about 1.0 mL/minute or more. In some cases, and 25-hydroxy-(3β)-cholest-5-en-3-sulfate has a retention time of about 7.7 minutes under the same HPLC conditions such that sulfated desmosterol has a relative retention time of about 2.4 (=18.3/7.7) and the compound believed to be desmosterol has a relative retention time of about 4.9 (=37.7/7.7).

Aspects of the present disclosure also include compositions having 25-hydroxy-(3β)-cholest-5-en-3-sulfate and one or more byproducts of sulfating the 25-hydroxy-(3β)-cholest-5-en-3-ol. In some cases, the one or more byproducts are present in the composition in an amount relative to the 25-hydroxy-(3β)-cholest-5-en-3-sulfate of 10 wt % or less, such as 9 wt % or less, such as 8 wt % or less, such as 7 wt % or less, such as 6 wt % or less, such as 5 wt % or less, such as 4 wt % or less, such as 3 wt % or less, such as 2 wt % or less, such as 1 wt % or less, such as 0.5 wt % or less, such as 0.1 wt % or less, such as 0.01 wt % or less, such as 0.001 wt % or less, and including 0.001 wt % or less, and may range from 0.1 wt % to 5 wt %, such as 0.2 wt % to 10 wt % or 0.3 wt % to 15 wt %. In some cases, compositions include 25-hydroxy-(3β)-cholest-5-en-3-sulfate and the one or more byproducts in an amount relative to the 25-hydroxy-(3β)-cholest-5-en-3-sulfate that ranges from 0.0001 wt % to 10 wt %, such as from 0.005 wt % to 9.5 wt %, such as from 0.001% to 9.0 wt %, such as from 0.05 wt % to 8.5 wt %, such as from 0.1 wt % to 8.0 wt %, such as from 0.5 wt % to 7.5 wt %, such as from 1 wt % to 7 wt %, such as from 1.5 wt % to 6.5 wt %, and including from 2 wt % to 6 wt %.

In some cases, the ratio by weight of the 25-hydroxy-(3β)-cholest-5-en-3-sulfate to the one or more byproducts formed is 10:1 or more, such as 25:1 or more, such as 50:1 or more, such as 100:1 or more, such as such as 250:1 or more, such as 500:1 or more, such as 1000:1 or more, such as 2500:1 or more, such as 5000:1 or more, such as 10,000:1 or more, such as 25,000:1 or more, such as 50,000:1 or more, such as 100,000:1 or more, such as 10⁶:1 or more, such as 10⁷:1 or more, such as 10⁸:1 or more, and including where the ratio by weight of the 25-hydroxy-(3β)-cholest-5-en-3-sulfate to the one or more byproducts formed is 10⁹:1 or more. In some cases, the ratio by weight of the 25-hydroxy-(3β)-cholest-5-en-3-sulfate and the one or more byproducts formed ranges from 10:1 to 10⁹:1, such as from 100:1 to 10⁸:1, such as from 1000:1 to 10⁷:1, and including from 10000:1 to 10⁶:1.

In some cases, the 25-hydroxy-(3β)-cholest-5-en-3-sulfate organic cationic salt is a 25-hydroxy-(3β)-cholest-5-en-3-sulfate pyridinium salt (Scheme IA2).

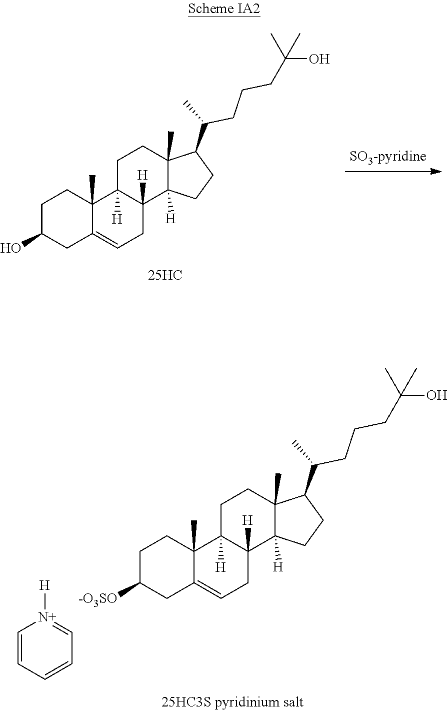

In certain cases, the sulfating agent is contacted with an anhydride prior to contacting with the 25-hydroxy-(3β)-cholest-5-en-3-ol. In some cases, the anhydride is chosen from acetic anhydride, trifluoroacetic anhydride and triflic anhydride. The amount of anhydride relative to the 25-hydroxy-(3β)-cholest-5-en-3-ol may vary and may be 0.001 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more, and including 10 equivalents or more, and may range from 0.001 equivalents to 10 equivalents, such as 0.1 equivalents to 10 equivalents, 0.1 equivalents to 8 equivalents, 0.1 equivalents to 5 equivalents, 0.5 equivalents to 10 equivalents, 0.5 equivalents to 8 equivalents, 0.5 equivalents to 5 equivalents, 0.9 equivalents to 10 equivalents, 0.9 equivalents to 8 equivalents, 0.9 equivalents to 5 equivalents, 1.3 equivalents to 10 equivalents, 1.3 equivalents to 8 equivalents, 1.3 equivalents to 5 equivalents, 1.5 equivalents to 10 equivalents, 1.5 equivalents to 8 equivalents, 1.5 equivalents to 5 equivalents, 2 equivalents to 10 equivalents, 2 equivalents to 8 equivalents, 2 equivalents to 5 equivalents, 0.1 equivalent to 1.5 equivalents, 0.5 equivalents to 1.1 equivalents, or 0.1 equivalent to 1 equivalent relative to the 25-hydroxy-(3β)-cholest-5-en-3-ol.

In some cases, methods include quenching (i.e., deactivating) unreacted sulfating agent after producing the 25-hydroxy-(3β)-cholest-5-en-3-sulfate organic cationic salt. In some cases, quenching the sulfating agent includes adding water to the reaction mixture. The amount of water added to the reaction mixture relative to the amount of sulfating agent contacted with the 25-hydroxy-(3β)-cholest-5-en-3-ol may vary and may be 1 equivalent or more, such as 2 equivalents or more, such as 3 equivalents or more, such as 4 equivalents or more, such as 5 equivalents or more, such as 6 equivalents or more, such as 7 equivalents or more, such as 8 equivalents or more, such as 9 equivalents or more, such as 10 equivalents or more, such as 15 equivalents or more, such as 20 equivalents or more and including 25 equivalents or more.

In certain cases, quenching the reactivity of unreacted sulfating agent includes adding water to the reaction mixture followed by the addition of at least one base. In some cases, the at least one base is a trialkylamine, such as trimethylamine or triethylamine. In some cases, the at least one base is 2,6-lutidine. In certain cases, the at least one base is pyridine. The pyridine may be added to the reaction mixture 1 minute or more after adding the water, such as 5 minutes or more, such as 10 minutes or more, such as 15 minutes or more, such as 30 minutes or more, such as 45 minutes or more, such as 60 minutes or more, such as 90 minutes or more, such as 120 minutes or more, such as 150 minutes or more, such as 180 minutes or more, such as 210 minutes or more and including 240 minutes or more after adding the water to the reaction mixture. In certain cases, pyridine is added to the reaction mixture 60 minutes after adding the water. The amount of pyridine added to the reaction mixture relative to the amount of sulfating agent may vary and may be 0.001 equivalents or more, such as 0.005 equivalents or more, such as 0.01 equivalents or more, such as 0.05 equivalents or more, such as 0.1 equivalents or more, such as 0.5 equivalents or more, such as 1 equivalent or more, such as 2 equivalents or more, such as 3 equivalents or more, such as 4 equivalents or more, such as 5 equivalents or more, such as 6 equivalents or more and including 10 equivalents or more.

In some cases, the unreacted sulfating agent in the reaction mixture is quenched under slow agitation. In certain cases, quenching the unreacted sulfating agent under slow agitation includes stirring the reaction mixture in a manner sufficient to maintain agglomerates of the unreacted sulfating agent in the reaction mixture. In some cases, slow agitation of the reaction mixture is sufficient such that agglomerates of unreacted sulfating agent reduce in size during quenching by 10% or less, such as by 9% or less, such as by 8% or less, such as by 7% or less, such as by 6% or less, such as by 5% or less, such as by 4% or less, such as by 3% or less, such as by 2% or less, such as by 1% or less and including where the reaction mixture is slowly agitated such that agglomerates of unreacted sulfating agent reduce in size during quenching by 0.1% or less. In certain cases, slow agitation of the reaction mixture is sufficient such that agglomerates of unreacted sulfating agent remain at the bottom of the reaction flask during quenching. In certain cases, slow agitation of the reaction mixture is sufficient such that little to no agglomerates of unreacted sulfating agent is present in the stirring vortex of the agitated reaction mixture.

In some cases, methods include purifying the 25-hydroxy-(3β)-cholest-5-en-3-sulfate organic cationic salt prior to contacting the 25-hydroxy-(3β)-cholest-5-en-3-sulfate organic cationic salt with the at least one metal salt. In some cases, the purified 25-hydroxy-(3β)-cholest-5-en-3-sulfate organic cationic salt has a purity of 97% or greater, such as a purity of 98% or greater, such as a purity of 99% or greater, such as purity of 99.5% or greater, such as purity of 99.7% or greater, such as a purity of 99.9% or greater and including a purity of 99.99% or greater. In certain cases, the purified 25-hydroxy-(3β)-cholest-5-en-3-sulfate organic cationic salt has one or more by-products of sulfation (e.g., by-products from sulfating the 25-hydroxy-(3β)-cholest-5-en-3-ol) where the one or more by-products is present in an amount of 5% w/w or less relative to the 25-hydroxy-(3β)-cholest-5-en-3-sulfate organic cationic salt, such as 4% w/w or less, such as 3% w/w or less, such as 2% w/w or less, such as 1% w/w or less, such as in an amount of 0.9% w/w or less, such as 0.8% w/w or less, such as 0.7% w/w or less, such as 0.6% w/w or less, such as 0.5% w/w or less, such as 0.4% w/w or less, such as 0.3% w/w or less, such as 0.2% w/w or less, such as 0.1% w/w or less, such as 0.05% w/w or less, such as 0.01% w/w or less and including being present in an amount of 0.001% w/w or less relative to the 25-hydroxy-(3β)-cholest-5-en-3-sulfate organic cationic salt. In some cases, the bis-sulfated product (i.e., 5-cholesten-3β-25-diol-disulfate) is present in the purified 25-hydroxy-(3β)-cholest-5-en-3-sulfate organic cationic salt composition in an amount of 1% w/w or less relative to the 25-hydroxy-(3β)-cholest-5-en-3-sulfate organic cationic salt, such as in an amount of 0.9% w/w or less, such as 0.8% w/w or less, such as 0.7% w/w or less, such as 0.6% w/w or less, such as 0.5% w/w or less, such as 0.4% w/w or less, such as 0.3% w/w or less, such as 0.2% w/w or less, such as 0.1% w/w or less, such as 0.05% w/w or less, such as 0.01% w/w or less and including being present in an amount of 0.001% w/w or less relative to the 25-hydroxy-(3β)-cholest-5-en-3-sulfate organic cationic salt.

In some cases, the 25-hydroxy-(3β)-cholest-5-en-3-sulfate organic cationic salt is purified by liquid chromatography. In some cases, purifying the 25-hydroxy-(3β)-cholest-5-en-3-sulfate organic cationic salt includes liquid chromatography using a silica gel stationary phase (e.g., a silica gel plug column, ≥5 mass equivalents). In some cases, the 25-hydroxy-(3β)-cholest-5-en-3-sulfate organic cationic salt is purified using the silica gel stationary phase and a mobile phase that includes pyridine. In certain cases, the mobile phase includes methylene chloride, methanol, and pyridine. In certain cases, the mobile phase includes a mixture of methylene chloride-methanol (85:15) and pyridine (1%).

In some cases, one or more fractions collected from the stationary phase may be combined. In some cases, the combined fractions may be concentrated. In certain cases, the combined fractions are concentrated by distillation. In certain cases, the combined fractions are concentrated under vacuum. In certain cases, the combined fractions are concentrated by distillation under vacuum.

In some cases, the combined fractions are contacted with one or more particles of the 25-hydroxy-(3β)-cholest-5-en-3-sulfate organic cationic salt (e.g., particles from a previously purified sample of the 25-hydroxy-(3β)-cholest-5-en-3-sulfate organic cationic salt). In some cases, contacting the particles of the 25-hydroxy-(3β)-cholest-5-en-3-sulfate organic cationic salt with the combined fractions is sufficient to precipitate 25-hydroxy-(3β)-cholest-5-en-3-sulfate organic cationic salt in the combined fractions. In some cases, contacting particles of the 25-hydroxy-(3β)-cholest-5-en-3-sulfate organic cationic salt with the combined fractions includes adding the particles during distillation of the combined fractions. In some cases, the particles of 25-hydroxy-(3β)-cholest-5-en-3-sulfate organic cationic salt are added to the combined fractions before distilling the combined fractions. In some cases, the particles of 25-hydroxy-(3β)-cholest-5-en-3-sulfate organic cationic salt are added to the combined fractions while distilling the combined fractions, such as 1 minute or more after beginning the distillation, such as 5 minutes or more, such as 10 minutes or more, such as 15 minutes or more, such as 20 minutes or more, such as 30 minutes or more, such as 40 minutes or more, such as 50 minutes or more and including adding the particles of 25-hydroxy-(3β)-cholest-5-en-3-sulfate organic cationic salt to the combined fractions 60 minutes or more after beginning the distillation of the combined fractions. In certain cases, the combined fractions are distilled under constant pressure, such as where the pressure changes by 10% or less, such as by 9% or less, such as by 8% or less, such as by 7% or less, such as by 6% or less, such as by 5% or less, such as by 4% or less, such as by 3% or less, such as by 2% or less, such as by 1% or less and including by 0.1% or less. In some cases, the pressure during distillation changes by 10 inHg or less, such as by 9 inHg or less, such as by 8 inHg or less, such as by 7 inHg or less, such as by 6 inHg or less, such as by 5 inHg or less, such as by 4 inHg or less, such as by 3 inHg or less, such as by 2 inHg or less, such as by 1 inHg or less, such as by 0.5 inHg or less, such as by 0.1 inHg or less, such as by 0.05 inHg or less and including by 0.01 inHg or less. In some cases, the combined fractions are distilled under a reduced pressure wherein the pressure is maintained between 15 inHg to 30 inHg, such as from 17.5 inHg to 27.5 inHg, such as from 20 inHg to 25 inHg, such as from 21 inHg and 24 inHg and including maintained at a pressure of from 22 inHg to 23 inHg.

In some cases, the combined fractions are concentrated under vacuum and the concentrated combined fractions are contacted with a composition containing particles of the 25-hydroxy-(3β)-cholest-5-en-3-sulfate organic cationic salt. In certain cases, the concentrated combined fractions are contacted with a composition containing particles of the 25-hydroxy-(3β)-cholest-5-en-3-sulfate organic cationic salt and at least one solvent. In certain cases, the at least one solvent is chosen from tetrahydrofurans, such as 2-methyltetrahydrofuran. The concentrated combined fractions may be contacted with the composition containing the particles of the 25-hydroxy-(3β)-cholest-5-en-3-sulfate organic cationic salt over a duration of 0.001 minutes or more, such as over 0.005 minutes or more, such as over 0.01 minutes or more, such as over 0.05 minutes or more, such as over 0.1 minutes or more, such as over 0.5 minutes or more, such as over 1 minute or more, such as over 2 minutes or more, such as over 3 minutes or more, such as over 4 minutes or more, such as over 5 minutes or more, such as over 10 minutes or more, such as over 15 minutes or more, such as over 30 minutes or more, such as over 45 minutes or more and including over 60 minutes or more. In certain cases, the combined fractions are added dropwise to a composition containing 25-hydroxy-(3β)-cholest-5-en-3-sulfate organic cationic salt in 2-methyltetrahydrofuran.

In some cases, the 25-hydroxy-(3β)-cholest-5-en-3-sulfate organic cationic salt is contacted with a metal salt to produce the 25-hydroxy-(3β)-cholest-5-en-3-sulfate metal salt (Scheme IB1).

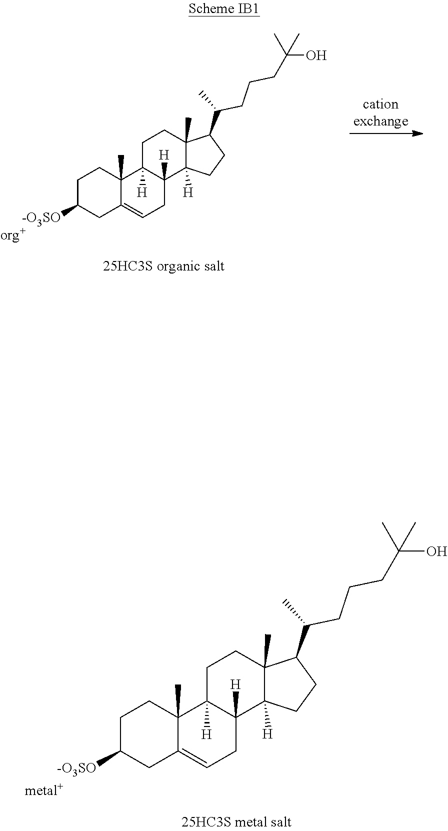

In some cases, methods to produce the 25-hydroxy-(3β)-cholest-5-en-3-sulfate metal salt includes contacting the 25-hydroxy-(3β)-cholest-5-en-3-sulfate organic cationic salt with at least one sodium salt. In some cases, the at least one sodium salt is chosen from sodium acetate, sodium iodide, sodium chloride, sodium hydroxide and sodium methoxide. The 25-hydroxy-(3β)-cholest-5-en-3-sulfate organic cationic salt may be contacted with the metal salt at a temperature that ranges from −10° C. to 75° C., such as from −5° C. to 70° C., such as from −4° C. to 65° C., such as from −3° C. to 60° C., such as from −2° C. to 55° C., such as from −1° C. to 50° C., such as from 0° C. to 45° C., such as from 5° C. to 40° C., and including from 10° C. to 35° C.

The reaction may be carried out for a duration that ranges from 0.1 hours to 72 hours, such as from 0.2 hours to 48 hours, such as from 0.3 hours to 24 hours, such as from 0.4 hours to 21 hours, such as from 0.5 hours to 20 hours, such as from 0.6 hours to 19 hours, such as from 0.7 hours to 18 hours, such as from 0.8 hours to 17 hours, such as from 0.9 hours to 16 hours, and including from 1 hours to 15 hours. The amount of metal salt used relative to the 25-hydroxy-(3β)-cholest-5-en-3-sulfate organic cationic salt may vary and may be 0.0001 equivalents or more, such as 0.001 equivalents or more, such as 0.01 equivalents or more, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more, and including 10 equivalents or more, and may range from 0.001 equivalents to 10 equivalents, such as 0.1 equivalents to 10 equivalents, 0.1 equivalents to 8 equivalents, 0.1 equivalents to 6 equivalents, 0.1 equivalents to 4 equivalents, 0.1 equivalents to 3 equivalents, 1 equivalents to 10 equivalents, 1 equivalents to 8 equivalents, 1 equivalents to 6 equivalents, 1 equivalents to 4 equivalents, 1 equivalents to 3 equivalents, 1.5 equivalents to 10 equivalents, 1.5 equivalents to 8 equivalents, 1.5 equivalents to 6 equivalents, 1.5 equivalents to 4 equivalents, 1.5 equivalents to 3 equivalents, 2 equivalents to 10 equivalents, 2 equivalents to 8 equivalents, 2 equivalents to 6 equivalents, 2 equivalents to 4 equivalents, or 2 equivalents to 3 equivalents, 1 equivalent to 100 equivalents, 1 equivalent to 5 equivalents, 1 equivalent to 2 equivalents.

In some cases, methods include contacting the 25-hydroxy-(3β)-cholest-5-en-3-sulfate pyridinium salt with sodium iodide to produce a 25-hydroxy-(3β)-cholest-5-en-3-sulfate sodium salt (Scheme IB2).

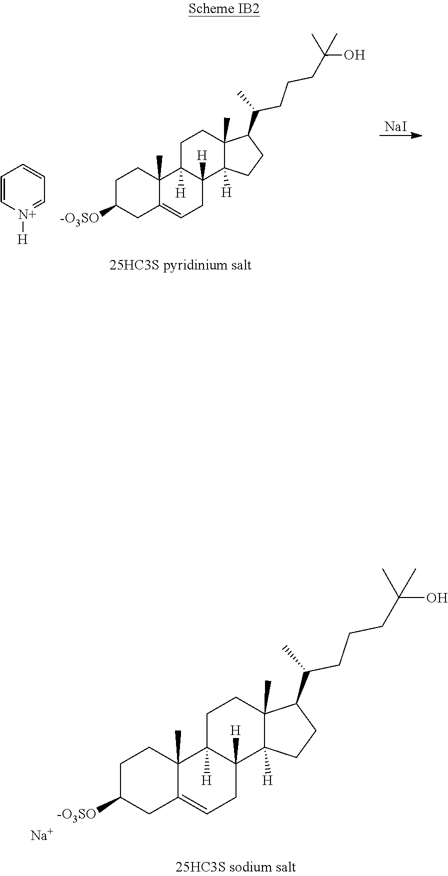

In some cases, methods for preparing 25-hydroxy-3β-cholesten-5-en-3-sulfate include contacting 25-hydroxy-(3β)-cholest-5-en-3-ol with a sulfur trioxide-pyridine complex to produce a 25-hydroxy-(3β)-cholest-5-en-3-sulfate pyridinium salt; and contacting the 25-hydroxy-(3β)-cholest-5-en-3-sulfate pyridinium salt with a sodium salt to produce the 5-cholesten-3β,25-diol 3-sulfate sodium salt (Scheme Ib).

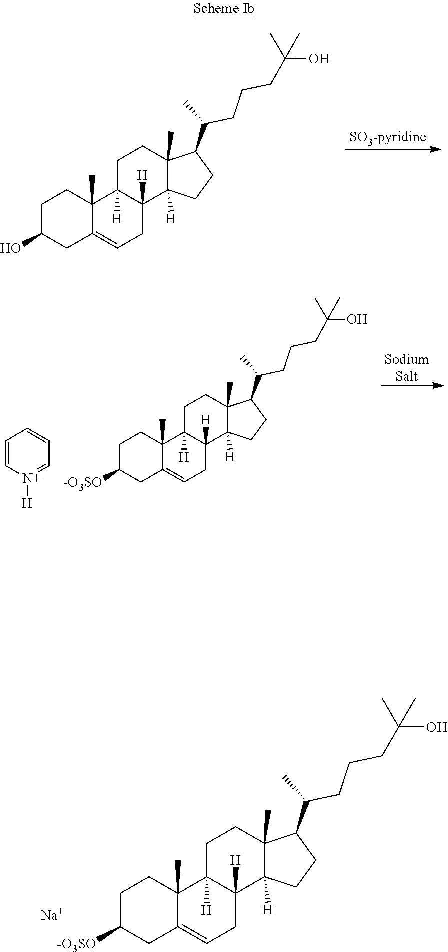

In some cases, methods for preparing 25-hydroxy-3β-cholesten-5-en-3-sulfate include contacting (3β)-cholest-5-en-3-ol with a sulfating agent to produce a first (3β)-cholest-5-en-3-sulfate organic cationic salt; contacting the first (3β)-cholest-5-en-3-sulfate organic cationic salt with an organic base to produce a second (3β)-cholest-5-en-3-sulfate organic cationic salt; oxidizing the second (3β)-cholest-5-en-3-sulfate organic cationic salt in the presence of at least one surfactant to produce a 25-hydroxy-(3β)-cholest-(5,6-epoxy)-3-sulfate organic cationic salt; generating a 25-hydroxy-(3β)-cholest-5-en-3-sulfate organic cationic salt from the 25-hydroxy-(3β)-cholest-(5,6-epoxy)-3-sulfate organic cationic salt by deoxygenation; and contacting the 25-hydroxy-(3β)-cholest-5-en-3-sulfate organic cationic salt with at least one metal salt to produce the 5-cholesten-3β,25-diol 3-sulfate metal salt (Scheme IIa).

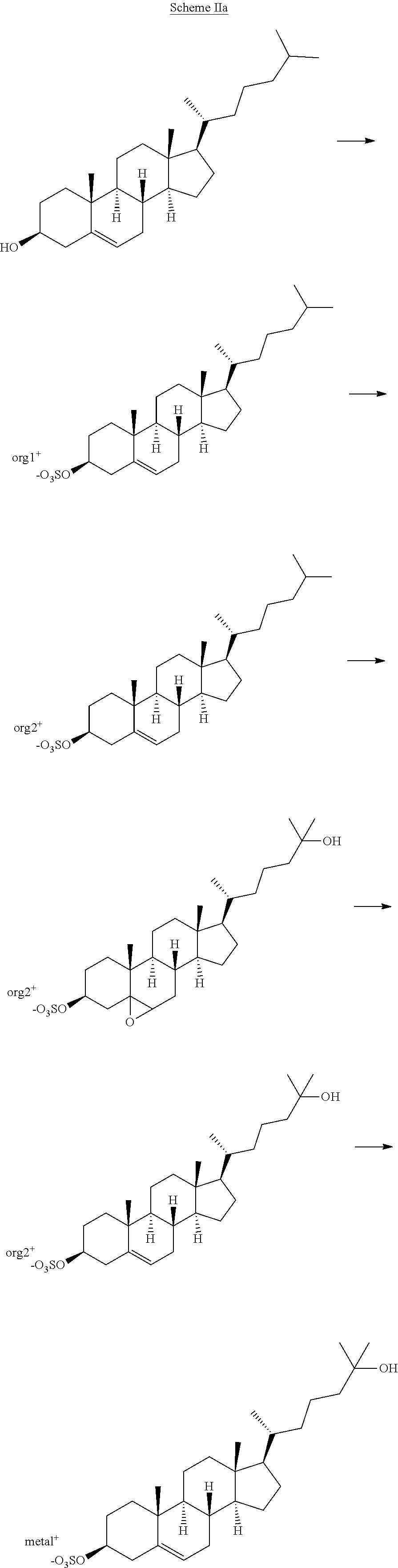

In some cases, cholesterol is sulfated with a sulfating agent (Scheme IIA1). In some cases, the sulfating agent is chosen from sulfur trioxide complexes, sulfuric acid compounds, sulfonic acid compounds, and sulfonate compounds. In some cases, the sulfating agent is a sulfur trioxide-pyridine complex. In some cases, the sulfating agent is chosen from sulfur trioxide dimethyl formamide, sulfur trioxide triethylamine, and sulfur trioxide trimethylamine. In some cases, the sulfating agent is sulfuric acid and acetic anhydride and pyridine. In some cases, the sulfating agent is chosen from chlorosulfonic acid and pyridine. In some cases, the sulfating agent is chosen from chlorosulfonic acid and 2,6-lutidine. In some cases, the sulfating agent is chosen from ethyl chlorosulfonate.

Cholesterol may be sulfated at a temperature that ranges from 0° C. to 100° C., such as from 5° C. to 95° C., such as from 10° C. to 90° C., such as from 15° C. to 85° C., such as from 20° C. to 80° C., such as from 25° C. to 75° C., and including from 30° C. to 70° C. The reaction may be carried out for a duration that ranges from 0.1 hours to 72 hours, such as from 0.2 hours to 48 hours, such as from 0.3 hours to 24 hours, such as from 0.4 hours to 21 hours, such as from 0.5 hours to 20 hours, such as from 0.6 hours to 19 hours, and including from 0.7 hours to 18 hours. The amount of sulfating agent used relative to cholesterol may vary and may be 0.0001 equivalents or more, such as 0.001 equivalents or more, such as 0.01 equivalents or more, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more, and including 10 equivalents or more, and may range from 0.001 equivalents to 10 equivalents, such as 0.1 equivalents to 10 equivalents, 0.1 equivalents to 8 equivalents, 0.1 equivalents to 6 equivalents, 0.1 equivalents to 4 equivalents, 0.1 equivalents to 3 equivalents, 1 equivalents to 10 equivalents, 1 equivalents to 8 equivalents, 1 equivalents to 6 equivalents, 1 equivalents to 4 equivalents, 1 equivalents to 3 equivalents, 1.5 equivalents to 10 equivalents, 1.5 equivalents to 8 equivalents, 1.5 equivalents to 6 equivalents, 1.5 equivalents to 4 equivalents, 1.5 equivalents to 3 equivalents, 2 equivalents to 10 equivalents, 2 equivalents to 8 equivalents, 2 equivalents to 6 equivalents, 2 equivalents to 4 equivalents, 2 equivalents to 3 equivalents, 1 equivalent to 30 equivalents, 1 equivalent to 5 equivalents, or 1 equivalent to 2 equivalents.

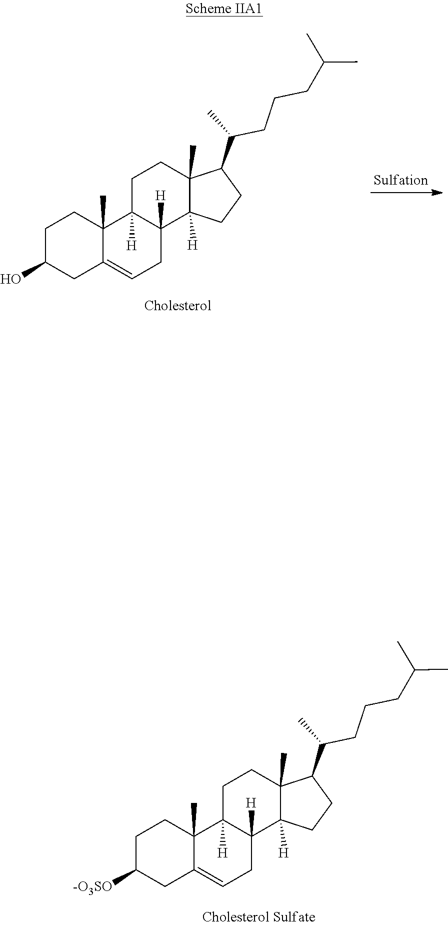

In some cases, the first (3β)-cholest-5-en-3-sulfate organic cationic salt is a (3β)-cholest-5-en-3-sulfate pyridinium salt (Scheme IIA2).

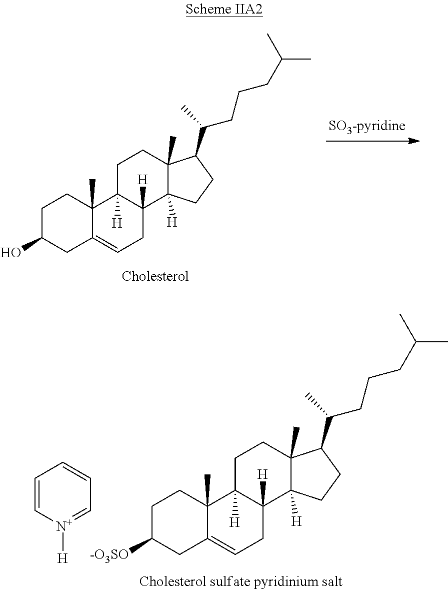

In some cases, the first (3β)-cholest-5-en-3-sulfate organic cationic salt (Structure IIA) is contacted with an organic base to produce a second (3β)-cholest-5-en-3-sulfate organic cationic salt (Structure IIB) (Scheme IIB1).

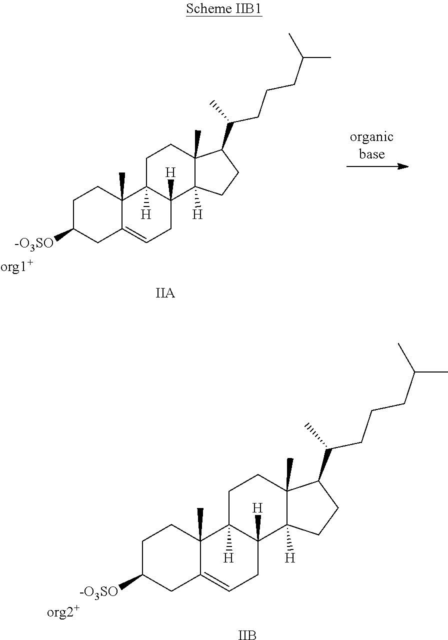

In some cases, the organic base contacted with the first (3β)-cholest-5-en-3-sulfate organic cationic salt is chosen from a hydroxide base. In some cases, the hydroxide base is chosen from tetraethylammonium hydroxide, tetrabutylammonium hydroxide, tetrapropylammonium hydroxide and tetramethylammonium hydroxide. In some cases, the second (3β)-cholest-5-en-3-sulfate organic cationic salt is chosen from a tetraethylammonium cationic salt, a tetrabutylammonium cationic salt, a tetrapropylammonium cationic salt and a tetramethylammonium cationic salt. In some cases, the organic base is contacted with the first (3β)-cholest-5-en-3-sulfate organic cationic salt at a temperature that ranges from −10° C. to 75° C., such as from −5° C. to 70° C., such as from −4° C. to 65° C., such as from −3° C. to 60° C., such as from −2° C. to 55° C., such as from −1° C. to 50° C. and including from 0° C. to 15° C. The reaction may be carried out for a duration that ranges from 0.1 hours to 72 hours, such as from 0.2 hours to 48 hours, such as from 0.3 hours to 24 hours, such as from 0.4 hours to 21 hours, such as from 0.5 hours to 20 hours, such as from 0.6 hours to 19 hours, such as from 0.7 hours to 18 hours, such as from 0.8 hours to 17 hours, such as from 0.9 hours to 16 hours, and including from 1 hour to 15 hours. The amount of the organic base used relative to the first (3β)-cholest-5-en-3-sulfate organic cationic salt may vary and may be 0.0001 equivalents or more, such as 0.001 equivalents or more, such as 0.01 equivalents or more, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more, and including 10 equivalents or more, and may range from 0.001 equivalents to 10 equivalents, such as 0.1 equivalents to 10 equivalents, 0.1 equivalents to 8 equivalents, 0.1 equivalents to 6 equivalents, 0.1 equivalents to 4 equivalents, 0.1 equivalents to 3 equivalents, 1 equivalents to 10 equivalents, 1 equivalents to 8 equivalents, 1 equivalents to 6 equivalents, 1 equivalents to 4 equivalents, 1 equivalents to 3 equivalents, 1.5 equivalents to 10 equivalents, 1.5 equivalents to 8 equivalents, 1.5 equivalents to 6 equivalents, 1.5 equivalents to 4 equivalents, 1.5 equivalents to 3 equivalents, 2 equivalents to 10 equivalents, 2 equivalents to 8 equivalents, 2 equivalents to 6 equivalents, 2 equivalents to 4 equivalents, 2 equivalents to 3 equivalents, 1 equivalent to 10 equivalents, 1 equivalent to 5 equivalents, or 1 equivalent to 2 equivalents.

In certain cases, methods include contacting the first (3β)-cholest-5-en-3-sulfate organic cationic salt with tetrabutylammonium hydroxide to generate a (3β)-cholest-5-en-3-sulfate tetrabutylammonium cationic salt (Structure IIB1) (Scheme IIB2).

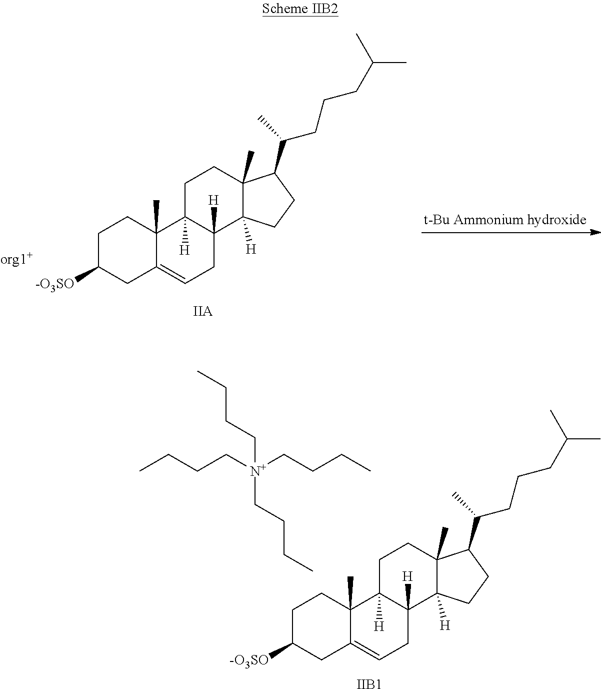

In some cases, the second (3β)-cholest-5-en-3-sulfate organic cationic salt is oxidized to produce a 25-hydroxy-(3β)-cholest-(5,6-epoxy)-3-sulfate organic cationic salt (Structure IIC) (Scheme IIC1).

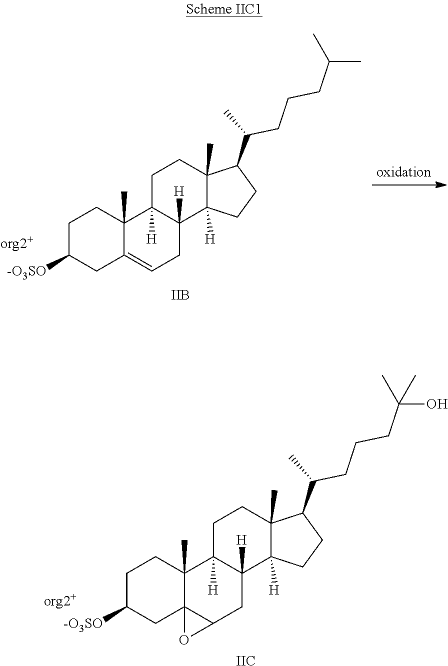

In some cases, oxidizing the second (3β)-cholest-5-en-3-sulfate organic cationic salt includes contacting the second (3β)-cholest-5-en-3-sulfate organic cationic salt with a composition having an oxidizing agent and at least one surfactant.

In some cases, the at least one surfactant is chosen from non-ionic surfactants, anionic surfactants, cationic surfactants and zwitterionic surfactants. Non-ionic surfactants may be chosen from polyoxyethylene glycol ethers (e.g., polyoxyethylene glycol octylphenol ether), polyoxyethylene glycol sorbitan alkyl esters, sorbitan alkyl esters, block copolymers of polyethylene glycol and polypropylene glycol, among other non-ionic surfactants. Anionic surfactants may be chosen from surfactants having an anionic functional head group, such as a sulfonate, phosphate, sulfate or carboxylate head group-containing surfactant. For example, anionic surfactants may be chosen from alkyl sulfates such as ammonium lauryl sulfate, dioctyl sodium sulfosuccinate, perfluorooctanesulfonate, perfluorononanoate, perfluorooctanoate, a linear alkylbenzene sulfonate, an alkyl-aryl ether phosphate, sodium lauryl ether sulfate, lignosulfonate or sodium stearate, among other anionic surfactants. Cationic surfactants may be chosen from surfactants having a cationic functional head group, such as a pyridinium or a quaternary ammonium head group. For example, cationic surfactants may be chosen from cetyltrimethylammonium hydrogen sulfate, tetrabutylammonium hydrogen sulfate, cetyltrimethylammonium bromide, tetrabutylammonium bromide, tetrabutylammonium iodide, tetrabutylphosphonium bromide, tetraoctylammonium bromide, tetraoctylammonium iodide, benzyltriethylammonium chloride, benzyltriethylammonium bromide, benzylcetyldimethylammonium chloride or benzylcetyldimethylammonium bromide. Zwitterionic surfactants include both cationic and anionic centers, such as a sultaine (e.g., 3-[(3-cholamidopropyl)dimethylammonio]-1-propanesulfonate) or a betaine (e.g., cocamidopropyl betaine). In certain cases, the at least one surfactant is an Extran laboratory soap, La Parisienne soap or DL-α-tocopherol methoxypolyethylene glycol succinate (e.g., TPGS-750-M-2).

The amount of surfactant used relative to the second (3β)-cholest-5-en-3-sulfate organic cationic salt may vary, where in some instances, 0.0001 equivalents or more of the surfactant is used, such as 0.001 equivalents or more, such as 0.01 equivalents or more, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more, and including 10 equivalents or more of the surfactant, and may range from 0.001 equivalents to 10 equivalents, such as 0.1 equivalents to 10 equivalents, 0.1 equivalents to 8 equivalents, 0.1 equivalents to 6 equivalents, 0.1 equivalents to 4 equivalents, 0.1 equivalents to 3 equivalents, 1 equivalents to 10 equivalents, 1 equivalents to 8 equivalents, 1 equivalents to 6 equivalents, 1 equivalents to 4 equivalents, 1 equivalents to 3 equivalents, 1.5 equivalents to 10 equivalents, 1.5 equivalents to 8 equivalents, 1.5 equivalents to 6 equivalents, 1.5 equivalents to 4 equivalents, 1.5 equivalents to 3 equivalents, 2 equivalents to 10 equivalents, 2 equivalents to 8 equivalents, 2 equivalents to 6 equivalents, 2 equivalents to 4 equivalents, 2 equivalents to 3 equivalents, 0.1 equivalent to 5 equivalents, 0.15 equivalents to 1 equivalent, or 0.2 equivalents to 0.3 equivalents.

In some cases, oxidizing the second (3β)-cholest-5-en-3-sulfate organic cationic salt includes contacting the second (3β)-cholest-5-en-3-sulfate organic cationic salt with an oxidizing agent and at least one ketone in the presence of at least one surfactant.

In some instances, the at least one ketone is chosen from tetrahydrothiopyran-4-one 1,1-dioxide and halogenated ketones. In some cases, the halogenated ketones are chosen from 1,1,1-trifluoro-2-butanone, 4,4-difluorocyclohexanone, 2-2-2-4′-tetrafluoroacetophenone, and 1,1,1-trifluoroacetone. In certain cases, the at least one ketone is 1,1,1-trifluoro-2-butanone. The amount of ketone used relative to the oxidizing agent in the subject reaction may vary, and may be 1 equivalent or more, such as 2 equivalents or more, such as 3 equivalents or more, such as 4 equivalents or more, such as 5 equivalents or more, such as 6 equivalents or more, such as 7 equivalents or more, such as 8 equivalents or more, such as 9 equivalents or more, such as 10 equivalents or more, such as 15 equivalents or more, such as 20 equivalents or more, such as 25 equivalents or more, such as 30 equivalents or more, such as 35 equivalents or more, and including 50 equivalents or more of the ketone, and may range from 1 equivalent to 50 equivalents, such as 1 equivalent to 35 equivalents, 1 equivalent to 25 equivalents, 1 equivalent to 15 equivalents, 1 equivalent to 10 equivalents, 1 equivalent to 8 equivalents, 1 equivalent to 5 equivalents, 2 equivalent to 50 equivalents, 2 equivalent to 35 equivalents, 2 equivalent to 25 equivalents, 2 equivalent to 15 equivalents, 2 equivalent to 10 equivalents, 2 equivalent to 8 equivalents, 2 equivalent to 5 equivalents, 4 equivalent to 50 equivalents, 4 equivalent to 35 equivalents, 4 equivalent to 25 equivalents, 4 equivalent to 15 equivalents, 4 equivalent to 10 equivalents, 4 equivalent to 8 equivalents, 1 equivalent to 50 equivalents, 2 equivalent to 25 equivalents, or 5 equivalents to 10 equivalents.

In certain cases, the ketone is further purified before use. For example, the ketone may be purified by distillation prior to use. In some instances, the reactivity of the ketone is tested (e.g., tested for impurities by ¹H-NMR) in order to determine whether purification may be required.

In certain cases, oxidizing the second (3β)-cholest-5-en-3-sulfate organic cationic salt includes contacting the second (3β)-cholest-5-en-3-sulfate organic cationic salt with an oxidizing agent and at least one ketone in the presence of at least one surfactant and water. The amount of water present may vary, ranging from 0.0000001% w/v or more of the reaction mixture, such as 0.000001% w/v or more, such 0.00001% w/v or more, such as 0.0001% w/v or more, such as 0.001% w/v, such as 0.01% w/v or more, such as 0.1% w/v, such as 0.05% w/v or more, such as 0.1% w/v or more, such as 0.5% w/v or more, such as 1% w/v or more, such as 5% w/v or more, such as 10% w/v or more, such as 15% w/v or more, and including 25% w/v or more of the reaction mixture, and may range from 0.0000001% w/v to 25% w/v, such as 0.0000001% w/v to 15% w/v, 0.0000001% w/v to 10% w/v, 0.0000001% w/v to 5% w/v, 0.0000001% w/v to 1% w/v, 0.001% w/v to 25% w/v, 0.001% w/v to 15% w/v, 0.001% w/v to 10% w/v, 0.001% w/v to 5% w/v, 0.001% w/v to 1% w/v, 0.1% w/v to 25% w/v, 0.1% w/v to 15% w/v, 0.1% w/v to 10% w/v, 0.1% w/v to 5% w/v, 0.1% w/v to 10% w/v, 1% w/v to 25% w/v, 1% w/v to 15% w/v, 1% w/v to 10% w/v, 1% w/v to 5% w/v, 0.1% w/v to 50% w/v, 0.1% w/v to 10% w/v, or 0.5% w/v to 1% w/v.

The second (3β)-cholest-5-en-3-sulfate organic cationic salt may be oxidized at a temperature that ranges from −25° C. to 50° C., such as from −20° C. to 45° C., such as from −15° C. to 40° C., such as from −10° C. to 35° C., such as from −5° C. to 30° C., such as from −1° C. to 25° C., and including from 0° C. to 15° C. In certain cases, the second (3β)-cholest-5-en-3-sulfate organic cationic salt is oxidized at a temperature of from 0° C. to 5 CC. Where the reaction mixture includes an amount of water, the reaction may be conducted at a temperature that is from −10° C. to 50° C., such as from −5° C. to 45° C., such as from 0° C. to 40° C., such as from 0° C. to 35° C., such as from 0° C. to 30° C., such as from 0° C. to 25° C., such as from 0° C. to 20° C., such as from 0° C. to 15° C., and including from 0° C. to 10° C.

The second (3β)-cholest-5-en-3-sulfate organic cationic salt may be oxidized at a pH that ranges from 5 to 7.5, such as a pH of from 5.5 to 7.0 and including a pH of from 5.5 to 6.5. In some cases, where the reaction mixture contains water (e.g., in a biphasic solvent system), the pH ranges from 5.0 to 6.0, such as a pH of from 5.0 to 5.9, such as a pH of from 5.0 to 5.8, such as a pH of from 5.0 to 5.7, such as a pH from 5.0 to 5.6, and including a pH of from 5.0 to 5.5.

The reaction may be carried out for a duration that ranges from 0.1 hours to 72 hours, such as from 0.2 hours to 48 hours, such as from 0.3 hours to 24 hours, such as from 0.4 hours to 21 hours, such as from 0.5 hours to 20 hours, such as from 0.6 hours to 19 hours, such as from 0.7 hours to 18 hours, such as from 0.8 hours to 17 hours, such as from 0.9 hours to 16 hours, and including from 1 hours to 15 hours.

In some instances, the second (3β)-cholest-5-en-3-sulfate organic cationic salt is contacted in situ with a composition having potassium peroxymonosulfate and at least one ketone in the presence of at least one surfactant. In some cases, methods include contacting the potassium peroxymonosulfate with at least one ketone in the presence of at least one surfactant to form a separate oxidative reactive mixture and adding the oxidative reactive mixture to the second (3β)-cholest-5-en-3-sulfate organic cationic salt. In these cases, the potassium peroxymonosulfate may be contacted with the at least one ketone in the presence of the at least one surfactant for a duration of 0.1 minute or more before contacting the oxidative reactive mixture with the second (3β)-cholest-5-en-3-sulfate organic cationic salt, such as 1 minute or more, such as 2 minutes or more, such as 3 minutes or more, such as 5 minutes or more, and including 10 minutes or more, and the time may range from 2 minutes to 180 minutes, such as 3 minutes to 120 minutes or 4 minutes to 60 minutes. In certain instances, the potassium peroxymonosulfate may be contacted with the at least one ketone in the presence of the at least one surfactant to form a separate oxidative reactive mixture and immediately contacting the oxidative reactive mixture with the second (3β)-cholest-5-en-3-sulfate organic cationic salt. The oxidative reactive mixture may be formed at a temperature that ranges from −10° C. to 50° C., such as from −5° C. to 45° C., such as from −4° C. to 40° C., such as from −3° C. to 35° C., such as from −2° C. to 30° C., such as from −1° C. to 25° C. and including from 0° C. to 15° C. Where the oxidative reactive mixture is not immediately contacted with the second (3β)-cholest-5-en-3-sulfate organic cationic salt, the oxidative reactive mixture may be maintained at a temperature that ranges from −10° C. to 50° C., such as from −5° C. to 45° C., such as from −4° C. to 40° C., such as from −3° C. to 35° C., such as from −2° C. to 30° C., such as from −1° C. to 25° C., and including from 0° C. to 15° C.

In some cases, methods further include adding the oxidative reactive mixture to the second (3β)-cholest-5-en-3-sulfate organic cationic salt. In some instances, methods include adding dropwise the oxidative reactive mixture to the second (3β)-cholest-5-en-3-sulfate organic cationic salt. In some instances, the oxidative reactive mixture is added to the second (3β)-cholest-5-en-3-sulfate organic cationic salt in metered amounts. The metered amounts may be added continuously or at predetermined time intervals (e.g., every 30 seconds, 1 minute, 2 minutes, 3 minutes, 5 minutes, or some other interval). In some instances, the oxidative reactive mixture is added to the second (3β)-cholest-5-en-3-sulfate organic cationic salt by controlled addition, such as with a mechanically or computer controlled pump, e.g., syringe pump. In some cases, methods include generating the oxidative reactive mixture and adding a composition containing the second (3β)-cholest-5-en-3-sulfate organic cationic salt to the oxidative reactive mixture. In some instances, methods include adding dropwise the second (3β)-cholest-5-en-3-sulfate organic cationic salt to the oxidative reactive mixture. In some instances, the second (3β)-cholest-5-en-3-sulfate organic cationic salt is added to the oxidative reactive mixture in metered amounts. The metered amounts may be added continuously or at predetermined time intervals (e.g., every 30 seconds, 1 minute, 2 minutes, 3 minutes, 5 minutes, or some other interval). In some instances, the second (3β)-cholest-5-en-3-sulfate organic cationic salt is added to the oxidative reactive mixture by controlled addition, such as with a mechanically or computer-controlled pump, e.g., syringe pump.

In certain cases, oxidizing the second (3β)-cholest-5-en-3-sulfate organic cationic salt includes contacting the second (3β)-cholest-5-en-3-sulfate organic cationic salt with at least one oxidative species. In some instances, the at least one oxidative species is chosen from dioxiranes. In some instances, the dioxiranes are generated in situ in a composition having the second (3)-cholest-5-en-3-sulfate organic cationic salt. In some instances, the dioxiranes are generated separately (e.g., in a separate reaction container, e.g., flask) and added to the composition having the second (3β)-cholest-5-en-3-sulfate organic cationic salt.

In certain cases, the second (3β)-cholest-5-en-3-sulfate organic cationic salt is oxidized in the presence of at least one base. In certain cases, the at least one base is chosen from weak bases. In some cases, the at least one base is chosen from potassium hydrogen carbonate, sodium hydrogen carbonate, potassium phenoxide, sodium citrate buffer, sodium phosphate buffer, potassium formate and potassium acetate. In certain cases, the at least one base is potassium hydrogen carbonate. In some cases, the at least one base may be added to the reaction mixture over time, such as in metered amounts where the base is added at predetermined time intervals (e.g., every 30 seconds, 1 minute, 2 minutes, 3 minutes, 5 minutes, or some other interval). In some cases, the at least one base may be a composition having water where the base present in the composition may be 0.0000001% w/v or more of the composition, such as 0.000001% w/v or more, such as 0.00001% w/v or more, such as 0.0001% w/v or more, such as 0.001% w/v or more, such as 0.01% w/v or more, such as 0.05% w/v or more, such as 0.1% w/v or more, such as 0.5% w/v or more, such as 1% w/v or more, such as 5% w/v or more, such as 10% w/v or more, such as 15% w/v or more, and including 25% w/v or more of the composition, and may range from 0.0000001% w/v to 25% w/v, such as 0.0000001% w/v to 15% w/v, 0.0000001% w/v to 10% w/v, 0.0000001% w/v to 5% w/v, 0.0000001% w/v to 1% w/v, 0.001% w/v to 25% w/v, 0.0010% w/v to 15% w/v, 0.0010% w/v to 10% w/v, 0.0010% w/v to 5% w/v, 0.0010% w/v to 10% w/v, 0.1% w/v to 25% w/v, 0.1% w/v to 15% w/v, 0.1% w/v to 10% w/v, 0.1% w/v to 5% w/v, 0.1% w/v to 1% w/v, 1% w/v to 25% w/v, 1% w/v to 15% w/v, 1% w/v to 10% w/v, 1% w/v to 5% w/v, 0.1% w/v to 20% w/v, 0.2% w/v to 15% w/v, or 0.3% w/v to 10% w/v. In certain cases, the at least one base may be an aqueous potassium hydrogen carbonate composition.

In certain cases, the second (3β)-cholest-5-en-3-sulfate organic cationic salt is oxidized by contacting with oxone in the presence of cetyltrimethylammonium hydrogen sulfate (CTAHS) followed by adding trifluorobutanone and potassium hydrogen sulfate to form 25-hydroxy-(3β)-cholest-(5,6-epoxy)-3-sulfate organic cationic salt (Scheme IIC2).

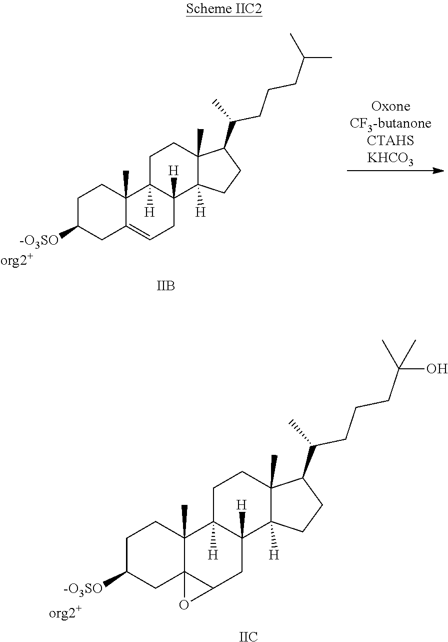

In certain instances, methods include forming an oxidative species in situ with the second (3β)-cholest-5-en-3-sulfate organic cationic salt, such as by contacting potassium peroxymonosulfate and trifluorobutanone in the presence of cetyltrimethylammonium hydrogen sulfate (CTAHS) in a reaction mixture with the second (3β)-cholest-5-en-3-sulfate organic cationic salt. In certain cases, forming an oxidative species in situ with the second (3β)-cholest-5-en-3-sulfate organic cationic salt includes forming a dioxirane in situ with the second (3β)-cholest-5-en-3-sulfate organic cationic salt.

In certain cases, methods include forming a dioxirane in a separate reaction and adding the dioxirane to the second (3β)-cholest-5-en-3-sulfate organic cationic salt. In these cases, the potassium peroxymonosulfate may be contacted with the trifluorobutanone in the presence of cetyltrimethylammonium hydrogen sulfate (CTAHS) for a duration of 0.1 minute or more before contacting the reactive composition with the second (3β)-cholest-5-en-3-sulfate organic cationic salt, such as 1 minute or more, such as 2 minutes or more, such as 3 minutes or more, such as 5 minutes or more, and including 10 minutes or more), and the time may range from 0.01 minutes to 120 minutes, such as 0.1 minutes to 90 minutes or 0.5 minutes to 60 minutes. In certain instances, the potassium peroxymonosulfate may be contacted with trifluorobutanone in the presence of cetyltrimethylammonium hydrogen sulfate (CTAHS) to form the oxidative reactive composition, which is immediately contacted with the second (3β)-cholest-5-en-3-sulfate organic cationic salt.

The 25-hydroxy-(3β)-cholest-(5,6-epoxy)-3-sulfate organic cationic salt may be deoxygenated to produce a 25-hydroxy-(3β)-cholest-5-en-3-sulfate organic cationic salt (Structure IID) (Scheme IID1).

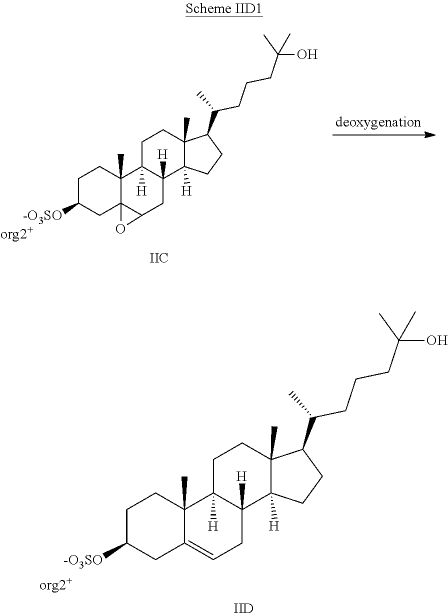

In some cases, generating 25-hydroxy-(3β)-cholest-5-en-3-sulfate organic cationic salt from the 25-hydroxy-(3β)-cholest-(5,6-epoxy)-3-sulfate organic cationic salt includes deoxygenation by contacting the 25-hydroxy-(3β)-cholest-(5,6-epoxy)-3-sulfate organic cationic salt with zinc. In certain instances, the 25-hydroxy-(3β)-cholest-(5,6-epoxy)-3-sulfate organic cationic salt is contacted with zinc in the presence of at least one halide and at least one acid. In some cases, the at least one halide is chosen from iodine and metal halides. In some cases, the metal halide is chosen from sodium iodide and lithium iodide. In some cases, the at least one acid is chosen from weak acids. In some cases, the at least one acid is chosen from acetic acid, hydrochloric acid, citric acid, para-toluene sulfonic acid, formic acid and methane sulfonic acid.

The amount of reagent used to deoxygenate the 25-hydroxy-(3β)-cholest-(5,6-epoxy)-3-sulfate organic cationic salt may vary, where in some instances, 0.0001 equivalents or more of reagent relative to the 25-hydroxy-(3β)-cholest-(5,6-epoxy)-3-sulfate organic cationic salt is used, such as 0.001 equivalents or more, such as 0.01 equivalents or more, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more, and including 10 equivalents or more, and may range from 0.001 equivalents to 10 equivalents, such as 0.1 equivalents to 10 equivalents, 0.1 equivalents to 8 equivalents, 0.1 equivalents to 6 equivalents, 0.1 equivalents to 4 equivalents, 0.1 equivalents to 3 equivalents, 1 equivalents to 10 equivalents, 1 equivalents to 8 equivalents, 1 equivalents to 6 equivalents, 1 equivalents to 4 equivalents, 1 equivalents to 3 equivalents, 1.5 equivalents to 10 equivalents, 1.5 equivalents to 8 equivalents, 1.5 equivalents to 6 equivalents, 1.5 equivalents to 4 equivalents, 1.5 equivalents to 3 equivalents, 2 equivalents to 10 equivalents, 2 equivalents to 8 equivalents, 2 equivalents to 6 equivalents, 2 equivalents to 4 equivalents, 2 equivalents to 3 equivalents, 1 equivalent to 20 equivalents, 1 equivalent to 10 equivalents, or 4 equivalents to 6 equivalents.

The 25-hydroxy-(3β)-cholest-(5,6-epoxy)-3-sulfate organic cationic salt may be deoxygenated at a temperature that ranges from −10° C. to 75° C., such as from −5° C. to 70° C., such as from −4° C. to 65° C., such as from −3° C. to 60° C., such as from −2° C. to 55° C., such as from −1° C. to 50° C. and including from 0° C. to 25° C. The reaction may be carried out for a duration that ranges from 0.1 hours to 72 hours, such as from 0.2 hours to 48 hours, such as from 0.3 hours to 24 hours, such as from 0.4 hours to 21 hours, such as from 0.5 hours to 20 hours, such as from 0.6 hours to 19 hours, such as from 0.7 hours to 18 hours, such as from 0.8 hours to 17 hours, such as from 0.9 hours to 16 hours, and including from 1 hours to 15 hours.

In certain instances, methods include contacting the 25-hydroxy-(3β)-cholest-(5,6-epoxy)-3-sulfate organic cationic salt with zinc in the presence of iodine and acetic acid to generate the 25-hydroxy-(3β)-cholest-5-en-3-sulfate organic cationic salt (Scheme IID2).

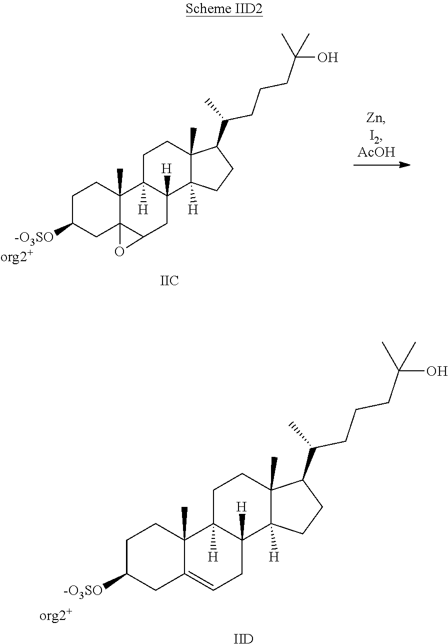

In some cases, the 25-hydroxy-(3β)-cholest-5-en-3-sulfate organic cationic salt (Structure IID) is contacted with a metal salt to produce the 25-hydroxy-(3β)-cholest-5-en-3-sulfate metal salt (Structure IIE) (Scheme IIE1).

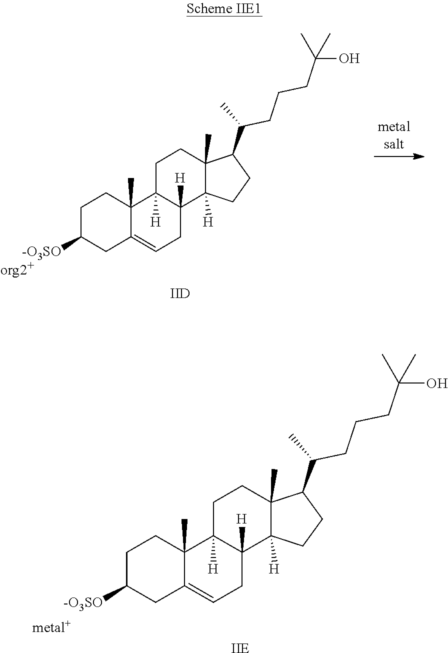

In some cases, methods to produce the 25-hydroxy-(3β)-cholest-5-en-3-sulfate metal salt include contacting the 25-hydroxy-(3β)-cholest-5-en-3-sulfate organic cationic salt with at least one sodium salt. In some cases, the at least one sodium salt is chosen from sodium acetate, sodium iodide, sodium chloride, sodium hydroxide and sodium methoxide. The 25-hydroxy-(3β)-cholest-5-en-3-sulfate organic cationic salt may be contacted with the metal salt at a temperature that ranges from −10° C. to 75° C., such as from −5° C. to 70° C., such as from −4° C. to 65° C., such as from −3° C. to 60° C., such as from −2° C. to 55° C., such as from −1° C. to 50° C., such as from 0° C. to 45° C., such as from 5° C. to 40° C., and including from 10° C. to 35° C.

The reaction may be carried out for a duration that ranges from 0.1 hours to 72 hours, such as from 0.2 hours to 48 hours, such as from 0.3 hours to 24 hours, such as from 0.4 hours to 21 hours, such as from 0.5 hours to 20 hours, such as from 0.6 hours to 19 hours, such as from 0.7 hours to 18 hours, such as from 0.8 hours to 17 hours, such as from 0.9 hours to 16 hours, and including from 1 hours to 15 hours. The amount of metal salt used relative to the 25-hydroxy-(3β)-cholest-5-en-3-sulfate organic cationic salt may vary and may be 0.0001 equivalents or more, such as 0.001 equivalents or more, such as 0.01 equivalents or more, such as 0.1 equivalents or more, such as 0.2 equivalents or more, such as 0.3 equivalents or more, such as 0.4 equivalents or more, such as 0.5 equivalents or more, such as 0.6 equivalents or more, such as 0.7 equivalents or more, such as 0.8 equivalents or more, such as 0.9 equivalents or more, such as 1 equivalent or more, such as 1.1 equivalents or more, such as 1.2 equivalents or more, such as 1.3 equivalents or more, such as 1.4 equivalents or more, such as 1.5 equivalents or more, such as 1.6 equivalents or more, such as 1.7 equivalents or more, such as 1.8 equivalents or more, such as 1.9 equivalents or more, such as 2 equivalents or more, such as 3 equivalents or more, such as 4 equivalents or more, such as 5 equivalents or more, and including 10 equivalents or more, and may range from 0.001 equivalents to 10 equivalents, such as 0.1 equivalents to 10 equivalents, 0.1 equivalents to 8 equivalents, 0.1 equivalents to 6 equivalents, 0.1 equivalents to 4 equivalents, 0.1 equivalents to 3 equivalents, 1 equivalents to 10 equivalents, 1 equivalents to 8 equivalents, 1 equivalents to 6 equivalents, 1 equivalents to 4 equivalents, 1 equivalents to 3 equivalents, 1.5 equivalents to 10 equivalents, 1.5 equivalents to 8 equivalents, 1.5 equivalents to 6 equivalents, 1.5 equivalents to 4 equivalents, 1.5 equivalents to 3 equivalents, 2 equivalents to 10 equivalents, 2 equivalents to 8 equivalents, 2 equivalents to 6 equivalents, 2 equivalents to 4 equivalents, 2 equivalents to 3 equivalents, 1 equivalent to 20 equivalents, 1 equivalent to 10 equivalents, or 1 equivalent to 7 equivalents.

In some cases, methods include contacting the 25-hydroxy-(3β)-cholest-5-en-3-sulfate pyridinium salt with sodium iodide to produce a 25-hydroxy-(3β)-cholest-5-en-3-sulfate sodium salt (Scheme IIE2).

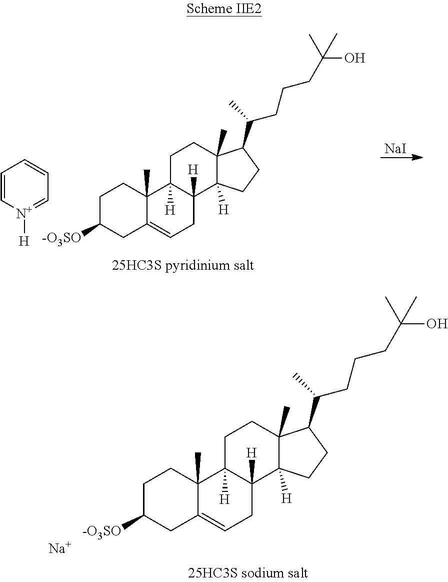

In some embodiments, the 25HC3S choline has relatively low solubility, which may be useful, e.g., in controlled release formulations such as injectable or oral controlled release formulations. As shown in the Examples, crystalline 25HC3S choline may be useful in controlled release formulations at least because of their low solubility in saline. Crystalline 25HC3S choline may also be useful in controlled release formulations at least because of its low solubility in Fasted State Simulated Gastric Fluid (FaSSGF).

In some embodiments, crystalline 25HC3S choline may be orally bioavailable. For example, salts of 25HC3S that have high solubility in Fasted State Simulated Intestinal Fluid (FaSSIF) may be orally bioavailable. As shown in the Examples, crystalline 25HC3S choline has relatively high solubility in FaSSIF. Salts of 25HC3S that have high solubility in Fed State Simulated Intestinal Fluid (FeSSIF) may also be orally bioavailable. Crystalline 25HC3S choline has relatively high solubility in FeSSIF.

In some embodiments, salts of 25HC3S are non-hygroscopic, which facilitates handling of the drug substance at ambient conditions and avoids the need for special precautions, such as the need to handle in low humidity conditions, or handle in a dry environment, or keep in a tightly closed container. The manufacturing step of weighing these drug substance salts at ambient conditions is non-problematic, since there is no concern of the weighing changes on the balance due to moisture uptake. Also, the containers of these salts can be opened and closed multiple times at ambient conditions without the concerns of the powder changing composition due to water absorptions. The non-hygroscopic nature of these salts also allows for the preparation of wet granulations for oral tablet and capsule products, and minimizes the possibility for a polymorph or other solid-form conversion such as hydrate formation. For instance, as shown in the Examples, crystalline 25HC3S choline gains less than 0.5% water at 95% relative humidity. In addition, as shown in the DVS isotherms, when the crystalline 25HC3S choline gains small amounts of water as the relative humidity is increased to 95%, they reversibly lose all that water as the relative humidity is reduced to 5%.

In some embodiments, 25HC3S choline is highly crystalline, which can be advantageous from a processing perspective, for example. Crystalline 25HC3S choline is highly crystalline. The XRPD patterns were successfully indexed by single unit cells and provide a robust description of the crystalline forms through tentative crystallographic unit cell parameters. The formula unit volumes from the indexing results are all consistent with anhydrous forms and the expected salt stoichiometry.

In some embodiments, the 25HC3S choline has a relatively high DSC (differential scanning calorimetry) endothermic transition (indicative of thermal degradation or solid state transformation). While not wishing to be bound by theory, this property may allow for dry heat sterilization (e.g., 1600 for 2 hours) of the drug substance, to facilitate preparation of sterilized dosage forms. For instance, as shown in the Examples, the first significant endothermic transition for choline is near 198° C., indicating that it may be sterilized by dry heat processing.

In some embodiments, the 25HC3S choline has good temperature stability. As shown in the Examples, crystalline 25HC3S choline has good temperature stability.

In some embodiments, the choline counterion of 25HC3S choline may have beneficial effects in vivo. For instance, the choline salt may be beneficial because choline deficiency has also been implicated in such conditions related to fat accumulation and inflammation, with choline supplementation being suggested as potentially desirable in the treatment and/or management of such conditions (see, e.g., Zeisel et al. Nutr Rev. 2009 November; 67(11): 615-623, Corbin et al. Curr Opin Gastroenterol. 2012 March; 28(2): 159-165).

### CLAUSES

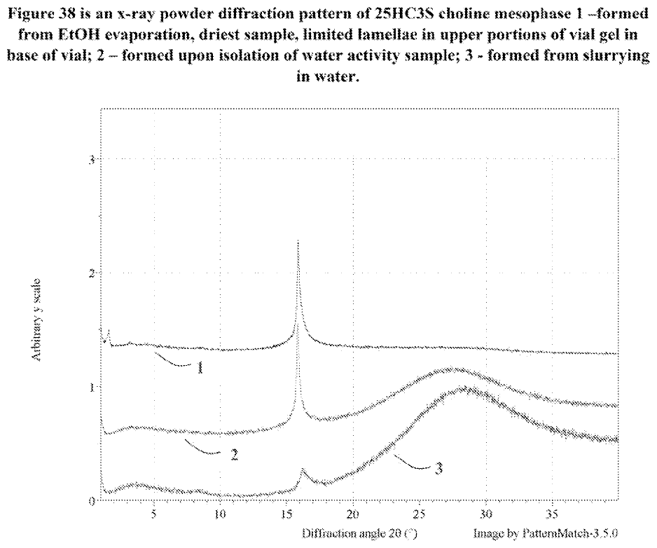

Clause 1. A pharmaceutical composition comprising 25HC3S choline and one or more fillers.
Clause 2. The pharmaceutical composition of clause 1, wherein the one or more fillers comprise one or more of talc, calcium carbonate, calcium phosphate, calcium sulfate, calcium lactate, magnesium carbonate, magnesium oxide, sodium chloride, simethicone, polysaccharide, pullulan, cellulose, cellaburate, ethylcellulose, cellulose acetate, a copolymer of ethyl acrylate and methyl methacrylate, a copolymer of methacrylic acid and methyl methacrylate, amino methacrylate copolymer, dextrate, dextrin, kaolin, mannitol, silicic acid, sorbitol, starch, alpha-lactalbumin, a sugar, a sugar alcohol, ammonium alginate, calcium silicate, fumaric acid, glyceryl palmitostearate, triglyceride, polymethacrylate, sodium alginate, sulfobutylether β-cyclodextrin, and tragacanth.
Clause 3. The pharmaceutical composition of any one of clauses 1 or 2, wherein the one or more fillers comprise one or more of dibasic calcium phosphate, tribasic calcium phosphate, microcrystalline cellulose, silicified microcrystalline cellulose, powdered cellulose, Starch1500, corn starch, modified starch, sterilizable maize starch, pregelatinized starch, pregelatinized modified starch, pea starch, hydroxypropyl pea starch, potato starch, hydroxypropyl potato starch, tapioca starch, wheat starch, hydrogenated starch hydrolysate, mannitol, isomalt, sorbitol, dextrose, polydextrose, xylitol, sucrose, lactose, lactose monohydrate, anhydrous lactose, and spray-dried lactose, compressible sugar, confectioner's sugar, sugar spheres, corn syrup, corn syrup solids, glucose, fructose, galactose, trehalose, maltose, maltodextrin, raffinose, maltitol, melezitose, stachyose, lactitol, sorbitol, erythritol, xylitol, myo-inositol, and medium-chain triglyceride.
Clause 4. The pharmaceutical composition of clause 3, wherein the dibasic calcium phosphate is anhydrous or hydrated.
Clause 5. The pharmaceutical composition of clause 4, wherein the dibasic calcium phosphate is dihydrate dibasic calcium phosphate.
Clause 6. The pharmaceutical composition of any of clauses 1 to 5, wherein the one or more fillers comprise one or more of microcrystalline cellulose and lactose monohydrate.
Clause 7. The pharmaceutical combination of any one of clauses 1 to 6, wherein the filler comprises a polysaccharide.
Clause 8. The pharmaceutical composition of clauses 1 to 7, wherein the one or more fillers comprises cellulose.
Clause 9. The pharmaceutical composition of clauses 1 to 8, wherein the one or more fillers comprises microcrystalline cellulose.
Clause 10. The pharmaceutical composition of clause 1 to 9, wherein the one or more fillers comprises a sugar.
Clause 11. The pharmaceutical composition of clause 10, wherein the sugar comprises a lactose.
Clause 12. The pharmaceutical composition of clause 11, wherein the lactose comprises lactose monohydrate.
Clause 13. The pharmaceutical composition of any one of clauses 1 to 12, wherein the one or more fillers are present in a total amount ranging from 5 wt % to 95 wt %, based on weight of the pharmaceutical composition.
Clause 14. The pharmaceutical composition of any one of clauses 1 to 13, wherein the one or more fillers are present in a total amount ranging from 40 wt % to 80 wt %, based on weight of the pharmaceutical composition.
Clause 15. The pharmaceutical composition of any one of clauses 1 to 14, wherein the one or more fillers comprise microcrystalline cellulose present in an amount ranging from 5 wt % to 95 wt %, based on weight of the pharmaceutical composition.
Clause 16. The pharmaceutical composition of any one of clauses 1 to 15, wherein the one or more fillers comprise microcrystalline cellulose present in an amount ranging from 20 wt % to 60 wt %, based on weight of the pharmaceutical composition.
Clause 17. The pharmaceutical composition any one of clauses 1 to 16, wherein the one or more fillers comprise lactose monohydrate present in an amount ranging from 5 wt % to 95 wt %, based on weight of the pharmaceutical composition.
Clause 18. The pharmaceutical composition any one of clauses 1 to 17, wherein the one or more fillers comprise lactose monohydrate present in an amount ranging from 5 wt % to 40 wt %, based on weight of the pharmaceutical composition.
Clause 19. A pharmaceutical composition comprising 25HC3S choline and one or more surfactants.
Clause 20. The pharmaceutical composition of any one of clauses 1 to 18, further comprising one or more surfactants.
Clause 21. The pharmaceutical composition of clause 19 or 20, wherein the one or more surfactants comprise one or more of poloxamers, pluronics, polyethylene glycols, polyethylene glycol monostearate, polysorbates, docusate sodium, benzalkonium chloride, sodium lauryl sulfate, sodium dodecyl sulfate, glyceryl monooleate, polyoxyethylene hydrogenated castor oil, wax, emulsifying wax, phospholipid, benzethonium chloride, cetrimide, cetylpyridinium chloride, xanthan gum, lauric acid, myristyl alcohol, butylparaben, ethylparaben, methylparaben, propylparaben, sorbic acid, polyoxyethylene alkyl ether, polyoxyethylene castor oil, polyoxyethylene sorbitan fatty acid ester, polyoxyethylene stearate, sorbitan ester, triethyl citrate, vitamin E polyethylene glycol succinate, and glycine.
Clause 22. The pharmaceutical composition of any one of clauses 19 to 21, wherein the one or more surfactants comprise one or more of poloxamer 335, poloxamer 407, poloxamer 188, polysorbate 80, polysorbate 60, polysorbate 40, and polysorbate 20.
Clause 23. The pharmaceutical composition of any one of clauses 19 to 22, wherein the one or more surfactants comprise one or more of polyoxylglyceride, behenoyl polyoxylglyceride, caprylocaproyl polyoxylglyceride, cetylpyridinium chloride, lauroyl polyoxylglyceride, linoleoyl polyoxylglyceride, octoxynol 9, oleoyl polyoxylglyceride, polyoxyl 15 hydroxystearate, nonoxynol 9, pullulan, polyoxyl lauryl ether, polyoxyl stearyl ether, polyoxyl 10 oleyl ether, polyoxyl 20 cetostearyl ether, polyoxyl 20 cetyl ether, polyoxyethylene hydrogenated castor oil, sorbitan monolaurate, sorbitan monooleate, sorbitan monopalmitate, sorbitan monostearate, stearoyl polyoxylglyceride, sorbitan sesquioleate, sorbitan trioleate, tyloxapol, and polyoxyl stearate.
Clause 24. The pharmaceutical composition any one of clauses 19 to 23, wherein the one or more surfactants comprise one or more of polyoxyl 40 stearate, polyoxyl 50 stearate, polyoxyethylene polyoxypropylene glycol, and a sucrose fatty acid ester.
Clause 25. The pharmaceutical composition of any one of clauses 19 to 24, wherein the one or more surfactants comprise sodium lauryl sulfate.
Clause 26. The pharmaceutical composition of any one of clauses 19 to 25, wherein the one or more surfactants are present in a total amount ranging from 0.5 wt % to 25 wt %, based on weight of the pharmaceutical composition.
Clause 27. The pharmaceutical composition of any one of clauses 19 to 26, wherein the one or more surfactants are present in a total amount ranging from 2 wt % to 10 wt %, based on weight of the pharmaceutical composition.
Clause 28. The pharmaceutical composition of any one of clauses 19 to 27, wherein the one or more surfactants comprise sodium lauryl sulfate present in an amount ranging from 0.5 wt % to 25 wt %, based on weight of the pharmaceutical composition.
Clause 29. The pharmaceutical composition of clause 28, wherein the sodium lauryl sulfate is present in an amount ranging from 2 wt % to 10 wt %, based on weight of the pharmaceutical composition.
Clause 30. A pharmaceutical composition comprising 25HC3S choline and one or more disintegrants.
Clause 31. The pharmaceutical composition of any one of clauses 1 to 29, further comprising one or more disintegrants.
Clause 32. The pharmaceutical composition of clause 30 or 31, wherein the one or more disintegrants comprise one or more of cross-linked starch, sodium starch glycolate, cross-linked cellulose, cross-linked carboxymethylcellulose, the sodium salt of cross-linked carboxymethylcellulose, polyvinylpyrrolidone, cross-linked polyvinylpyrrolidone, chitosan hydrochloride, corn starch, docusate sodium, magnesium aluminum silicate, starch, modified starch, pregelatinized starch, pregelatinized modified starch, hydroxypropyl starch, cellulose, methylcellulose, sodium carboxymethyl cellulose, calcium carboxymethyl cellulose, substituted hydroxypropyl cellulose, microcrystalline cellulose, calcium cellulose glycolate, carmellosum calcium, alginates, alginic acid, silicon dioxide, colloidal silicon dioxide, glycine, guar gum, polacrilin potassium, and sodium bicarbonate.
Clause 33. The pharmaceutical composition of any one of clauses 30 to 32, wherein the one or more disintegrants comprise one or more of powdered cellulose, low-substituted hydroxypropyl cellulose, sodium alginate, calcium alginate, and calcium sodium alginate.
Clause 34. The pharmaceutical composition of any one of clauses 30 or 33, wherein the one or more disintegrants comprise polyvinylpyrrolidone.
Clause 35. The pharmaceutical composition of clause 34, wherein the polyvinylpyrrolidone comprises cross-linked polyvinylpyrrolidone.
Clause 36. The pharmaceutical composition of clause 35, wherein the cross-linked polyvinylpyrrolidone comprises crospovidone.
Clause 37. The pharmaceutical composition of any one of clauses 30 to 36, wherein the one or more disintegrants comprise sodium bicarbonate.
Clause 38. The pharmaceutical composition of any one of clauses 30 to 37, comprising two or more disintegrants.
Clause 39. The pharmaceutical composition of clause 38, wherein the two or more disintegrants comprise one or more of poloxamer, croscarmellose sodium, meglumine, and cross-linked polyvinylpyrrolidone.
Clause 40. The pharmaceutical composition of any one of clauses 30 to 39, comprising three or more disintegrants.
Clause 41. The pharmaceutical composition of any one of clauses 30 to 40, wherein the one or more disintegrants are present in a total amount ranging from 0.5 wt % to 25 wt %, based on weight of the pharmaceutical composition.
Clause 42. The pharmaceutical composition of any one of clauses 30 to 41, wherein the one or more disintegrants are present in a total amount ranging from 0.5 wt % to 15 wt %, based on weight of the pharmaceutical composition.
Clause 43. The pharmaceutical composition of any one of clauses 30 to 42, wherein the one or more disintegrants comprise crospovidone present in an amount ranging from 0.5 wt % to 25 wt %, based on weight of the pharmaceutical composition.
Clause 44. The pharmaceutical composition of clause 43, wherein the crospovidone is present in an amount ranging from 0.5 wt % to 15 wt %, based on weight of the pharmaceutical composition.
Clause 45. The pharmaceutical composition of any one of clauses 30 to 44, wherein the one or more disintegrants are present in an amount sufficient to provide for release of 20% or more of the 25HC3S choline within 45 minutes of administration to a human or being placed in a USP Apparatus 2 having 900 mL of dissolution medium that is 2.0% sodium dodecyl sulfate in 0.1 N HCl with a paddle speed of 75 rpm and a vessel temperature of 37° C.
Clause 46. The pharmaceutical composition of any one of clauses 30 to 45, wherein the one or more disintegrants are present in an amount sufficient to provide for release of 30% or more of the 25HC3S choline within 45 minutes of administration to a human or being placed in a USP Apparatus 2 having 900 mL of dissolution medium that is 2.0% sodium dodecyl sulfate in 0.1 N HCl with a paddle speed of 75 rpm and a vessel temperature of 37° C.
Clause 47. The pharmaceutical composition of any one of clauses 30 to 46, wherein the one or more disintegrants are present in an amount sufficient to provide for release of 50% or more of the 25HC3S choline within 45 minutes of administration to a human or being placed in a USP Apparatus 2 having 900 mL of dissolution medium that is 2.0% sodium dodecyl sulfate in 0.1 N HCl with a paddle speed of 75 rpm and a vessel temperature of 37° C.
Clause 48. A pharmaceutical composition comprising 25HC3S choline and one or more glidants.
Clause 49. The pharmaceutical composition of any one of clauses 1 to 47, further comprising one or more glidants.
Clause 50. The pharmaceutical composition of clause 48 or 49, wherein the one or more glidants comprise one or more of silicon dioxide, colloidal silicon dioxide, hydrophobic colloidal silica, colloidal anhydrous silica, magnesium trisilicate, tribasic calcium phosphate, calcium silicate, magnesium silicate, colloidal silicon dioxide, magnesium oxide, cellulose, sodium stearate, cellulose, powdered cellulose, starch, and talc.
Clause 51. The pharmaceutical composition of any one of clauses 48 to 50, wherein the one or more glidants comprise silicon dioxide.
Clause 52. The pharmaceutical composition of any one of clauses 48 to 51, wherein the one or more glidants comprise colloidal silicon dioxide.
Clause 53. The pharmaceutical composition of any one of clauses 48 to 52, wherein the one or more glidants are present in a total amount ranging from 0.2 wt % to 10 wt %, based on weight of the pharmaceutical composition.
Clause 54. The pharmaceutical composition of any one of clauses 48 to 53, wherein the one or more glidants are present in a total amount ranging from 0.5 wt % to 3 wt %, based on weight of the pharmaceutical composition.
Clause 55. The pharmaceutical composition of any one of clauses 48 to 54, wherein the one or more glidants comprise silicon dioxide present in an amount ranging from 0.2 wt % to 10 wt %, based on weight of the pharmaceutical composition.
Clause 56. The pharmaceutical composition of clause 55, wherein the silicon dioxide is present in an amount ranging from 0.5 wt % to 3 wt %, based on weight of the pharmaceutical composition.
Clause 57. A pharmaceutical composition comprising 25HC3S choline and one or more lubricants.
Clause 58. The pharmaceutical composition of any one of clauses 1 to 56, further comprising one or more lubricants.
Clause 59. The pharmaceutical composition of clause 57 or 58, wherein the one or more lubricants comprise one or more of magnesium stearate, aluminum stearate, calcium stearate, zinc stearate, sodium stearate, stearic acid, magnesium silicate, polyethylene glycol, glyceryl behenate, mineral oil, light mineral oil, sodium stearyl fumarate, talc, hydrogenated vegetable oil, sodium lauryl sulfate, magnesium lauryl sulfate, behenoyl polyoxylglyceride, glyceryl dibehenate, lauric acid, glyceryl monostearate, glyceryl tristearate, myristic acid, palmitic acid, poloxamer, polysorbate 20, polyoxyl 10 oleyl ether, polyoxyl 15 hydroxystearate, polysorbate 40, polyoxyl 20 cetostearyl ether, polyoxyl 40 stearate, polysorbate 60, polysorbate 80, potassium benzoate, sodium benzoate, sorbitan monolaurate, sorbitan monooleate, sorbitan monopalmitate, sorbitan monostearate, sorbitan sesquioleate, sorbitan trioleate, glyceryl palmitostearate, hydrogenated castor oil, triglyceride, medium-chain triglyceride, and sodium chloride.
Clause 60. The pharmaceutical composition of any one of clauses 57 to 59, wherein the one or more lubricants comprise polyethylene glycol 3350.
Clause 61. The pharmaceutical composition of any one of clauses 57 to 60, wherein the one or more lubricants comprise magnesium stearate.
Clause 62. The pharmaceutical composition of any one of clauses 57 to 61, wherein the one or more lubricants are present in a total amount ranging from 0.1 wt % to 5 wt %, based on weight of the pharmaceutical composition.
Clause 63. The pharmaceutical composition of any one of clauses 57 to 62, wherein the one or more lubricants are present in a total amount ranging from 0.5 wt % to 2 wt %, based on weight of the pharmaceutical composition.
Clause 64. The pharmaceutical composition of any one of clauses 57 to 63, wherein the one or more lubricants comprise magnesium stearate present in an amount ranging from 0.1 wt % to 5 wt %, based on weight of the pharmaceutical composition.
Clause 65. The pharmaceutical composition of clause 64, wherein the magnesium stearate is present in an amount ranging from 0.5 wt % to 2 wt %, based on weight of the pharmaceutical composition.
Clause 66. A pharmaceutical composition comprising 25HC3S choline and one or more binders.
Clause 67. The pharmaceutical composition of any one of clauses 1 to 65, further comprising one or more binders.
Clause 68. The pharmaceutical composition of clause 66 or 67, wherein the one or more binders comprise one or more of polyvinylpyrrolidone, copovidone, carbomer, corn starch, pregelatinized starch, carboxymethylcellulose sodium, hydroxypropyl methylcellulose, polyethylene glycol, hydroxyethyl cellulose, hydroxypropyl cellulose, hydroxyethylmethyl cellulose, calcium carboxymethylcellulose, calcium cellulose glycolate, guar galactomannan, ethylcellulose, chitosan, chitosan hydrochloride, dextrin, low-substituted hydroxypropyl cellulose, hydroxypropyl starch, ceratonia, inulin, magnesium aluminum silicate, maltodextrin, methylcellulose, dextrate, polyethylene oxide, povidone, sodium alginate, starch, glucose, sucrose, compressible sugar, zein, gelatin, polymethacrylate, sorbitol, liquid glucose, acacia, agar, alginic acid, calcium carbonate, calcium lactate, carrageenan, cellulose acetate phthalate, cottonseed oil, dextrose, glyceryl behenate, guar gum, hydrogenated vegetable oil, hypromellose, lactose, maltose, microcrystalline cellulose, pectin, poloxamer, polycarbophil, polydextrose, stearic acid, sunflower oil, tricaprylin, and vitamin E polyethylene glycol succinate.
Clause 69. The pharmaceutical composition of any one of clauses 66 to 68, wherein the one or more binders are present in a total amount ranging from 2 wt % to 40 wt %, based on weight of the pharmaceutical composition.
Clause 70. A pharmaceutical composition comprising 25HC3S choline and one or more plasticizers.
Clause 71. The pharmaceutical composition of any one of clauses 1 to 69, further comprising one or more plasticizers.
Clause 72. The pharmaceutical composition of clause 70 or 71, wherein the one or more plasticizers comprise one or more of acetyltributyl citrate, acetyltriethyl citrate, benzyl benzoate, cellulose acetate phthalate, chlorbutanol, dextrin, dibutyl phthalate, dibutyl sebacate, diethyl phthalate, dimethyl phthalate, glycerin, glycerin monostearate, hypromellose phthalate, mannitol, mineral oil, lanolin alcohol, palmitic acid, petrolatum, polyethylene glycol, polymethacrylate, polyvinyl acetate phthalate, propylene glycol, pyrrolidone, sorbitol, stearic acid, triacetin, tributyl citrate, triethanolamine, and triethyl citrate.
Clause 73. The pharmaceutical composition of any one of clauses 70 to 72, wherein the one or more plasticizers are present in a total amount ranging from 0.1 wt % to 50 wt %, based on weight of the pharmaceutical composition.
Clause 74. The pharmaceutical composition of any one of clauses 1 to 73, further comprising a coating comprising one or more coating materials.
Clause 75. The pharmaceutical composition of clause 74, wherein the one or more coating materials comprise one or more of a polymer, a colorant, a plasticizer, and a solvent.
Clause 76. The pharmaceutical composition of clause 74 or 75, wherein the one or more coating materials comprise one or more of a film-former and an opaquant-extender.
Clause 77. The pharmaceutical composition of clause 76, wherein the film-former is enteric.
Clause 78. The pharmaceutical composition of clause 76, wherein the film-former is non-enteric.
Clause 79. The pharmaceutical composition of any one of clauses 74 to 78, wherein the one or more coating materials comprise one or more of carnauba wax, cellulose acetate, cellulose acetate phthalate, ceresin, cetyl alcohol, chitosan, ethylcellulose, fructose, gelatin, glycerin, glyceryl behenate, glyceryl palmitostearate, hydroxyethyl cellulose, hydroxyethylmethyl cellulose, hydroxypropyl cellulose, hypromellose, hypromellose phthalate, isomalt, latex particles, glucose, liquid glucose, macrogol 400, maltitol, maltodextrin, methylcellulose, microcrystalline wax, paraffin, poloxamer, polydextrose, polyethylene glycol, polyethylene oxide, poly-DL-(lactic acid), polyvinyl acetate phthalate, polyvinyl alcohol, povidone, shellac, shellac with stearic acid, surface color agents, titanium oxide, tributyl citrate, triethyl citrate, vanillin, wax, white wax, xylitol, yellow wax, zein, polysorbate, acetyltriethyl citrate, carboxymethylcellulose calcium, carboxymethylcellulose sodium, and sucrose.
Clause 80. The pharmaceutical composition of any one of clauses 74 to 79, wherein the one or more coating materials are present in a total amount ranging from 0.1 wt % to 10 wt %, based on weight of the pharmaceutical composition.
Clause 81. A pharmaceutical composition comprising 25HC3S choline and one or more colorants.
Clause 82. The pharmaceutical composition of any one of clauses 1 to 80, further comprising one or more colorants.
Clause 83. The pharmaceutical composition of clause 81 or 82, wherein the one or more colorants comprise one or more of dyes, lakes, inorganic pigments, and natural colorants.
Clause 84. The pharmaceutical composition of any one of clauses 81 to 83, wherein the one or more colorants comprise titanium dioxide, an iron oxide, or both.
Clause 85. The pharmaceutical composition of any one of clauses 81 to 84, wherein the one or more colorants comprise riboflavin.
Clause 86. The pharmaceutical composition of any one of clauses 81 to 85, wherein the one or more colorants comprise one or more of caramel, ferric oxide, titanium dioxide, ferroferric oxide, aluminum oxide, FD & C Red #40/Allura Red AC, amaranth, FD & C Blue #1/Brilliant Blue FCF, canthaxanthin, carmine, carmoisine (azorubine), curcumin (tumeric), FD & C Red #3/erythrosine, Fast Green FCF, Green S (Lissamine Green), D & C Red #30/helendon pink, FD & C Blue #2/indigo carmine, iron oxide black, iron oxide red, D & C Red #7/Lithol Rubin BK, Patent Blue V, D & C Red #28/Phloxine B, iron oxide yellow, D & C Red #27/Phloxine O, Ponceau 4R (Cochineal Red A), Quinoline Yellow WS, D & C Yellow #10, riboflavin (lactoflavin), FD & C Yellow #5/tartrazine, and FD & C Yellow #6/Sunset Yellow FCF.
Clause 87. The pharmaceutical composition of any one of clauses 81 to 86, wherein the one or more colorants are present in a total amount ranging from 0.01 wt % to 5 wt %, based on weight of the pharmaceutical composition.
Clause 88. A pharmaceutical composition comprising 25HC3S choline and one or more flavoring agents.
Clause 89. The pharmaceutical composition of any one of clauses 1 to 87, further comprising one or more flavoring agents.
Clause 90. The pharmaceutical composition of clause 88 or 89, wherein the one or more flavoring agents comprise one or more of vanillin, peppermint flavor powder, berry flavor powder, strawberry flavor powder, orange flavor powder, lemon flavor powder, orange essence, ethyl maltol, eucalyptus oil, isobutyl alcohol, sodium succinate, adipic acid, almond oil, anethole, benzaldehyde, denatonium benzoate, ethyl acetate, ethyl vanillin, ethylcellulose, fructose, fumaric acid, L-glutamic acid hydrochloride, lactitol, leucine, malic acid, maltol, menthol, racementhol, methionine, methyl salicylate, monosodium glutamate, peppermint oil, liquid strawberry flavor, peppermint spirit, racemethionine, rose oil, rose water, sodium acetate, sodium lactate, tartaric acid, thymol, inulin, isomalt, and neohesperidin dihydrochalcone.
Clause 91. The pharmaceutical composition of any one of clauses 88 to 90, wherein the one or more flavoring agents are present in a total amount ranging from 0.1 wt % to 10 wt %, based on weight of the pharmaceutical composition.
Clause 92. A pharmaceutical composition comprising 25HC3S choline and one or more sweeteners.
Clause 93. The pharmaceutical composition of any one of clauses 1 to 91, further comprising one or more sweeteners.
Clause 94. The pharmaceutical composition of clause 92 or 93, wherein the one or more sweeteners comprise one or more of sucralose, saccharin, saccharin calcium, saccharin sodium, neotame, sucrose, acesulfame potassium, aspartame, aspartame acesulfame, corn syrup, corn syrup solids, dextrate, dextrose, erythritol, fructose, galactose, glucose, glycerin, inulin, invert sugar, isomalt, lactitol, maltitol, maltose, mannitol, sorbitol, hydrogenated starch hydrolysate, compressible sugar, confectioner's sugar, tagatose, trehalose, and xylitol.
Clause 95. The pharmaceutical composition of any one of clauses 92 to 94, wherein the one or more sweeteners are present in a total amount ranging from 0.1 wt % to 20 wt %, based on weight of the pharmaceutical composition.
Clause 96. The pharmaceutical composition of any one of clauses 1 to 95, wherein the 25HC3S choline is spray dried.
Clause 97. The pharmaceutical composition of any one of clauses 1 to 96, wherein the 25HC3S choline is micronized.
Clause 98. The pharmaceutical composition of any one of clauses 1 to 97 in tablet form.
Clause 99. The pharmaceutical composition of any one of clauses 1 to 97 in capsule form.
Clause 100. A method of treating or preventing one or more of nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, alcoholic fatty liver disease (AFLD), alcoholic steatohepatitis (ASH), leptin resistance, leptin deficiency, a diabetes condition, an autoimmune condition, an inflammatory condition, a neurological condition, Epstein Barr virus-related growth, and conditions related to fat accumulation and inflammation, comprising administering to a patient in need thereof an effective amount of a pharmaceutical composition of any one of clauses 1 to 99.
Clause 101. The pharmaceutical composition of any one of clauses 1 to 99, wherein the 25HC3S choline is crystalline.
Clause 102. The pharmaceutical composition of clause 101, wherein the 25HC3S choline has an x-ray powder diffraction pattern comprising a peak at about 3.9°2θ.
Clause 103. The pharmaceutical composition of clause 101, wherein the 25HC3S choline has an x-ray powder diffraction pattern comprising a peak at about 7.8°2θ.
Clause 104. The pharmaceutical composition of clause 101, wherein the 25HC3S choline has an x-ray powder diffraction pattern comprising a peak at about 9.5°2θ.
Clause 105. The pharmaceutical composition of clause 101, wherein the 25HC3S choline has an x-ray powder diffraction pattern comprising a peak at about 10.1°2θ.
Clause 106. The pharmaceutical composition of clause 101, wherein the 25HC3S choline has an x-ray powder diffraction pattern comprising a peak at about 11.0°2θ.
Clause 107. The pharmaceutical composition of clause 101, wherein the 25HC3S choline has an x-ray powder diffraction pattern comprising a peak at about 12.2°2θ.
Clause 108. The pharmaceutical composition of clause 101, wherein the 25HC3S choline has an x-ray powder diffraction pattern comprising a peak at about 13.7°2θ.
Clause 109. The pharmaceutical composition of clause 101, wherein the 25HC3S choline has an x-ray powder diffraction pattern comprising a peak at about 14.7°2θ.
Clause 110. The pharmaceutical composition of clause 101, wherein the 25HC3S choline has an x-ray powder diffraction pattern comprising a peak at about 15.1°2θ.
Clause 111. The pharmaceutical composition of clause 101, wherein the 25HC3S choline has an x-ray powder diffraction pattern comprising a peak at about 15.8°2θ.
Clause 112. The pharmaceutical composition of clause 101, wherein the 25HC3S choline has an x-ray powder diffraction pattern comprising a peak at about 16.3°2θ.
Clause 113. The pharmaceutical composition of clause 101, wherein the 25HC3S choline has an x-ray powder diffraction pattern comprising a peak at about 19.1°2θ.
Clause 114. The pharmaceutical composition of clause 102, wherein the 25HC3S choline has an x-ray powder diffraction pattern further comprising a peak at about 7.8°2θ.
Clause 115. The pharmaceutical composition of clause 102, wherein the 25HC3S choline has an x-ray powder diffraction pattern further comprising a peak at about 9.5°2θ.
Clause 116. The pharmaceutical composition of clause 102, wherein the 25HC3S choline has an x-ray powder diffraction pattern further comprising a peak at about 10.1°2θ.
Clause 117. The pharmaceutical composition of clause 102, wherein the 25HC3S choline has an x-ray powder diffraction pattern further comprising a peak at about 11.0°2θ.
Clause 118. The pharmaceutical composition of clause 102, wherein the 25HC3S choline has an x-ray powder diffraction pattern further comprising a peak at about 12.2°2θ.
Clause 119. The pharmaceutical composition of clause 102, wherein the 25HC3S choline has an x-ray powder diffraction pattern further comprising a peak at about 13.7°2θ.
Clause 120. The pharmaceutical composition of clause 102, wherein the 25HC3S choline has an x-ray powder diffraction pattern further comprising a peak at about 14.7°2θ.
Clause 121. The pharmaceutical composition of clause 102, wherein the 25HC3S choline has an x-ray powder diffraction pattern further comprising a peak at about 15.1°2θ.
Clause 122. The pharmaceutical composition of clause 102, wherein the 25HC3S choline has an x-ray powder diffraction pattern further comprising a peak at about 15.8°2θ.
Clause 123. The pharmaceutical composition of clause 102, wherein the 25HC3S choline has an x-ray powder diffraction pattern further comprising a peak at about 16.3°2θ.
Clause 124. The pharmaceutical composition of clause 102, wherein the 25HC3S choline has an x-ray powder diffraction pattern further comprising a peak at about 19.1°2θ.
Clause 125. The pharmaceutical composition of clause 114, wherein the 25HC3S choline has an x-ray powder diffraction pattern further comprising a peak at about 9.5°2θ.
Clause 126. The pharmaceutical composition of clause 114, wherein the 25HC3S choline has an x-ray powder diffraction pattern further comprising a peak at about 10.1°2θ.
Clause 127. The pharmaceutical composition of clause 114, wherein the 25HC3S choline has an x-ray powder diffraction pattern further comprising a peak at about 11.0°2θ.
Clause 128. The pharmaceutical composition of clause 114, wherein the 25HC3S choline has an x-ray powder diffraction pattern further comprising a peak at about 12.2°2θ.
Clause 129. The pharmaceutical composition of clause 114, wherein the 25HC3S choline has an x-ray powder diffraction pattern further comprising a peak at about 13.7°2θ.
Clause 130. The pharmaceutical composition of clause 114, wherein the 25HC3S choline has an x-ray powder diffraction pattern further comprising a peak at about 14.7°2θ.
Clause 131. The pharmaceutical composition of clause 114, wherein the 25HC3S choline has an x-ray powder diffraction pattern further comprising a peak at about 15.1°2θ.
Clause 132. The pharmaceutical composition of clause 114, wherein the 25HC3S choline has an x-ray powder diffraction pattern further comprising a peak at about 15.8°2θ.
Clause 133. The pharmaceutical composition of clause 114, wherein the 25HC3S choline has an x-ray powder diffraction pattern further comprising a peak at about 16.3°2θ.
Clause 134. The pharmaceutical composition of clause 114, wherein the 25HC3S choline has an x-ray powder diffraction pattern further comprising a peak at about 19.1°2θ.
Clause 135. The pharmaceutical composition of clause 125, wherein the 25HC3S choline has an x-ray powder diffraction pattern further comprising a peak at about 10.1°2θ.
Clause 136. The pharmaceutical composition of clause 125, wherein the 25HC3S choline has an x-ray powder diffraction pattern further comprising a peak at about 11.0°2θ.
Clause 137. The pharmaceutical composition of clause 125, wherein the 25HC3S choline has an x-ray powder diffraction pattern further comprising a peak at about 12.2°2θ.
Clause 138. The pharmaceutical composition of clause 125, wherein the 25HC3S choline has an x-ray powder diffraction pattern further comprising a peak at about 13.7°2θ.
Clause 139. The pharmaceutical composition of clause 125, wherein the 25HC3S choline has an x-ray powder diffraction pattern further comprising a peak at about 14.7°2θ.
Clause 140. The pharmaceutical composition of clause 125, wherein the 25HC3S choline has an x-ray powder diffraction pattern further comprising a peak at about 15.1°2θ.
Clause 141. The pharmaceutical composition of clause 125, wherein the 25HC3S choline has an x-ray powder diffraction pattern further comprising a peak at about 15.8°2θ.
Clause 142. The pharmaceutical composition of clause 125, wherein the 25HC3S choline has an x-ray powder diffraction pattern further comprising a peak at about 16.3°2θ.
Clause 143. The pharmaceutical composition of clause 125, wherein the 25HC3S choline has an x-ray powder diffraction pattern further comprising a peak at about 19.1°2θ.
Clause 144. The pharmaceutical composition of clause 135, wherein the 25HC3S choline has an x-ray powder diffraction pattern further comprising a peak at about 11.0°2θ.
Clause 145. The pharmaceutical composition of clause 135, wherein the 25HC3S choline has an x-ray powder diffraction pattern further comprising a peak at about 12.2°2θ.
Clause 146. The pharmaceutical composition of clause 135, wherein the 25HC3S choline has an x-ray powder diffraction pattern further comprising a peak at about 13.7°2θ.
Clause 147. The pharmaceutical composition of clause 135, wherein the 25HC3S choline has an x-ray powder diffraction pattern further comprising a peak at about 14.7°2θ.
Clause 148. The pharmaceutical composition of clause 135, wherein the 25HC3S choline has an x-ray powder diffraction pattern further comprising a peak at about 15.1°2θ.
Clause 149. The pharmaceutical composition of clause 135, wherein the 25HC3S choline has an x-ray powder diffraction pattern further comprising a peak at about 15.8°2θ.
Clause 150. The pharmaceutical composition of clause 135, wherein the 25HC3S choline has an x-ray powder diffraction pattern further comprising a peak at about 16.3°2θ.
Clause 151. The pharmaceutical composition of clause 135, wherein the 25HC3S choline has an x-ray powder diffraction pattern further comprising a peak at about 19.1°2θ.
Clause 152. The pharmaceutical composition of clause 144, wherein the 25HC3S choline has an x-ray powder diffraction pattern further comprising a peak at about 12.2°2θ.
Clause 153. The pharmaceutical composition of clause 144, wherein the 25HC3S choline has an x-ray powder diffraction pattern further comprising a peak at about 13.7°2θ.
Clause 154. The pharmaceutical composition of clause 144, wherein the 25HC3S choline has an x-ray powder diffraction pattern further comprising a peak at about 14.7°2θ.
Clause 155. The pharmaceutical composition of clause 144, wherein the 25HC3S choline has an x-ray powder diffraction pattern further comprising a peak at about 15.1°2θ.
Clause 156. The pharmaceutical composition of clause 144, wherein the 25HC3S choline has an x-ray powder diffraction pattern further comprising a peak at about 15.8°2θ.
Clause 157. The pharmaceutical composition of clause 144, wherein the 25HC3S choline has an x-ray powder diffraction pattern further comprising a peak at about 16.3°2θ.
Clause 158. The pharmaceutical composition of clause 144, wherein the 25HC3S choline has an x-ray powder diffraction pattern further comprising a peak at about 19.1°2θ.
Clause 159. The pharmaceutical composition of clause 152, wherein the 25HC3S choline has an x-ray powder diffraction pattern further comprising a peak at about 13.7°2θ.
Clause 160. The pharmaceutical composition of clause 152, wherein the 25HC3S choline has an x-ray powder diffraction pattern further comprising a peak at about 14.7°2θ.
Clause 161. The pharmaceutical composition of clause 152, wherein the 25HC3S choline has an x-ray powder diffraction pattern further comprising a peak at about 15.1°2θ.
Clause 162. The pharmaceutical composition of clause 152, wherein the 25HC3S choline has an x-ray powder diffraction pattern further comprising a peak at about 15.8°2θ.
Clause 163. The pharmaceutical composition of clause 152, wherein the 25HC3S choline has an x-ray powder diffraction pattern further comprising a peak at about 16.3°2θ.
Clause 164. The pharmaceutical composition of clause 152, wherein the 25HC3S choline has an x-ray powder diffraction pattern further comprising a peak at about 19.1°2θ.
Clause 165. The pharmaceutical composition of clause 159, wherein the 25HC3S choline has an x-ray powder diffraction pattern further comprising a peak at about 14.7°2θ.
Clause 166. The pharmaceutical composition of clause 159, wherein the 25HC3S choline has an x-ray powder diffraction pattern further comprising a peak at about 15.1°2θ.
Clause 167. The pharmaceutical composition of clause 159, wherein the 25HC3S choline has an x-ray powder diffraction pattern further comprising a peak at about 15.8°2θ.
Clause 168. The pharmaceutical composition of clause 159, wherein the 25HC3S choline has an x-ray powder diffraction pattern further comprising a peak at about 16.3°2θ.
Clause 169. The pharmaceutical composition of clause 159, wherein the 25HC3S choline has an x-ray powder diffraction pattern further comprising a peak at about 19.1°2θ.
Clause 170. The pharmaceutical composition of clause 165, wherein the 25HC3S choline has an x-ray powder diffraction pattern further comprising a peak at about 15.1°2θ.
Clause 171. The pharmaceutical composition of clause 165, wherein the 25HC3S choline has an x-ray powder diffraction pattern further comprising a peak at about 15.8°2θ.
Clause 172. The pharmaceutical composition of clause 165, wherein the 25HC3S choline has an x-ray powder diffraction pattern further comprising a peak at about 16.3°2θ.
Clause 173. The pharmaceutical composition of clause 165, wherein the 25HC3S choline has an x-ray powder diffraction pattern further comprising a peak at about 19.1°2θ.
Clause 174. The pharmaceutical composition of clause 170, wherein the 25HC3S choline has an x-ray powder diffraction pattern further comprising a peak at about 15.8°2θ.
Clause 175. The pharmaceutical composition of clause 170, wherein the 25HC3S choline has an x-ray powder diffraction pattern further comprising a peak at about 16.3°2θ.
Clause 176. The pharmaceutical composition of clause 170, wherein the 25HC3S choline has an x-ray powder diffraction pattern further comprising a peak at about 19.1°2θ.
Clause 177. The pharmaceutical composition of clause 174, wherein the 25HC3S choline has an x-ray powder diffraction pattern further comprising a peak at about 16.3°2θ.
Clause 178. The pharmaceutical composition of clause 174, wherein the 25HC3S choline has an x-ray powder diffraction pattern further comprising a peak at about 19.1°2θ.
Clause 179. The pharmaceutical composition of clause 101, wherein the 25HC3S choline has an x-ray powder diffraction pattern comprising one or more of the following peaks at about 3.9°2θ, about 7.8°2θ, about 9.5°2θ, about 10.1°2θ, about 11.0°2θ, about 12.2°2θ, about 13.7°2θ, about 14.7°2θ, about 15.1°2θ, about 15.8°2θ, about 16.3°2θ, and about 19.1°2θ.
Clause 180. The pharmaceutical composition of clause 101, wherein the 25HC3S choline has an x-ray powder diffraction pattern comprising one or more of the following peaks at about 7.8°2θ, about 9.5°2θ, about 10.1°2θ, about 11.0°2θ, about 12.2°2θ, about 13.7°2θ, about 14.7°2θ, about 15.1°2θ, about 15.8°2θ, about 16.3°2θ, and about 19.1°2θ.
Clause 181. The pharmaceutical composition of clause 101, wherein the 25HC3S choline has an x-ray powder diffraction pattern comprising one or more of the following peaks at about 9.5°2θ, about 10.1°2θ, about 11.0°2θ, about 12.2°2θ, about 13.7°2θ, about 14.7°2θ, about 15.1°2θ, about 15.8°2θ, about 16.3°2θ, and about 19.1°2θ.
Clause 182. The pharmaceutical composition of clause 101, wherein the 25HC3S choline has an x-ray powder diffraction pattern comprising one or more of the following peaks at about 10.1°2θ, about 11.0°2θ, about 12.2°2θ, about 13.7°2θ, about 14.7°2θ, about 15.1°2θ, about 15.8°2θ, about 16.3°2θ, and about 19.1°2θ.
Clause 183. The pharmaceutical composition of clause 101, wherein the 25HC3S choline has an x-ray powder diffraction pattern comprising one or more of the following peaks at about 11.0°2θ, about 12.2°2θ, about 13.7°2θ, about 14.7°2θ, about 15.1°2θ, about 15.8°2θ, about 16.3°2θ, and about 19.1°2θ.
Clause 184. The pharmaceutical composition of clause 101, wherein the 25HC3S choline has an x-ray powder diffraction pattern comprising one or more of the following peaks at about 12.2°2θ, about 13.7°2θ, about 14.7°2θ, about 15.1°2θ, about 15.8°2θ, about 16.3°2θ, and about 19.1°2θ.
Clause 185. The pharmaceutical composition of clause 101, wherein the 25HC3S choline has an x-ray powder diffraction pattern comprising one or more of the following peaks at about 13.7°2θ, about 14.7°2θ, about 15.1°2θ, about 15.8°2θ, about 16.3°2θ, and about 19.1°2θ.
Clause 186. The pharmaceutical composition of clause 101, wherein the 25HC3S choline has an x-ray powder diffraction pattern comprising one or more of the following peaks at about 14.7°2θ, about 15.1°2θ, about 15.8°2θ, about 16.3°2θ, and about 19.1°2θ.
Clause 187. The pharmaceutical composition of clause 101, wherein the 25HC3S choline has an x-ray powder diffraction pattern comprising one or more of the following peaks at about 15.1°2θ, about 15.8°2θ, about 16.3°2θ, and about 19.1°2θ.
Clause 188. The pharmaceutical composition of clause 101, wherein the 25HC3S choline has an x-ray powder diffraction pattern comprising one or more of the following peaks at about 15.8°2θ, about 16.3°2θ, and about 19.1°2θ.
Clause 189. The pharmaceutical composition of clause 101, wherein the 25HC3S choline has an x-ray powder diffraction pattern comprising one or more of the following peaks at about 16.3° 20 and about 19.1°2θ.
Clause 190. The pharmaceutical composition of clause 101, wherein the 25HC3S choline has an x-ray powder diffraction pattern comprising a peak at about 19.1°2θ.
Clause 191. The pharmaceutical composition of clause 101, wherein the 25HC3S choline has an x-ray powder diffraction pattern substantially the same as that found in FIG. 1.
Clause 192. The pharmaceutical composition of clauses 101 to 191, wherein the 25HC3S choline has an orthorhombic unit cell.
Clause 193. The pharmaceutical composition of clauses 101 to 192, wherein the 25HC3S choline has a unit cell with lengths of about 7.9 Å, about 9.5 Å, and about 45. IA.
Clause 194. The pharmaceutical composition of clauses 101 to 193, wherein the water uptake by the crystalline choline salt is less than 0.5 wt % between a relative humidity range of about 5% to about 95%.
Clause 195. The pharmaceutical composition of clause 101, wherein the 25HC3S choline has an x-ray powder diffraction pattern comprising peaks at about 3.9°2θ, about 7.8°2θ, about 9.5°2θ, about 10.1°2θ, about 11.0°2θ, about 12.2°2θ, about 13.7°2θ, about 14.7°2θ, about 15.1°2θ, about 15.8°2θ, and about 16.3°2θ.
Clause 196. The pharmaceutical composition of clause 195, further comprising a peak at about 19.1°2θ.
Clause 197. The pharmaceutical composition of clause 101, wherein the 25HC3S choline has an x-ray powder diffraction pattern comprising peaks at about 3.9°2θ, about 7.8°2θ, about 9.5°2θ, about 10.1°2θ, about 11.0°2θ, about 12.2°2θ, about 13.7°2θ, about 14.7°2θ, about 15.1°2θ, about 15.8°2θ, about 16.3°2θ, and about 19.1°2θ.
Clause 198. The pharmaceutical composition of any one of clauses 1 to 197 wherein the 25HC3S choline is substantially pure based on the amount of 25HC3S choline present.
Clause 199. The pharmaceutical composition of clause 198, wherein the pharmaceutical composition comprises an impurity in an amount of 2 wt % or less based on the amount of 25HC3S choline present.
Clause 200. The pharmaceutical composition of clause 199, wherein the impurity comprises sulfated desmosterol.
Clause 201. The pharmaceutical composition of clause 200, wherein when the composition is stored at 25° C./60% RH for 2 weeks, the sulfated desmosterol is present in the composition in an amount of 2 wt % or less.
Clause 202. The pharmaceutical composition of clause 200, wherein when the composition is stored at 40° C./75% RH for 2 weeks, the sulfated desmosterol is present in the composition in an amount of 2 wt % or less.
Clause 203. The pharmaceutical composition of clause 199, wherein the impurity comprises 25-hydroxycholesterol.
Clause 204. The pharmaceutical composition of clause 203, wherein when the composition is stored at 25° C./60% RH for 2 weeks, the 25-hydroxycholesterol is present in the composition in an amount of 2 wt % or less.
Clause 205. The pharmaceutical composition of clause 203, wherein when the composition is stored at 40° C./75% RH for 2 weeks, the 25-hydroxycholesterol is present in the composition in an amount of 2 wt % or less.
Clause 206. Amorphous 25HC3S choline.
Clause 207. The amorphous choline of clause 206, having an x-ray powder diffraction pattern substantially the same as FIG. 13A.
Clause 208. The amorphous choline of clause 206 or 207 prepared, or obtainable, by spray drying.
Clause 209. The amorphous 25HC3S choline of clause 208, wherein the spray drying comprises forming a composition of 25HC3S choline in alcohol and water and spray drying the composition.
Clause 210. The amorphous 25HC3S choline of clause 209, wherein the alcohol is methanol.
Clause 211. The pharmaceutical composition of any one of clauses 1 to 99, wherein the 25HC3S choline comprises the amorphous 25HC3S choline of any one of clauses 206 to 210.
Clause 212. A spray-dried dispersion of a composition comprising amorphous 25HC3S choline.
Clause 213. The spray-dried dispersion of clause 212, further comprising one or more plasticizers.
Clause 214. The spray-dried dispersion of clause 213, wherein the one more plasticizers comprise a polymer.
Clause 215. The spray-dried dispersion of clause 214, wherein the polymer is selected from copovidone and HPMC.
Clause 216. The spray-dried dispersion of clause 215, wherein the copovidone is a copolymer of 1-vinyl-2-pyrrolidone and vinyl acetate at a weight ratio of 6:4 and the HPMC has a methoxyl content ranging from 28 wt % to 30 wt % and a hydroxypropoxyl content ranging from 7 wt % to 12 wt %.
Clause 217. The spray-dried dispersion of clause 216, wherein the polymer is a copolymer of 1-vinyl-2-pyrrolidone and vinyl acetate at a weight ratio of 6:4.
Clause 218. The spray-dried dispersion of any one of clauses 212 to 217, having an x-ray powder diffraction pattern substantially the same as FIG. 16A at T=0.
Clause 219. The spray-dried dispersion of any one of clauses 212 to 218, having a differential scanning calorimetry thermogram substantially the same as FIG. 18.
Clause 220. The spray-dried dispersion of clause 216, wherein the polymer is HPMC having a methoxyl content ranging from 28 wt % to 30 wt % and a hydroxypropoxyl content ranging from 7 wt % to 12 wt %.
Clause 221. The spray-dried dispersion of any one of clauses 214 to 216 and 220, having an x-ray powder diffraction pattern substantially the same as FIG. 19.
Clause 222. The spray-dried dispersion of any one of clauses 214 to 221 having a differential scanning calorimetry thermogram as FIG. 18 or FIG. 19.
Clause 223. The pharmaceutical composition of any one of clauses 1 to 99, comprising the spray-dried dispersion of any one of clauses 212 to 222.
Clause 224. A composition comprising spray-dried 25HC3S choline.
Clause 225. The composition of clause 224, wherein the composition comprises a spray-dried 25HC3S choline that is configured to release 80% or more of the 25HC3S choline within 15 minutes after administration to a human or being placed in a USP Apparatus 2 having 900 mL of dissolution medium that is 2.0% sodium dodecyl sulfate in 0.1 N HCl with a paddle speed of 75 rpm and a vessel temperature of 37° C.
Clause 226. The composition of clause 224, wherein the composition comprises a spray-dried 25HC3S choline that is configured to release 40% or less of the 25HC3S choline in the first 5 minutes after administration to a human or being placed in a USP Apparatus 2 having 900 mL of dissolution medium that is 2.0% sodium dodecyl sulfate in 0.1 N HCl with a paddle speed of 75 rpm and a vessel temperature of 37° C.
Clause 227. The composition of any one of clauses 224 to 226, further comprising one or more pharmaceutically acceptable excipients.
Clause 228. The composition of clause 227, wherein the one or more pharmaceutical excipients comprise one or more of microcrystalline cellulose, lactose monohydrate, crospovidone, sodium lauryl sulfate, colloidal silicon dioxide, and magnesium stearate.
Clause 229. The composition of clause 227, wherein the one or more pharmaceutical excipients comprise microcrystalline cellulose, lactose monohydrate, crospovidone, sodium lauryl sulfate, colloidal silicon dioxide, and magnesium stearate.
Clause 230. The composition of any one of clauses 224 to 229, in the form of granules wherein the pharmaceutically acceptable excipients are intragranular excipients.
Clause 231. The composition of clause 230, wherein the intragranular excipients are selected from microcrystalline cellulose, lactose monohydrate, crospovidone, sodium lauryl sulfate, colloidal silicon dioxide, and magnesium stearate.
Clause 232. The pharmaceutical composition of any one of clauses 1 to 99, comprising the composition of any one of clauses 224 to 231.
Clause 233. A tablet comprising a pharmaceutical composition comprising spray-dried 25HC3S choline and one or more pharmaceutically acceptable excipients.
Clause 234. The tablet of clause 233, wherein the pharmaceutically acceptable excipients comprise one or more intragranular excipients and one or more extragranular excipients.
Clause 235. The tablet of clause 234, wherein the one or more intragranular excipients comprise one or more of microcrystalline cellulose, lactose monohydrate, crospovidone, sodium lauryl sulfate, colloidal silicon dioxide, magnesium stearate, and sodium chloride
Clause 236. The tablet of clause 234 or 235, wherein the one or more extragranular excipients comprise one or more of colloidal silicon dioxide, crospovidone, and magnesium stearate.
Clause 237. The tablet of any one of clauses 233 to 236, wherein the pharmaceutical composition is a pharmaceutical composition of any one of clauses 1 to 99.
Clause 238. Micronized 25HC3S choline.
Clause 239. A composition comprising micronized 25HC3S choline.
Clause 240. The composition of clause 239, wherein the composition comprises a micronized 25HC3S choline that is configured to release 70% or more of the 25HC3S choline within 15 minutes after administration to a human or being placed in a USP Apparatus 2 having 900 mL of dissolution medium that is 2.0% sodium dodecyl sulfate in 0.1 N HCl with a paddle speed of 75 rpm and a vessel temperature of 37° C.
Clause 241. The composition of clause 239, wherein the composition comprises a micronized 25HC3S choline that is configured to release 90% or more of the 25HC3S choline within 30 minutes after administration to a human or being placed in a USP Apparatus 2 having 900 mL of dissolution medium that is 2.0% sodium dodecyl sulfate in 0.1 N HCl with a paddle speed of 75 rpm and a vessel temperature of 37° C.
Clause 242. The composition of clause 239, wherein the composition comprises a micronized 25HC3S choline that is configured to release 40% or less of the 25HC3S choline in the first 10 minutes after administration to a human or being placed in a USP Apparatus 2 having 900 mL of dissolution medium that is 2.0% sodium dodecyl sulfate in 0.1 N HCl with a paddle speed of 75 rpm and a vessel temperature of 37° C.
Clause 243. The composition of any one of clauses 239 to 242, further comprising one or more pharmaceutically acceptable excipients.
Clause 244. The composition of clause 243, in the form of granules further wherein the pharmaceutically acceptable excipients are intragranular excipients.
Clause 245. The composition of clause 244, wherein the intragranular excipients comprise one or more of microcrystalline cellulose, lactose monohydrate, sodium lauryl sulfate, copovidone, colloidal silicon dioxide, magnesium stearate, and sodium bicarbonate.
Clause 246. The pharmaceutical composition of any one of clauses 1 to 99, comprising the composition of any one of clauses 239 to 245.
Clause 247. A tablet comprising a pharmaceutical composition comprising micronized 25HC3S choline and one or more pharmaceutically acceptable excipients.
Clause 248. The tablet of clause 247, wherein the pharmaceutically acceptable excipients comprise one or more intragranular excipients and one or more extragranular excipients.
Clause 249. The tablet of clause 248, wherein the one or more intragranular excipients comprise one or more of meglumine, microcrystalline cellulose, lactose monohydrate, sodium lauryl sulfate, polysorbate 80, polyethylene glycol, crospovidone, copovidone, colloidal silicon dioxide, magnesium stearate, and sodium bicarbonate.
Clause 250. The tablet of clause 248 or 249, wherein the one or more extragranular excipients comprise one or more of croscarmellose sodium, colloidal silicon dioxide, crospovidone, and magnesium stearate.
Clause 251. The tablet of any one of clauses 247 to 250, wherein the pharmaceutical composition is a pharmaceutical composition of any one of clauses 1 to 99.
Clause 252. A method of treating or preventing one or more of nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, alcoholic fatty liver disease (AFLD), alcoholic steatohepatitis (ASH), leptin resistance, leptin deficiency, a diabetes condition, an autoimmune condition, an inflammatory condition, a neurological condition, Epstein Barr virus-related growth, and conditions related to fat accumulation and inflammation, comprising administering to a patient in need thereof an effective amount of a pharmaceutical composition of any one of clauses 101 to 205, 211, 223, 232, and 246; amorphous 25HC3S choline of any one of clauses 206 to 210; spray-dried dispersion of any one of clauses 212 to 222; composition of any one of clauses 224 to 231 and 239 to 245; tablet of any one of clauses 233 to 237 and 247 to 251; and micronized 25HC3S choline of clause 238.
Clause 253. The method of any one of clauses 100 or 252 wherein the patient is fasting.
Clause 254. A mesophase of 25HC3S choline.
Clause 255. Spray-dried particles of 25HC3S choline
Clause 256. The particles of clause 255 having a particle size distribution of 2 microns to 4 microns at D50.
Clause 257. The pharmaceutical composition of any one of clauses 1 to 99, 101 to 205, 211, 223, 232, and 246; amorphous 25HC3S choline of any one of clauses 206 to 210; spray-dried dispersion of any one of clauses 212 to 222; composition of any one of clauses 224 to 231, and 239 to 245; tablet of any one of clauses 233 to 237 and 246 to 251; or micronized 25HC3S choline of clause 238, comprising the particles of clause 255 or 256.
Clause 258. A process for making a spray-dried dispersion of 25HC3S choline comprising (a) combining 25HC3S choline with a polymer and a solvent to form a composition and (b) spray drying the composition.
Clause 259. The process of clause 258, wherein the polymer is copovidone.
Clause 260. The process of clauses 258 or 259 wherein the solvent comprises water and methanol.
Clause 261. A process for making granules comprising 25HC3S choline comprising (a) combining a filler with 25HC3S choline to make a pre-blend; (b) sieving the pre-blend; (c) adding one or more intragranular excipients to the pre-blend to make a blend; (d) compacting the blend to make granules.
Clause 262. The process of clause 261, wherein the 25HC3S choline is selected from amorphous 25HC3S choline, spray-dried 25HC3S choline, crystalline 25HC3S choline, and micronized 25HC3S choline.
Clause 263. The process of clauses 261 or 262 further comprising the step of blending the granules with one or more extragranular excipients to make a formulation blend and compressing the formulation blend into one or more tablets.
Clause 264. The mesophase of clause 254 having an x-ray powder diffraction pattern substantially the same as the top pattern of FIG. 38.
Clause 265. The mesophase of clause 254 having an x-ray powder diffraction pattern substantially the same as the middle pattern of FIG. 38.
Clause 266. The mesophase of clause 254 having an x-ray powder diffraction pattern substantially the same as the bottom pattern of FIG. 38.
Clause 267. The process of any one of clauses 258-260 further comprising the step of drying the spray-dried dispersion.
Clause 268. The process of clause 267, wherein the spray-dried dispersion has a residual solvent content of less than 3000 ppm.
Clause 269. Use of: a pharmaceutical composition of any one of claims 1 to 99, 101 to 205, 211, 223, 232, 246, and 257; amorphous 25HC3S choline of any one of claims 206 to 210; a spray-dried dispersion of any one of claims 212 to 222; a composition of any one of claims 224 to 231, and 239 to 245; a tablet of any one of claims 233 to 237 and 246 to 251; micronized 25HC3S choline of claim 238; a mesophase of any one of claims 254 and 264 to 266; or particles of claim 255 or 256; in the manufacture of a medicament for use in a method of treating or preventing one or more of nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, alcoholic fatty liver disease (AFLD), alcoholic steatohepatitis (ASH), leptin resistance, leptin deficiency, a diabetes condition, an autoimmune condition, an inflammatory condition, a neurological condition, Epstein Barr virus-related growth, and conditions related to fat accumulation and inflammation.
Clause 270. A pharmaceutical composition of any one of claims 1 to 99, 101 to 205, 211, 223, 232, 246, and 257; amorphous 25HC3S choline of any one of claims 206 to 210; a spray-dried dispersion of any one of claims 212 to 222; a composition of any one of claims 224 to 231, and 239 to 245; a tablet of any one of claims 233 to 237 and 246 to 251; micronized 25HC3S choline of claim 238; a mesophase of any one of claims 254 and 264 to 266; or particles of claim 255 or 256; for use as a medicament.
Clause 271. A pharmaceutical composition of any one of claims 1 to 99, 101 to 205, 211, 223, 232, 246, and 257; amorphous 25HC3S choline of any one of claims 206 to 210; a spray-dried dispersion of any one of claims 212 to 222; a composition of any one of claims 224 to 231, and 239 to 245; a tablet of any one of claims 233 to 237 and 246 to 251; micronized 25HC3S choline of claim 238; a mesophase of any one of claims 254 and 264 to 266; or particles of claim 255 or 256; for use in a method of treating or preventing one or more of nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, alcoholic fatty liver disease (AFLD), alcoholic steatohepatitis (ASH), leptin resistance, leptin deficiency, a diabetes condition, an autoimmune condition, an inflammatory condition, a neurological condition, Epstein Barr virus-related growth, and conditions related to fat accumulation and inflammation.
Clause 272. The tablet of any one of clauses 247 to 251, wherein when the tablet is stored in a sealed bottle at 25° C./60% RH for 6 months, the tablet has total impurities less than 3%, based on total area of impurities and 25HC3S choline assayed by HPLC at 205 nm.
Clause 273. The tablet of any one of clauses 247 to 251, wherein when the tablet is stored in a sealed bottle at 25° C./60% RH for 6 months, the tablet has total impurities ranging from 1% to 3%, based on total area of impurities and 25HC3S choline assayed by HPLC at 205 nm.
Clause 274. The tablet of any one of clauses 247 to 251 and 272 to 273, wherein when the tablet is stored in a sealed bottle at 40° C./75% RH for 6 months, the tablet has total impurities less than 3%, based on total area of impurities and 25HC3S choline assayed by HPLC at 205 nm.
Clause 275. The tablet of any one of clauses 247 to 251 and 272 to 273, wherein when the tablet is stored in a sealed bottle at 40° C./75% RH for 6 months, the tablet has total impurities ranging from 1% to 3%, based on total area of impurities and 25HC3S choline assayed by HPLC at 205 nm.
Clause 276. The tablet of any one of clauses 247 to 251 and 272 to 275, wherein when the tablet is stored in a sealed bottle at 25° C./60% RH for 6 months, the tablet has 25-hydroxy cholesterol at less than 1%, based on total area of impurities and 25HC3S choline assayed by HPLC at 205 nm.
Clause 277. The tablet of any one of clauses 247 to 251 and 272 to 275, wherein when the tablet is stored in a sealed bottle at 25° C./60% RH for 6 months, the tablet has 25-hydroxy cholesterol ranging from 0.5% to 1%, based on total area of impurities and 25HC3S choline assayed by HPLC at 205 nm.
Clause 278. The tablet of any one of clauses 247 to 251 and 272 to 277, wherein when the tablet is stored in a sealed bottle at 40° C./75% RH for 6 months, the tablet has 25-hydroxy cholesterol at less than 1%, based on total area of impurities and 25HC3S choline assayed by HPLC at 205 nm.
Clause 279. The tablet of any one of clauses 247 to 251 and 272 to 277, wherein when the tablet is stored in a sealed bottle at 40° C./75% RH for 6 months, the tablet has 25-hydroxy cholesterol ranging from 0.5% to 1%, based on total area of impurities and 25HC3S choline assayed by HPLC at 205 nm.
Clause 280. The tablet of any one of clauses 247 to 251 and 272 to 279, wherein when the tablet is stored in a sealed bottle at 25° C./60% RH for 6 months, the tablet has sulfated desmosterol at less than 1.5%, based on total area of impurities and 25HC3S choline assayed by HPLC at 205 nm.
Clause 281. The tablet of any one of clauses 247 to 251 and 272 to 279, wherein when the tablet is stored in a sealed bottle at 25° C./60% RH for 6 months, the tablet has sulfated desmosterol ranging from 1% to 1.5%, based on total area of impurities and 25HC3S choline assayed by HPLC at 205 nm.
Clause 282. The tablet of any one of clauses 247 to 251 and 272 to 281, wherein when the tablet is stored in a sealed bottle at 40° C./60% RH for 6 months, the tablet has sulfated desmosterol at less than 1.5%, based on total area of impurities and 25HC3S choline assayed by HPLC at 205 nm.
Clause 283. The tablet of any one of clauses 247 to 251 and 272 to 281, wherein when the tablet is stored in a sealed bottle at 40° C./75% RH for 6 months, the tablet has sulfated desmosterol ranging from 1% to 1.5%, based on total area of impurities and 25HC3S choline assayed by HPLC at 205 nm.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

General Synthetic Procedures for Preparing 25-hydroxy-(3β)-cholest-5-en-3-sulfate

25HC3S may be prepared by various methods. Enclosed herein are exemplary methods of making 25HC3S. It should be noted that the methods deployed herein were not necessarily used during the synthesis of 25HC3S choline or crystalline 25HC3S choline described herein. However, they could be so used in such preparations.

All temperatures are in degrees Celsius (° C.) and are uncorrected. Reagent grade chemicals and anhydrous solvents were purchased from commercial sources and, unless otherwise mentioned, used without further purification. The names of the products were determined using the naming software included in Biovia electronic lab notebook. Silica gel chromatography was performed on Teledyne Isco instruments using pre-packaged disposable SiO₂ stationary phase columns with eluent flow rates of 15 to 200 mL/min. The analytical HPLC chromatograms were performed using an Agilent 1100 series instrument with DAD detector (190 nm to 300 nm). The mass spectra were recorded with a Waters Micromass ZQ detector at 130° C. The mass spectrometer was equipped with an electrospray ion source (ESI) operated in a positive ion mode and was set to scan between n z 150-750 with a scan time of 0.3 s. Products and intermediates were analyzed by HPLC/MS on a Gemini-NX (5 μM, 2.0×30 mm) using a high pH buffer gradient of 5% to 100% of MeCN in H₂O (0.03% (NH₄)₂CO₃/0.375% NH₄OH) over 2.5 min at 1.8 mL/min for a 3.5 min run (B05), and EVO C18 (5 μM, 3.0×50 mm) using a low pH buffer gradient of 5% to 100% of MeCN in H₂O (0.1% HCOOH) over 2.5 min at 2.2 mL/min for a 3.5 min run (A05). The ¹H NMR spectra were recorded on a Bruker UltraShield 500 MHz/54 mm instrument (BZH 43/500/70B, D221/54-3209). The chemical shifts are referenced to solvent peaks which, in ¹H NMR, appear at 7.26 ppm for CDCl₃, 2.50 ppm for DMSO-d₆, and 3.31 ppm for CD₃OD.

### Example 1. Synthesis of Sodium [(3S,10R,13R,17R)-17-[(1R)-5-hydroxy-1,5-dimethyl-hexyl]-10,13-dimethyl-2,3,4,7,8,9,11,12,14,15,16,17-dodecahydro-1H-cyclopenta[a]phenanthren-3-yl]sulfate

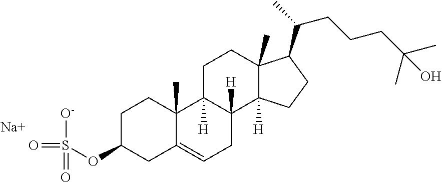

A dry 3-necked flask was charged with pyridine sulfur trioxide complex (12.45 g, 78 mmol), and the solid was suspended in toluene (1.5 L) and acetic anhydride (7.2 mL, 74.5 mmol). The mixture was stirred at 20° C. for 40 min, and pyridine (60 mL, 745 mmol) was added. The mixture was stirred at 20° C. for 20 min. (3S,8S,9S,10R,13R,14S,17R)-17-[(1R)-5-hydroxy-1,5-dimethyl-hexyl]-10,13-dimethyl-2,3,4,7,8,9,11,12,14,15,16,17-dodecahydro-1H-cyclopenta[a]phenanthren-3-ol (30 g, 74.5 mmol) was added in a single portion as a solid. The mixture was stirred at 20° C. for 23 h. Aqueous sodium acetate solution (10 wt %, 123 mL, 149 mmol) was added dropwise with vigorous stirring over 5 min. The resultant mixture was stirred at 20° C. for 1 h. The solvent was pumped out of the reactor, collecting any solids onto a glass frit. ACN (700 mL) was added, and the slurry was stirred vigorously for 3 h. The slurry was pumped out of the reactor onto the same frit, and the remaining solids in the reactor were again suspended in ACN (700 mL) and stirred for 1 h before pumping out of the reactor to the glass frit. The solids in the frit were rinsed with diethyl ether (750 mL) and then suspended in DMF (800 mL). The mixture was stirred for 1 h at 20° C. The suspension was filtered, and the filtrate collected. To the filtrate, with stirring, was added diethyl ether (3.2 L). The resulting solids were collected by vacuum filtration, and the filter cake rinsed with diethyl ether (1 L). The solids were dried under reduced pressure to provide the title compound as a solid (15 g, 40%). ¹H NMR (500 MHz, MeOD) δ 5.56-5.32 (m, 1H), 4.17 (tt, J=11.5, 4.8 Hz, 1H), 2.55 (dd, J=4.9, 2.2 Hz, 1H), 2.47-2.29 (m, 1H), 2.14-2.06 (m, 2H), 2.01 (ddd, J=12.4, 7.7, 5.1 Hz, 1H), 1.97-1.85 (m, 2H), 1.73-1.22 (m, 15H), 1.20 (s, 6H), 1.19-1.08 (m, 4H), 1.07 (s, 3H), 1.04-0.95 (m, 1H), 1.00 (d, J=6.5 Hz, 3H), 0.76 (s, 3H); m/z: ES−[M]⁻ 481.3; LCMS (B05); t_{R}=1.18 m.

### Example 2. Synthesis of Sodium [(3S,10R,13R,17R)-17-[(1R)-5-hydroxy-1,5-dimethyl-hexyl]-10,13-dimethyl-2,3,4,7,8,9,11,12,14,15,16,17-dodecahydro-1H-cyclopenta[a]phenanthren-3-yl]sulfate

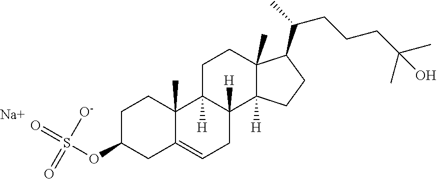

A dry 3-necked flask was charged with sulfur trioxide pyridine complex (4.74 g, 29.8 mmol). The solid was suspended in toluene (500 mL), and acetic anhydride (2.61 mL, 27.67 mmol) was added in a single portion. The resultant mixture stirred at 23° C. for 1 h. Pyridine (20 mL, 248.4 mmol) was added, and the mixture was stirred at 23° C. for 5 min. (3S,10R,13R,17R)-17-[(1R)-5-hydroxy-1,5-dimethyl-hexyl]-10,13-dimethyl-2,3,4,7,8,9,11,12,14,15,16,17-dodecahydro-1H-cyclopenta[a]phenanthren-3-ol (10 g, 24.83 mmol) was added in a single portion as a solid. The mixture stirred at 23° C. for 23 h. The reaction was diluted with MeOH (2.01 mL, 49.7 mmol) and stirred at 23° C. for 1 h. The suspension was filtered, and the solids washed with toluene (2×200 mL). The solids were collected and dried under high vacuum to provide a solid. The solids were partially dissolved in ACN (600 mL), and sodium iodide (14.9 g, 99.3 mmol) was added. The mixture was stirred at 23° C. for 10 min before being cooled to 0° C. with an ice-bath and stirred for 1.5 h. The suspension was filtered, and the solids washed with cold ACN (2×275 mL) and acetone (2×200 mL). The solids were collected and dried under high vacuum to provide the title compound as a solid (7.24 g, 57%). ¹H NMR (500 MHz, MeOD) δ 5.56-5.32 (m, 1H), 4.17 (tt, J=11.5, 4.8 Hz, 1H), 2.55 (dd, J=4.9, 2.2 Hz, 1H), 2.47-2.29 (m, 1H), 2.14-2.06 (m, 2H), 2.01 (ddd, J=12.4, 7.7, 5.1 Hz, 1H), 1.97-1.85 (m, 2H), 1.73-1.22 (m, 15H), 1.20 (s, 6H), 1.19-1.08 (m, 4H), 1.07 (s, 3H), 1.04-0.95 (m, 1H), 1.00 (d, J=6.5 Hz, 3H), 0.76 (s, 3H); m/z: ES− [M]⁻ 481.3; LCMS (B05); t_{R}=1.18 m.

### Example 3. Synthesis of Sodium [(3S,10R,13R,17R)-17-[(1R)-5-hydroxy-1,5-dimethyl-hexyl]-10,13-dimethyl-2,3,4,7,8,9,11,12,14,15,16,17-dodecahydro-1H-cyclopenta[a]phenanthren-3-yl]sulfate

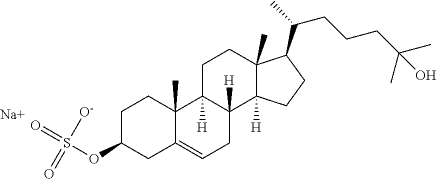

A 15 L jacketed reactor was heated to 60° C. and purged with nitrogen for 1.5 h. The jacket temperature was set to 30° C. and 2-MeTHF (7 L) was charged. (3S,10R,13R,17R)-17-[(1R)-5-hydroxy-1,5-dimethyl-hexyl]-10,13-dimethyl-2,3,4,7,8,9,11,12,14,15,16,17-dodecahydro-1H-cyclopenta[a]phenanthren-3-ol (495 g, 1.23 mol) was charged, and the manway/glassware was rinsed with 2-MeTHF (6 L). The solution was cooled to 25° C., additional 2-MeTHF (1 L) was added, and sulfur trioxide pyridine complex (234.8 g, 1.47 mol) was added. The mixture was stirred at 28° C. for 24 h. 2-MeTHF (2 L) was added, the mixture was stirred for a further 16 h, cooled to 20° C. and filtered. The solids were rinsed with 2-MeTHF (3.5 L). The solids were taken up in a solution of NaOH (118 g, 2.95 mmol) in MeOH (6 L). The mixture was stirred at 25° C. for 1 h and then filtered on a plug of Celite. The filtrate was concentrated to 3.5 L and diluted with diethyl ether (8 L). The suspension was chilled to 15° C. and filtered to provide the title compound as a solid (146.8 g, 24%). The filtrate was concentrated to 1 L and again mixed with diethyl ether (4 L). The solids were collected by vacuum filtration to provide the title compound as a solid (68.5 g, 11%). The Celite was extracted with MeOH (2 L), which was concentrated to 500 mL and diluted with diethyl ether (3 L) and the solids were collected by vacuum filtration to provide the title compound as a solid (53.3 g, 8.6%). A fourth crop was isolated from the filtrates (11.88 g, 2%). Total yield: 280.5 g, 45%. ¹H NMR (500 MHz, MeOD) δ 5.56-5.32 (m, 1H), 4.17 (tt, J=11.5, 4.8 Hz, 1H), 2.55 (dd, J=4.9, 2.2 Hz, 1H), 2.47-2.29 (m, 1H), 2.14-2.06 (m, 2H), 2.01 (ddd, J=12.4, 7.7, 5.1 Hz, 1H), 1.97-1.85 (m, 2H), 1.73-1.22 (m, 15H), 1.20 (s, 6H), 1.19-1.08 (m, 4H), 1.07 (s, 3H), 1.04-0.95 (m, 1H), 1.00 (d, J=6.5 Hz, 3H), 0.76 (s, 3H); m/z: ES− [M]⁻ 481.3; LCMS (B05); t_{R}=1.18 in.

### Example 4. Synthesis of Ammonium [(3S,10R,13R,17R)-17-[(1R)-5-hydroxy-1,5-dimethyl-hexyl]-10,13-dimethyl-2,3,4,7,8,9,11,12,14,15,16,17-dodecahydro-1H-cyclopenta[a]phenanthren-3-yl]sulfate

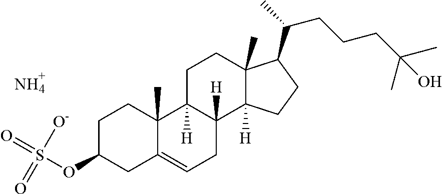

Sulfur trioxide dimethyl formamide complex (42 mg, 0.273 mmol) was added to a stirred solution of (3S,10R,13R,17R)-17-(5-hydroxy-1,5-dimethyl-hexyl)-10,13-dimethyl-2,3,4,7,8,9,11,12,14,15,16,17-dodecahydro-1H-cyclopenta[a]phenanthren-3-ol (100 mg, 0.25 mmol) in anhydrous DCM (20 mL) at 0° C. The mixture was stirred at 0° C. for 5 h, and then the reaction was warmed to 20° C. The mixture was concentrated under reduced pressure to afford a crude solid which was purified by column chromatography on silica gel (12 g cartridge) eluting with mixtures of DCM and MeOH (0-20%) to afford impure title compound. m/z: ES− [M−H]⁻ 481.

### Example 5. Synthesis of Pyridin-1-ium [(3S,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[a]phenanthren-3-yl]sulfate

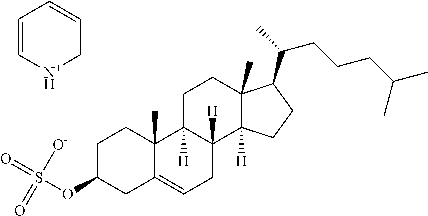

In an oven-dried round-bottom flask, sulfur trioxide pyridine complex (4.53 g, 28.5 mmol) was suspended in toluene (240 mL). Acetic anhydride (2.44 mL) was added, followed by pyridine (20.8 mL). The reaction was stirred at 23° C. for 1 h, and cholesterol (10 g, 25.9 mmol) was added in a single portion as a solid. The suspension was stirred at 23° C. for 18 h and, filtered on a glass frit, and the solids rinsed with toluene (100 mL) followed by hexanes (100 mL). The solids were suspended in chloroform (400 mL) and filtered on the same frit. The frit was rinsed with chloroform (200 mL) and the filtrate collected. The filtrate was diluted to 1.8 L with hexanes and refrigerated for 1 h. The suspension was filtered; the solids were rinsed with diethyl ether (100 mL) and dried under high vacuum to provide the title compound as a solid (10.06 g, 71%). ¹H NMR (500 MHz, MeOD) δ 8.89 (dd, J=6.6, 1.4 Hz, 2H), 8.79-8.61 (m, 1H), 8.27-8.05 (m, 2H), 5.38 (d, J=5.3 Hz, 1H), 4.13 (tt, J=11.5, 4.7 Hz, 1H), 2.53 (ddd, J=13.3, 5.0, 2.3 Hz, 1H), 2.43-2.28 (m, 1H), 2.12-2.02 (m, 2H), 2.01-1.94 (m, 1H), 1.94-1.80 (m, 2H), 1.70-0.83 (m, 20H), 1.03 (s, 3H), 0.95 (d, J=6.6 Hz, 3H), 0.88 (dd, J=6.6, 1.9 Hz, 6H), 0.72 (s, 3H); m/z: ES− [M]⁻ 465.3; LCMS (B05); t_{R}=1.40 in.

### Example 6. Synthesis of Pyridin-1-ium [(3S,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[a]phenanthren-3-yl]sulfate

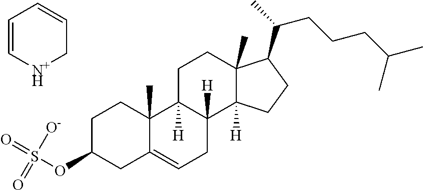

Cholesterol sulfate pyridinium salt was prepared by adding sulfur trioxide pyridine complex (4.53 g, 28.5 mmol) to a solution of cholesterol (10 g, 25.9 mmol) in 2-MeTHF (250 mL) at 30° C. and stirring the mixture for 16 h. The suspension was then filtered, and the solids rinsed with 2-MeTHF (50 mL) to afford the title compound.

### Example 7. Synthesis of Sodium [(3S,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[a]phenanthren-3-yl]sulfate

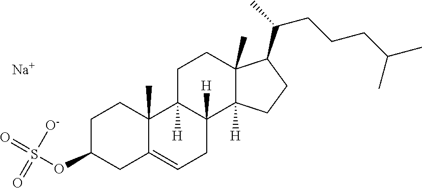

Chlorosulfonic acid (0.03 mL, 0.45 mmol) was added to a solution of 2,6-lutidine (0.08 mL, 0.69 mmol) in acetone (2.5 mL) over molecular sieves. The solution was stirred at 20° C. for 2 min before being cooled to 0° C. A solution of cholesterol (100 mg, 0.26 mmol) in acetone (5 mL), which was previously dried over molecular sieves, was added dropwise. The mixture stirred at 0° C. for 2 h before warming to 20° C. over 16 h. The mixture was filtered and the solid was collected. The solid was then suspended in acetone (10 mL) and aqueous sodium bicarbonate was added until bubbling subsided. The suspension was filtered and the solid triturated with MeOH (10 mL) and DCM (10 mL). The solvent was removed under reduced pressure to afford a solid. The solid was triturated with ACN (30 mL), filtered, and the filtrate was lyophilized to afford the title compound as a solid (7.3 mg, 5.8%). ¹H NMR (500 MHz, DMSO) δ 5.31-5.19 (m, 1H), 4.10 (s, 1H), 3.87-3.78 (m, 1H), 2.42-2.31 (m, 1H), 2.13 (dd, J=14.5, 7.6 Hz, 1H), 2.02-1.69 (m, 5H), 1.62-0.95 (m, 20H), 0.94 (s, 3H), 0.89 (d, J=6.5 Hz, 4H), 0.84 (dd, J=6.6, 2.5 Hz, 7H), 0.65 (s, 3H).

### Example 8. Synthesis of Ammonium [(3S,5S,8R,9S,10S,13R,14S,17R)-17-[(1R)-1,5-dimethylhexyl]-10,13-dimethyl-2,3,4,5,6,7,8,9,11,12,14,15,16,17-tetradecahydro-1H-cyclopenta[a]phenanthren-3-yl]sulfate

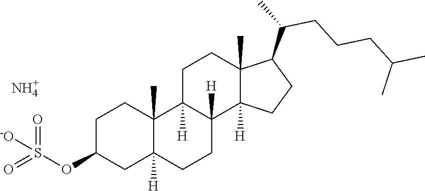

Sulfur trioxide pyridine complex (300 mg, 1.88 mmol) was added to a solution of cholestanol (300 mg, 0.772 mmol) in pyridine (5.00 mL), and the suspension was stirred at 20° C. for 16 h. The residue was purified by silica gel chromatography (24 g cartridge) with MeOH (5% NH₄OH) in DCM eluting with mixtures of DCM and MeOH (0-30%) to afford the title compound as a solid (314 mg, 84%). ¹H NMR (500 MHz, DMSO-d6) δ 7.08 (s, 4H), 3.97-3.86 (m, 1H), 1.91 (dd, J=12.5, 3.5 Hz, 1H), 1.86-1.71 (m, 2H), 1.69-1.55 (m, 3H), 1.55-1.41 (m, 3H), 1.38-1.25 (m, 5H), 1.25-0.90 (m, 15H), 0.88 (d, J=6.6 Hz, 4H), 0.84 (dd, J=6.6, 2.4 Hz, 7H), 0.74 (s, 3H), 0.62 (s, 3H); m/z: ES [M−NH₄]⁻ 467.3; HPLC (BEH Ambicarb/ACN 5-100%) t_{R}=7.48 min.

### Example 9. Synthesis of Ammonium [(3R,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[a]phenanthren-3-yl]sulfate

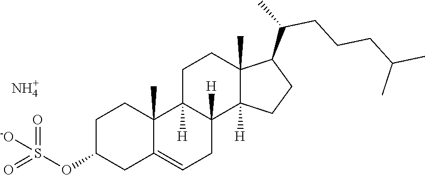

Sulfur trioxide pyridine complex (206 mg, 1.29 mmol) was added to a solution of (3R,8S,9S,10R,13R,14S,17R)-17-[(1R)-1,5-dimethylhexyl]-10,13-dimethyl-2,3,4,7,8,9,11,12,14,15,16,17-dodecahydro-1H-cyclopenta[a]phenanthren-3-ol (200 mg, 0.517 mmol) in pyridine (5.00 mL). The suspension was stirred at 20° C. for 16 h, then concentrated under reduced pressure. The residue was purified by silica gel chromatography (24.0 g cartridge) eluting with mixtures of DCM and 5% NH₄OH in MeOH (0-30%) to afford the title compound as a solid (160 mg, 64%). ¹H NMR (500 MHz, DMSO) δ 7.07 (s, 4H), 5.18-5.14 (m, 1H), 4.32-4.27 (m, 1H), 2.40-2.29 (m, 1H), 2.16 (dt, J=14.9, 2.4 Hz, 1H), 2.01-1.72 (m, 4H), 1.60-0.96 (m, 22H), 0.94 (s, 3H), 0.90 (d, J=6.5 Hz, 3H), 0.84 (dd, J=6.6, 2.4 Hz, 6H), 0.65 (s, 3H); m/z: ES [M-NH₄]⁻ 465.6; HPLC (BEH AmForm/ACN 5-100%) t_{R}=2.76 min.

### Example 10. Synthesis of Pyridinium [(3S,8S,9S,10R,13R,14S,17R)-17-[(1R)-5-hydroxy-1,5-dimethyl-hexyl]-10,13-dimethyl-2,3,4,7,8,9,11,12,14,15,16,17-dodecahydro-1H-cyclopenta[a]phenanthren-3-yl]sulfate

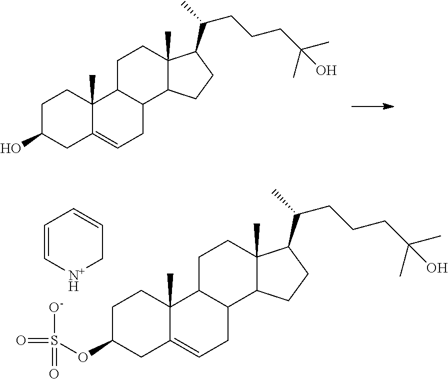

Acetic anhydride (0.0704 mL, 0.745 mmol) was added to a suspension of sulfur trioxide pyridine complex (125 mg, 0.782 mmol) in anhydrous toluene (15.0 mL). The suspension was stirred at 20° C. for 40 min, and pyridine (0.600 mL) was added. The suspension was stirred at 20° C. for 20 min. (3S,8S,9S,10R,13R,14S,17R)-17-[(1R)-5-hydroxy-1,5-dimethyl-hexyl]-10,13-dimethyl-2,3,4,7,8,9,11,12,14,15,16,17-dodecahydro-1H-cyclopenta[a]phenanthren-3-ol (300 mg, 0.745 mmol) was added in a single portion as a solid. The suspension was stirred at 20° C. for 20 h. The mixture was filtered on a glass frit to afford title compound as a solid (329 mg, 92% purity, 72% yield). ¹H NMR (500 MHz, DMSO-d6) δ 8.99-8.88 (m, 2H), 8.65-8.53 (m, 1H), 8.13-7.97 (m, 2H), 5.30-5.20 (m, 1H), 3.93-3.71 (m, 1H), 2.41-2.32 (m, 1H), 2.18-2.08 (m, 1H), 2.02-1.71 (m, 5H), 1.59-0.95 (m, 20H), 1.05 (s, 6H), 0.94 (s, 3H), 0.90 (d, J=6.4 Hz, 3H), 0.65 (s, 3H); m/z ES⁺ [M+H]⁺ 481.32; HPLC (DUR B) t_{R}=1.36 min.

### Example 11. Synthesis of 3β-25-hydroxycholest-5-ene sulfate (1, as Sodium Salt)

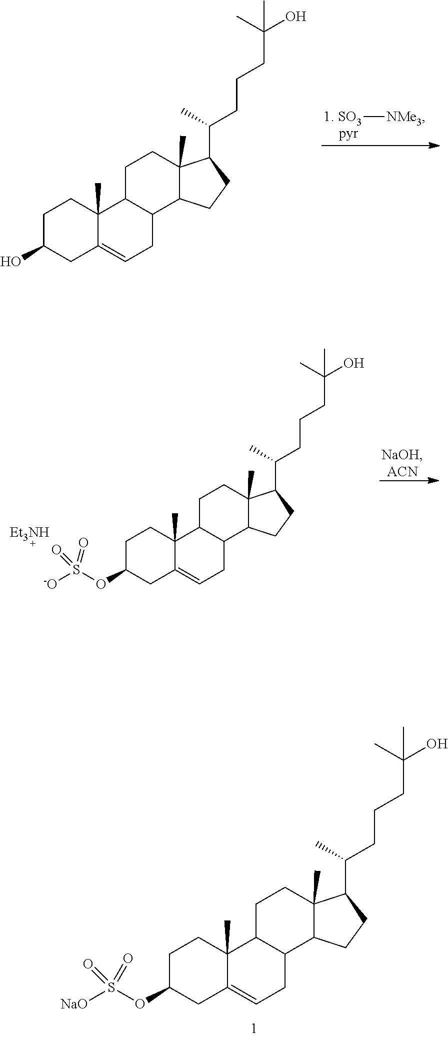

### Example 11A. Preparation of 3β-25-hydroxycholest-5-ene sulfate—Route 1

3β, 25-dihydroxycholest-5-ene (4.6 g, 0.011 mol) and triethylamine (1.7 ml, 0.023 mol) were suspended in pyridine (57 ml) and heated to 50° C. The sulfur trioxide trimethylamine complex (3.2 g, 0.023 mol) was added and the mixture agitated for 24 hours. A further charge of sulfur trioxide trimethylamine complex (0.77 g, 0.006 mol) was made and the mixture agitated for an additional 4 hours. With jacket at 50° C., the reaction mixture was distilled to −20% of initial volume. The residue was purified by silica gel chromatography (11 0 g), eluting with an ethyl acetate/methanol/triethylamine (90/9/1 v/v) mixture; fractions were analyzed by TLC (4:1 methylene chloride:methanol) using a phosphomolybdic acid stain. Fractions containing the 3- and 25-sulfate regioisomers were combined and evaporated (bath temp <35° C.). The residue (4.2 g, 0.0072 mol) was slurried in acetonitrile (25 g), treated with 1 N sodium hydroxide (7.2 ml, diluted from 30% sodium hydroxide solution) for 1 hour, and then filtered. Solids were rinsed through with acetonitrile (25 g) and dried to a constant weight (2.77 g). The solids, containing a mixture of 3- and 25-sodium sulfate salts (2.77 g), were triturated with ethanol (27.7 g, 10S) at 50° C. for 1 hour and then filtered at 5° C. The isolated solids were dried to a constant weight (1.2 g). The solids (1.2 g) were suspended in 6:1 acetonitrile/water (10 S) at 30° C. for 30 minutes and then filtered. Filtration required about 40 minutes. Solids were dried to a constant weight (0.86 g) and analyzed.

### Example 11B. Preparation of 3β-25-hydroxycholest-5-ene sulfate Route 2 Excess Sulfating Agent

An excess of sulfur trioxide trimethylamine complex was used to drive the reaction toward formation of the disulfate. The 3-hydroxy group of 3β, 25-dihydroxycholest-5-ene is about 6 times more reactive towards sulfation than the 25-hydroxyl. Providing excess sulfating agent and allowing the reaction to proceed to high conversion will provide monosulfate of higher regioisomeric purity. This result was observed during this synthesis. A solution of 3β, 25-dihydroxycholest-5-ene (4.1 g) in pyridine (75 ml) was vacuum distilled to reduce the volume to 50 ml. This was performed to remove isopropanol (from the recrystallization of the diol) and any moisture present. Triethylamine (2 equivalents) and then a total of 1.75 equivalents of sulfur trioxide trimethylamine complex was added in portions (1.0, 0.5 and 0.25 equivalents) to the reaction at 50° C. cover a period of 18 hours and the reaction was allowed run for a total of 43 hours. The reaction mixture was concentrated by vacuum distillation and the residue was absorbed onto SiO₂ (10 g). The loaded SiO₂ was placed on a SiO₂ column and eluted with 2-50% methanol/ethyl acetate/i % triethylamine. The appropriate fractions from the column were combined and evaporated to yield the disulfate (3.1 g, 39.7%) and the monosulfate (2.6 g, 44.7%). The monosulfate was obtained as a 22:1 mixture of the 3-sulfate and 25-sulfate. The solids were suspended in acetonitrile (25 g), treated with 1 N sodium hydroxide (4.44 ml), and then filtered. A thick gel formed, which was difficult to manipulate and was not filtered. The product was a suspension in acetonitrile/water. The solvents were removed by rotary evaporation at 40° C. and the residue was dried in a vacuum oven at 40° C. Trituration of the solid with acetone yielded a white solid: 1.27 g, 24.9%. This product showed only the 3β-sulfated product, but was contaminated with peaks at RRT 8.18 (unknown, 2.0%), RRT 15.17 (diol, 2.2%) and RRT 16.70 (unknown, 1.8%).

### Example 12. Preparatory Scale Synthesis of 3β-25-hydroxycholest-5-ene sulfate (1, as Sodium Salt)

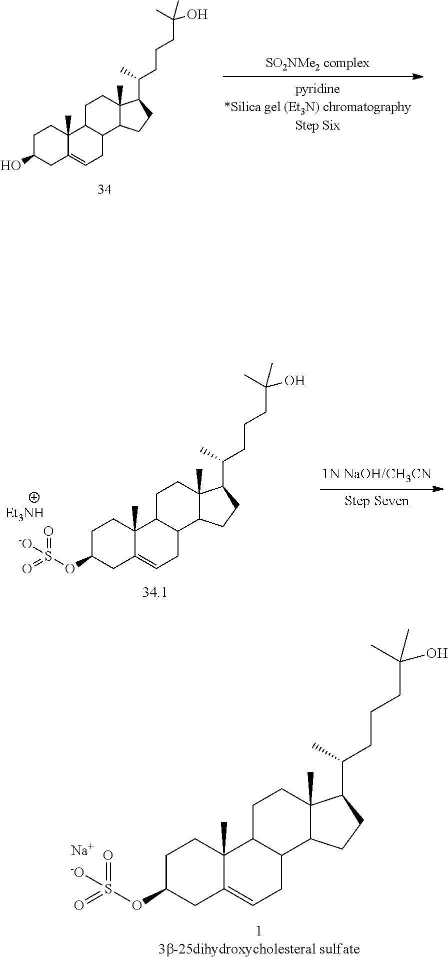

A 2 L, three-necked, round-bottomed flask with an overhead stirrer was charged with 3β, 25-dihydroxycholest-5-ene (34) (30 g, 74.5 mmol) and dry pyridine (500 mL, Sigma-Aldrich, cat. #270970-1L, lot #SHBC6287V). Sulfur trioxide-trimethylamine complex (12.2 g, 89.4 mmol, Sigma-Aldrich, cat. #135879-100G, lot #MKBH5585V) was added in one portion. The suspension was stirred at room temperature overnight. The reaction mixture was concentrated and the residue was purified by column chromatography to give 25.9 g (59%) of white solid as the triethylamine salt (HPLC: 98.6% purity). To a suspension of triethylamine salt 34.1 (64 g, 110.1 mmol) in ACN (1 L) was added 1 N NaOH (110 mL, 110.1 mmol, NaOH, Fisher, cat. #S318-3, lot #034906), and the mixture was stirred for 1 h at room temperature. The solid was filtered, washed with ACN (1 L), and dried under vacuum (P₂O₅) overnight, yield: 51.5 g, 93% (HPLC: 98.6% purity).

After overnight stirring, the reaction was a gel-like mixture. TLC showed the expected product as the major spot (TLC: 20% MeOH in DCM, R_{f}=0.4), with both the starting material (R_{f}>0.9) and 3b-25-hydroxycholesterol disulfate (R_{f}<0.1) as minor spots. Silica gel (1 kg, Sorbent Technologies, cat. #40930-2.5 kg) was packed to form a column of dimension 10 cm×42 cm. The column equilibration was accomplished with 1% triethylamine (Et₃N, Fisher, cat #04885-4, lot #062833) in DCM (2.8 L). The crude residue was dissolved in DCM (200 mL) and Et₃N (20 mL), which was directly loaded into the column. Triethylamine was used at this stage to avoid decomposition of the product and of the disulfate (which forms an olefin that is then very difficult to remove from the product). Initial elution was DCM (1% Et₃N) (2 L), followed by 1% MeOH in DCM (1% Et₃N)(1 L), 2% MeOH in DCM (1% Et₃N)((3 L), 5% MeOH in DCM (1% Et₃N)((1 L). The product began to elute in 2% MeOH in CH₂Cl₂ (1% Et₃N). The collected fractions were concentrated via rotary evaporation below 36° C. (if the temperature is higher than 45° C., decomposition of the product in the presence of MeOH is observed). Both TLC and NMR were checked for the selected fractions. HPLC (Zorbax SB-18, 4.6×150 mm, 5 μm, 202 nm, flow rate 0.8 mL/min): Solvent A: MeOH/5% ACN/7.4 mM NH₄OAc; Solvent B: H₂O/5% ACN/7.4 mM NH₄OAc. Gradient 75% A and 25% B to 100% A. Product: 98.6% purity; 1.4% (starting material 34). HPLC: Durashell C18 (Agela Technologies, 4.6×50 mm, 3 mm, 100 Å); Solvent A: MeOH/5% ACN/7.4 mM NH₄OAc; Solvent B: H₂O/5% ACN/7.4 mM NH₄OAc. Product: 98.6% purity; 1.4% (starting material 34).

### Example 13. Large Scale Synthesis of 3β-25-hydroxycholest-5-ene sulfate (1, as Sodium Salt)

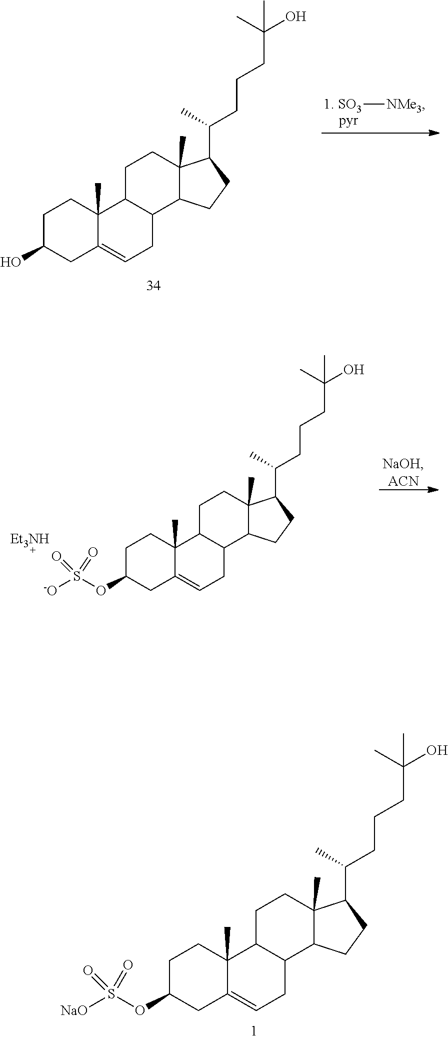

### Summary of Kilogram-Scale Preparation

3β, 25-dihydroxycholest-5-ene (34) (2.6 kg) and pyridine (39.2 kg) was combined and the mixture heated to 40° C. with agitation in two 50 L reactors. Sulfur trioxide-trimethylamine (1.1 kg) was added to the mixture and stirred at 40° C. for 6-12 hours until the reaction was complete. The mixture was concentrated to minimum stir volume under vacuum distillation and then diluted with methylene chloride and triethylamine.

The crude reaction mixture in methylene chloride was loaded onto a 2.33 ft³ stainless steel column (C-105) packed with silican gel and eluted with methylene chloride (containing 1% methanol and 1% triethylamine). Fractions containing undesired product were collected in waste drums. Fractions containing desired product are collected and concentrated in the reactor.

Acetonitrile, water and sodium hydroxide were added to the reactor containing the desired product and the mixture was agitated until the reaction was deemed complete. The resulting slurry was cooled to 10-15° C. and filtered to isolate compound 1. The cake of isolated compound 1 was washed with acetonitrile and then dried at 40° C. under vacuum until a constant weight was achieved.

The solid was filtered, washed with acetonitrile (1 L), and dried under vacuum (P₂O₅) overnight, yield: 51.5 g, 93% (HPLC: 98.6% purity).

### Discussion

Analysis of the reaction mixture by HPLC after 6 h showed 44.1% remaining starting material. The reaction was considered complete and distilled under vacuum to a minimum stir volume (Step 5.3). To the resulting thick residue were added methylene chloride and triethylamine, and the solution was transferred to a clean 5 gallon glass carboy. Thick solids precipitated in the glass carboy after holding the solution overnight. The solids were filtered away using the benchtop filter. Approximately ⅓ of the clear filtrate was charged to the top of a C-105 column. The silica in the C-105 column was previously flushed with ethyl acetate and methanol, and then equilibrated with 1% triethylamine in methylene chloride eluent.

Once the crude solution was loaded to the top of the column, eluent was charged to maintain a pressure of ~10 psi. Eluent was sampled as it exited the column every 10-15 minutes. Pyridine and 3β, 25-dihydroxycholest-5-ene were present in the first two samples, but the 3β-sulfate, 25-hydroxycholest-5-ene triethylamine salt and 25-sulfate regioisomer were detected in the third sample in addition to pyridine and 3β, 25-dihydroxycholest-5-ene. Since minimal separation occurred, all remaining material was eluted from the column using the polar eluent (1% MeOH, 1% NEt3, and 98% DCM). The filtrate was concentrated and combined with the remaining two-thirds of the crude solution from the carboy. After distillation, the crude solution was transferred to a clean carboy. The eluent exiting the column was analyzed and contained 1.7% methanol (¹H NMR area %). The column was equilibrated with eluent (1% triethylamine in methylene chloride) and analyzed for methanol (0.25% methanol, ¹H NMR area %). Solids began to form in the carboy during this time. The slurry was filtered, and the filtrate was collected in a clean carboy.

Approximately one-third of the crude solution was loaded onto a second C-105 column. Eluent was charged to the column to maintain <5 psi. Analysis of the eluent leaving the column by thin layer chromatography (TLC) showed that separation was taking place. Once 3β, 25-dihydroxycholest-5-ene was no longer detected by TLC, the eluent was analyzed by ¹H NMR to ensure that the 3β-sulfate, 25-hydroxycholest-5-ene triethylamine salt was separated from the 25-sulfate regioisomer. A sample was removed from the eluent containing drum, and the purity of the 3β-sulfate, 25-hydroxycholest-5-ene triethylamine salt was 85% with 15% 25-sulfate regioisomer present (¹H NMR). An HPLC weight percentage assay showed that 127 g of 3β-sulfate, 25-hydroxycholest-5-ene triethylamine salt /25-sulfate regioisomer was collected in the drum (85% 3β-sulfate, 25-hydroxycholest-5-ene triethylamine salt). The purified material was set aside and the remaining two-thirds of the crude solution was purified by chromatography. The silica in the C-105 column was flushed with methanol and then equilibrated with 1% triethylamine in methylene chloride (0.2% methanol by ¹H NMR area % in the eluent after regeneration).

The solids that precipitated from the carboy were analyzed by ¹H NMR and identified as the quaternary ammonium salt produced from the reaction of methylene chloride with triethylamine (from the SO₃NMe₃ reagent) and methylene chloride with triethylamine. The methylene chloride-triethylamine complex was separated by filtration, while methylene chloride-triethylamine complex was formed in the chromatography. Formation of the salt occurred under ambient conditions and was rapid in certain instances in a pressurized environment. The establishment of an equilibrium in which the triethylammonium moiety of the 3β-sulfate, 25-hydroxycholest-5-ene triethylamine salt may be exchanging for the quaternary ammonium salt to give the quaternary ammonium complex and the triethylamine hydrochloride. Equilibrium favors the formation of the quaternary ammonium complex since there is more methylene chloride-triethylamine complex present. Triethylamine hydrochloride was isolated and characterized.

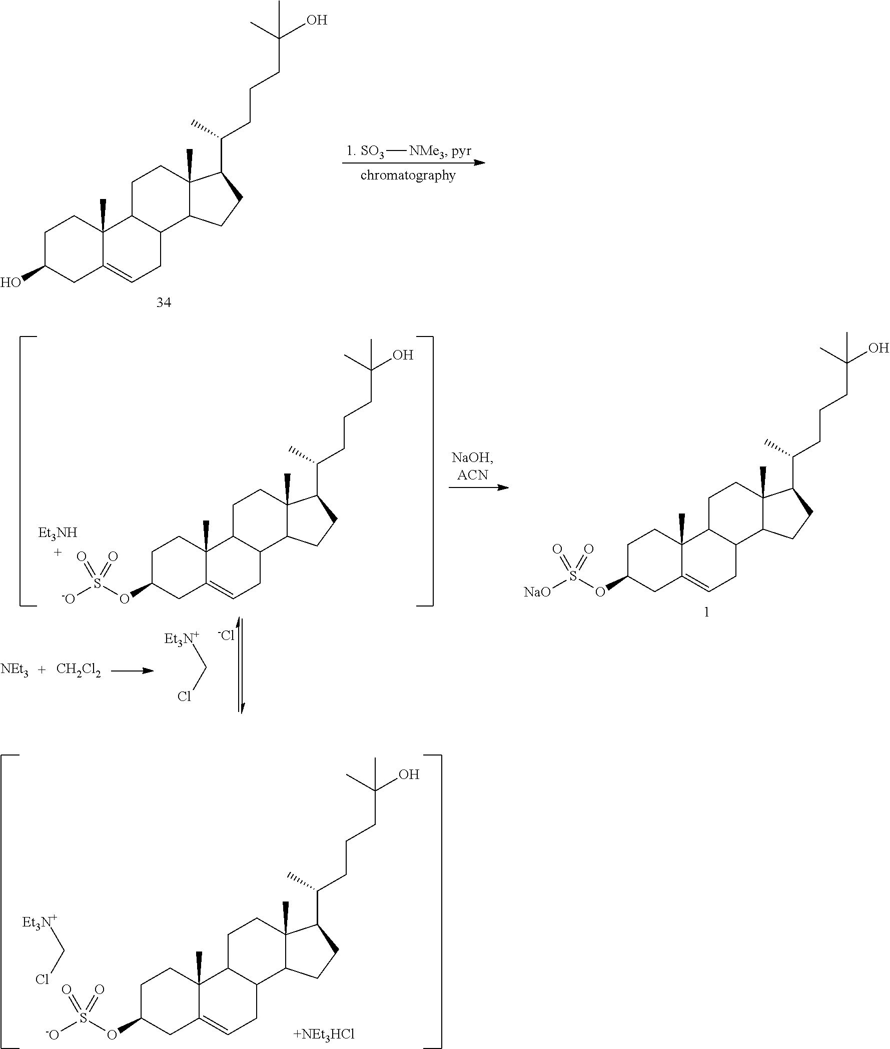

Of the remaining two-thirds of the crude mixture in the carboy, one-third was subjected to chromatography on a fourth column. Pressure was maintained at 0-1 psi during the entire purification. Separation of 3β-sulfate, 25-hydroxycholest-5-ene triethylamine salt from the 25-sulfate regioisomer was successful: The purity of the 3β-sulfate, 25-hydroxycholest-5-ene triethylamine salt in the drum was 99.79% by HPLC. Approximately 0.050 kg of 34.1 was isolated from the column (HPLC weight percent assay). The silica gel was cleaned with methanol and regenerated with 1% triethylamine in methylene chloride. The amount of methanol present after regeneration was 0.44% (¹H NMR area %). No separation occurred for this column. No further purifications were done with the material from the fourth column. Eluent from the third column (~50 g of the 3β-sulfate, 25-hydroxycholest-5-ene triethylamine salt) was subjected to the cation exchange beginning with a solvent swap to acetonitrile. After the addition of acetonitrile, water, and 30% sodium hydroxide, the slurry was agitated and then held overnight. Solids were present in the reactor after the post-stir. The mixture was cooled and filtered using a new 8.5″ benchtop filter. The cake was washed with fresh acetonitrile and dried. A sample was analyzed by ¹H NMR and peaks consistent with a quaternary ammonium salt were present in the spectrum. The 25-sulfate regioisomer was present by HPLC.

Eluent from the second column was concentrated under vacuum and dried to a constant weight. Analysis of the yellow powder (540 g) by ¹H NMR showed a ratio (3:1) of the methylene chloride-triethylamine quaternary ammonium salt to the monosulfate compound. All of the crude material (540 g) was charged to a 3 L jacketed reactor. Acetonitrile (1400 g) was charged, and the slurry was heated to 50±5° C. for 30 min. The slurry was cooled to 26° C. and then filtered. The wet cake was analyzed and the ratio of 3β-sulfate, 25-hydroxycholest-5-ene triethylamine salt /25-sulfate regioisomer to quaternary ammonium salt was ~1:1. The purified solids and fresh acetonitrile (1400 g) were charged back to the reactor. Water (200 g) was charged after 45 min, agitated for 15 min, and then filtered. The granular powder was dried in a vacuum oven at 40° C. overnight. The filtrate was concentrated to dryness, and the residue was combined with the dried material and both were charged to a 3 L reactor. Acetonitrile (1500 g), 1 N sodium hydroxide (600 g), and 30% sodium hydroxide (40 g) were sequentially charged to the reactor. The slurry was agitated for 48 hours and then filtered at ambient temperature. The cake was dried to a constant weight (173 g) and analyzed by HPLC.

### Example 13A. Purification to Separate the 25-Sulfate Regioisomer from Compound 1

Several solvents were explored to purge the 25-sulfate sodium salt from compound 1. No solids were recovered after dissolving impure compound 1 in polar solvents and then charging anti-solvents (Entries 1 and 2, Table 3). Minimal solids were formed after dissolving the material in methanol and then adding acetonitrile (Entry 3, Table 3). Using 2-propanol (Entry 4, Table 3) and a mixture of methanol and water resulted in a form change, which caused the material to become a thick paste that would not transfer or filter. Trituration of impure compound 1 with ethanol at 40-50° C. was sufficient to purge the majority of the 25-sulfate regioisomer (Entry 6, Table 3). A mixture of compound 1 (1 g) and ethanol (10 mL) was heated to reflux, cooled, and filtered. The isolated material (55% recovery) was 99.6% pure with the 25-sulfate and 3β, 25-dihydroxycholest-5-ene products reduced to 0.1% and 0.3%, respectively.

TABLE 3
Trituration/recrystallization of Compound 1

| | | Unpurified | | | 3β, 25- | |
|---|---|---|---|---|---|---|
| | | Compound 1 | Compound 1 | 25-sulfate | dihydroxy- | |
| Sample | Solvent | (g) | Purity | regioisomer | cholest-5-ene | Comment |
| 1 | Ethyl | 0.5 | N/A | N/A | N/A | No Solids |
| | acetate/methanol | | | | | |
| 2 | Dimethyl | 0.5 | N/A | N/A | N/A | No Solid |
| | sulfoxide/iso- | | | | | |
| | propanol | | | | | |
| 3 | Methanol/ | 0.1 | N/A | N/A | N/A | Minimal |
| | acetonitrile | | | | | Solids |
| 4 | Isopropanol | 0.1 | N/A | N/A | N/A | Gel/paste |
| 5 | Methanol/water | 0.5 | 96.3 | 1.8 | 1.9 | Gel/paste |
| 6 | Ethanol | 0.1 | 97.0 | 0.4 | 2.6 | Trituration |
| 7 | Ethanol | 1.0 | 99.6 | 0.1 | 0.3 | 55% |
| | | | | | | recovery |

### Example 13B. Purification of the 3β-sulfate, 25-hydroxycholest-5-ene Triethylamine Salt

The 3β-sulfate, 25-hydroxycholest-5-ene triethylamine salt was purified to eliminate methylene chloride due to reactivity with trimethylamine and triethylamine. Purification was achieved using an isocratic solvent system that includes 90% ethyl acetate, 9% methanol and 1% triethylamine.

### Example 13C. Optimization of SO₃NMe₃ Equivalents

The amount of added SO₃NMe₃ complex needed to either completely consume 3β, 25-dihydroxycholest-5-ene or arrive at a point at which the bis-sulfate and unreacted starting material byproducts were minimal was determined. A solution of 3β, 25-dihydroxycholest-5-ene (0.5 g, LOS) in pyridine (18.65) containing triethyl amine (0.55) was heated to 50° C. A sample was removed from the reaction every 30 min, which was followed by the addition of the SO₃NMe₃ complex. Following the final charge of the SO₃NMe₃ complex, the vial was allowed to stir at 50° C. for a total of 24 h. (Table 4). Approximately 1.75 equivalents of SO₃NMe₃ complex were sufficient to consume 86.6% of starting material 3β, 25-dihydroxycholest-5-ene (Sample 7, Table 4). 3β, 25-dihydroxycholest-5-ene was completely consumed after 2.9 equivalents of SO₃NMe₃ complex were added. Formation of the bis-sulfate will out-compete mono-sulfation of 3β, 25-dihydroxycholest-5-ene as the reaction progresses. The 3β-sulfate, 25-hydroxycholest-5-ene triethylamine salt was completely converted to the bis-sulfate after 24 hours.

TABLE 4
Equivalents of sulfur trioxide-trimethylamine complex

| | | 3β-sulfate, 25- | 3β, 25- | |
|---|---|---|---|---|
| | Equivalents of | hydroxycholest-5-ene | dihydroxycholest- | Bis- |
| Sample | SO₃NMe₃ | triethylamine salt | 5-ene | sulfate |
| 1 | 0.25 | 18.0 | 75.9 | 6.1 |
| 2 | 0.5 | 28.6 | 71.4 | 0.0 |
| 3 | 0.75 | 39.8 | 60.2 | 0.0 |
| 4 | 1.00 | 48.6 | 46.7 | 4.7 |
| 5 | 1.25 | 53.1 | 39.4 | 7.4 |
| 6 | 1.50 | 58.4 | 28.6 | 12.9 |
| 7 | 1.75 | 68.1 | 13.4 | 18.5 |
| 8 | 2.00 | 60.9 | 13.1 | 26.0 |
| 9 | 2.30 | 58.3 | 7.2 | 34.4 |
| 10 | 2.60 | 49.6 | 1.0 | 49.4 |
| 11 | 2.90 | 46.6 | 0.0 | 53.4 |
| 12 | 3.20 | 37.7 | 0.0 | 62.3 |
| 13 | 3.50 | 32.3 | 0.0 | 67.7 |

### Example 14. One-Hundred Gram Scale Synthesis of 3 #-25-hydroxycholest-5-ene sulfate (1, as Sodium Salt)

A slurry of 3β, 25-dihydroxycholest-5-ene (100 g, 1.0S) and triethylamine (0.55) in pyridine (15.65) was heated to 50° C. The SO₃NMe₃ complex (1.75 equivalents, 0.65) was charged in one portion. The mixture was agitated for 5 hours and then analyzed for reaction completion by HPLC (Sample 1—3β-sulfate, 25-hydroxycholest-5-ene triethylamine salt /25-sulfate regioisomer (67.1%); 3β, 25-dihydroxycholest-5-ene (12.20%); bis-sulfate (20.8%)). The jacket was set to 70° C. and the reaction was concentrated to <20% of the initial volume. A sample was removed and analyzed by HPLC for stability (Sample 2-3β-sulfate, 25-hydroxycholest-5-ene triethylamine salt /25-sulfate regioisomer (60.5%); 3β, 25-dihydroxycholest-5-ene (10.0%); bis-sulfate (29.5%)). The amount of monosulfate decreased from 67.1% to 60.5% during the distillation, while the amount of bisulfate increased ~9%. The amount of 3β, 25-dihydroxycholest-5-ene did not decrease much during the distillation.

Solids were present in the reactor following a 48 h post stir, and the addition of methanol (0.5S) did not dissolve the solids. The crude material (300 g) was subjected to purification by silica gel chromatography eluting with 90% ethyl acetate, 9% methanol, and 1% triethylamine. Silica gel (2.4 kg) was slurried in the eluent and packed to form a 5.25″×28″ column. The crude mixture was transferred to the column, and the purification was carried on over three days. The eluent was collected in 1 L fractions. Fractions 1-7 contained no material detected by TLC; Fractions 8-11 contained pyridine and 3β, 25-dihydroxycholest-5-ene; Fractions 12-20 contained no material detected by TLC; Fractions 21-22 contained an undetermined compound and Fractions 23-59 contained 3β-sulfate, 25-hydroxycholest-5-ene triethylamine salt /25-sulfate regioisomer.

Approximately 82 g of 3β-sulfate, 25-hydroxycholest-5-ene triethylamine salt /25-sulfate regioisomer (56.5% yield) was isolated after the column determined by weight percentage analysis. After chromatography, the eluent containing a mixture of 3β-sulfate, 25-hydroxycholest-5-ene triethylamine salt /25-sulfate regioisomer was concentrated to a slurry and transferred to a 2 liter reactor. The solvent was swapped to acetonitrile, the slurry was cooled to 10° C., and 1 N sodium hydroxide (1.8S, 1 equivalent based on 82 g of 3β-sulfate, 25-hydroxycholest-5-ene triethylamine salt /25-sulfate regioisomer) was charged over 10 minutes. The slurry was agitated for 1 hour and then filtered. The filtration was very fast, requiring <5 minutes. The solids were dried at 40° C. under vacuum to a constant weight (70 g, 99% yield for the cation exchange). A sample was analyzed by HPLC (Sample 1, Table 5) which indicated that the 25-sulfate regioisomer was present at 5.1%. The white powder (70 g) was transferred to a 2 liter reactor and slurried with ethanol (700 g) at 50° C. for 1 hour. A form change was observed after 30 minutes of stirring by the thickening of the slurry mixture. The slurry was cooled to 10° C., stirred for 1 hour, and then filtered at 10° C. The reactor was rinsed with ethanol (170 g), cooled to 10° C. and then transferred to the filter as a cake wash. The solids were dried to a constant weight (64.6 g, 92.3% recovery) and analyzed by HPLC (Sample 2, Table 5). After trituration, the purity of compound 1 improved to 97.4%, but the 25-sulfate regioisomer was 1.6%. Impure compound 1 (64.6 g, 1.0S) was slurried in ethanol (581 g, 9S) at 55° C. for 1.5 hours. The slurry was cooled to 10° C. and then filtered. The reactor and cake were rinsed with ethanol (84 g) at 10° C., and the resulting solid was dried at 40° C. under vacuum to a constant weight (60.4 g, ethanol present at 5.9%, 87.9% recovery).

A sample of compound 1 following ethanol trituration was analyzed by HPLC (Sample 3, Table 5). The 25-sulfate regioisomer was purged, but the amount of unknown 1 increased to 0.9%. The purified material (56.8 g) was slurried in acetonitrile (5S) and water (0.9S) at 30° C. for 30 minutes in a 1 liter reactor. The slurry formed stiff peaks during this time, but the paste was easily transferred to the filtration setup using an FMI pump. The reactor and cake were rinsed with fresh acetonitrile (30 g), and the material was dried to a constant weight (54.5 g, 90.2% recovery). Analysis by ¹H NMR showed that ethanol was absent, but water was present at 1.2 wt %. The purity of the final material improved to >99% (Sample 4, Table 5). The unknown impurities at RRT 1.68 and 1.85 were present at 0.6% and 0.2%, respectively. Taking into account the residual water, the final isolated yield of compound 1 in the 100 g demonstration run was 43.2%.

TABLE 5
Purification of Crude Compound 1

| | | 3β-sulfate, 25- | | | |
|---|---|---|---|---|---|
| | 25-sulfate | hydroxycholest-5- | Unknown 1 | Unknown 2 | Unknown 3 |
| | regioisomer (%) | ene triethylamine | (%) RRT | (%) RRT | (%) RRT |
| Sample | RRT 0.78 | salt (%) RRT 1.00 | 1.68 | 1.85 | 1.99 |
| 1 | 5.1 | 93.1 | 0.9 | 0.6 | 0.3 |
| 2 | 1.6 | 97.4 | 0.6 | 0.4 | ND |
| 3 | ND | 98.9 | 0.9 | 0.2 | ND |
| 4 | ND | 99.2 | 0.6 | 0.2 | ND |

### Example 15. Azeotropic Removal of Water from 3β, 25-dihydroxycholest-5-ene

A slurry of 3p, 25-dihydroxycholest-5-ene (5 g, LOS) and pyridine (15.6S, 0.016% water, Entry 1, Table 6) was heated to 50° C. A sample of the reaction was removed for water content analysis (0.29%, Entry 2, Table 6). The reaction volume was reduced 50% and sampled for water content (0.042%, Entry 3, Table 6). The amount of pyridine that was collected in the distillate (39 g) was replaced with fresh pyridine in the reactor and sampled again for water (0.027%, Entry 4, Table 6). Once the internal temperature reached 50° C., triethylamine (0.5S) and SO₃NMe₃ (0.6S) were charged to the reactor. The thin white slurry became a clear solution within 15 minutes, and the reaction was agitated at 50° C. A sample was removed at 2 hours and 3 hours for IPC analysis (Entries 1 and 2, Table 7). Only 7.1% 3p, 25-dihydroxycholest-5-ene remained after 2 h. Azeotropically removing the water prior to the addition of SO₃NMe₃ improves the consumption of starting material.

TABLE 6
Water content analysis

| Sample | Comment | % Water |
|---|---|---|
| 1 | Pyridine solvent | 0.016 |
| 2 | Reaction solution before pyridine distillation | 0.285 |
| 3 | Reaction solution after pyridine distillation | 0.042 |
| 4 | Reaction solution after addition of pyridine | 0.027 |

TABLE 7
Reaction completion profile by HPLC

| | | 3β-sulfate, 25- | |
|---|---|---|---|
| | | hydroxycholest- | |
| | | 5-ene | |
| | 25-sulfate | triethylamine | 3β, 25- |
| | regioisomer | salt/25-sulfate | dihydroxycholest- |
| Sample | (%) | regioisomer (%) | 5-ene (%) |
| 1 | 30.8 | 62.2 | 7.1 |
| 2 | 38.9 | 55.6 | 5.6 |

### Example 16. Ethanol Trituration of Crude Compound 1

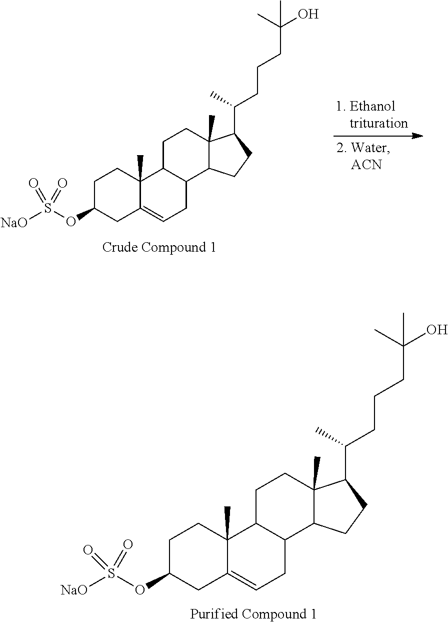

Crude compound 1 was suspended in ethanol and heated to 55° C. and stirred for 1 hour. The slurry mixture is cooled, filtered and washed with ethanol. The resulting cake is dried overnight at 50° C. The cake was charged back into the reactor and suspended in acetonitrile and water. The mixture is heated to 30° C. and stirred for 1 hour. The mixture is then cooled to 15° C., filtered and washed with acetonitrile and water (90:10). The resulting cake is dried for not longer than 24 hours at 50° C. until a constant weight is achieved. Impurity content in purified compound 1 was determined by HPLC. (RRT 0.67<0.05%; RRT 0.77<0.05%; RRT 0.79<0.05%; RRT 0.95<0.05%; RRT 1.13<0.05%; RRT 1.22<0.05%; RRT 1.31<0.05%; RRT 1.95=0.09%; RRT 2.09<0.05%; RRT 2.67<0.05%; RRT 2.75=0.05%; RRT 3.04<0.05%; RRT 3.23=0.09%; RRT 3.64=0.3%; RRT 5.00<0.05%; Total impurities=1.1%.

### Example 17. Identification of Byproducts from Sulfating 25-hydroxy-(3β)-cholest-5-en-3-ol to produce 25-hydroxy-(3β)-cholest-5-en-3-sulfate

A composition of 25-hydroxy-(3β)-cholest-5-en-3-ol was sulfated with a sulfur-trioxide pyridine complex in toluene at 23° C. for 1 h to produce 25-hydroxy-(3β)-cholest-5-en-3-sulfate. Compounds formed in a reaction mixture when preparing the 25-hydroxy-(3β)-cholest-5-en-3-sulfate product were analyzed by high performance liquid chromatography. Tables 8 and 9 provide the HPLC chromatography conditions. Table 10 lists retention times of compounds identified as being formed in the reaction mixture when sulfating 25-hydroxy-(3β)-cholest-5-en-3-ol with a sulfur-trioxide pyridine complex.

TABLE 8
Chromatographic Conditions

| | |
|---|---|
| Column | Aglient Zorbax Eclipse XDB-C8, |
| | 3.5 μm (4.6 × 150) mm |
| Mobile Phase A | 2.5 mM sodium phosphate, pH 2.1 |
| | with 0.2% SDS |
| Mobile Phase B | 10:85:5 MPA:ACN:MeOH |
| Detection | Impurities and calculated purity: |
| | 205 nm; Assay: 220 nm |
| Column | 45° C. |
| Temperature | |
| Injection Volume | 25.0 μL |
| Flow Rate | 1.0 mL/min |
| Acquisition Time | 50.0 minutes (including 5.0 min. |
| | re-equilibration) |

TABLE 9
Chromatographic Conditions-Gradient

| Time (minutes) | % A | % B |
|---|---|---|
| 0.0 | 62 | 38 |
| 35 | 0 | 100 |
| 45 | 0 | 100 |
| 45.1 | 62 | 38 |
| 50.0 | 62 | 38 |

TABLE 10
Retention Times

| | Retention |
|---|---|
| | Time |
| Compound | (min) |
| Pyridine | 3.2 |
| 25-sulfated cholesterol | 6.6 |
| 25-hydroxy-(3β)-cholest-5-en-3-sulfate | 7.7 |
| Unknown Byproduct #1 | 18.3 |
| 25-hydroxy-(3β)-cholest-5-en-3-ol | 26.5 |
| Unknown Byproduct #2 | 37.7 |

### Example 18. Determining Purity of Sulfur Trioxide Pyridine Sulfating Agent

Proton nuclear magnetic resonance spectroscopy (¹H-NMR) was conducted on samples of sulfur trioxide pyridine in a deuterated solvent. Sulfur trioxide pyridine is a colorless solid that can degrade due to the presence of moisture, which can impact the overall yield and reproducibility of sulfating 25-hydroxy-(3β)-cholest-5-en-3-ol. A sample of sulfur trioxide pyridine from three lots (A-C) was dissolved in deuterated acetone ((CD₃)₂CO) and proton NMR spectra were recorded using a 500 MHz Bruker spectrometer. The NMR spectrum of lot A exhibits a smaller set of peaks at 9.25 ppm than the NMR spectrum of lots. Based on the integrated peak at 9.25 ppm in each spectrum, an impurity level of 21% was calculated for the sulfating agent of lot A, an impurity level of 33% was calculated for the sulfating agent of lot B and an impurity level of 36% was calculated for the sulfating agent of lot C.

### Example 19. Process Parameters for Sulfating 25-hydroxy-(3β)-cholest-5-en-3-ol

A sulfation reaction study to minimize and control the formation of bis-sulfated product 5-cholesten-3β-25-diol-disulfate was conducted.

### Example 19A. Sulfation with particles of 25-hydroxy-(3β)-cholest-5-en-3-ol in Reaction Mixture

During the sulfation reaction, it was observed that the 25-hydroxy-(3β)-cholest-5-en-3-sulfate organic cationic salt precipitates as a gel-like solid during the reaction. Some of this colloidal material may be solubilized in the reaction mixture due to its particle size. To minimize this solubility effect, the addition of seed crystals of 25-hydroxy-(3β)-cholest-5-en-3-sulfate organic cationic salt to the reaction to modify the product crystal shape was tested. As the reaction proceeds by charging the sulfur trioxide-pyridine complex, the gel-like solids of 25-hydroxy-(3β)-cholest-5-en-3-sulfate organic cationic salt produced during the sulfation reaction turned into an amorphous slurry with a larger particle size. This allowed for control of the solubility of the generated 25-hydroxy-(3β)-cholest-5-en-3-sulfate organic cationic salt in the reaction mixture. This also resulted in minimizing the formation of bis-sulfated product 5-cholesten-3β-25-diol-disulfate in the reaction mixture.

25-hydroxy-(3β)-cholest-5-en-3-ol was dissolved with 2-methyl tetrahydrofuran (30V); and heated to about 35-40° C. The solution was cooled to about 20±5° C. and seed crystals of 25-hydroxy-(3β)-cholest-5-en-3-sulfate organic cationic salt were added. The sulfating agent sulfur-trioxide-pyridine complex was added in four portions held 2 hours apart from each other. Water (2 equivalents) was added to the slurry and held for 1 hour. At this point, agitation was reduced to a minimum vortex deep. Pyridine (2 equivalents) in 2-methyl tetrahydrofuran was added and the slurry was held for 12 hours or longer. Crude 25-hydroxy-(3β)-cholest-5-en-3-sulfate organic cationic salt product was collected by filtration and washed with 2-methyl tetrahydrofuran-pyridine (5%). The presence of bis-sulfated product 5-cholesten-3R-25-diol-disulfate was estimated to be about 2-5% in the crude product.

### Example 19B. Quenching of Unreacted Sulfur Trioxide-Pyridine Sulfation Reagent

Quenching excess unreacted sulfur-trioxide pyridine sulfation reagent was evaluated using two equivalents of water and pyridine to keep basic conditions and to avoid hydrolysis of the 25-hydroxy-(3β)-cholest-5-en-3-sulfate organic cationic salt product. In Items 1-3 of Table 11, water and pyridine were added simultaneously and held for one hour; then, the product was isolated by vacuum filtration. In item 4 of Table 11, the holding time was extended to mimic time expansion. To control the competition reactions between reagent hydrolysis and bis-sulfation, reagent hydrolysis was evaluated by adding water and holding it for one hour. This approach maximized excess hydrolysis. Pyridine was then added to minimize product hydrolysis (item5, Table 11). As summarized in Table 11, the addition of water for 1 hour followed by mixing with pyridine overnight afforded the highest yield of the 25-hydroxy-(3β)-cholest-5-en-3-sulfate organic cationic salt product and the lowest amount of bis-sulfated product and desmosterol impurity.

During quenching of excess unreacted sulfation reagent, it was determined that the agitation speed can play a role in competition between formation of bis-sulfated product 5-cholesten-3β-25-diol-disulfate and reagent quench. At high agitation speed, the unquenched sulfur trioxide-pyridine complex agglomerates brake apart, allowing further reaction with the 25-hydroxy-(3β)-cholest-5-en-3-sulfate organic cationic salt product. At slow agitation speed agglomerated complex remains at the bottom of the reactor minimizing this side reaction. Bis-sulfated product 5-cholesten-3β-25-diol-disulfate formation is observed under these reaction conditions in a range of 2-5%. Isolated crude 25-hydroxy-(3β)-cholest-5-en-3-sulfate organic cationic salt product was stable enough for further purification.

TABLE 11
Quenching of unreacted Sulfur Trioxide-Pyridine Sulfation Reagent

| | HPLC area (%) | 25-hydroxy- | | | | |
|---|---|---|---|---|---|---|
| | Bis-sulfated | (3β)-cholest- | | 25- | | |
| | product 5- | 5-en-3-sulfate | | hydroxy- | | |
| | cholesten-3β- | organic | Des- | (3β)- | | |
| | 25-diol- | cationic | mosterol | cholest- | | |
| Item | disulfate | salt | sulfate | 5-en-3-ol | Stage | Time |
| 1 | 4.4 | 92.9 | 1.329 | 1.26 | IPC | 1 h |
| | 5.0 | 92.0 | 1.46 | 1.54 | Solid | |
| 2 | 1.4 | 97.3 | 0.58 | 0.67 | IPC | 1 h |
| | 2.8 | 94.3 | 0.68 | 2.16 | Solid | |
| 3 | 1.9 | 96.6 | 0.79 | 0.61 | IPC | 1 h |
| | 2.6 | 95.8 | 0.46 | 1.11 | Solid | |
| 4 | 1.1 | 96.4 | 0.77 | 1.67 | IPC | overnight |
| | 3.5 | 95.4 | 0.27 | 0.86 | Solid | |
| 5 | 1.8 | 97.0 | 0.69 | 0.57 | IPC | Water |
| | | | | | | 1 h, |
| | | | | | | pyridine |
| | | | | | | overnight |
| | 2.6 | 96.1 | 0.62 | 0.59 | Solid | |

*IPC—in process control

### Example 19C. Liquid Chromatography and Recrystallization of 25-Hydroxy-(3β)-cholest-5-en-3-Sulfate Organic Cationic Salt Product

The 25-hydroxy-(3β)-cholest-5-en-3-sulfate organic cationic salt product was purified using a plug column employing a silica gel (≥mass equivalent) stationary phase and a mixture of methylene chloride-methanol (85:15) and pyridine (1%) mobile phase. The chromatographic column was prepared with silica gel (5 mass-eq)/DCM-Pyridine (1%), with a 1:2 ratio diameter-silica gel. The column was carefully prepared to avoid disturbing the silica gel top layer. Crude 25-hydroxy-(3β)-cholest-5-en-3-sulfate organic cationic salt product was dissolved in methylene chloride-methanol (1:1)-pyridine (1%) (2.4V), the solution charged to the column, and rinsed with methylene chloride-methanol (15%)-Pyridine (1%) (2V). The column was eluted with methylene chloride-methanol (15%)-Pyridine (1%) (~75V). Samples of about 10V were taken and monitored by thin layer chromatography (mobile phase methylene chloride-methanol 7:3 one drop pyridine and CAM stain). Fractions containing the 25-hydroxy-(3β)-cholest-5-en-3-sulfate organic cationic salt product were combined and the fractions containing bis-sulfated product were excluded.

The 25-hydroxy-(3β)-cholest-5-en-3-sulfate organic cationic salt product was isolated and purified from collected fractions by two different processes:

Isolation and Recrystallization Process (IP)-A. Fractions with the product from the plug column were concentrated under the constant volume technique. The 25-hydroxy-(3β)-cholest-5-en-3-sulfate organic cationic salt product solution was added to the initial constant volume (28V) mixture of 2-methyl tetrahydrofuran-heptane (1:2)—Particle seeds of 25-hydroxy-(3β)-cholest-5-en-3-sulfate organic cationic salt product were added while synchronizing distillation and addition. Pressure was maintained between 20-25 in. Hg. Under these conditions, the product precipitated out immediately and remains as a slurry during the distillation. The slurry temperature is adjusted to 20-25° C. and held for a minimum of 1 hour. The product is collected by filtration and rinsed with 2-methyl tetrahydrofuran-heptane (1:2) followed by heptane. The collected material was dried at 30-35° C. under vacuum for 24 hours.

Isolation Process (IP)-B. Fractions with the product from the plug column are concentrated under vacuum to ~7V. If the solution remained or turned cloudy or solids were observed, methylene chloride was added until a clear solution was obtained. This concentrated 25-hydroxy-(3β)-cholest-5-en-3-sulfate organic cationic salt product solution was added dropwise to a mixture of 2-methyl tetrahydrofuran-heptane (1:3) containing seeds of the 25-hydroxy-(3β)-cholest-5-en-3-sulfate organic cationic salt for about 1 hour to 1.5 hours. The product was rinsed in the container with methylene chloride-methanol (1:1) (0.5V) for 1 hour at 20-25° C. After aging the slurry, the product was collected by filtration and rinsed with 2-methyl tetrahydrofuran-heptane (1:3), followed by heptane. The solids were dried at 30-35° C. under vacuum for 24 hours.

The purity of 25-hydroxy-(3β)-cholest-5-en-3-sulfate organic cationic salt product obtained by each isolation and recrystallization process is summarized in Table 12.

TABLE 12
Purity 25-hydroxy-(3B)-cholest-5-en-3-sulfate organic cationic salt product after
Isolation and recrystallization

| | | HPLC area (%) | | | | | |
|---|---|---|---|---|---|---|---|
| | | Bis-sulfated | 25-hydroxy- | | 25- | | |
| | | product 5- | (3β)-cholest- | | hydroxy- | | |
| | | cholesten-3β- | 5-en-3-sulfate | Des- | (3β)- | | |
| | Scale | 25-diol- | organic | mosterol | cholest-5- | Isolation | |
| Step | (g) | disulfate | cationic salt | sulfate | en-3-ol | Process | Yield |
| Sulfation | 430 | 3.89 | 95.23 | 0.40 | 0.43 | — | — |
| Purified | 540 | ND | 99.88 | 0.12 | ND | (IP)-A | 75.1 |
| Sulfation | 40 | 2.28 | 96.83 | 0.75 | 0.14 | — | — |
| Purified | 45 | ND | 99.45 | 0.51 | ND | (IP)-B | 84.0 |

### Example 20. XRPD Indexing

Indexing and structure refinement are computational studies. Within FIG. 4, agreement between the allowed peak positions, marked with bars, and the observed peaks indicates a consistent unit cell determination. Successful indexing of a pattern indicates that the sample is composed primarily of a single crystalline phase unless otherwise stated. Space groups consistent with the assigned extinction symbol, unit cell parameters, and derived quantities are tabulated below FIG. 4 and in Table 2. To confirm the tentative indexing solution, the molecular packing motifs within the crystallographic unit cells must be determined. No attempts at molecular packing were performed.

### Example 21. Differential Scanning Calorimetry (DSC)

DSC was performed using a Mettler-Toledo DSC3+ differential scanning calorimeter. A tau lag adjustment is performed with indium, tin, and zinc. The temperature and enthalpy are adjusted with octane, phenyl salicylate, indium, tin and zinc. The adjustment is then verified with octane, phenyl salicylate, indium, tin, and zinc. The sample was placed into a hermetically sealed aluminum DSC pan, and the weight was accurately recorded. The pan was then inserted into the DSC cell. A weighed aluminum pan configured as the sample pan was placed on the reference side of the cell. The pan lid was pierced prior to sample analysis. Samples were analyzed from 30° C. to 350° C. or 30° C. to 250° C., both at 10° C./min.

### Example 22. Proton NMR Spectroscopy

The ¹H-NMR spectrum is, other than a peak at 5.3 ppm, consistent with structure as seen in FIG. 6. There is no evidence of residual solvent. An overlap of a methylene from chlorine at about 3.8 ppm appears to be 1 mol/mol choline. The solution NMR spectra were acquired with an Agilent DD2-400 spectrometer. Samples were prepared by dissolving approximately 2-10 mg of sample in DMSO-d₆ containing TMS. The data acquisition parameters are displayed below in Table 13.

TABLE 13
NMR Parameters

| OS: CentOS Linux release 7.6.1810 (Core) | |
|---|---|
| Comp: nmr600-1 | |
| Processed on: TopSpin 4.0.8 | |
| Acquired on: TopSpin 4.0.8 | |
| Current Data Parameters | |
| EXPNO | 1 |
| PROCNO | 1 |

F1-Acquisition Parameters

| INSTRUM | AV4 NEO | |
|---|---|---|
| PROBHD | z149002_002 | |
| SOLVENT | DMSO | |
| TE | 298.1 | K |
| RO | 0 | Hz |
| PULPROG | zg | |
| D1 | 10.000 | sec |
| P1 | 12.0 | usec |
| PLW1 | 25.093 | W |
| AQ | 5.000 | sec |
| SWH | 10000.000 | Hz |
| SW | 16.6629 | ppm |
| NS | 16 | |
| DS | 0 | |
| TD | 100000 | |
| NUC1 | 1H | |
| SFO1 | 600.1339636 | MHz |

Fl-Processing parameters

| S1 | 524288 | |
|---|---|---|
| WDW | EM | |
| SSB | 0 | |
| LB | 0.20 | Hz |

| | |
|---|---|
| GB | 0 |

### Example 23. Thermogravimetric Analysis (TGA)

TG analysis was performed using a Mettler-Toledo TGA/DSC3 analyzer. Temperature and enthalpy adjustments were performed using indium, tin, and zinc, and then verified with indium. The balance was verified with calcium oxalate. The sample was placed in an open aluminum pan. The pan was hermetically sealed, the lid pierced, then inserted into the TG furnace. A weighed aluminum pan configured as the sample pan was placed on the reference platform. The furnace was heated under nitrogen. Each sample was heated from ambient temperature to 350° C. at 10° C./min. Although thermograms are plotted by reference temperature (x-axis), results are reported according to sample temperatures.

### Example 24. Transmission XRPD

XRPD patterns were collected with a PANalytical X'Pert PRO MPD or a PANalytical Empyrean diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640f) was analyzed to verify the observed position of the Si 111 peak is consistent with the NIST-certified position. A specimen of the sample was sandwiched between 3-μm-thick films and analyzed in transmission geometry. A beam-stop, short antiscatter extension, and antiscatter knife edge were used to minimize the background generated by air. Soller slits for the incident and diffracted beams were used to minimize broadening and asymmetry from axial divergence. Diffraction patterns were collected using a scanning position-sensitive detector (X'Celerator) located 240 mm from the specimen and Data Collector software v. 5.5. All images have the instrument labeled as X'Pert PRO MPD regardless of the instrument used.

### Example 25. Reflection Geometry (Samples in Limited Quantity)

To the extent made, XRPD patterns were collected with a PANalytical X'Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640f) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning position-sensitive detector (X'Celerator) located 240 mm from the sample and Data Collector software v. 5.5.

### Example 26. Dynamic Vapor Sorption (DVS)

Dynamic vapor sorption data was collected on a Surface Measurement System DVS Intrinsic instrument. The samples were not dried prior to analysis. Sorption and desorption data were collected over a range from 5% to 95% RH in 10% RH increments under a nitrogen purge. The equilibrium criteria used for the analyses were 0.001 dm/dt weight change in 5 minutes with a minimum step time of 30 minutes and maximum equilibration time of 180 minutes with a 3 minute data logging interval. Data were not corrected for the initial moisture content of the sample.

### Example 27. Preparing 25HC3S Triethylammonium

A column containing ~145 g “wet” Dowex 50WX8 (50-100 mesh, H+ form) was washed with distilled water until the eluate was colorless. A volume of 600 mL 1.5 M triethylammonium hydrochloride in H₂O/methanol (1/1), was treated with 12.6 mL triethylamine (pH now >8.5) and then passed through the column. The resin was then rinsed with methanol until the eluate was neutral (~200 mL).

41.2 g of 25HC3S sodium was dissolved in 820 mL methanol, and, after sonication, the cloudy solution was filtered through Celite. The filtrate was then passed through the above column and collected in ~200 mL fractions. Complete elution was achieved with an additional 600 mL MeOH. Drying under high vacuum overnight afforded 39 g 25HC3S triethylammonium as a chunky white solid (81% recovery). Homogenation with a mortar/pestle provided the target as a fine, white powder.

Proton NMR analysis (CDCl₃, d6-DMSO) indicated a quantitative conversion to the triethylammonium salt.

### Example 28. Preparing Crystalline 25HC3S Choline

A suspension of the 25HC3S triethylammonium of Example 27 (4.0 g, 6.9 mmol, 1 eq.) in 70 mL of acetonitrile was treated with 1 eq. choline hydroxide (6.9 mL of a 1 M solution in H₂O) and stirred 2-3 hours at room temperature. The solid was collected and rinsed with acetonitrile, then dried under high vacuum to afford 3.08 g of 25HC3S choline as a white solid (76%).

Proton NMR analysis (d6-DMSO) indicated no residual triethylamine.

### Example 29. Solubility of 25HC3S Choline at Room Temperature

### 1. Summary

The solubility of 25HC3S choline in aqueous and organic solvents was determined after one day at room temperature. The aqueous solvents were water, 5% dextrose, 0.9% sodium chloride, Fasted State Simulated Intestinal Fluid (FaSSIF), Fed State Simulated Intestinal Fluid (FeSSIF) and Fasted State Simulated Gastric Fluid (FaSSGF). The organic solvents were methanol, ethanol, isopropanol and acetonitrile.

### 2. Introduction and Purpose

The objective of this study was to determine the solubility of 25HC3S choline at room temperature in 4 organic solvents and 6 aqueous solutions.

### 3. Materials

Materials included the following.
3.1. 0.9% Sodium Chloride: Hospira
3.2. 5% dextrose: Hospira
3.3. Water: DURECT USP purified
3.4. Methanol: EMD
3.5. Ethanol: Pharmco
3.6. Acetonitrile: Honeywell
3.7. Isopropyl alcohol: Honeywell
3.8. FaSSIF, FeSSIF, FaSSGF powder: Biorelevant
3.9. FeSSIF buffer concentrate: Biorelevant
3.10. FaSSIF buffer concentrate: Biorelevant
3.11. FaSSGF buffer concentrate: Biorelevant
3.12. 25HC3S choline used in the study is listed in Table A1.

TABLE A1
25HC3S Choline in Solubility Study

| | Molecular |
|---|---|
| | Weight |
| Compound | (g/mol) |
| 25HC3S choline | 585.88 |

### 4. Study Experimental Procedures

### 4.1 Preparation of Simulated Intestinal and Gastric Fluids

These fluids were prepared per the instruction listed by Biorelevant (Biorelevant.com). Each buffer concentrate was diluted with water and mixed well. FaSSIF, FeSSIF, FaSSGF powder was added to each buffer and mixed to completely dissolve the powder. Buffers were stored at room temperature and used within 48 hours of preparation.

TABLE A2
Preparation of Simulated Intestinal and Gastric Fluids

| | | | Buffer | | Purified | | FaSSIF, FeSSIF, | |
|---|---|---|---|---|---|---|---|---|
| Target | | | Concentrate, g | | Water, g | | FaSSGF Powder, g | |
| Volume, | Simulated | Buffer | Target | Actual | Target | | Target | |
| mL | Fluid | Concentrate | Wt. | Wt. | Wt. | Actual Wt. | Wt. | Actual Wt. |
| 450 | FaSSIF | FaSSIF | 18.74 | 18.76 | 432.5 | 432.51 | 1.008 | 1.009 |
| | FeSSIF | FeSSIF | 36.64 | 36.65 | 413.75 | 413.83 | 5.04 | 5.04 |
| | FaSSGF | FaSSGF | 16.55 | 16.58 | 432.85 | 432.86 | 0.027 | 0.028 |

### 4.2 Solubility Sample Preparation

25HC3S choline was weighed in 8 mL or 4 mL glass vials with Teflon coated screw caps. Aliquots of each solvent were added to each sample and vortexed for about one minute until excess solid remained. Samples were placed on shaker at room temperature with 210 motion per minute shaking.

After one day, samples were pulled and checked if excess solid was present. About 1.5 mL aliquot of each sample was centrifuged at 12000 rpm for 12 to 15 minutes. Some samples required 2× centrifugation. Aliquots of the top clear solution of each centrifuged sample in MeOH, EtOH, IPA and ACN were diluted with diluent (water:MeOH 95:5) and assayed by HPLC. Samples of 25HC3S choline in aqueous media were assayed by HPLC without dilution.

Note: some samples did not have excess solid present after 1 day, solubility of drug in these samples is reported as ≥concentration values.

### 4.3 Analytical Testing Procedures

All solubility sample solutions of 25HC3S choline were determined using HPLC. A linear standard curve was constructed using standards with concentration between 11 μg/mL to 1.5 mg/mL as free acid forcing the curve to origin. Solubilities are reported as mg/mL free acid 25HC3S. Detection limit (LOD) of the HPLC method was calculated as 0.0007 mg/mL.

### 5. Results and Discussion

Table A1 lists the relevant information regarding the 25HC3S choline used in the solubility study. Solubility was calculated and reported as the 25HC3S free acid. All solubilities were measured after one day at room temperature.

Table A3 shows solubility results of the 25HC3S choline. The final pH of the sample solutions in aqueous media is listed in Table A4.

TABLE A3
Solubility in Aqueous Solutions and Organic Solvents at Room Temperature (mg/mL as Free Acid)

| Crystalline | | 0.9% Sodium | 5% | | | | | | | |
|---|---|---|---|---|---|---|---|---|---|---|
| 25HC3S Salt | Water | Chloride | Dextrose | FaSSGF¹ | FaSSIF² | FeSSIF³ | MeOH⁴ | EtOH⁵ | IPA⁶ | ACN⁷ |
| 25HC3S choline | 0.107 | ND | 0.093 | 0.008 | 0.152 | 0.827 | 17.5 | 3.4 | 0.6 | 0.5 |

¹FaSSIF = Fasted State Simulated Intestinal Fluid, ²FeSSIF = Fed State Simulated Intestinal Fluid, ³FaSSGF = Fasted State Simulated Gastric Fluid, ⁴MeOH = methanol, ⁵EtOH = ethanol, ⁶IPA = isopropyl alcohol, and ⁷ACN = acetonitrile.
⁸ND = Not Detected, Detection Limit = 0.7 ug/mL

TABLE A4
pH the Solubility Samples of Crystalline
25HC3S Choline in Aqueous Media¹

| | | 0.9% Sodium | 5% | | | |
|---|---|---|---|---|---|---|
| Compound | Water | Chloride | Dextrose | FaSSGF | FaSSIF | FeSSIF |
| Medium | 6.3 | 5.8 | 4.7 | 1.5, 1.6* | 6.5, 6.5* | 4.9, 5.0* |
| Blank | | | | | | |
| 25HC3S | 8.8 | 8.2 | 7.3 | 1.6 | 6.5 | 5.0 |
| choline | | | | | | |

*Two values were from initial preparation of the simulated fluids and measured at the time of samples measurements.
¹FaSSIF = Fasted State Simulated Intestinal Fluid, FeSSIF = Fed State Simulated Intestinal Fluid, FaSSGF = Fasted State Simulated Gastric Fluid

### Example 30. Physical and Chemical Solid-State Stability of 25HC3S Choline

### 1. Summary

The stability of 25HC3S choline was determined after 3 weeks and 5 weeks at 2-8° C. and 80° C.

### 2. Introduction and Purpose

The objective of this study was to determine the solid state physical and chemical stability of 25HC3S choline at 2-8° C. and 80° C.

### 3. Equipment and Materials

Materials are listed in the following.
3.1. Analytical Balance, Mettler MT5
3.2. Sonicator, Branson 8510
3.3. Centrifuge, Eppendorf 5417C
3.4. Refrigerator (2-8° C. storage)
3.5. Incubator (80° C. storage)
3.6. Incubator (60° C. storage)
3.7. HPLC, Agilent 1100
3.8. Methanol: EMD
3.9. Diluent (95% Methanol/5% Water)
3.10. 20 mL Glass Vials with Fluoropolymer Resin/Silicone Septa Lined Screw Caps
3.11. 25HC3S choline used in the study is listed in Table B1.

TABLE B1
25HC3S Choline in Stability Study

| | Molecular |
|---|---|
| Compound Number/ | Weight |
| Name | (g/mol) |
| 25HC3S choline | 585.88 |

### 4. Study Experimental Procedures

### 4.1 Stability Sample Preparation for Stability Setup and Testing Schedule

Approximately 25 mg of 25HC3S choline was added to 20 mL glass vials and each powder weight was recorded. Four vials were prepared and stored at 2-8° C. or 80° C. according to Table B2. The samples were scheduled initially to pull for testing at 3 weeks and 6 weeks time points.

TABLE B2
Allocation of Stability Samples for 25HC3S
Choline

| Temperature | Number of Vials |
|---|---|
| 80° C. | 2 |
| 2-8° C. | 2 |

### 4.2 Analytical Testing

### 4.2.1 Appearance

For appearance, each unopened vial was visually examined for color, clumping, and flowability.

### 4.2.2 Determination of Assay and Degradation Products

### Preparation of Standard Solutions

Based on the molecular weights, and expected UV response factors used in this study, the expected analytical sample concentrations were within the range of the 25HC3S sodium reference standards (0.75 mg/mL to 1.25 mg/mL as 25HC3S sodium salt).

### Preparation of 25HC3S Choline (as Free-Flowing Powder)

Samples were prepared by pipetting 20 mL of 100% methanol into each vial and mixing well, with sonication and vortexing to fully dissolve the samples for HPLC analysis.

### 4.3 HPLC Analysis

HPLC analysis was performed.

### 5. Results and Discussion

Table B1 lists the relevant information regarding the 25HC3S choline used in the stability study. Table B4 shows the stability results of the 25HC3S choline and Table B5 presents stability for the 25HC3S choline with associated comments.

TABLE B4
Stability of 25HC3S Choline at 2-8° C. and 80° C.

| | | | | | % | | | | |
|---|---|---|---|---|---|---|---|---|---|
| | | | | | Recovery | | | | |
| | | | | | of 2- | | | | |
| | | | | | 8° C./3 wk | | | | |
| | | | | | VS | | | | |
| | | | | | 80° C../3 wk; | | | | |
| | | Time | | | 2-8º C./5 wk | % Impurities | | | |
| | | Point | | Assay | VS | ¹25- | | ³25- | |
| Compound | Condition | (wk) | Appearance | (% w/w) | 80° C./5 wk | SC | ²SD | HC | Total |
| 25HC3S | 2-8° C. | 3 | White, free flowing powder with clumping | 101.6 | | ND | 0.4 | 0.2 | 1.0 |
| choline | | 5 | White, free flowing powder with clumping | 108.1 | NA | ND | 0.3 | 0.2 | 0.9 |
| | 80° C. | 3 | White, free flowing powder with clumping | 102.4 | 100.8 | ND | 0.4 | 0.4 | 1.2 |
| | | 5 | White, free flowing powder with clumping | 105.2 | 97.3 | ND | 0.8 | 0.2 | 2.2 |

¹25-Sulfate-Cholesterol;
²Sulfate Desmosterol;
³25-Hydroxy-Cholesterol
ND = Not Detected

TABLE B5
Physical and Chemical Solid-State Stability of
Crystalline 25HC3S Choline

| Compound | Comments |
|---|---|
| 25HC3S | Stable after 5 weeks at 80° C. |
| Choline | physically and chemically. |

### Example 31. Measured Powder Flow Properties of 25HC3S Choline

### Background

The pharmaceutical industry relies heavily on the use of powders during the manufacture of dosage forms, especially tablets and capsules. There are numerous references in the pharmaceutical literature, attempting to correlate the various measures of powder flow to manufacturing properties. There are a variety of methods for characterizing powder flow. In addition, while it is clear that no single and simple test method can adequately characterize the flow properties of pharmaceutical powders, certain test methods may be valuable during pharmaceutical development. Two commonly reported methods for testing powder flow are angle of repose and compressibility index or Hausner ratio.

### Angle of Repose

Angle of repose is measured as follows. Powder is poured from a funnel onto a flat surface until a cone of powder is created. The angle of repose is calculated according to the following equation:

$$Angle of repose = arctan ( h / r )$$

Where:
   h: height of the cone (mm)
   r: radius of the cone (mm)

Table C1, from USP<1174> Powder Flow, describes the powder flow properties correlating to the angles of repose.

TABLE C1
Flow Properties of Powders and the Corresponding Angles of
Repose According to USP<1174> Powder Flow

| Flow Property | Angle of Repose (degrees) |
|---|---|
| Excellent | 25-30 |
| Good | 31-35 |
| Fair-aid not needed | 36-40 |
| Passable-may hang up | 41-45 |
| Poor-must agitate, vibrate | 46-55 |
| Very poor | 56-65 |
| Very, very poor | >66 |

The lower the angle of repose, the more flowable is a material. Powder with an angle of repose between 25° to 30° will show excellent flow through a hopper, feed frame, die table and die bore smoothly. From 41° to greater than 66°, you may experience powder flow difficulties like rat-holing or bridging within the hopper.

Product Bridging or “arching” and rat-holing are both issues that result in a no-flow condition. Bridging is a case where material that is being discharged or fed forms a bridge or arch over the feed auger or discharge point in a silo cone/hopper. Rat-holing is a condition where the material forms a hole or narrow channel above the feed auger or outlet in a hopper while the remaining material is stationary against the hopper wall. Both of these conditions result in the product not flowing as desired.

### Experimental

The angle of repose was determined by pouring approximately 2 grams of 25HC3S choline through a 7 mm inner diameter plastic tube into the center of a 21.14 mm diameter rubber O-ring, resulting in the formation of a powder cone of fixed diameter. The height of the cone was estimated with calipers.

### Results

Table C2 shows a summary of the angle of repose measurements for the 25HC3S choline, and their classification according to USP<1174> Powder Flow.

TABLE C2
Angle of Repose for 25HC3S Choline and Classification of
Flow Properties According to USP<1174> Powder Flow

| | | Angle of | |
|---|---|---|---|
| | | Repose | Flow |
| | | (degrees) | Property per |
| | Angle of | Rounded | USP<1174> |
| Compound Number/ | Repose | Whole | Powder |
| Name | (degrees) | Number | Flow |
| 25HC3S choline | 34.9 | 35 | Good |

### Compressibility Index and Hausner Ratio

The compressibility index and the closely related Hausner ratio are simple, fast, and popular methods of predicting powder flow characteristics. The compressibility index has been proposed as an indirect measure of bulk density, size and shape, surface area, moisture content, and cohesiveness of materials because all of these can influence the observed compressibility index. The compressibility index and the Hausner ratio are determined by measuring both the bulk volume and the tapped volume of a powder.

Although there are some variations in the method of determining the compressibility index and Hausner ratio, the basic procedure is to measure the unsettled apparent volume, V_{O}, and the final tapped volume, V_{f}, of the powder after tapping the material until no further volume changes occur. The compressibility index and the Hausner ratio are calculated as follows:

$$Compressibility Index = 100 \times [ ( {V}_{O} - {V}_{f} ) / {V}_{O} ]$$

$$Hausner Ratio = ( {V}_{O} / {V}_{f} )$$

Both the Hausner ratio and the compressibility index are empirically established. The Hausner ratio and compressibility index are not absolute properties of a material; its value can vary depending on the methodology used to determine them.

Table C3, from USP <1174> Powder Flow, describes the flow character of the powder based on the compressibility index and Hausner ratio values.

TABLE C3
Flow Properties of Powders and the Corresponding
Compressibility Index and Hausner Ratios According
to USP<1174> Powder Flow

| Compressibility | | |
|---|---|---|
| Index (%) | Flow Character | Hausner Ratio |
| ≤10 | Excellent | 1.00-1.11 |
| 11-15 | Good | 1.12-1.18 |
| 16-20 | Fair | 1.19-1.25 |
| 21-25 | Passable | 1.26-1.34 |
| 26-31 | Poor | 1.35-1.45 |
| 32-37 | Very poor | 1.46-1.59 |
| >38 | Very, very poor | >1.60 |

### Experimental

Powder was carefully poured into a 5 mL glass graduated cylinder (to contain) until it was close to or at 5.0 mL. This initial volume was recorded as Vₒ. The graduated cylinder was tapped on a hard surface for 200 times and the volume was recorded. The cylinder was tapped 100 times more, and the final volume V_{f} was recorded. Typically, the volume after 200 taps was identical to after 300 taps, or within 0.1 mL.

### Results

Table C4 shows a summary of the Hausner ratio and compressibility index measurements for the 25HC3S choline, and their flow properties classification according to USP<1174> Powder Flow.

TABLE C4
Hausner Ratio and Compressibility Index for 25HC3S
Choline and Classification of Flow Properties According to
USP<1174> Powder Flow

| | | | Scale of |
|---|---|---|---|
| | | | Flowability per |
| | Hausner | Compressibility | USP<1174> |
| Compound | Ratio | Index | (Flow Character) |
| 25HC3S | 1.72 | 42 | Very, very poor |
| choline | | | |

### Example 32. Spray-Dried Dispersions of 25HC3S Choline

Table D1 indicates 3 solutions containing 25HC3S choline which were prepared for spray drying in the amounts indicated.

TABLE D1
25HC3S Choline Dispersions

| Component | (% w/w) | (% w/w) | (% w/w) |
|---|---|---|---|
| 25HC3S Choline¹ | 25% | 25% | 100% |
| (Non-GMP API) | | | |
| Copovidone | 75% | — | — |
| HPMC E5 | — | 75% | — |
| Total Solids | 100 | 100 | 100 |
| Methanol² | 80.6% w/w | 80.6% w/w | 80.6% w/w |
| Water² | 19.4% w/w | 19.4% w/w | 19.4% w/w |
| Batch Size (g) | 30 | 30 | 1 |

¹25HC3S Choline percent referred as a salt form and salt adjustment was not performed.
²Methanol and water evaporated during secondary drying and absent in the SDD.

Table D2 indicates the spray drying parameters used.

TABLE D2
Spray Drying Parameters

| | |
|---|---|
| Parameter | Set value |

Spray Drying Parameter

| Equipment | ProCepT | |
|---|---|---|
| Drying air flowrate | 0.45 | L min⁻¹ |
| Air inlet temperature | 80.0° C. | |
| Air outlet temperature | Target 45.0° C. | |
| | (40-48.0° C.) | |
| Atomizing nozzle diameter | 0.8 | mm |
| Atomization air pressure | 1.5 | bar |
| Atomization air flow | 15.0 | L min⁻¹ |
| Spray rate | 4.0 | g min⁻¹ |
| Cyclone | Large cyclone | |
| Cyclone Differential | 12.0 | bar |

Pressure

Secondary Drying Parameters

| Equipment | Lab scale | |
|---|---|---|
| | convection oven | |
| Temperature | 40° C. | |
| Drying time | 24 | hours |
| Powder bed thickness | ~4.0 | mm |

X-ray powder diffraction patterns of each dispersion are in FIGS. 13A, 13B and 13C. DSC thermograms of individual ingredients can be found in FIGS. 12A, 12B, and 12C. The XRPD diffractograms were collected using a Rigaku MiniFLex diffractometer with the settings set forth in Table D2.1 below.

TABLE D2.1
XRPD Parameters

| X-ray generator | 40 kV, 15 mA | |
|---|---|---|
| Wavelength | Cu-Kα/1.54186 Å | |
| Goniometer | MiniFlex 300/600 | |
| Attachment | ASC-8 | |
| Attachment base | None | |
| Detector name | D/teX Ultra2 | |
| Optics attribute | None | |
| Scan mode | 1D(scan) | |
| Scan speed | 20.0000°/min | |
| Scan step | 0.0200° | |
| Scan axis | θ/2θ | |
| Scan range | 3.0000-45.0000° | |
| Incident primary | No unit | |
| Selection slit | None | |
| Incident Soller | Soller slit 5.0° | |
| DS | 1.25deg | |
| IHS | 10 | mm |
| SS | 8.0 | mm |

| | |
|---|---|
| Incident filter | None |
| Receiving Optical | None |
| Device | |
| Receiving Soller | Soller slit 5.0° |
| RS | Open |
| Attenuator | Open |
| Detector | None |
| Monochrometer | |
| Detector slit | None |

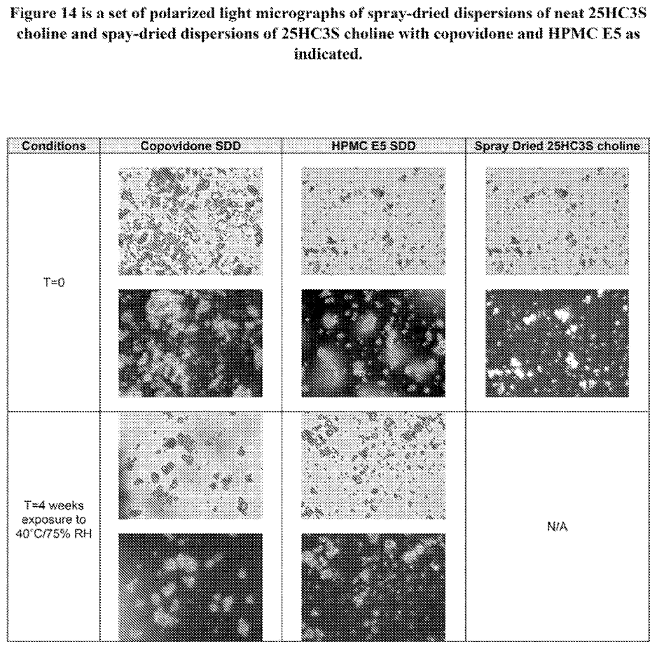

Drug release of copovidone SDD, HPMC E5 SDD and 25HC3S choline neat is given in FIG. 15. 25HC3S choline is a weak acid and along with SDDs showed higher solubility in intestinal pH compared to gastric pH. 25HC3S choline showed ~10% drug release at 90 minutes, and both SDDs showed higher drug release compared to 25HC3S choline. This shows that 25HC3S choline solubility was significantly increased after spray drying (i.e., 10 times with copovidone SDD and 5 times with HPMC E5 SDD compared to neat API at 90 min). Biorelevant dissolution drug release results showed that SDDs prepared by spray drying can significantly enhance solubility of 25HC3S choline. As used herein, the biorelevant drug release was conducted using a USP Apparatus 2 with (1) 0-30 minutes in 450 mL FaSSGF; and (2) 30-90 minutes FaSSGF+double strength FaSSIF in a total of 900 mL of media with pH adjusted to 6.5; with a paddle speed of 75 rpm (0-75 minutes) and 250 rpm (75-90 minutes) and a vessel temperature of 37° C. FIG. 14 is a set of polarized light microscope micrographs of spray-dried dispersions of neat 25HC3S choline and spray-dried dispersions of 25HC3S choline with copovidone and HPMC E5 as indicated.

### Example 33. Physical and Chemical Stability of Spray-Dried Dispersions

Spray Dried Dispersions (“SDDs”) of Example 32 containing 25HC3S choline and copovidone or HPMC E5 were placed in tightly closed vials as per Table D3 and analyzed for physical and chemical stability at each timepoint. XRPD and DSC of copovidone and HPMC SDD are shown in FIGS. 16A, 16B, 16C, 17A, 17B, 17C, 18, and 19, and dynamic vapor sorption is shown in FIGS. 20 and 21. The XRPD diffractograms were collected using a Rigaku MiniFLex diffractometer with the settings set forth in Table D2.1. Stability measurements were done in closed vials due to the hygroscopic nature of the dispersions.

In SDD, amorphous drug form is desirable to achieve higher dissolution rates. To maintain dissolution advantages during shelf-life, a drug must remain in the amorphous form. 25HC3S choline SDDs were shown to be amorphous. No crystallization was observed in SDDs after 28 days exposure to 2-8° C., 25° C./60% RH or 40° C./75% RH. Polarized microscopy showed absence of crystallinity. DSC thermograms also corroborate amorphous form during stability after 14-days exposure to 3 storage conditions. Both SDDs were shown to absorb moisture with approximately 10% weight gain at 75% RH and further increment in humidity, up to 35% weight gain was observed at 95% RH.

TABLE D3
Stability Conditions

| Condition | Timepoint (weeks) | | |
|---|---|---|---|
| 2-8° C. | 0 | 2 | 4 |
| 25° C./60% RH | | 2 | 4 |
| 40° C./75% RH | | 2 | 4 |

Chemical assays revealed that the spray dried dispersions were chemically stable.

### Example 34. Tablet of Spray-Dried Dispersions

Using a spray-dried dispersion of a copovidone solid dispersion of 25HC3S choline of Example 32 as a pre-blend with intragranular excipients, the pre-blend was slugged with 22 mm tooling and a compression force of 22,500 N. The slugs were crushed using a mortar and pestle and passed through a #16 mesh screen. Additional blending with extragranular excipients and compressing using 7 mm tooling to achieve a 7 kp hardness was performed.

Tablets made according to this process were tested for drug release with results shown in FIG. 23. The components of the tablets made and tested are set forth in Table D4.

TABLE D4
(all values in weight %)

| | | | |
|---|---|---|---|
| | Tablet | Tablet | Tablet |
| Components | S1 | S2 | S3 |

Intragranular

| | | | |
|---|---|---|---|
| Copovidone 25HC3S | 40.0 | 40.0 | 40.0 |
| Choline Dispersion | | | |
| Microcrystalline Cellulose | 36.6 | 31.6 | 34.6 |
| (Avicel PH-101) | | | |
| Lactose Monohydrate | 12.40 | 11.4 | 12.4 |
| (Fast Flo) | | | |
| Crospovidone | 6.0 | 5.0 | 5.0 |
| Sodium Lauryl Sulfate | — | 4.0 | — |
| Colloidal Silicon Dioxide, | 1.0 | 1.0 | 1.0 |
| Aerosil 200 | | | |
| Magnesium Stearate | 0.5 | 0.5 | 0.5 |
| (Vegetable Grade) | | | |
| Total Intragranular | 96.5 | 93.5 | 93.5 |

Extragranular

| | | | |
|---|---|---|---|
| Crospovidone | 2.0 | 5.0 | 5.0 |
| Colloidal Silicon Dioxide, | 1.0 | 1.0 | 1.0 |
| Aerosil 200 | | | |
| Magnesium Stearate | 0.5 | 0.5 | 0.5 |
| (Vegetable Grade) | | | |
| Total Extragranular | 3.5 | 6.5 | 6.5 |
| Total | 100.0 | 100.0 | 100.0 |
| Tablet weight target (mg) | 125.0 | 125.0 | 125.0 |
| Tablet hardness target (kP) | 7.5 | 7.5 | 7.5 |

### Example 35. Tablets of Spray-Dried Dispersions

The process of Example 34 was performed, but with final compression using 8 mm tooling and a hardness of 6-7 kp.

Tablets made according to this process were tested for drug release with results shown in FIG. 24. The components of the tablets made and tested are set forth in Table D5.

TABLE D5
(all values in weight %)

| | | |
|---|---|---|
| | Tablet | Tablet |
| Components | S4 | S5 |

Intragranular

| | | |
|---|---|---|
| Copovidone 25HC3S Choline | 22.22 | 22.22 |
| Solid Dispersion | | |
| Microcrystalline Cellulose | 33.58 | 37.32 |
| (Avicel PH-101) | | |
| Lactose Monohydrate (Fast Flo) | 11.2 | 12.46 |
| Crospovidone | 5.0 | 5.0 |
| Sodium Lauryl Sulfate | — | 5.0 |
| Sodium Chloride | 10.0 | — |
| Colloidal Silicon Dioxide, | 1.0 | 1.0 |
| Aerosil 200 | | |
| Magnesium Stearate | 0.5 | 0.5 |
| (Vegetable Grade) | | |
| Total Intragranular | 83.5 | 83.5 |

Extragranular

| | | |
|---|---|---|
| Crospovidone | 15.0 | 15.0 |
| Colloidal Silicon Dioxide, | 1.0 | 1.0 |
| Aerosil 200 | | |
| Magnesium Stearate | 0.5 | 0.5 |
| (Vegetable Grade) | | |
| Total Extragranular | 16.5 | 16.5 |
| Total | 100.0 | 100.0 |
| Tooling size (mm) | 8.0 | 8.0 |
| Tablet weight target (mg) | 225.0 | 225.0 |
| Tablet hardness target (kP) | 6-7 | 6-7 |

### Example 36. Tablets of Spray-Dried Dispersions

Following a similar process as Examples 34 and 35, tablets were made as set forth in Table D6. The drug release profiles were shown in FIG. 25.

TABLE D6
(all values in weight %)

| | | | |
|---|---|---|---|
| | Theoretical | | |
| Component | % w/w | mg/Tablet | g/batch |

Intragranular

| | | | |
|---|---|---|---|
| Copovidone + 25HC3S | 22.22 | 50.00 | 4.15 |
| Choline Dispersion¹ | | | |
| Microcrystalline Cellulose | 18.66 | 41.99 | 1.98 |
| (Avicel PH-101) (Portion I) | | | |
| Microcrystalline Cellulose | 18.66 | 41.99 | 2.80 |
| (Avicel PH-101) (Portion II) | | | |
| Lactose Monohydrate | 12.46 | 28.04 | 1.87 |
| (Fast Flo) | | | |
| Crospovidone | 5.00 | 11.25 | 0.75 |
| Sodium Lauryl Sulphate | 5.00 | 11.25 | 0.75 |
| Colloidal Silicon Dioxide, | 1.00 | 2.25 | 0.15 |
| Aerosil 200 | | | |
| Magnesium Stearate | 0.50 | 1.13 | 0.08 |
| (Vegetable Grade) | | | |
| Total Intragranular | 83.5 | 187.87 | 12.52 |

Extragranular

| | | | |
|---|---|---|---|
| Crospovidone | 15.00 | 33.75 | 2.25 |
| Colloidal Silicon Dioxide, | 1.00 | 2.25 | 0.15 |
| Aerosil 200 | | | |
| Magnesium Stearate | 0.5 | 1.13 | 0.08 |
| (Vegetable Grade) | | | |
| Total Extragranular | 16.5 | 37.13 | 2.48 |
| Total (Tablet) | 100 | 225 | 15.0 |

¹SDD intermediate was adjusted for salt factor and purity of API from CoA

### Example 37. Preparation of Micronized 25HC3S Choline Tablets

A pre-blend of 25HC3S choline and intragranular excipients was made and slugged using a mortar and pestle and passed through a #16 mesh screen. A final blend was made with extragranular excipients and compressed using 7 mm tooling to achieve 7.5 kp hardness.

Tablets made according to this process were tested in 0.5% SDS in 900 mL 0.1 N HCl with results shown in FIG. 28. The components of the tablets made and tested are set forth in Table D7. Drug release in biorelevant media is set forth in FIG. 29.

TABLE D7
(all values in weight %)
Round 1 Prototypes
Theoretical Composition (% w/w)

| | | | |
|---|---|---|---|
| Material Description | Tablet M1 | Tablet M2 | Tablet M3 |

Intragranular

| | | | |
|---|---|---|---|
| Micronized 25HC3S | 12.5 | 12.5 | 12.5 |
| Choline¹ | | | |
| Microcrystalline Cellulose | 44.7 | 35.7 | 38.7 |
| (Avicel PH-101) | | | |
| Lactose Monohydrate. | 29.8 | 23.8 | 25.8 |
| (Fast Flo) | | | |
| Croscarmellose Sodium | 3.0 | 3.0 | 3.0 |
| (Ac-Di-Sol) | | | |
| Sodium Lauryl Sulfate | 5.0 | — | — |
| Copovidone | — | 20.0 | — |
| (Kollidon VA 64) | | | |
| Poloxamer 188 | — | — | 15.0 |
| Colloidal Silicon Dioxide, | 1.0 | 1.0 | 1.0 |
| Aerosil 200 | | | |
| Magnesium Stearate | 0.5 | 0.5 | 0.5 |
| (Vegetable Grade) | | | |
| Total Intragranular | 96.5 | 96.5 | 96.5 |

Extragranular

| | | | |
|---|---|---|---|
| Croscarmellose Sodium | 2.0 | 2.0 | 2.0 |
| (Ac-Di-Sol) | | | |
| Colloidal Silicon Dioxide, | 1.0 | 1.0 | 1.0 |
| Aerosil 200 | | | |
| Magnesium Stearate | 0.5 | 0.5 | 0.5 |
| (Vegetable Grade) | | | |
| Total Extragranular | 3.5 | 3.5 | 3.5 |
| Total | 100.0 | 100.0 | 100.0 |
| Tablet Weight (mg) | 100.0 | 100.0 | 100.0 |
| Tablet Hardness Target (kP) | 7.5 | 7.5 | 7.5 |

¹The target weight shown is the free acid form, this is adjusted for the choline salt form and purity of the lot of drug substance; a corresponding adjustment is made to the microcrystalline cellulose.

### Example 38. Preparation of Micronized 25HC3S Choline Tablets

Tablets were prepared in accordance with Example 37 using the components in Table D8.

TABLE D8
(all values in weight %)
Round 2 Prototypes
Theoretical Composition (% w/w)

| | | | | |
|---|---|---|---|---|
| | Tablet | Tablet | Tablet | Tablet |
| Material Description | M4 | M4A | M5 | M6 |

Intragranular

| | | | | |
|---|---|---|---|---|
| Micronized 25HC3S | 12.5 | 12.5 | 12.5 | 12.5 |
| Choline¹ | | | | |
| Microcrystalline Cellulose | 52.1 | 52.1 | 44.6 | 44.6 |
| (Avicel PH-101) | | | | |
| Lactose Monohydrate | 17.4 | 17.4 | 14.9 | 14.9 |
| (Fast Flo) | | | | |
| Croscarmellose Sodium | 7.5 | — | — | — |
| (Ac-Di-Sol) | | | | |
| Crospovidone | — | 7.5 | 7.5 | 7.5 |
| Sodium Lauryl Sulfate | 5.0 | 5.0 | — | 5.0 |
| Copovidone | — | — | — | 10.0 |
| (Kollidon VA 64) | | | | |
| Meglumine | — | — | 15.0 | — |
| Colloidal Silicon Dioxide, | 1.0 | 1.0 | 1.0 | 1.0 |
| Aerosil 200 | | | | |
| Magnesium Stearate | 0.5 | 0.5 | 0.5 | 0.5 |
| (Vegetable Grade) | | | | |
| Total Intragranular | 96.0 | 96.0 | 96.0 | 96.0 |

Extragranular

| | | | | |
|---|---|---|---|---|
| Croscarmellose Sodium | 2.5 | — | — | — |
| (Ac-Di-Sol) | | | | |
| Crospovidone | — | 2.5 | 2.5 | 2.5 |
| Colloidal Silicon Dioxide, | 1.0 | 1.0 | 1.0 | 1.0 |
| Aerosil 200 | | | | |
| Magnesium Stearate | 0.5 | 0.5 | 0.5 | 0.5 |
| (Vegetable Grade) | | | | |
| Total Extragranular | 4.0 | 4.0 | 4.0 | 4.0 |
| Total | 100.0 | 100.0 | 100.0 | 100.0 |
| Table Weight target (mg) | 100.0 | 100.0 | 100.0 | 100.0 |
| Tablet Hardness target (kP) | 7.5 | 7.5 | 7.5 | 7.5 |

¹The target weight shown is the free acid form, this is adjusted for the choline salt form and purity of the lot of drug substance; a corresponding adjustment is made to the microcrystalline cellulose.

Drug release in 0.5% SDS in 900 mL 0.1 N HCl is set forth in FIG. 30 and in biorelevant media in FIG. 31.

### Example 39. Preparation of Micronized 25HC3S Choline Tablets

Tablets were prepared in accordance with Example 38 using the components in Table D9.

TABLE D9
(all values in weight %)
Round 3 Prototypes Theoretical Composition (% w/w)

| | | | |
|---|---|---|---|
| Material Description | Tablet M7 | Tablet M8 | Tablet M9 |

Intragranular

| | | | |
|---|---|---|---|
| 25HC3S choline | 12.5 | 12.5 | 12.5 |
| Micronized API¹ | | | |
| Microcrystalline Cellulose | 48.4 | 48.4 | 48.4 |
| (Avicel PH-101) | | | |
| Lactose Monohydrate | 16.1 | 16.1 | 16.1 |
| (Fast Flo) | | | |
| Crospovidone | 7.5 | 7.5 | 7.5 |
| Sodium bicarbonate | 10.0 | — | — |
| Sepitrap-80 | — | 10.0 | — |
| PEG-4000 | — | — | 10.0 |
| Colloidal Silicon Dioxide, | 1.0 | 1.0 | 1.0 |
| Aerosil 200 | | | |
| Magnesium Stearate | 0.5 | 0.5 | 0.5 |
| (Vegetable Grade) | | | |
| Total (Intragranular) | 96.0 | 96.0 | 96.0 |

Extragranular

| | | | |
|---|---|---|---|
| Crospovidone | 2.5 | 2.5 | 2.5 |
| Colloidal Silicon Dioxide, | 1.0 | 1.0 | 1.0 |
| Aerosil 200 | | | |
| Magnesium Stearate | 0.5 | 0.5 | 0.5 |
| (Vegetable Grade) | | | |
| Total (Extragranular) | 4.0 | 4.0 | 4.0 |
| Total | 100.0 | 100.0 | 100.0 |
| Table Weight target (mg) | 100.0 | 100.0 | 100.0 |
| Tablet Hardness target (kP) | 7.5 | 7.5 | 7.52 |

¹The target weight shown is the free acid form, this is adjusted for the choline salt form and purity of the lot of drug substance, a corresponding adjustment is made to the microcrystalline cellulose

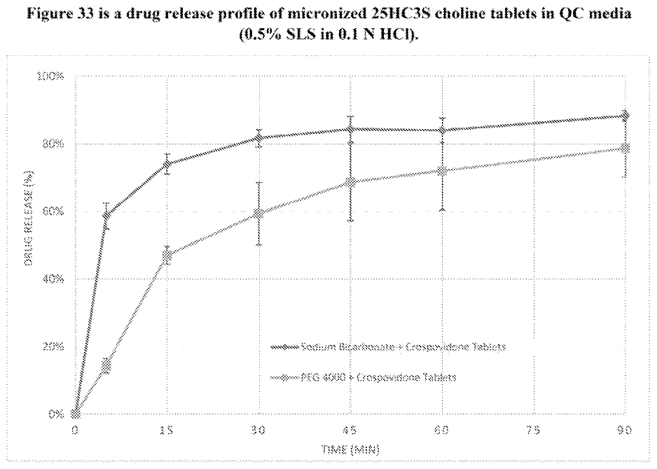

Drug release profiles for biorelevant media are shown in FIG. 32 and in 0.5% SLS in 0.1 N HCl in FIG. 33.

### Example 40. Micronized 25HC3S choline

Crystalline 25HC3S was placed in a JAM-2-2002 mill under nitrogen and milled. The materials were then placed into product containers. Jet milling was conducted at a feed rate of 300 g/hour, with a venturi pressure of 50 psi and mill pressure of 120 psi of nitrogen. The target specification was set to having a Dv (90) of less than 10 microns. The composite Dv (90) was measured to be 4 microns.

### Example 41. A Single Center, Open-Label, Non-Randomized, Two-Part Study to Assess the Pharmacokinetics and Food Effect of Immediate Release Formulations of 25-Hydroxycholesterol-3-Sulfate (25HC3S) in Healthy Subjects

### Objectives

The primary objectives of Part 1 of the study included:
To evaluate the pharmacokinetic (PK) profile of 25HC3S following administration of the choline salt immediate release (IR) test formulations in the fasted state
To evaluate the relative bioavailability of the 25HC3S choline salt IR test formulations in comparison with the reference choline salt powder in bottle (PiB) reference formulation in the fasted state
To evaluate the relative bioavailability of the 25HC3S sodium salt PiB formulation in comparison with the choline salt PiB reference formulation in the fasted state

The primary objectives of Part 2 of the study included:
To evaluate the effect of food on the PK of the selected 25HC3S choline salt test formulation
To evaluate the relative bioavailability of multiple units of the selected 25HC3S choline salt IR test formulation with the single unit of the same formulation dosed in Part 1, in the fasted state

The secondary objective for both Parts 1 and 2 of the study was:
To provide information on the safety and tolerability of oral doses of 25HC3S in healthy subjects

### Methods

The study was a single-center, non-randomized, 2-part study in healthy male and female subjects. Sixteen (16) healthy male and female subjects were enrolled to ensure data in an appropriate number of evaluable subjects (approximately 12 subjects). Subjects were to receive 7 regimens of 25HC3S in a sequential manner, over 2 clinical residences (Parts 1 and 2), with a minimum of 14 days between Parts 1 and 2 for interim decisions.

An evaluable subject for the primary objective of PK assessment in Part 1, was one that received at least 1 dose of the 25HC3S choline salt IR test formulations and had PK data up to 24 h post-dose and safety up to 48 h post dose. An evaluable subject for the primary objective of relative bioavailability assessment for comparison of the choline salt IR test formulation with the PiB reference, was one that received at least 1 dose of the 25HC3S choline salt IR formulations and the 25HC3S choline salt PiB reference formulation and had PK data up to 24 h post dose and safety up to 48 h post dose. An evaluable subject for the primary objective of relative bioavailability assessment for comparison of the sodium and choline salt PiB formulations, was one that received both 25HC3S choline and sodium salt PiB formulations and had PK data up to 24 h post dose and safety up to 48 h post dose. An evaluable subject for the food effect assessment, was one that received the selected 25HC3S choline salt IR formulation at the same dose level in both the fasted and fed state and had PK data up to 24 h post dose and safety up to 48 h post-dose. An evaluable subject for the comparison of the selected dose and formulation in Part 2 compared with the same formulation in Part 1, was one that received the selected 25HC3S choline salt IR formulation in Part 2 and the same formulation in Part 1 and had PK data up to 24 h post dose and safety up to 48 h post-dose.

The regimens received are summarized below in Table 41.1.

TABLE 41.1
Summary of Regimens for administering 25HC3S to
healthy male and female subjects

| | | | | | Route of |
|---|---|---|---|---|---|
| | | | Investigational | | Administration and |
| Part | Period | Regimen | Medicinal Product | Dose | Prandial State |
| 1 | 1 | A | 25HC3S choline salt PiB | 12.5 mg | Oral, Fasted |
| | | | in OraBlend SF | | |
| | | | (reference) | | |
| | 2 | B | Micronized 25HC3S | 12.5 mg | |
| | | | choline tablet M4A | (1 × 12.5 mg) | |
| | 3 | C | Micronized 25HC3S | | |
| | | | choline tablet M7 | | |
| | 4 | D | 25HC3S choline SDD | | |
| | | | tablet S5 | | |
| | 5 | E | 25HC3S sodium salt PiB | 12.5 mg | |
| | | | in OraBlend SF | | |
| 2 | 6 | F | The selected lead | 50 mg | Oral, Fed |
| | | | 25HC3S choline salt IR | (4 × 12.5 mg) | |
| | | | test formulation from | | |
| | | | Part 1ª | | |
| | 7 | G | The selected lead | 50 mg | Oral, Fasted |
| | | | 25HC3S choline salt IR | (4 × 12.5 mg) | |
| | | | test formulation from | | |
| | | | Part 1ª | | |

SDD; spray-dried dispersion,
SF; sugar free
The choline salt IR test formulations comprised the Micronized 25HC3S choline tablets and 25HC3S choline SDD tablet S5
ªThe selected lead 25HC3S choline salt IR test formulation from Part 1 was Micronized 25HC3S choline tablet M4A

Subjects underwent preliminary screening procedures for the study at the screening visit (Day −28 to Day −2).

### Part 1

For Part 1, subjects were admitted to the clinical unit in the morning on the day prior to investigational medicinal product (IMP) administration (Day −1). Subjects were to remain resident in the clinical unit for the dosing of the first 5 dosing periods (Part 1; Regimens A to E). Subjects were to fast from all food and drink (except water) for a minimum of 10 h prior to each IMP administration. Subjects were to receive 25HC3S (e.g., 25HC3S choline or 25HC3S sodium) on Day 1 of each period, with dosing for Regimens B to E taking place approximately 48 h after dose administration in the previous period.

No food was allowed until 4 h post-dose for each subject. No additional water was allowed between 1 h before and 1 h post-dose except for water used for administration. Additionally, subjects were restricted from lying down for the first 2 h post-dose. Subjects were to be discharged 48 h post-dose Regimen E. Blood samples were collected at specified intervals for PK and safety analysis from pre-dose Day 1 to discharge from the clinical unit.

Following Part 1 (Regimen E), there was an interim data review of safety and tolerability up to 48 h post-dose and PK data up to 24 h post-dose to decide on which formulation to use in Part 2 (Regimens F and G).

### Part 2

After a minimum 14-day period between completion of Part 1 (Regimen E) and commencement of Part 2 (Regimen F), subjects returned to the clinical unit for Part 2 (Regimens F and G). Subjects were admitted to the clinical unit in the morning of the day prior to IMP administration (Day −1). Subjects were to remain resident in the clinical unit for the remaining dosing periods (Regimens F and G). Subjects were to fast from all food and drink (except water) for a minimum of 10 h prior to each IMP administration (Regimen G) or prior to breakfast (Regimen F) as applicable. Subjects received a high-fat breakfast 30 min prior to dosing in Regimen F. Subjects received 25HC3S on Day 1 of both periods, with dosing for Regimen G taking place approximately 48 h after dose administration in Regimen F. No food was allowed until 4 h post-dose for each subject. No additional water was allowed between 1 h before and 1 h post-dose except for water used for administration. Additionally, subjects were restricted from lying down for the first 2 h post-dose. Subjects were to be discharged 48 h post-dose Regimen G. Blood samples were collected at specified intervals for PK and safety analysis from pre-dose Day 1 to discharge from the clinical unit. A follow-up phone call was to take place 7 to 9 days post-final dose to ensure the ongoing wellbeing of the subjects.

### Number of Subjects (Planned and Analyzed):

Planned: 16, Enrolled: 16, Completed: 13, Discontinued: 3.
Safety population: 16.
Safety Analysis sets: 16 (100.0%) for Regimens A and B, 15 (93.8%) for Regimen C and D and E, 13 (81.3%) for Regimens F and G.
PK population: 16.
PK Analysis sets: 16 (100.0%) for Regimens A and B, 15 (93.8%) for Regimen C and D and E, 13 (81.3%) for Regimens F and G.

### Diagnosis and Main Criteria for Inclusion:

Healthy males and non-pregnant, non-lactating healthy females aged 18 to 55 years inclusive at time of signing informed consent were included. Body mass index of 19.0 to 32.0 kg/m² or, if outside the range, considered not clinically significant by the investigator, and weight ≥50 kg as measured at screening.

### Tablet Formulations

Table 41.2 provides a list of excipients for the formulations described herein.

TABLE 41.2
List of excipients in tablet formulations

| Micronized | Micronized | 25HC3S choline Spray- |
|---|---|---|
| 25HC3S | 25HC3S | dried Dispersion (SDD) |
| choline tablet M4A | choline tablet M7 | tablet S5 |
| Microcrystalline | Microcrystalline | Microcrystalline |
| Cellulose | Cellulose | Cellulose |
| Lactose | Lactose | Lactose |
| Monohydrate | Monohydrate | Monohydrate |
| Sodium Lauryl | Sodium | Sodium Lauryl |
| Sulfate | Bicarbonate | Sulfate |
| Crospovidone | Crospovidone | Crospovidone |
| Colloidal Silicon | Colloidal Silicon | Colloidal Silicon |
| Dioxide | Dioxide | Dioxide |
| Magnesium | Magnesium | Magnesium |
| Stearate | Stearate | Stearate |

### Selection of Doses and Dose Timing in the Study

To date, 25HC3S has been evaluated in clinical trials in healthy subjects who have received single doses of 25HC3S up to 1000 mg orally. Part 1 of this study aimed to evaluate the relative bioavailability of the 25HC3S choline salt IR formulations compared to the choline salt PiB reference suspension formulation. Hence, a single unit tablet dose of 12.5 mg was explored in Part 1 of the study.

A sodium salt has been used previously. The current study is directed to the use of the choline salt of 25HC3S which is comparatively less hygroscopic and can minimize the polymorph changes compared to the sodium salt. The choline salt has a comparable solubility in water to that of the sodium salt, so it was not anticipated to have any significant impact on the drug's systemic exposure. However, the sodium salt PiB formulation was also dosed in Part 1 to provide a comparison of exposure from the two salt forms.

Subjects were dosed on the morning of Day 1 of each study period following an overnight fast (Part 1) or following an overnight fast or high-fat breakfast (Part 2). Subjects were allowed water up to 1 h before the scheduled dosing time and were provided with 240 mL of water at 1 h post dose. Water was allowed ad libitum after 1 h post-dose. Decaffeinated fluids were allowed ad libitum from lunch time on the day of dosing. Oral doses were administered with a total volume of 240 mL water.

The oral suspensions were administered in 20 mL of OraBlend SF (sugar free), followed by 2×60 mL water rinses of the dosing container and then an additional 100 mL to give a total volume of 240 mL.

Regimens B, C, D, E and G as shown in Table 41.1 were dosed approximately 48 h after dose administration in the previous period. There was a minimum 14-day period between Part 1 completion and commencement of Part 2 for interim decisions.

### Fasted Dosing (Part 1 Regimens a to E and Part 2 Regimen G)

The calorie/fat content of meals were not required to be controlled. Subjects were provided with a standardized menu. Subjects were provided with a light snack and were then to fasted from all food and drink (except water) for a minimum of 10 h on the day prior to dosing until approximately 4 h post-dose, at which time lunch was provided. For all dosing days, subjects received a standardized moderate-fat moderate calorie lunch at 4 h post-dose.

### Fed Dosing (Part 2, Regimen F)

The calorie/fat content of breakfast was controlled on Day 1 of Regimen F. Subjects were provided with a standardized menu for all other meals. Subjects were provided with a light snack and were then instructed to fast for a minimum of 10 h from all food and drink (except water) until the following morning, when they were provided with a high-fat breakfast (e.g., 2 eggs fried in 2 tsp of butter, 2 oz hash brown potatoes in 1 tsp butter, 2 slices of bacon, 2 slices of toast/1 pkg butter, 4 oz orange juice, 8 oz whole milk), to be given 30 min before dosing. The breakfast was to be consumed over a maximum period of 25 min, with dosing occurring 30 min after the start of breakfast. Subjects were encouraged to eat their meal evenly over the 25 min period. It was acknowledged that some subjects may take less time to eat, but dosing was still to occur 30 min after the start of breakfast. Subjects were to consume 100% to be eligible for dosing. The start and stop time and percentage of the breakfast consumed was recorded in the source.

### Reference Therapy, Dose and Mode of Administration:

In Part 1 of the study, subjects received reference IMP according to Table 41.3.

TABLE 41.3
Summary of 25HC3S reference administered to
subjects in Part 1 of the study

| | | | Route of Administration |
|---|---|---|---|
| Regimen | IMP | Dose | and Prandial State |
| A | 25HC3S choline | 12.5 mg | Oral, Fasted |
| | salt PiB | | |

A total of 240 mL of water was given immediately following administration.

### Test Product, Dose and Mode of Administration:

In Part 1 and Part 2 of the study, subjects received IMPs according to Table 41.4.

TABLE 41.4
Summary of 25HC3S administered to subjects
in Part 1 and Part 2 of the study

| | | | | Route of |
|---|---|---|---|---|
| | | | | Admin- |
| | | | | istration |
| | | | | and |
| Part | Regimen | IMP | Dose | Prandial State |
| 1 | B | Micronized 25HC3S | 12.5 mg | Oral, Fasted |
| | | choline tablet M4A | (1 × 12.5 mg) | |
| | C | Micronized 25HC3S | | |
| | | choline tablet M7 | | |
| | D | 25HC3S choline SDD | | |
| | | tablet S5 | | |
| | E | 25HC3S sodium salt | 12.5 mg | |
| | | PiB | | |
| 2 | F | Micronized 25HC3S | 50 mg | Oral, Fed |
| | | choline tablet M4A | (4 × 12.5 mg) | |
| | G | Micronized 25HC3S | 50 mg | Oral, Fasted |
| | | choline tablet M4A | (4 × 12.5 mg) | |

A total of 240 mL of water was given immediately following administration.

### Duration of Treatment:

Single oral doses of the 25HC3S compositions were administered on 7 separate occasions over 2 residences, with each dose administered approximately 48 h after dose administration in the previous period and a minimum of 14 days between Part 1 completion and Part 2. Subjects had a follow-up phone call 7 to 9 days post-final dose.

### Criteria for Evaluation

### Pharmacokinetics

The PK parameters in Table 41.5 for plasma concentrations of 25HC3S were estimated where possible and appropriate for each subject and regimen by non-compartmental analysis methods using Phoenix® WinNonlin® software (v8.3, Certara USA, Inc., USA):

TABLE 41.5
Pharmacokinetic parameters

| Parameter | Definition |
|---|---|
| Tlag | Time prior to the first measurable concentration |
| Tmax | Time of maximum observed concentration |
| Tlast | Time of the last measurable concentration |
| Cmax | Maximum observed concentration |
| Clast | Last measurable concentration |
| AUC₍₀₋₈₎ | Area under the curve from time 0 to 8 h post-dose |
| AUC₍₀₋ₗₐₛₜ₎ | Area under the curve from time 0 to the time of |
| | last measurable concentration |
| AUC_{(0-inf)} | Area under the curve from time 0 extrapolated to |
| | infinity |
| AUCₑₓₜᵣₐₚ | Area under the curve from time of the last |
| | measurable concentration to infinity as a |
| | percentage of the area under the curve |
| | extrapolated to infinity |
| T½ | Terminal elimination half-life |
| Lambd_{a-z} | First order rate constant associated with the |
| | terminal (log-linear) portion of the curve |
| CL/F | Total body clearance calculated after a single |
| | extravascular administration where F |
| | (fraction of dose bioavailable) is unknown |
| Vz/F | Apparent volume of distribution based on the |
| | terminal phase calculated using AUC_{(0-inf)} |
| | after a single extravascular administration |
| | where F (fraction of dose bioavailable) |
| | is unknown |
| Frel Cmax | Relative bioavailability based on Cmax |
| Frel AUC₍₀₋₈₎ | Relative bioavailability based on AUC₍₀₋₈₎ |
| Frel AUC₍₀₋ₗₐₛₜ₎ | Relative bioavailability based on AUC₍₀₋ₗₐₛₜ₎ |
| Frel AUC_{(0-inf)} | Relative bioavailability based on AUC_{(0-inf)} |

### Safety

The evaluation of safety parameters comprised analysis of adverse events, laboratory variables (hematology, clinical chemistry and urinalysis), vital signs, electrocardiograms (ECGs) and physical examination findings.

### Statistical Methods

Formal statistical analyses were performed on the PK parameters Cmax, AUC₍₀₋₈₎ and AUC₍₀₋ₗₐₛₜ₎; however, AUC_{(0-inf)} was not analyzed as planned. The PK parameters underwent a natural logarithmic transformation and were analyzed using mixed effect modelling techniques. A single model was used for all treatment comparisons. The model included terms for treatment fitted as a fixed effect and subject fitted as a random effect. The null hypothesis is that there is no difference between test and reference.

The following pairwise treatment comparisons (test vs reference) were included:
Regimen B vs Regimen A
Regimen C vs Regimen A
Regimen D vs Regimen A
Regimen C vs Regimen B
Regimen E vs Regimen A
Regimen G (50 mg fasted) vs Regimen B (12.5 mg fasted)
Regimen F (50 mg fed) vs Regimen G (50 mg fasted)

### Populations Analyzed

The following number of subjects were included in the safety and PK analysis sets for each treatment.

### Part 1

Period 1, 25HC3S choline salt PiB (reference): n=16 (100.0%)
Period 2, Micronized 25HC3S choline tablet M4A: n=16 (100.0%)
Period 3, Micronized 25HC3S choline tablet M7: n=15 (93.8%)
Period 4, 25HC3S choline SDD tablet S5: n=15 (93.8%)
Period 5, 25HC3S sodium salt PiB: n=15 (93.8%) Part 2
Period 6: Micronized 25HC3S choline tablet M4A, fed: n=13 (81.3%)
Period 7: Micronized 25HC3S choline tablet M4A, fasted: n=13 (81.3%)

### Results

### Pharmacokinetics

Key geometric mean (geometric coefficient of variation [CV %]) plasma PK parameters of 25HC3S following oral administration of single 12.5 mg/50 mg doses of 25HC3S formulations in the fed/fasted states are summarized in Tables 41.6 and 41.7.

TABLE 41
6-Pharmacokinetic parameters following oral administration of 25HC3S

| | | Micronized | | 25HC3S | | Micronized | Micronized |
|---|---|---|---|---|---|---|---|
| | | 25HC3S | Micronized | choline | | 25HC3S | 25HC3S |
| | 25HC3S | choline | 25HC3S | SDD | 25HC3S | choline | choline |
| | choline | Tablet | choline | Tablet | sodium | Tablet M4 | Tablet M4 |
| | PIB | M4A | Tablet M7 | S5 | PiB | A | A |
| | (N = 16) | (N = 16) | (N = 15) | (N = 15) | (N = 15) | (N = 13) | (N = 13) |
| Dose | 12.5 mg (1 × 12.5 mg) | | | | | 50 mg (4 × 12.5 mg) | |
| Prandial State | Fasted | | | | | Fed | Fasted |
| Tlagª (h) | 0.500 | 0.500 | 0.500 | 0.000 | 0.500 | 0.500 | 0.000 |
| | (0.00-0.50) | (0.00-3.00) | (0.00-3.00) | (0.00-0.50) | (0.00-0.50) | (0.50-2.00) | (0.00-1.00) |
| Tmaxª (h) | 3.250 | 3.000 | 4.000 | 3.500 | 3.000 | 4.000 | 3.500 |
| | (1.50-6.00) | (2.00-6.00) | (1.50-6.00) | (1.50-6.00) | (2.00-4.00) | (1.50-8.00) | (2.50-6.00) |
| Tlastª (h) | 8.000 | 8.000 | 8.000 | 8.000 | 6.000 | 12.000 | 10.000 |
| | (6.00-10.00) | (6.00-10.00) | (6.00-10.00) | (6.00-10.00) | (6.00-8.00) | (8.00-16.00) | (8.00-16.00) |
| Cmax | 23.2 | 31.7 (33.1) | 26.8 (37.3) | 25.7 | 22.8 | 52.3 (55.3) | 116 (31.7) |
| (ng/mL) | (40.4) | | | (37.5) | (45.1) | | |
| Clast | 3.17 | 3.45 (59.0) | 3.58 (56.1) | 3.77 | 4.80 | 4.77 (48.3) | 2.73 (37.3) |
| (ng/mL) | (43.0) | | | (73.3) | (77.5) | | |
| AUC₍₀₋₈₎ | 99.2 | 114 (38.7) | 103 (36.9) | 114 | 94.2 | 227 (43.2) | 440 (23.3) |
| (ng.h/mL) | (41.4) | | | (35.6) | (38.6) | | |
| AUC | 96.8 | 112 (40.5) | 100 (42.2) | 112 | 87.4 | 279 (33.9) | 461 (26.0) |
| (0-last) | (46.0) | | | (39.9) | (45.1) | | |
| (ng.h/mL) | | | | | | | |
| AUC | 123 | 136 (19.7) | 149 (34.6) | 135 | 105 | 309 (42.4) | 493 (21.0) |
| (0-inf) | (31.3) | [n = 9] | [n = 5] | (20.5) | (28.7) | [n = 8] | [n = 11] |
| (ng.h/mL) | [n = 6] | | | [n = 6] | [n = 6] | | |
| AUCextra | 4.319 | 3.382 (63.5) | 3.287 (92.1) | 4.055 | 5.413 | 3.552 (97.3) | 0.971 (48.8) |
| p (%) | (61.0) | [n = 9] | [n = 5] | (82.7) | (88.9) | [n = 8] | [n =11] |
| | [n = 6] | | | [n = 6] | [n = 6] | | |
| T1/2 (h) | 1.427 | 1.246 (26.7) | 1.167 (15.0) | 1.104 | 1.586 | 1.855 (48.6) | 1.272 (43.5) |
| | (33.5) | [n = 10] | [n = 5] | (6.6) | (48.3) | [n = 8] | [n = 11] |
| | [n = 7] | | | [n = 6] | [n = 8] | | |
| Lambda-z | 0.48575 | 0.55649 | 0.59387 | 0.62775 | 0.43715 | 0.37367 | 0.54475 |
| (1/h) | (33.5) | (26.7) | (15.0) | (6.6) | (48.3) | (48.6) | (43.5) |
| | [n = 7] | [n = 10] | [n = 5] | [n = 6] | [n = 8] | [n = 8] | [n = 11] |
| CL/F | 1690 | 1530 (19.7) | 1400 (34.6) | 1540 | 1990 | 2700 (42.4) | 1690 (21.0) |
| (mL/min) | (31.3) | [n = 9] | [n = 5] | (20.5) | (28.7) | [n = 8] | [n =11] |
| | [n = 6] | | | [n = 6] | [n = 6] | | |
| Vz/F (L) | 189 | 153 (28.8) | 142 (46.5) | 147 | 219 | 433 (58.4) | 186 (32.9) |
| | (42.3) | [n = 9] | [n = 5] | (25.6) | (51.2) | [n = 8] | [n = 11] |
| | [n = 6] | | | [n = 6] | [n = 6] | | |

ªMedian (range)

TABLE 41
7-Pharmacokinetic parameters following oral administration of 25HC3S

| | | Micronized | | 25HC3S | | Micronized | Micronized |
|---|---|---|---|---|---|---|---|
| | | 25HC3S | Micronized | choline | | 25HC3S | 25HC3S |
| | 25HC3S | choline | 25HC3S | SDD | 25HC3S | choline | choline |
| | choline | Tablet | choline | Tablet | sodium | Tablet M4 | Tablet M4 |
| | PIB | M4A | Tablet M7 | S5 | PiB | A | A |
| | (N = 16) | (N = 16) | (N = 15) | (N = 15) | (N = 15) | (N = 13) | (N = 13) |
| Dose | 12.5 mg (1 × 12.5 mg) | | | | | 50 mg (4 × 12.5 mg) | |
| Prandial State | Fasted | | | | | Fed | Fasted |
| Frel Cmax | NA | 136.858 | 114.442 | 110.037 | 97.635 | NA | NA |
| vs CS PiB | | (33.4) | (39.3) | (42.8) | (31.9) | | |
| (%) | | | | | | | |
| Frel | NA | 115.428 | 103.513 | 114.187 | 94.689 | NA | NA |
| AUC(0-8) | | (33.3) | (30.4) | (46.5) | (29.1) | | |
| vs CS PiB | | | | | | | |
| (%) | | | | | | | |
| Frel | NA | 115.552 | 103.388 | 115.046 | 90.066 | NA | NA |
| AUC(0-last) | | (34.9) | (29.2) | (48.1) | (33.7) | | |
| vs CS | | | | | | | |
| PiB (%) | | | | | | | |
| Frel | NA | 104.517 | 126.961 | 101.177 | 93.423 | NA | NA |
| AUC(0-inf) | | (15.3) | (30.3) | (23.5) | (26.2) | | |
| vs CS | | [n = 5] | [n = 4] | [n = 3] | [n = 3] | | |
| PiB (%) | | | | | | | |
| Frel Cmax | NA | NA | 82.855 | NA | NA | NA | 355.256 |
| vs | | | (36.6) | | | | (30.4) |
| 12.5 mg | | | | | | | |
| M4A (%) | | | | | | | |
| Frel | NA | NA | 88.957 | NA | NA | NA | 364.026 |
| AUC(0-8) | | | (35.1) | | | | (18.5) |
| vs 12.5 mg | | | | | | | |
| M4A (%) | | | | | | | |
| Frel | NA | NA | 88.747 | NA | NA | NA | 392.007 |
| AUC(0-last) | | | | | | | |
| vs 12.5 | | | (36.8) | | | | (22.8) |
| mg M4A | | | | | | | |
| (%) | | | | | | | |
| Frel | NA | NA | 101.090 | NA | NA | NA | 341.134 |
| AUC(0-inf) | | | (20.2) | | | | (10.9) |
| vs 12.5 | | | [n = 4] | | | | [n = 7] |
| mg M4A | | | | | | | |
| (%) | | | | | | | |
| Frel Cmax | NA | NA | NA | NA | NA | 45.090 | NA |
| VS M4A | | | | | | (57.0) | |
| Fasted (%) | | | | | | | |
| Frel | NA | NA | NA | NA | NA | 51.509 | NA |
| AUC(0-8) | | | | | | (45.0) | |
| vs M4A | | | | | | | |
| fasted (%) | | | | | | | |
| Frel | NA | NA | NA | NA | NA | 60.568 | NA |
| AUC(0-last) | | | | | | | |
| vs | | | | | | (28.1) | |
| M4A, | | | | | | | |
| Fasted (%) | | | | | | | |
| Frel | NA | NA | NA | NA | NA | 68.986 | NA |
| AUC(0-inf) | | | | | | (32.5) | |
| vs M4A, | | | | | | [n = 6] | |
| Fasted (%) | | | | | | | |

Following administration of 25HC3S choline salt PiB (reference), median Tmax for 25HC3S occurred at 3.25 h post-dose. Geometric mean T½ of 25HC3S was 1.4 h. The 25HC3S choline salt was dosed as tablet formulations micronized 25HC3S choline tablet M4A and M7, and 25HC3S choline spray-dried dispersion (SDD) tablet S5. Following administration of the 3 choline salt tablets, median Tmax was similar compared to the reference, occurring between 3.00 h and 4.00 h post-dose. The change to administration with tablet formulations resulted in an increase in Cmax of between approximately 10% and 37%, with the 37% increase following administration of the micronized 25HC3S choline tablet M4A deemed statistically significant. Smaller increases in AUC₍₀₋₈₎ and AUC₍₀₋ₗₐₛₜ₎ of between 3% and 16% were observed, for which statistical significance could not be concluded. Peak and overall exposure to 25HC3S, as measured by Cmax, AUC₍₀₋₈₎ and AUC₍₀₋ₗₐₛₜ₎, were reduced by between approximately 10% and 16% following administration of the 12.5 mg micronized 25HC3S choline tablet M7 when compared to the 12.5 mg micronized 25HC3S choline tablet M4A, for which statistical significance could not be concluded. Furthermore, the plasma half-lives of 25HC3S appeared unchanged compared to the reference formulation, with geometric mean T½ estimates of between 1.1 h and 1.2 h.

For the 12.5 mg 25HC3S sodium salt PiB, similar to all previous administrations, median Tmax occurred at 3.00 h post-dose. Peak and overall exposure to the 12.5 mg 25HC3S sodium salt PiB, as measured by the Cmax, AUC₍₀₋₈₎ and AUC₍₀₋ₗₐₛₜ₎ were similar, if not slightly reduced when compared with the 12.5 mg choline salt PiB, with statistical significance not concluded. The geometric mean T½ observed following 25HC3S sodium salt PiB was similar to that of 25HC3S choline salt PiB at approximately 1.6 h and 1.4 h, respectively.

Following administration of 50 mg micronized 25HC3S choline tablet M4A with a high-fat breakfast, median Tmax occurred at 4.00 h post-dose, compared to a median Tmax of 3.50 h at the same dose in the fasted state. Geometric mean Cmax, AUC₍₀₋₈₎ and AUC₍₀₋ₗₐₛₜ₎ showed a statistically significant decrease of approximately 55%, 48% and 39%, respectively, in the fed state compared with the fasted state. Elimination of 25HC3S was slightly slower in the fed state, with geometric mean terminal T½ of 1.9 h, compared with 1.3 h for the same dose level in the fasted state. Increasing the dose of the micronized 25HC3S choline tablet M4A formulation in the fasted state to 50 mg (a potential therapeutic dose level), resulted in peak and overall exposure based on Cmax, AUC₍₀₋₈₎ and AUC₍₀₋ₗₐₛₜ₎, to increase in an approximately dose proportional manner of between approximately 263% and 307% compared with the same formulation at 12.5 mg.

### Assessment of Relative Bioavailability

In comparison with the 25HC3S choline salt PiB, peak and overall exposure levels of 25HC3S saw the largest increase following administration of micronized 25HC3S choline tablet M4A, where exposure was increased by approximately 37%, 15% and 16%, as measured by Cmax, AUC(0-8) and AUC(0-last), respectively.

Smaller increases compared with 25HC3S choline salt PiB were also observed following dosing with micronized 25HC3S choline tablet M7 (average increase in exposure between 3% and 15%) and 25HC3S choline SDD tablet S5 (average increase in exposure between 10% and 15%) as measured by Cmax, AUC(0-8) and AUC(0-last).

With the exception of Cmax for the micronized 25HC3S choline tablet M4A, the 90% CIs for the GMRs for each comparison (micronized 25HC3S choline tablet M4A, micronized 25HC3S choline tablet M7 and 25HC3S choline SDD tablet S5) vs 25HC3S choline salt PiB included unity (i.e., 100%), indicating the differences were not statistically significant.

Peak and overall 25HC3S exposure, as measured by Cmax, AUC(0-8), and AUC(0-last) was similar for 25HC3S sodium salt PiB and 25HC3S choline salt PiB, i.e., ratios of approximately 98%, 95% and 90%, respectively. In each instance, the 90% confidence interval (CI) of the ratios contained the value 100%.

Peak and overall exposure to 25HC3S, as measured by Cmax, AUC(0-8) and AUC(0-last), were reduced for the 12.5 mg micronized 25HC3S choline tablet M7 when compared with the 12.5 mg micronized 25HC3S choline tablet M4A, i.e., ratios of between 10% and 16%. In each instance, the 90% CI of the ratios contained the value 100%.

Following a 300% increase in dose (4-fold increase), exposure of 25HC3S was increased by approximately 263%, 280% and 307% for 50 mg micronized 25HC3S choline tablet M4A, as measured by Cmax, AUC(0-8) and AUC(0-last), respectively (i.e., a 3.6-, 3.8- and 4.1-fold increase in exposure). The 90% Cis were (299.65%, 440.00%), (322.09%, 447.34%) and (346.40% 478.19%), respectively. As the increase in exposures were close to proportional to the increase in dose, the results indicate that peak and overall exposure for 25HC3S increased in a dose proportional manner over the 12.5 mg to 50 mg dose range of micronized 25HC3S choline tablet M4A.

Peak exposure levels of 25HC3S, as measured by Cmax, for 50 mg micronized 25HC3S choline tablet M4A administered in the fed state were approximately 45% of those of 50 mg micronized 25HC3S choline tablet M4A administered in the fasted state (i.e., approximately 55% lower), with the 90% CI of the ratio lying entirely below 100%. Overall exposure, as measured by AUC(0-8) and AUC(0-last) for 50 mg micronized 25HC3S choline tablet M4A administered in the fed state were approximately 52% and 61% of those for 50 mg micronized 25HC3S choline tablet M4A administered in the fasted state (i.e., approximately 48% and 39% lower, respectively), with the 90% Cis lying entirely below 100%. The difference between treatments in peak and overall exposure was statistically significant for all PK parameters (p<0.001).

### Safety

25HC3S was well tolerated when administered as choline salt IR formulations dosed up to 50 mg in the fed and fasted states and, sodium salt PiB and choline salt PiB formulations dosed at 12.5 mg in the fasted state. No serious, severe treatment-emergent adverse events (TEAEs) or deaths were reported. Overall, 2 (12.5%) subjects reported a total of 4 TEAEs; all TEAEs were mild in severity and were not related to IMP.

One TEAE of Coronavirus Disease 2019 (COVID-19) infection led to IMP withdrawal and was considered not related to 25HC3S.

There were no clinically significant changes in clinical laboratory, urinalysis, vital signs or ECG results.

### Discussion

Following administration of 25HC3S as a reference formulation of 25HC3S choline salt PiB, 25HC3S was absorbed with median Tmax occurring at 3.25 h post dose. Concentrations following Cmax exhibited a rapid decline, with a geometric mean T½ of approximately 1.4 h. The inter-subject variability (CV %) associated with exposure (Cmax and AUC) was moderate, at 31.3% to 46.0%.

The 25HC3S choline salt was dosed as tablet formulations micronized 25HC3S choline tablet M4A and M7, and 25HC3S choline SDD tablet S5. Following administration of the 3 choline salt tablets, the peak exposure times were similar compared with the 25HC3S choline salt PiB reference, with median Tmax of between 3.00 h and 4.00 h for all formulations. The change to administration of tablet formulations resulted in an increase in Cmax of between 10% and 37%, of which the 37% increase following micronized 25HC3S choline tablet M4A was statistically significant. Smaller increases in AUC(0-8) and AUC(0-last) of between 3% and 16% were observed, for which statistical significance could not be concluded. Peak and overall exposure to 25HC3S, as measured by Cmax, AUC(0-8) and AUC(0-last), were reduced by between 10% and 16% following administration of the 12.5 mg micronized 25HC3S choline tablet M7 when compared with the 12.5 mg micronized 25HC3S choline tablet M4A, for which statistical significance could not be concluded. The inter subject variability associated with exposure for all tablet formulations dosed at 12.5 mg was unchanged, at 19.7% to 42.2%, compared with the 25HC3S choline salt PiB reference formulation (31.1% to 46.0%). The plasma half-lives of 25HC3S also appeared unchanged compared with the reference formulation, with geometric mean T½ of between 1.1 h and 1.2 h, compared with 1.4 h.

For the 12.5 mg 25HC3S sodium salt PiB, similar to all previous administrations, median Tmax was maintained at 3.00 h post-dose. Peak and overall exposure to the 12.5 mg 25HC3S sodium salt PiB formulation, as measured by the Cmax, AUC(0-8) and AUC(0-last), were similar, if not slightly reduced when compared to the 12.5 mg 25HC3S choline salt PiB formulation, with statistical significance not being concluded. The plasma half-life observed following the 25HC3S sodium salt PiB was similar to that of the 25HC3S choline salt PiB, at approximately 1.6 h and 1.4 h, respectively.

Increasing the dose of the micronized 25HC3S choline tablet M4A formulation in the fasted state to 50 mg (a potential therapeutic dose level), resulted in peak and overall exposure based on Cmax, AUC(0-8) and AUC(0-last) to increase in an approximately dose proportional manner of between 263% and 307%, compared with the same formulation at 12.5 mg. The inter subject variability associated with exposure following 50 mg micronized 25HC3S choline tablet M4A (21.0% to 31.7%) was reduced compared with the 12.5 mg micronized 25HC3S choline tablet M4A (19.7% to 40.5%), following administration in the fasted state.

Administration of 50 mg micronized 25HC3S choline tablet M4A with a high fat breakfast resulted in a median Tmax of 4.00 h, compared with a median Tmax of 3.50 h at the same dose in the fasted state. Geometric mean Cmax, AUC(0-8) and AUC(0-last) showed a statistically significant decrease of approximately 55%, 48% and 39%, respectively, in the fed state compared with the fasted state. The inter-subject variability associated with exposure in the fed state (33.9% to 55.3%) was slightly increased compared with the fasted state (21.0% to 31.7%). Elimination of 25HC3S was slightly slower in the fed state, with geometric mean terminal T½ of approximately 1.9 h, compared with 1.3 h for the same dose level in the fasted state.

25HC3S was well tolerated when administered as choline salt IR formulations dosed up to 50 mg in the fed and fasted states, and sodium salt PiB and choline salt PiB formulations dosed at 12.5 mg in the fasted state. No serious, severe TEAEs or deaths were reported. The incidence of TEAEs was low, all TEAEs were mild in severity and there were no IMP-related TEAEs.

### Conclusions

The 25HC3S sodium salt PiB formulation was assessed in Part 1 of this study alongside the reference 25HC3S choline salt PiB to bridge back to previous clinical data. The comparative exposure was found to be similar for the two salt forms. Micronized 25HC3S choline tablet M4A was selected as a lead formulation in the study because of the comparatively higher PK exposure and relatively simpler manufacturing process compared with the 25HC3S choline SDD tablet S5. Following a 4-fold increase in dose, the increase in exposure (Cmax and AUC) of 25HC3S was between 3.6-fold and 4.1-fold; thus, the exposure increased in approximately proportional manner over the 12.5 mg (1×12.5 mg) to 50 mg (4×12.5 mg) dose range for the micronized 25HC3S choline tablet M4A. A negative food effect was observed when the selected micronized 25HC3S choline tablet M4A test formulation 50 mg (4×12.5 mg) was dosed with a high-fat breakfast. The IMP's administered in the study were considered to be well tolerated for all the formulations and dose levels under the conditions of the study.

### Pharmacokinetic Conclusions

### Part 1

Following single oral administration of micronized 25HC3S choline tablet M4A and M7 and 25HC3S choline SDD tablet S5, absorption of 25HC3S was similar (median Tmax 3.00 h to 4.00 h) to administration of 25HC3S choline salt PiB reference formulation (median Tmax 3.25 h), in the fasted state.

Peak (Cmax) and overall (AUC(0-8) and AUC(0-last)) 25HC3S exposure were increased following administration of the tablet formulations (micronized 25HC3S choline tablet M4A and M7 and the 25HC3S choline SDD tablet S5) compared with the 25HC3S choline salt PiB reference formulation, with only the increase in peak exposure following micronized 25HC3S choline tablet M4A deemed statistically significant.

Peak (Cmax) and overall (AUC(0-8) and AUC(0-last)) 25HC3S exposure were reduced by between approximately 10% and 16% following administration of the 12.5 mg micronized 25HC3S choline tablet M7 compared with the 12.5 mg micronized 25HC3S choline tablet M4A, with the reduction not deemed statistically significant for any parameter.

The inter-subject variability (CV %) associated with exposure (Cmax and AUC) was unchanged at 19.7% to 42.2% following administration of 12.5 mg 25HC3S choline salt tablet formulations compared with the 25HC3S choline salt PiB reference (31.3% to 46.0%).

Peak (Cmax) and overall exposure (AUC(0-8) and AUC(0-last)) exposure to the 12.5 mg 25HC3S sodium salt PiB were similar, if not slightly reduced when compared with the 12.5 mg 25HC3S choline salt PiB, with statistical significance not being concluded.

Elimination of 25HC3S following administration of 12.5 mg micronized 25HC3S choline tablet M4A and M7, 25HC3S sodium salt PiB and 25HC3S choline SDD tablet S5 appeared similar across all formulations in the fasted state, with geometric mean T½ estimates of between approximately 1.1 h and 1.6 h.

### Part 2

Increasing the dose of micronized 25HC3S choline tablet M4A to the potential therapeutic dose level of 50 mg (i.e., a 4-fold increase in dose) in the fasted state, resulted in an approximately proportional increase of between 3.6-fold and 4.1-fold in peak (Cmax) and overall (AUC(0-8) and AUC(0-last)) exposure to 25HC3S.

In the fasted state, the inter-subject variability (CV %) associated with exposure (Cmax and AUC) following administration of 50 mg of micronized 25HC3S choline tablet M4A (21.0% to 31.7%) was slightly reduced compared with the 12.5 mg micronized 25HC3S choline tablet M4A dose (19.7% to 40.5%).

Administration of 50 mg micronized 25HC3S choline tablet M4A with a high-fat breakfast resulted in a median Tmax of 4.00 h compared with 3.50 h for the same dose in the fasted state.

Exposure to 25HC3S following administration of 50 mg micronized 25HC3S choline tablet M4A in the fed state showed a statistically significant decrease of approximately 55%, 48% and 39% based on Cmax, AUC(0-8) and AUC(0-last), respectively, compared with the fasted state.

The inter-subject variability (CV %) associated with exposure (Cmax and AUC) in the fed state (33.9% to 55.3%) was slightly increased compared with the fasted state (21.0% to 31.7%).

Elimination of 25HC3S was slightly slower in the fed state, with geometric mean terminal T½ of approximately 1.9 h, compared with 1.3 h in the fasted state.

### Example 42. A Mesophase of 25HC3S Choline

A mesophase of 25HC3S choline was prepared in the following ways: (i) Dissolving 25HC3S choline in ethanol followed by rapid evaporation; or (ii) treating with 25HC3S choline with water followed by a fast, partial evaporation; or (iii) treating with a 50:50 mixture of water and methanol followed by filtration and drying, each yielded a mesophase of 25HC3S choline. X-ray powder diffraction patterns of (i) to (iii) are found in FIG. 38 with ethanol at the top and (iii) in the middle and (ii) at the bottom.

### Example 43. 25HC3S Choline Tablet Development

### Overview

Roller Compaction and Compression of the tableting process were evaluated. A total of ten roller compaction trials (Runs A to J) and five compression trials (Runs 1 to 5) were performed using the formulation of tablet M4A described in Example 38. Process parameters were investigated.

Roller Compaction (RC) process parameters were investigated by looking into the particle size distribution (PSD) data of all ten batches. Pre-blend PSD had too many fines. A more uniform distribution of granules and better flow properties are preferred.

Compressibility depends on the granules, the bigger the granules, the lower the Carr's index, which indicated better flow properties and better compressibility. Final blend should flow smooth into the hopper and fill dies accurately resulting in less weight variation.

### Roller Compaction

The formulation was subjected to roller compaction using a Gerteis Mini-Pactor® roller compactor at a roller speed of 2 rpm, a roller gap of 1 mm, except for Run J which involved a roll gap of 2 mm, a screen size of ranging from 0.8 mm to 1.25 mm, and a compaction force ranging from 5 kN/cm to 9 kN/cm. The results are shown below.

| | | Run# | | | | | | | | | |
|---|---|---|---|---|---|---|---|---|---|---|---|
| | | Run B | Run A | Run C | Run D | Run E | Run F | Run G | Run H | Run I | Run J |
| | Compaction | 7 kN | 5 kN | 7kN | 5KN | 7 kN | 9 kN | 5 kN | 7 kN | 9 kN | 9 kN |
| | Force | | | | | | | | | | |
| | Screen Size | 1.0 mm | 1.0 mm | 1.0 mm | 0.8 mm | 0.8 mm | 0.8 mm | 1.25 mm | 1.25 mm | 1.25 mm | 1.0 mm |
| | Bulk | 0.5 | 0.48 | 0.49 | 0.5 | 0.51 | 0.54 | 0.51 | 0.55 | 0.52 | 0.5 |
| | Density | | | | | | | | | | |
| | Tap Density | 0.577 | 0.55 | 0.55 | 0.6 | 0.58 | 0.61 | 0.54 | 0.61 | 0.57 | 0.57 |
| | Carrs Index | 13.3 | 12.7 | 10.9 | 16.7 | 12.6 | 11.5 | 5.5 | 9.8 | 8.8 | 12.3 |
| | Screen | | | | | | | | | | |
| μm | Number | | | | | | | | | | |
| 850 | 20 | 12.4 | 10.8 | 6.9 | 0.6 | 0.9 | 0 | 31 | 28.7 | 31.6 | 22.5 |
| 425 | 40 | 37.6 | 35.6 | 33.8 | 32.2 | 38.7 | 24.1 | 30 | 26.7 | 30.1 | 68.7 |
| 250 | 60 | 13.2 | 13.8 | 18.2 | 18.3 | 17.4 | 25.4 | 10.6 | 11.7 | 10.5 | 5.2 |
| 125 | 120 | 11 | 12.4 | 14.5 | 15.7 | 13.7 | 19.2 | 8.5 | 10.3 | 8.4 | 1 |
| 75 | 200 | 7.3 | 8.4 | 7.8 | 10.4 | 8.7 | 9 | 5.9 | 6.7 | 5.8 | 0.5 |
| 45 | 325 | 7.1 | 7 | 7.4 | 9 | 6.9 | 8.8 | 5.2 | 6.2 | 4.8 | 0.7 |
| | pan | 11 | 10.6 | 11 | 13.7 | 13.6 | 13.1 | 8.3 | 9.1 | 7.5 | 1 |

Additional force of 9 kN/cm vs 7 kN/cm did not show any significant difference, and the properties of milled granules did not change significantly. The above results suggested a compaction force of 7 kN/cm and screen size of 1.0 mm.

A good positive correlation was found between screen size and % retain of large particles (>425 μm). Larger screen size resulted in higher % retain of large particles.

A reasonable negative correlation was found between screen size and Carrs index. Larger screen size resulted in lower Carrs index. Large particles were associated with lower Carrs index which led to better flowability.

### Compression

Formulation was compacted as described above with a roller speed of 2 rpm, a roller gap of 1 mm, a screen size of 1 mm, and a compaction force of 7 kN/cm. The compacted formulation was subjected to compression using a Korsch XL-100 tablet press to form tablets under the following conditions:

| | Compression Force (KP) | Compression Speed (rpm) | Lot |
|---|---|---|---|
| Run 1 | 7-8 | 50 | A |
| Run 2 | 4 | 50 | B |
| Run 3 | 7-8 | 20 | C |
| Run 4 | 4 | 20 | D |
| Run 5 | 7 | 35 | E |

About 200 tablets were collected from each run.

### Tablet Characterization

The tablets from each lot had an average weight ranging from about 100 mg to about 102 mg. Tablet weights were comparable among the lots.

The tablets had an average thickness ranging from about 2.8 mm to about 3.4 mm. Tablet thicknesses were comparable among the lots.

The tablets had the following hardness:

| | Compression Force | Compression Speed | Mean Hardness |
|---|---|---|---|
| Lot | (KP) | (rpm) | (KP) |
| A | 7-8 | 50 | 7.24 |
| B | 4 | 50 | 3.91 |
| C | 7-8 | 20 | 7.67 |
| D | 4 | 20 | 4.77 |
| E | 7 | 35 | 6.92 |

Disintegration time was measured using a Sotax DT-50 Disintegration Tester (v1.0) set at 30 strokes per minute with a 55 mm stroke height setting. Purified water was chosen as the media, maintained at 37±2° C. The tablets had the following disintegration time:

| | Compression Force | Compression Speed | Disintegration (min) |
|---|---|---|---|
| Lotx | (KP) | (rpm) | Average (RSD) |
| A | 7-8 | 50 | 12.7 (3.9) |
| B | 4 | 50 | 5.6 (7.1) |
| C | 7-8 | 20 | 10.2 (18.6) |
| D | 4 | 20 | 6.2 (9.7) |
| E | 7 | 35 | 11.7(4.3) |

Dissolution experiments were performed using a USP Apparatus 2. The dissolution parameters were as follows: Dissolution medium (900 mL) 2.0% SDS (sodium dodecyl sulfate) in 0.1 N HCl; Paddle speed: 75 rpm for first 75 minutes, then 250 rpm (75 minutes to 105 minutes); Vessel temperature: 37° C. Sampling time points: 5, 15, 30, 45, 60, 75, and 105 minutes. Sampling volume: 5 mL.

Samples were assayed using HPLC. The percent dissolution is shown below:

| | Compression | Compression | Time (min) | | | | | | |
|---|---|---|---|---|---|---|---|---|---|
| Lot | Force (KP) | Speed (rpm) | 5 | 15 | 30 | 45 | 60 | 75 | 105 |
| A | 7-8 | 50 | 19 | 72 | 90 | 94 | 96 | 96 | 95 |
| B | 4 | 50 | 45 | 83 | 95 | 97 | 98 | 98 | 97 |
| C | 7-8 | 20 | 18 | 73 | 91 | 94 | 96 | 96 | 96 |
| D | 4 | 20 | 39 | 83 | 94 | 95 | 97 | 96 | 96 |
| E | 7 | 35 | 20 | 75 | 94 | 96 | 97 | 98 | 97 |

Dissolution variability was high at 5 minutes; however, good reproducibility was observed after 5 minutes. Lower hardness resulted in faster dissolution. Speed had no impact on dissolution of tablets made at 7-8 KP while having a slight impact on the 5-minute timepoint for tablets made at 4 KP.

### Conclusions

### Speed Effect

Speed had no impact on disintegration and dissolution of tablets made at 7-8 KP compression force. Speed appeared to have a slight impact on 5-minute dissolution of tablets made at a compression force of 4 KP, however, no impact after 15 min. Speed appeared to have a slight impact on disintegration relative standard deviation (RSD): higher speed resulted in lower RSD for both tablets made at compression force 7-8 KP and 4 KP.

### Compression Force Effect

Compression force had significant impact on disintegration time and mean % dissolution, especially for the early time points. Higher compression force resulted in longer disintegration time and slower dissolution. Compression force had no impact on RSD of disintegration and dissolution.

### Example 44. Physical and Chemical Stability of Micronized Choline Tablets

The stability of micronized 25HC3S tablet M4A made in accordance with Example 38 was tested.

The packaging of the tablets for this stability testing was as follows:

Bottle, 30 cc, Wide mouth round white
28 mm SecuRx Child Resistant White Closures Foilseal M1/0.035
Pulp PRTD“SFYP”-WHT
Performance Dry 1 gram Silica Gel Desiccant Canister (0.67 in × 0.55 in)
9 Gram Polyester Coil

The packaged tablets were analyzed for physical and chemical stability at T=0, 1 month, 3 months, and 6 months. The storage conditions were either 25° C./60% RH or 40° C./75% RH. The tablets and dissolution samples were assayed using HPLC at 205 nm.

The tablet dissolution conditions were as follows:

| | |
|---|---|
| Apparatus: | USP Apparatus 2 (Paddles) |
| Temperature: | 37.0° C. ± 0.5° C. |
| Medium: | 2.0% Sodium Lauryl Sulfate in 0.1N |
| | Hydrochloric Acid |
| Medium Volume: | 900 mL |
| Rotation Speed | 75 rpm (250 rpm from 90 minutes to 105 minutes) |
| Sampling | 5, 15, 30, 45, 60, 75, 90, and 105 minutes |
| Timepoints: | |
| Sampling Volume: | 5 mL |

The results were as follows:

TABLE 44.1
Stability Results for Condition 25° C./60% RH

| | Time Point | | | | | | | |
|---|---|---|---|---|---|---|---|---|
| | INITIAL | | 1 M | | 3 M | | 6 M | |
| | Appearance | | | | | | | |
| | Pantone | | Pantone | | Pantone | | Pantone | |
| | P20-6 C, Yellow | | P20-6 C, Yellow | | P20-6 C, Yellow | | P20-6 C, Yellow | |
| | Round Tablets | | Round Tablets | | Round Tablets | | Round Tablets | |
| Assay | % Label Claim | | % Label Claim | | % Label Claim | | % Label Claim | |
| Prep 1 | 100.0% | | 100.6% | | 101.4% | | 100.1 | |
| Prep 2 | 100.6% | | 99.9% | | 100.1% | | 101.2 | |
| Mean | 100.3% | | 100.3% | | 100.8% | | 100.6 | |
| Related Substances | RRT | % | RRT | % | RRT | % | RRT | % |
| 25-sulfate Cholesterol | 0.79 | BQL | 0.83 | 0.06% | 0.78 | BQL | 0.78 | BQL |
| 25-hydroxy Cholesterol | 3.60 | 0.64% | 3.63 | 0.66% | 3.58 | 0.71% | 3.57 | 0.69% |
| Sulfated desmosterol | 2.61 | 1.15% | 2.67 | 1.14% | 2.67 | 1.25% | 2.61 | 1.24% |
| Unknown Impurity 1 | 0.92 | BQL | 0.92 | ND | 0.92 | ND | 0.92 | ND |
| Unknown Impurity 2 | 1.89 | 0.16% | 1.89 | 0.17% | 1.85 | 0.17% | 1.86 | 0.17% |
| Unknown Impurity 3 | 2.19 | ND | 2.19 | ND | 2.19 | ND | 2.19 | ND |
| Unknown Impurity 4 | 4.88 | ND | 4.88 | ND | 4.88 | 0.08% | 4.87 | 0.08% |
| RRT 0.30 | 0.30 | ND | 0.30 | ND | 0.30 | 0.05% | 0.30 | 0.06% |
| RRT 0.86 | 0.86 | 0.12% | 0.86 | 0.09% | 0.86 | 0.06% | 0.86 | 0.05% |
| RRT 0.88 | 0.88 | ND | 0.88 | ND | 0.88 | 0.10% | 0.88 | 0.05% |
| RRT 4.72 | 4.72 | ND | 4.72 | ND | 4.72 | 0.07% | 4.72 | 0.16% |

| | | | | |
|---|---|---|---|---|
| Total | 2.07% | 2.12% | 2.49% | 2.50% |

TABLE 44
2. Stability Results for Condition 25°C/60% RH (Cont.)

| Time Point | INITIAL | | 1 M | | 3 M | | 6 M | |
|---|---|---|---|---|---|---|---|---|
| Dissolution | Mean % | Range % | Mean % | Range % | Mean % | Range % | Mean % | Range % |
| 5.0 Minutes | 15 | 9-20) | 16 | 12-21 | 13 | 10-19 | 21 | 17-24 |
| 15.0 Minutes | 72 | 68-77 | 72 | 67-76 | 76 | 69-81 | 77 | 75-79 |
| 30.0 Minutes | 90 | 87-93 | 91 | 87-93 | 94 | 92-95 | 92 | 90-96 |
| 45.0 Minutes | 93 | 91-95 | 95 | 93-96 | 98 | 96-100 | 96 | 93-98 |
| 60.0 Minutes | 94 | 91-98 | 95 | 93-97 | 98 | 96-100 | 95 | 92-99 |
| 75.0 Minutes | 94 | 91-96 | 96 | 93-99 | 99 | 98-100 | 96 | 92-99 |
| 105.0 Minutes | 93 | 92-95 | 96 | 94-97 | 98 | 97-99 | 95 | 93-98 |

| | | | | |
|---|---|---|---|---|
| Water Content | 3.6% | 2.6% | 3.2% | 3.1% |

TABLE 44
3. Stability Results for Condition 40°C/75% RH

| | Time Point | | | | | | | |
|---|---|---|---|---|---|---|---|---|
| | INITIAL | | 1 M | | 3 M | | 6 M | |
| | Pantone P20-6 C, | | Pantone P20-6 C, | | Pantone P20-6 C, | | Pantone P20-6 C, | |
| Appearance | YellowR ound Tablets | | Yellow Round Tablets | | Yellow Round Tablets | | Yellow Round Tablets | |
| Assay | % Label Claim | | % Label Claim | | % Label Claim | | % Label Claim | |
| Prep 1 | 100.0% | | 100.2% | | 100.0% | | 100.2 | |
| Prep 2 | 100.6% | | 99.7% | | 99.0% | | 98.6 | |
| Mean | 100.3% | | 99.9% | | 99.5% | | 99.4 | |
| Related Substances | RRT | % | RRT | % | RRT | % | RRT | % |
| 25-sulfate Cholesterol | 0.79 | BQL | 0.83 | 0.05% | 0.78 | BQL | 0.78 | BQL |
| 25-hydroxy Cholesterol | 3.60 | 0.64% | 3.63 | 0.67% | 3.58 | 0.67% | 3.57 | 0.75% |
| Sulfated desmosterol | 2.61 | 1.15% | 2.67 | 1.15% | 2.67 | 1.23% | 2.61 | 1.26% |
| Unknown Impurity 1 | 0.92 | BQL | 0.92 | ND | 0.92 | ND | 0.92 | ND |
| Unknown Impurity 2 | 1.89 | 0.16% | 1.89 | 0.17% | 1.85 | 0.17% | 1.86 | 0.17% |
| Unknown Impurity 3 | 2.19 | ND | 2.19 | ND | 2.19 | ND | 2.19 | ND |
| Unknown Impurity 4 | 4.88 | ND | 4.73 | 0.06% | 4.88 | 0.08% | 4.87 | 0.08% |
| RRT 0.30 | 0.30 | ND | 0.30 | ND | 0.30 | 0.06% | 0.30 | 0.07% |
| RRT 0.51 | 0.51 | ND | 0.51 | ND | 0.51 | ND | 0.51 | 0.07% |
| RRT 0.84 | 0.84 | ND | 0.84 | NI | 0.84 | ND | 0.84 | 0.05% |
| RRT 0.86 | 0.86 | 0.12% | 0.86 | 0.09% | 0.86 | 0.07% | 0.86 | 0.05% |
| RRT 0.88 | 0.88 | ND | 0.88 | ND | 0.88 | 0.09% | 0.88 | 0.05% |
| RRT 4.72 | 4.72 | ND | 4.72 | ND | 4.72 | ND | 4.72 | 0.19% |

| | | | | |
|---|---|---|---|---|
| Total | 2.07% | 2.19% | 2.37% | 2.74% |

TABLE 44
4. Stability Results for Condition 40°C/75%RH (Continued)

| | Time Point | | | | | | | |
|---|---|---|---|---|---|---|---|---|
| | INITIAL | | 1 M | | 3 M | | 6 M | |
| Dissolution | Mea n% | Range % | Mean % | Range % | Mean % | Range % | Mean % | Range % |
| 5.0 Minutes | 15 | 9-20 | 18 | 13-24 | 16 | 10-24 | 20 | 18-23 |
| 15.0 Minutes | 72 | 68-77 | 76 | 72-79 | 76 | 74-77 | 78 | 72-83 |
| 30.0 Minutes | 90 | 87-93 | 93 | 89-96 | 94 | 92-95 | 91 | 88-94 |
| 45.0 Minutes | 93 | 91-95 | 95 | 93-97 | 96 | 95-99 | 92 | 90-94 |
| 60.0 Minutes | 94 | 91-98 | 96 | 96-98 | 97 | 95-101 | 93 | 91-94 |
| 75.0 Minutes | 94 | 91-96 | 96 | 94-97 | 97 | 96-99 | 93 | 92-94 |
| 105.0 Minutes | 93 | 92-95 | 97 | 94-100 | 98 | 97-99 | 94 | 92-95 |

| | | | | |
|---|---|---|---|---|
| Water Content | 3.6% | 2.7% | 3.4% | 3.7% |

The results revealed that the tablets are relatively stable.

### Example 45. Dose Uniformity of Micronized Choline Tablets

The dose uniformity of micronized 25HC3S tablet M4A made in accordance with Example 38 was assayed via HPLC at 205 nm.

The results were as follows:

TABLE 45.1
Uniformity of Dosage (Initial) Results

| Preparation # | % Assay |
|---|---|
| CU # 1 | 99.3 |
| CU # 2 | 98.9 |
| CU # 3 | 96.6 |
| CU # 4 | 98.4 |
| CU # 5 | 100.4 |
| CU # 6 | 96.6 |
| CU # 7 | 98.1 |
| CU # 8 | 98.2 |
| CU # 9 | 101.6 |
| CU # 10 | 99.6 |
| Average | 98.8 |
| SD | 1.56 |
| % RSD | 1.6 |
| Min | 96.6 |
| Max | 101.6 |
| AV | 3.7 |

The results revealed that the tablets are relatively uniform.

1. A pharmaceutical composition comprising 25HC3S choline and one or more disintegrants.

2. The pharmaceutical composition of claim 1, further comprising at least one of one or more filler, one or more surfactants, one or more glidants, one or more lubricants, one or more binders, one or more plasticizers, one or more antioxidants, one or more pH adjusting agents, one or more colorants, one or more flavoring agents, and one or more sweeteners.

3. The pharmaceutical composition of claim 1 or 2, wherein the one or more disintegrants comprise one or more of cross-linked starch, sodium starch glycolate, cross-linked cellulose, cross-linked carboxymethylcellulose, the sodium salt of cross-linked carboxymethylcellulose, polyvinylpyrrolidone, cross-linked polyvinylpyrrolidone, chitosan hydrochloride, corn starch, docusate sodium, magnesium aluminum silicate, starch, modified starch, pregelatinized starch, pregelatinized modified starch, hydroxypropyl starch, cellulose, methylcellulose, sodium carboxymethyl cellulose, calcium carboxymethyl cellulose, substituted hydroxypropyl cellulose, microcrystalline cellulose, calcium cellulose glycolate, carmellosum calcium, alginates, alginic acid, silicon dioxide, colloidal silicon dioxide, glycine, guar gum, polacrilin potassium, and sodium bicarbonate.

4. The pharmaceutical composition of any one of claims 1 to 2, wherein the one or more disintegrants comprise one or more of powdered cellulose, low-substituted hydroxypropyl cellulose, sodium alginate, calcium alginate, and calcium sodium alginate.

5. The pharmaceutical composition of any one of claim 1 or 4, wherein the one or more disintegrants comprise polyvinylpyrrolidone.

6. The pharmaceutical composition of claim 5, wherein the polyvinylpyrrolidone comprises cross-linked polyvinylpyrrolidone.

7. The pharmaceutical composition of claim 6, wherein the cross-linked polyvinylpyrrolidone comprises crospovidone.

8. The pharmaceutical composition of any one of claims 1 to 7, wherein the one or more disintegrants comprise sodium bicarbonate.

9. The pharmaceutical composition of any one of claims 1 to 8, wherein the one or more disintegrants are present in a total amount ranging from 0.5 wt % to 25 wt %, based on weight of the pharmaceutical composition.

10. The pharmaceutical composition of any one of claims 1 to 9, wherein the one or more disintegrants are present in a total amount ranging from 0.5 wt % to 15 wt %, based on weight of the pharmaceutical composition.

11. The pharmaceutical composition of any one of claims 1 to 10, wherein the 25HC3S choline is spray dried.

12. The pharmaceutical composition of any one of claims 1 to 11, wherein the 25HC3S choline is micronized.

13. The pharmaceutical composition of any one of claims 1 to 12 in tablet form.

14. Amorphous 25HC3S choline.

15. A tablet comprising a pharmaceutical composition comprising spray-dried 25HC3S choline and one or more pharmaceutically acceptable excipients.

16. Micronized 25HC3S choline.

17. A method of treating or preventing one or more of nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, alcoholic fatty liver disease (AFLD), alcoholic steatohepatitis (ASH), leptin resistance, leptin deficiency, a diabetes condition, an autoimmune condition, an inflammatory condition, a neurological condition, Epstein Barr virus-related growth, and conditions related to fat accumulation and inflammation, comprising administering to a patient in need thereof an effective amount of a pharmaceutical composition of any one of claims 1 to 13; amorphous 25HC3S choline of claim 14; a tablet of claim 15; or micronized 25HC3S choline of claim 16.

18. Use of: a pharmaceutical composition of any one of claims 1 to 13; amorphous 25HC3S choline of claim 14; a tablet of claim 15; or micronized 25HC3S choline of claim 16; in the manufacture of a medicament for use in a method of treating or preventing one or more of nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, alcoholic fatty liver disease (AFLD), alcoholic steatohepatitis (ASH), leptin resistance, leptin deficiency, a diabetes condition, an autoimmune condition, an inflammatory condition, a neurological condition, Epstein Barr virus-related growth, and conditions related to fat accumulation and inflammation.

19. A pharmaceutical composition of any one of claims 1 to 13; amorphous 25HC3S choline of claim 14; a tablet of claim 15; or micronized 25HC3S choline of claim 16; for use as a medicament.

20. A pharmaceutical composition of any one of claims 1 to 13; amorphous 25HC3S choline of claim 14; a tablet of claim 15; or micronized 25HC3S choline of claim 16; for use in a method of treating or preventing one or more of nonalcoholic fatty liver disease (NAFLD), nonalcoholic steatohepatitis (NASH), alcoholic hepatitis, acute kidney injury (AKI), psoriasis, atherosclerosis, hypercholesterolemia, hypertriglyceridemia, alcoholic fatty liver disease (AFLD), alcoholic steatohepatitis (ASH), leptin resistance, leptin deficiency, a diabetes condition, an autoimmune condition, an inflammatory condition, a neurological condition, Epstein Barr virus-related growth, and conditions related to fat accumulation and inflammation.
